(19) United States
(12) Patent Application Publication
(10) Pub. No.: US 2023/0265524 A1
(43) Pub. Date: Aug. 24, 2023

(54) IMMUNOGENIC RETROELEMENTS AND THEIR USE IN CANCER THERAPY

(71) Applicant: UNIVERSITY HEALTH NETWORK, Toronto (CA)

(72) Inventors: Daniel DINIZ DE CARVALHO, Toronto (CA); Parinaz MEHDIPOUR, Toronto (CA); Sajid MARHON, Toronto (CA)

(21) Appl. No.: 18/008,053

(22) PCT Filed: Jun. 3, 2021
(86) PCT No.: PCT/CA2021/050765

§ 371 Date: Dec. 2, 2022

Related U.S. Application Data
(60) Provisional application No. 63/035,081, filed on Jun. 5, 2020.

(51) Int. Cl. C12Q 1/6886; A61K 31/706; A61P 35/00

(52) U.S. Cl. CPC C12Q 1/6886; A61K 31/706; A61P 35/00; C12Q 2600/106; C12Q 2600/158; C12Q 2600/154

(57) ABSTRACT

There is described herein a method of assessing a subject's responsiveness to cancer therapy, comprising: providing a sample from the subject comprising cancers cells or suspected cancer cells; measuring or estimating the expression levels of inverted repeats (IR) Alus in the cells; and determining that the subject would be responsive to 5 cancer therapy if the subject cells exhibit expression levels of inverted repeats (IR) Alus with reference to expression levels in control samples.

### FIELD OF THE INVENTION

The invention relates to immunogenic retroelements and more particularly to their use in cancer therapy.

### BACKGROUND OF THE INVENTION

Aging and disease are generally associated with epigenetic reprogramming and aberrant gene expression. Cancer therapeutics, such as DNA methyltransferase inhibitors (DNMTi), are often designed to restore the expression of inappropriately silenced genes, such as tumour suppressors. Many of these ‘epigenetic therapies’ are currently in clinical use or in pre-clinical development¹⁶. Intriguingly, epigenetic therapies targeting DNMTs, HDACs, LSD1, EZH2, G9a and SETDB1 can mediate their anti-tumour effects by activating retroelements^{1,2,4,6-10}, which represent about 40% of the human genome. Retroelement activation can also contribute to the therapeutic efficacy of other classes of drugs, such as cyclin-dependent kinase inhibitors^{3,5}.

Active retroelements generate double stranded RNA (dsRNA), which in turn stimulates the pattern recognition receptor MDA5^{1,2,4,6-10}. This “viral mimicry” state is characterized by a type I/III interferon response, loss of cancer cell fitness and activation of both innate and adaptive immune responses^{11,12}. However, the identity of these immunogenic retroelements are unknown. We and others have previously shown that inhibition of repressive epigenetic machinery induces the expression of endogenous retroviruses (ERVs), a type of long-terminal repeat (LTR) retroelement^{1,2,4,6-9}. Sense and anti-sense transcription of ERVs has been proposed to generate the immunogenic dsRNA that activate MDA5 after epigenetic therapies⁶, but direct evidence is lacking. Identifying the precise nature of immunogenic retroelements can have several implications, including understanding their physiological roles, developing biomarkers for predicting and monitoring response to epigenetic therapies and to identify vulnerabilities that could act in synergy with epigenetic therapies.

### SUMMARY OF THE INVENTION

In an aspect, there is provided a method of assessing a subject's responsiveness to cancer therapy, comprising: providing a sample from the subject comprising cancers cells or suspected cancer cells; measuring or estimating the expression levels of inverted repeats (IR) Alus in the cells; and determining that the subject would be responsive to cancer therapy if the subject cells exhibit expression levels of inverted repeats (IR) Alus with reference to expression levels in control samples.

In an aspect, there is provided a method of cancer therapy in a subject in need thereof, the method comprising inhibiting ADAR1 in a patient.

In an aspect, there is provided an ADAR1 inhibitor for use in the treatment of cancer in a subject.

In an aspect, there is provided a use of an ADAR1 inhibitor in the preparation of a medicament for the treatment of cancer in a subject.

### BRIEF DESCRIPTION OF FIGURES

These and other features of the preferred embodiments of the invention will become more apparent in the following detailed description in which reference is made to the appended drawings wherein:

FIG. 1. Detecting dsRNA forming repeat element transcription after 5-AZA-CdR treatment.

A, Effect of 5-AZA-CdR on repeat element expression in patient-derived xenograft colorectal cancer cells after 5 days of treatment. Plots show log (fold change) for 5-AZA-CdR treated cells over mock-treated cells for anti-sense (y-axis) and sense (x-axis) transcripts of each SINE, LINE and ERV repeat element (CPM). Triangle shape dots in the upper-right quadrant represent repeats that are significantly upregulated in 5-AZA-CdR treated condition at both the antisense and sense strands as compared to the mock. Square shape dots in the upper-right quadrant represent repeats that have baseline expression (CPM≥1) at the sense strand and are significantly upregulated at the antisense strand upon 5-AZA-CdR treatment. Diamond shape dots in the upper-right quadrant represent the repeats that have baseline expression (CPM≥1) at the antisense strand and are significantly upregulated at the sense strand upon 5-AZA-CdR treatment. The significance was determined as p≤0.05 and |log FC|≥1 at each strand.

FIG. 2. Inverted Repeats Alus are the major source of immunogenic RNAs induced by 5-AZA-CdR treatment.

A-C, Percentage of non-repeats and several families of Repetitive Elements in the total cytoplasmic fraction (‘Total CytoRNA’) and in the MDA5-protected fraction (‘MDA5-protected’) at the mock-treated baseline condition (A) and after 5-AZA-CdR treatment at 300 nM for 5 days, p<2.2e-16 (B). Treatment-induced immunogenic RNAs' refers to transcripts present in 5-AZA-CdR treated condition but not present in the baseline mock-treated condition (i.e. induced by the treatment), p<2.2e-16 (C). Each plot is the average percentage of two replicates. P-values represent the Fisher Exact test for enrichment of SINEs and depletion of non-repeats in immunogenic RNAs (5-AZA-CdR) and Treatment-induced immunogenic RNAs as compared to the MDA5-protected baseline immunogenic RNAs.

D-E, Counts of the expressed Alus in terms of the Log₁₀ Fold change of their MDA5-protected expression over their Total CytoRNA expression. IR-Alus that are MDA5 dsRNA agonists have positive values on the x-axis. The histogram on the left shows the count of the IR-Alus from the Mock-treated cells while the histogram on the right shows the counts from the 5-AZA-CdR treated cells. In Mock-treated cells, most IR-Alus we identified as treatment-induced do not produce dsRNA and therefore are exactly at the 0 position. In 5-AZA-CdR treated cells, the distribution is skewed to the right and shows that after treatment these IR-Alus are able to make dsRNA and activate MDA5. The color code represents Alus making Inverted Repeats pairs (gray) and Non-IR pairs (black) for Baseline immunogenic RNAs (n=187 Non-IR Alus and 1,602 IR-Alus) (D) and for the Treatment-induced immunogenic RNAs (n=992 Non-IR Alus and 5,199 IR-Alus) (E).

F-G, Scatterplots showing the Log₁₀ Fold change of MDA5-protected over Total CytoRNA for each repeat in the pair of Alus (y-axis represent the fold change in the first repeat, x-axis represent the fold change in the second repeat in the pair) identified as an immunogenic RNA at the baseline condition (n=1,040 IR-Alu pairs) (F) and as Treatment-induced immunogenic RNA (n=3,687 IR-Alu pairs) (G).

H-I, Transcriptional orientation for each pair of IR-Alus identified as an immunogenic RNA at the baseline condition (n=1,040 IR-Alu pairs) (H) and as Treatment-induced immunogenic RNA (n=3,687 IR-Alu pairs) (I). Each bar shows the count of IR-pairs where both repeats are in the sense strand (+/+), both repeats are in the antisense strand (−/+), one repeat is in the sense strand and the other is in the antisense strand (Discordant (+/−) or (−/+)). The plot on the left shows the counts in the RNA-seq data from the mock-treated cells and the plot on the right shows the counts in the RNA-seq data from the 5-AZA-CdR-treated cells.

FIG. 3, Detecting immunogenic RNAs which are induced upon treatment with 5-AZA-CdR using MDA5/RNase Protection assay.

A, Venn-diagram showing the IR-Alu pairs at the Baseline (n=1,040), Immunogenic (5-AZA-CdR) (n=4,482) and Treatment-induced (n=3,687) conditions.

B, Number of Baseline-immunogenic RNAs (IR-Alu pairs=1,040, Non-IR Alu pairs=97).

C, Number of Treatment-induced immunogenic RNAs (IR-Alu pairs=3,687, Non-IR Alu pairs=547).

D-E, Average RPM profile of the baseline MDA5-protedcted RNA transcripts (n=1,176) that include Baseline IR-Alus (D) and the average RPM profile of the Treatment-induced MDA5-protected transcripts (n=3,895) that include treatment-induced IR-Alus (E). The boxplots include the RPM scores of these transcripts. Wilcoxon signed-rank test.

FIG. 4. Genomic distribution and regulatory features of Inverted Repeats Alus induced by 5-AZA-CdR treatment.

A, Genomic distribution of the baseline immunogenic IR-Alu pairs (n=1,040), treatment-induced immunogenic IR-Alu pairs (n=3,687), and of IR-Alu pairs present in the human genome (n=746,470). Odd-Ratio shows depletion or enrichment for each genomic category (Intron, Exon, 3′UTR, 5′UTR and Intergenic) at baseline immunogenic IR-Alu pairs or treatment-induced immunogenic IR-Alu pairs as compared to the background genomic distribution of all IR-Alu pairs present in the human genome. **** p<0.0001; Fisher Exact test.

B-C, Average CpG density (B) or bona-fide CpG Island (CGI) intersection density (C) flanking (−50 kb/+50 kb; Upstream/downstream) the Treatment-induced IR-Alu pairs (n=3,687, Black line or light gray line respectively) and all existing IR-Alu pairs in the human genome (n=746,470, Gray line or dash line respectively). For Treatment-induced IR-Alu pairs, the orientation was based on the transcriptional orientation in the MDA5-protected RNA-seq data and shows a higher CpG density and CpG Island frequency immediately upstream to the first repeat.

D, Heatmaps and average CUT & RUN profiles of H3K4me3 (left) and H3K27ac (right) signal at −50 kb/+50 kb (Upstream/downstream) of the Treatment-induced IR-Alu pairs regions. The orientation was based on the transcriptional orientation in the MDA5-protected RNA-seq data

E-G, Illustrative genome tracks of Treatment-induced IR-Alu pair located at an intronic (E) and intergenic region (F) and a baseline IR-Alu pair located at a 3′UTR region (G).

H, Schematic representation of Baseline and Treatment-induced immunogenic IR-Alu pairs.

FIG. 5, Genomic distribution based on the genomic orientation and regulatory features of the baseline and treatment-induced IR-Alus.

A-B, Genomic distribution of the IR Alu pairs based on their genomic orientation for the Baseline immunogenic RNAs (A) and for the Treatment-induced immunogenic RNAs (B). Arrows represent the genomic orientation (5′ to 3′) and arrow color represent the transcriptional orientation (Black for sense and White for antisense).

C, Alu subfamily distribution of the baseline immunogenic IR-Alus (n=1,602), treatment-induced immunogenic IR-Alus (n=5,199), and of IR-Alus present in the human genome (n=641,262). Odd-Ratio shows depletion or enrichment for each Alu subfamily (AluS, AluJ and AluY) at baseline immunogenic IR-Alus or treatment-induced immunogenic IR-Alus as compared to Alu subfamily distribution of all IR-Alus present in the human genome **** p<0.0001; Fisher Exact test.

D, Average CpG density 50 kb upstream and 50 kb downstream of the Baseline IR-Alu pairs and all the existing IR-Alu pairs in the hg19 genome.

E, Average intersection of IR-Alu pairs region (Baseline and all IR-Alu pairs) with the CpG islands.

F, Genome track plot of a Treatment-induced intronic IR-Alu pair, Genome track plot of a Treatment-induced intergenic IR-Alu pair and Genome track plot of a Treatment-induced intergenic IR-Alu pairs.

FIG. 6. DNA methylation status of CGI adjacent to immunogenic IR-Alus and correlation of methylation score of immunogenic IR-Alus regulatory regions with Viral mimicry signature.

A-B, Percentage of CGIs (n=1,338) directly upstream of 5-AZA-CdR induced immunogenic IR-Alus with at least one fully methylated (light gray), partially methylated (dark gray), or fully unmethylated (black) CpG site in (A) colorectal cancer cell lines (n=51) or (B) pan-cancer cell lines (n=988) from the GDSC project.

C-D, Regions that following epigenetic therapy become H3K4me3 marked directly upstream of induced immunogenic IR-Alus (n=991 peaks) with at least one fully methylated (light gray), partially methylated (dark gray), or fully unmethylated (black) CpG site in (C) colorectal cancer cell lines or (D) pan-cancer cell lines from the GDSC project.

E, Scatter plot showing inverse correlation (r=−0.23, p=7.58-13) between the DNA methylation score of regulatory regions that following epigenetic therapy become H3K4me3 marked directly upstream to the induced immunogenic IR-Alus (n=991 H3K4me3 peaks) and viral mimicry ISG signature score (n=22 ISGs). Each dot is one pan-cancer cell line (n=988) from the GDSC project.

FIG. 7. Characterization of the cytoplasmic immunogenic IR-Alus.

A-B, Distribution of each known human polyA signal (PAS) motif with respect to the distance from the end of the downstream Alu of each IR-Alu pair in the set of (A) baseline immunogenic IR-Alus (n=1,040 pairs) and the set of (B) treatment-induced immunogenic IR-Alus (n=3,687 pairs). The y-axis is the counts of the IR-Alu pairs that include the motif in the MDA5-protected RNA-seq, and the x-axis is the distance in bp from the end of the downstream Alu in an IR-Alu pair.

C-D, Heatmap and average profile of the MDA5-protected RNA-seq signal centered at the PAS locations detected in (A-B). Each row represents the downstream Alu for each IR-Alu pair. The orientation and the strand are based on the MDA5-protected transcriptional orientation.

E, Percentage of non-repeats and several families of repetitive elements in total RNA-seq (nuclear and cytoplasmic RNA) and in RNA-seq from the cytoplasmic fraction (‘Total CytoRNA-seq’) after 5-AZA-CdR treatment at 300 nM for 5 days. The Total CytoRNA-Seq donut plot (on right) is the same as in FIG. 2B and plotted here for reference. Odd-ratio was calculated between Total CytoRNA-Seq to Total RNA-seq. **** P<0.0001; Fisher Exact test.

F, Confocal microscopy of Mock-treated and 5-AZA-CdR treated ADAR1WT patient-derived xenograft CRCs. DNA was stained by DAPI (left) and dsRNA was staining using the J2 antibody (middle).

FIG. 8. ADAR1 suppresses immunogenic Alus and limits viral mimicry response to 5-AZA-CdR treatment.

A, Knock-down efficiency of ADAR1 using two distinct shRNAs (ADAR1^{KDA} and ADAR1^{KDB}) at mRNA (left) and protein (right) levels in patient-derived xenograft colorectal cancer cells (CRCs). The mRNA quantification was performed by qRT-PCR. Results were normalized to RPLP0. The protein quantification was performed by western-blot and the two ADAR1 isoforms (p110 and p150) are marked by an arrowhead. Alpha-Tubulin served as a loading control.

B, dsRNA quantification based on J2/DAPI staining measured by ImageJ. Data are mean±s.d. from n=20 randomly sampled regions of two independent experiments. ***P<0.001, ****P<0.0001, Dunnett-corrected ordinary one-way ANOVA.

C, qPCR analysis of total ADAR1 mRNA level after treatment with 5-AZA-CdR (300 nM, for 5 days) or transfection with 100 ng ml-1 poly(I:C) over a course of 24 days. Cells were washed out at day 5 and seeded in drug-free medium. Data are mean±s.e.m.****P<0.0001, Sidak-corrected two-way ANOVA.

D-F, Percentage of non-repeats and several families of Repetitive Elements in the total cytoplasmic fraction (‘Total CytoRNA’) and in the MDA5-protected fraction (‘MDA5-protected’) at the baseline condition (ADAR1^{WT}) (D) and after ADAR1 knockdown (‘ADAR1^{KD}-baseline immunogenic RNAs’), p<2.2e-16 E). ‘ADAR1^{KD}-induced immunogenic RNAs’ refers to transcripts present in ADAR1^{KD} condition but not present in the baseline ADAR1^{WT} condition (i.e. induced by the knockdown), p<2.2e-16 F). Each plot is the average percentage of two replicates. P-values represent the Fisher Exact test for enrichment of SINEs and depletion of non-repeats in ADAR1^{KD}-baseline immunogenic RNAs and ADAR1^{KD}-induced immunogenic RNAs as compared to the MDA5-protected ADAR1^{WT} baseline immunogenic RNAs.

G, Genome-wide A-to-I editing loci counts in Alus in the RNA-seq data performed in the total cytoplasmic fraction (‘Total CytoRNA’) and in the MDA5-protected fraction (‘MDA5-protected’) at the mock-treated (MT) ADAR1^{WT}, 5-AZA-CdR-treated ADAR1^{WT}, mock-treated ADAR1^{KD}, and 5-AZA-CdR-treated ADAR1^{KD} conditions.

H, Geneset Enrichment Analysis showing an enrichment of the geneset ‘Hallmark_Interferon_Alpha_Response’ in ADAR1^{KD} 5-AZA-treated cells compared to ADAR1^{WT} 5-AZA-CdR treated cells.

I, Interferon-responsive geneset enrichment score in mock-treated ADAR1^{WT}, 5-AZA-CdR-treated ADAR1^{WT}, mock-treated ADAR1^{KD}, and 5-AZA-CdR-treated ADAR1^{KD} conditions. Samples were collected 5, 14, and 24 days post-treatment.

J, MAVS aggregation analyzed by SDS-AGE and MAVS protein level analyzed by SDS-PAGE. VDAC served as a loading control in SDS-PAGE.

FIG. 9, ADAR1 induces immunogenic inverted Alus.

A, dsRNA quantification based on J2/DAPI staining measured by ImageJ. Data are mean±s.d. from n=20 randomly sampled regions of two independent experiments. ***P<0.001, ****P<0.0001, Dunnett-corrected ordinary one-way.

B, Venn-diagram showing the number of IR-Alu pairs at the baseline (n=1,040), ADAR1^{KD}-baseline immunogenic (n=9,030) and ADAR1^{KD}-induced immunogenic conditions.

C-D, Log₁₀ Fold change of MDA5-protected to Total CytoRNA enriched inverted (IR)-Alus and Non-IR Alus for Baseline immunogenic RNAs (Top) and for the ADAR1^{KD}-induced immunogenic RNAs (Bottom).

E-F, Correlation in the enrichment score of each pair of IR-Alus which make dsRNA in ADAR1^{WT} (baseline) and ADAR1^{KD} patient-derived colorectal cancer cells.

G-H, Strand specific transcription (+/+ or −/−) of repeat element pairs which form immunogenic dsRNA in ADAR1^{WT}(LACZ as baseline) and ADAR1^{KD} patient-derived xenograft colorectal cancer cells versus pairs appeared with discordant transcription strandness (+/− or −/+).

I-J, Average RPM profile of the baseline MDA5-protedcted RNA transcripts (n=1,176) that include Baseline IR-Alus (I) and the average RPM profile of the ADAR1^{KD}-induced MDA5-protected transcripts that include Treatment-induced IR-Alus (J). The boxplots indicate the RPM scores of these transcripts. Wilcoxon signed-rank test.

FIG. 10, ADAR1 depletion synergizes with anti-tumour effects of DNMTi through loss of its catalytic activity.

A, ADAR1 isoforms (p110 and p150) relative expression analysed by qPCR. LACZ denotes ADAR1WT; PIC denotes 100 ng/ml poly (I:C) for 5 days; AZA denotes 300 nM 5-AZA-CdR for 5 days). Data are mean±s.e.m. (n=3 from three independent experiments). ***P<0.001, ****P<0.0001, Tukey-corrected two-way ANOVA.

B, Kinetics of interferon stimulated genes (ISG15, DDX58 and IRF7) relative expression in ADAR1^{WT} (Black) and ADAR1^{KD} (Gray) patient-derived colorectal cancer cells (CRCs) treated with 5-AZA-CdR analyzed by qRT-PCR. Data are mean±s.e.m. (n=3 from three independent experiments). *P<0.05, **P<0.01, ****P<0.0001, Sidak-corrected two-way ANOVA.

C, Interferon-responsive gene expression in ADAR1^{WT} mock-treated (MT), 5-AZA-CdR treated, ADAR1^{KD} NT and ADAR1^{KD} p(I:C) transfected samples collected 5 days, 14 days and 24 days post-treatment, measured by RNA-seq.

D, Kinetics of interferon stimulated genes (ISG15, DDX58 and IRF7) relative expression in ADAR1^{WT} (Black) and ADAR1^{KD} (Gray) patient-derived CRCs transfected with p(I:C), analyzed by qRT-PCR. Data are mean±s.e.m. **P<0.01, ****P<0.0001, Sidak-corrected two-way ANOVA.

E, Overexpression of ADAR1 p150 isoform at mRNA (analyzed by qRT-PCR) and protein levels in patient-derived CRCs (analyzed by western-blot). Data are mean±s.e.m. (n=3 from three independent experiments). P=0.0144, Wilcoxon test.

F, Interferon stimulated genes (ISG15, DDX58 and IRF7) relative expression in CTRL (LUC) (Black) and ADAR1^{p150CE} (Gray) patient-derived CRCs treated with 5-AZA-CdR, analyzed by qRT-PCR. Data are mean±s.e.m. **P<0.01, ***P<0.001, Sidak-corrected two-way ANOVA.

G, IFN enrichment score in CTRL(LUC) Mock-treated, 5-AZA-CdR treated, ADAR1-p150 overexpressing NT and ADAR1-p150 overexpressing 5-AZA-CdR treated samples collected 5 days post-treatment and measured by RNA-seq.

H, dsRNA quantification by ImageJ in CTRL (LUC) and ADAR1^{p150OE} cells Mock-treated (MT) or treated with 5-AZA-CdR. Data are mean±s.d. from n=20 randomly sampled regions of two independent experiments. ****P<0.0001, Tukey-corrected ordinary one-way ANOVA. ns, non significant.

FIG. 11. ADAR1 depletion synergizes with DNMTi in induction of interferon stimulated genes in colorectal cancer cell lines.

A) ADAR1 p150 and p110 isoforms relative expression in LIM1215 CRC cells.

B) Interferon-stimulated genes (MDA5, IRF7 and DDX58) relative expression in ADAR1^{WT} and ADAR1^{KD} LIM1215 colorectal cancer cell lines treated with 300 nM 5-AZA-CdR for 5 days.

C) ADAR1 p150 and p110 isoforms relative expression in HT29 CRC cells.

D) Interferon-stimulated genes (MDA5, ISG15 and DDX58) relative expression in ADAR1^{WT} and ADAR1^{KD} HT29 colorectal cancer cell lines treated with 300 nM 5-AZA-CdR for 5 days. Data are mean±s.e.m. (n=3 from three independent experiments). *P<0.05, **P<0.01, ***P<0.001, ****P<0.0001, Tukey-corrected two-way ANOVA.

FIG. 12. ADAR1 depletion synergizes with anti-tumour effects of 5-AZA-CdR.

A, Survival of wild-type ADAR1 (black) and ADAR1-knockdown (gray) patient-derived CRC cells after treatment with 5-AZA-CdR. Luminescence signal was normalized, and dose-response curves and EC50 values were calculated using a nonlinear regression curve fit.

B, Schematic representation of in vivo experimental scheme of non-infected, ADAR1^{WT}, ADAR1^{KDA} and ADAR1^{KDB} patient-derived xenograft CRC cells injected subcutaneously into flanks of NSG mice (n=10 mice per group; 2 tumours per mouse). Arrows represent days of treatment (0.5 mg kg-1 5-AZA-CdR by intraperitoneal injection or vehicle).

C, Tumour volume measured at the indicated time points. Arrows represent days of treatment. ADAR1^{KDA} 5-AZA-CdR (n=20 tumours) and ADAR1^{KDB} 5-AZA-CdR (n=18 tumours at day 12 and n=14 tumours at day15) compared with non-infected (NI) 5-AZA-CdR (n=19 tumours) cohort. Data are mean±s.e.m. ****P<0.0001, Tukey-corrected two-way ANOVA

D, Colorectal cancer tumours dissected from Vehicle and 5-AZA-CdR cohorts at day 14 post-treatment are depicted for each condition (n=5) (NI=non-infected)

E, Tumour weight for each group at the time of dissection (day 14 after treatment). Data are mean±s.d. ****P<0.0001, Tukey-corrected ordinary-one-way ANOVA.

F Schematic representation of in vivo LDA measuring tumour-initiation potential. NSG mice were injected with 10,000 cells (n=8 tumours), 1,000 cells (n=8 tumours), 100 cells (n=8 tumours) and 10 cells (n=16 tumours).

G, Tumour-initiating cell frequency from the in vivo LDA of patient-derived CRC cells for each condition. ADAR1WT 5-AZA-CdR vs ADAR1KDA 5-AZA-CdR P=0.00862; ADAR1WT 5-AZA-CdR vs ADAR1KDB 5-AZA-CdR P=0.009; chi-square test.

FIG. 13, Epigenetic therapies which induce dsRNA can synergize with ADAR1 inhibition.

A, Change in mean body weight of NSG mice transplanted with non-infected, ADAR1^{WT} and ADAR1^{KDA/B} patient-derived CRCs treated with vehicle or 5-AZA-CdR (0.5 mg kg-1 through intraperitoneal injection, for two cycles of 4 days with a 3 day break).

B, The normalized percentage of reduction in sphere forming ability was calculated in each condition compared to Mock-treated.

C, Representative confocal microscopy images from two independent experiments of ADAR1^{WT} and ADAR1^{KDA} cells treated with CDK4/6 inhibitor (250 nM palbociclib) for 7 days. DNA was stained with DAPI, and dsRNA was stained using the J2 antibody.

D, dsRNA quantification using ImageJ. Data are mean±s.d. from n=15 randomly sampled regions of two independent experiments. **P<0.01, ***P<0.001, Tukey-corrected ordinary one-way ANOVA

E, qPCR analysis of relative expression of ISGs (ISG15, DDX58 and IRF7) in cells treated with 250 nM palbociclib. Data are mean±s.e.m. ***P<0.001, ****P<0.0001, Tukey-corrected twoway ANOVA.

FIG. 14, The level of A to I editing in IR-Alus predicts sensitivity to ADAR1 inhibitors.

A, A-to-I editing loci counts in IR-Alus in the total RNA-seq data of three CRC cell lines. NCIH716 and COLO678 cell linsea are classified as resistant to ADAR1 inhibitors, while NCIH747 is classified as sensitive to ADAR1 inhibitors.

B, Scatter plot of the ADAR1 decency of the CRC cell lines. ADAR1 dependent cell lines have more negative CERES scores.

FIG. 15, computer device for implanting the methods.

A suitable configured computer device, and associated communications networks, devices, software and firmware to provide a platform for enabling one or more embodiments as described herein.

### DETAILED DESCRIPTION

In the following description, numerous specific details are set forth to provide a thorough understanding of the invention. However, it is understood that the invention may be practiced without these specific details.

Cancer therapies targeting epigenetic repressors can mediate their effects by activating retroelements within the human genome. Retroelement transcripts can form double-stranded RNAs (dsRNAs) that activate the pattern recognition receptor MDA5¹⁻¹⁰. This viral mimicry state leads to loss of cancer cell fitness and stimulates innate and adaptive immune responses^{11,12}. Nevertheless, the clinical efficacy of epigenetic therapies has been limited. To uncover targets that would synergize with the viral mimicry response, we set out to identify the immunogenic retroelements that are activated by epigenetic therapies. We discovered that intronic and intergenic SINE elements, specifically inverted repeats (IR) Alus, are the major source of drug-induced immunogenic dsRNAs. These IR-Alus are frequently located downstream of ‘orphan’ CpG Islands (CGIs)¹³. We show that orphan CGIs acquire active histone modifications after drug treatment, suggesting that they are evolutionarily conserved cryptic promoters. In mammals, the enzyme Adenosine Deaminases Acting on RNA (ADAR1) targets and destabilizes IR-Alu dsRNAs¹⁴, which prevents activation of MDA5¹⁵. We found that ADAR1 establishes a negative feedback loop, restricting the viral mimicry response to epigenetic therapy. Depletion of ADAR1 in patient-derived cancer cells potentiates the efficacy of epigenetic therapy, restraining tumor growth and reducing cancer initiation. Thus, epigenetic therapies trigger viral mimicry by inducing a subset of IR-Alus, which is enhanced by depleting ADAR1. Our findings suggest that combining epigenetic therapies with ADAR1 inhibitors represents a promising new strategy for cancer treatment.

In an aspect, there is provided a method of assessing a subject's responsiveness to cancer therapy, comprising: providing a sample from the subject comprising cancers cells or suspected cancer cells; measuring or estimating the expression levels of inverted repeats (IR) Alus in the cells; and determining that the subject would be responsive to cancer therapy if the subject cells exhibit expression levels of inverted repeats (IR) Alus with reference to expression levels in control samples.

The term “level of expression” or “expression level” as used herein refers to a measurable level of expression of the products of biomarkers, such as, without limitation, the level of messenger RNA transcript expressed or of a specific exon or other portion of a transcript, or in the context estimating expression, whether promoters of that biomarker are activated, such as through the methylation of CpG islands in the case of the IR Alus.

As used herein, the term “control” refers to a specific value or dataset that can be used to prognose or classify the value e.g. expression level or reference expression profile obtained from the test sample associated with an outcome class. A person skilled in the art will appreciate that the comparison between the expression of the biomarkers in the test sample and the expression of the biomarkers in the control will depend on the control used.

The term “differentially expressed” or “differential expression” as used herein refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example as measured by the amount of messenger RNA transcript in a sample as compared with the measurable expression level of a given biomarker in a control.

The term “responsiveness” as used herein in respect of cancer therapy refers to a subject's relative response to a therapy compared to a general or control population.

The term “sample” as used herein refers to any fluid, cell or tissue sample from a subject that can be assayed for biomarker expression products and/or a reference expression profile, e.g. genes differentially expressed in subjects.

In some embodiments, the method comprises measuring the expression levels of IR Alus by measuring the transcription product of IR Alus.

In some embodiments, the IR Alus to be measured are a majority of the IR Alus shown in Table B. In a preferred embodiment, the IR Alus to be measured are at least 60%, 70%, 80%, 90%, or 95% of the IR Alus shown in Table B. In a further preferred embodiment, the IR Alus to be measured are all or substantially all of the IR Alus shown in Table B.

In some embodiments, the measuring comprises use of RNA sequencing or PCR/digital PCR.

In some embodiments, the method comprises estimating expression levels of IR Alus by detecting the presence of methylated CpG islands upstream of IR Alus.

In some embodiments, the methylated CpG islands upstream to be detected are a majority of the CpG islands shown in Table A. In a preferred embodiment, the methylated CpG islands upstream to be detected are at least 60%, 70%, 80%, 90%, or 95% of the CpG islands shown in Table A. In a further preferred embodiment, the methylated CpG islands upstream to be detected are all or substantially all of the CpG islands shown in Table A.

In some embodiments, the method further comprises the treating the patient with the cancer therapy. The cancer therapy is preferably epigenetic therapy.

In some embodiments, the epigenetic therapy targets at least one of DNMTs, HDACs, LSD1, EZH2, G9a and SETDB1.

In some embodiments, the epigenetic therapy is a DNA methyltransferase inhibitor (DNMTi).

In some embodiments, the treatment further comprises decreasing, knocking or inhibiting Adenosine Deaminases Acting on RNA, preferably ADAR1.

In some embodiments, the measured level of A to I editing in treatment-induced IR Alus in the subject are high when compared to a reference level of A to I editing in treatment-induced IR Alus.

In an aspect, there is provided a method of cancer therapy in a subject in need thereof, the method comprising inhibiting ADAR1 in a patient.

In some embodiments, the inhibiting comprises administration of an ADAR1 inhibitor.

In some embodiments, the subject had been determined to be responsive to cancer therapy according to a method described herein.

In some embodiments, said cancer therapy comprises combination therapy with at least one other cancer therapy. Preferably, the at least one other cancer therapy is any of epigenetic therapy, surgery, endocrine therapy, chemotherapy, radiotherapy, hormone therapy, gene therapy, thermal therapy, or ultrasound therapy.

In an aspect, there is provided an ADAR1 inhibitor for use in the treatment of cancer in a subject.

In an aspect, there is provided a use of an ADAR1 inhibitor in the preparation of a medicament for the treatment of cancer in a subject.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The present system and method may be practiced in various embodiments. A suitably configured computer device, and associated communications networks, devices, software and firmware may provide a platform for enabling one or more embodiments as described above. By way of example, FIG. 15 shows a generic computer device 100 that may include a central processing unit (“CPU”) 102 connected to a storage unit 104 and to a random access memory 106. The CPU 102 may process an operating system 101, application program 103, and data 123. The operating system 101, application program 103, and data 123 may be stored in storage unit 104 and loaded into memory 106, as may be required. Computer device 100 may further include a graphics processing unit (GPU) 122 which is operatively connected to CPU 102 and to memory 106 to offload intensive image processing calculations from CPU 102 and run these calculations in parallel with CPU 102. An operator 107 may interact with the computer device 100 using a video display 108 connected by a video interface 105, and various input/output devices such as a keyboard 115, mouse 112, and disk drive or solid state drive 114 connected by an I/O interface 109. In known manner, the mouse 112 may be configured to control movement of a cursor in the video display 108, and to operate various graphical user interface (GUI) controls appearing in the video display 108 with a mouse button. The disk drive or solid state drive 114 may be configured to accept computer readable media 116. The computer device 100 may form part of a network via a network interface 111, allowing the computer device 100 to communicate with other suitably configured data processing systems (not shown). One or more different types of sensors 135 may be used to receive input from various sources.

The present system and method may be practiced on virtually any manner of computer device including a desktop computer, laptop computer, tablet computer or wireless handheld. The present system and method may also be implemented as a computer-readable/useable medium that includes computer program code to enable one or more computer devices to implement each of the various process steps in a method in accordance with the present invention. In case of more than computer devices performing the entire operation, the computer devices are networked to distribute the various steps of the operation. It is understood that the terms computer-readable medium or computer useable medium comprises one or more of any type of physical embodiment of the program code. In particular, the computer-readable/useable medium can comprise program code embodied on one or more portable storage articles of manufacture (e.g. an optical disc, a magnetic disk, a tape, etc.), on one or more data storage portioned of a computing device, such as memory associated with a computer and/or a storage system.

The advantages of the present invention are further illustrated by the following examples. The examples and their particular details set forth herein are presented for illustration only and should not be construed as a limitation on the claims of the present invention.

### Examples

DNMTi Induces Immunogenic Inverted Alus

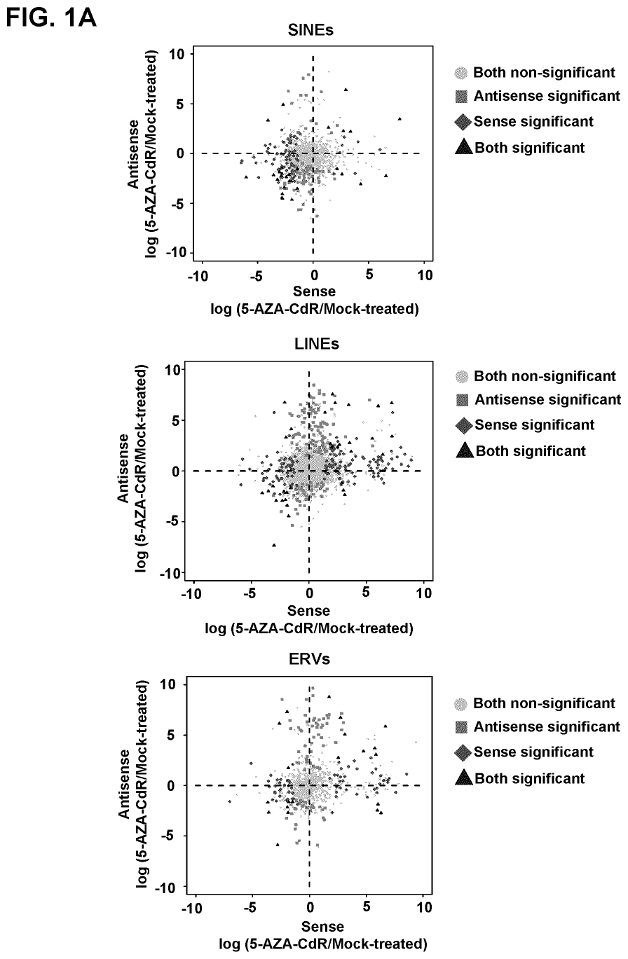

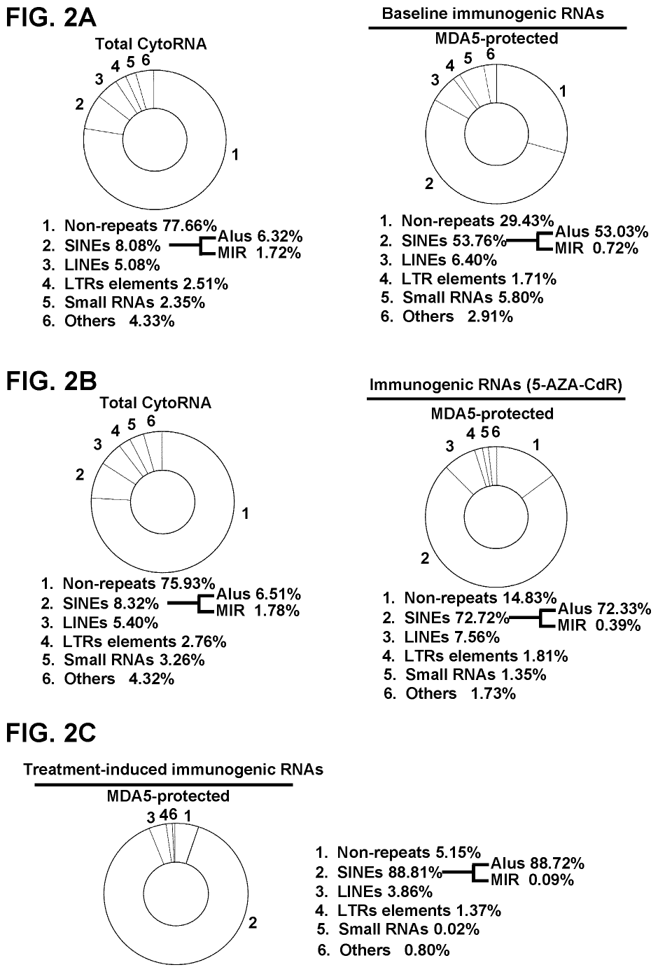

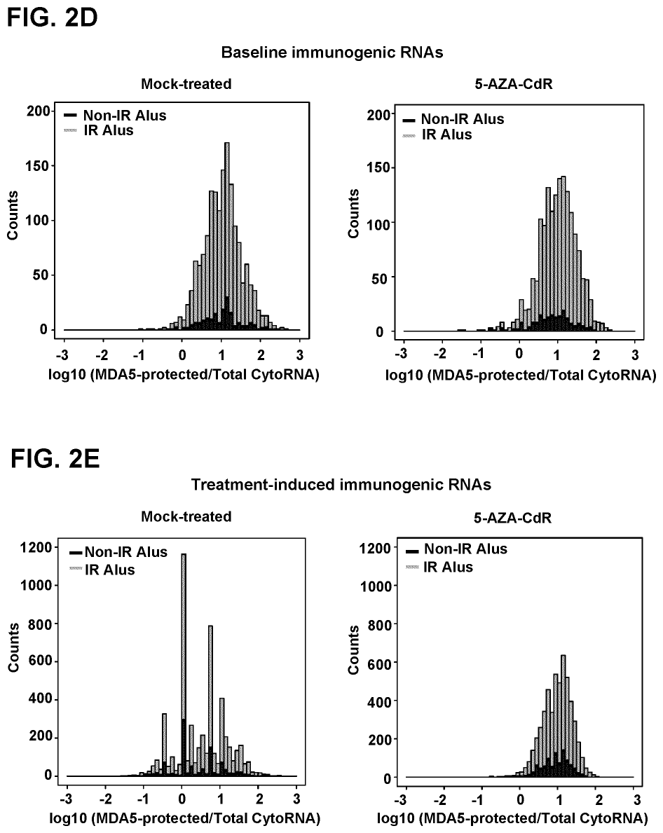

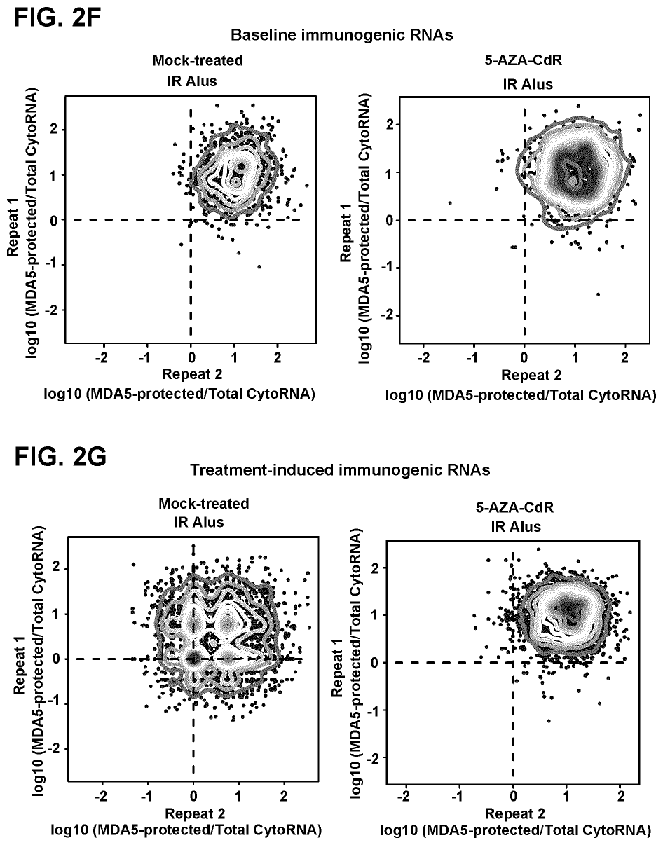

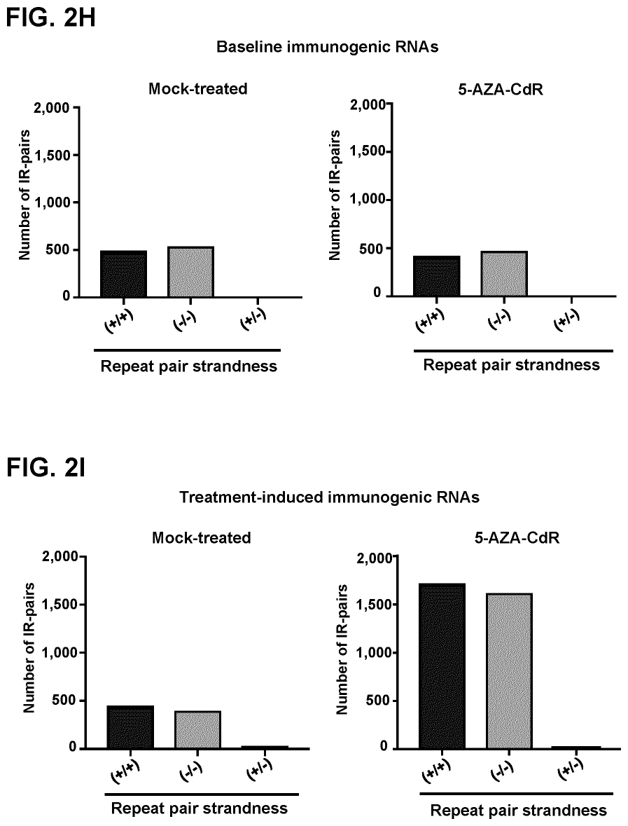

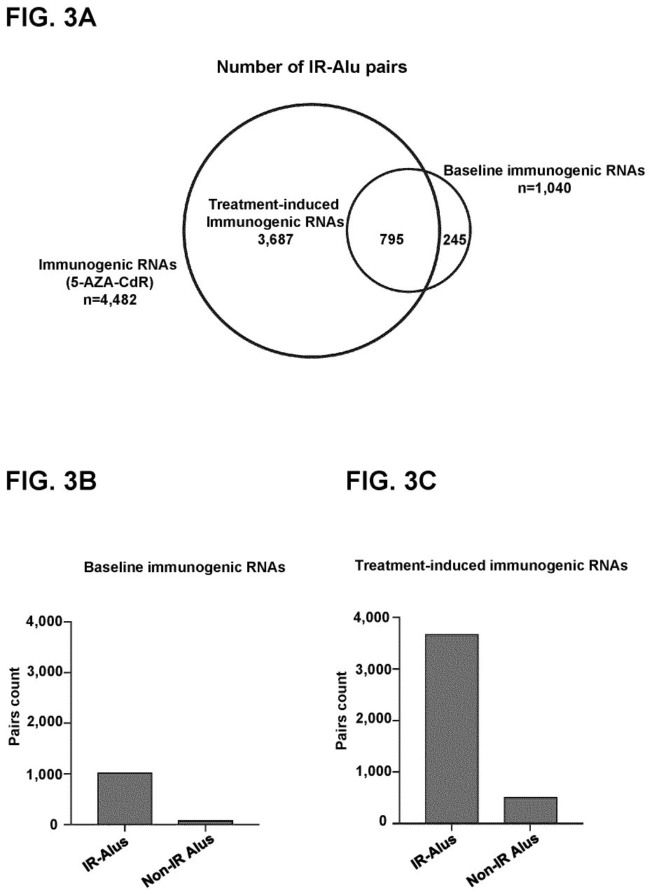

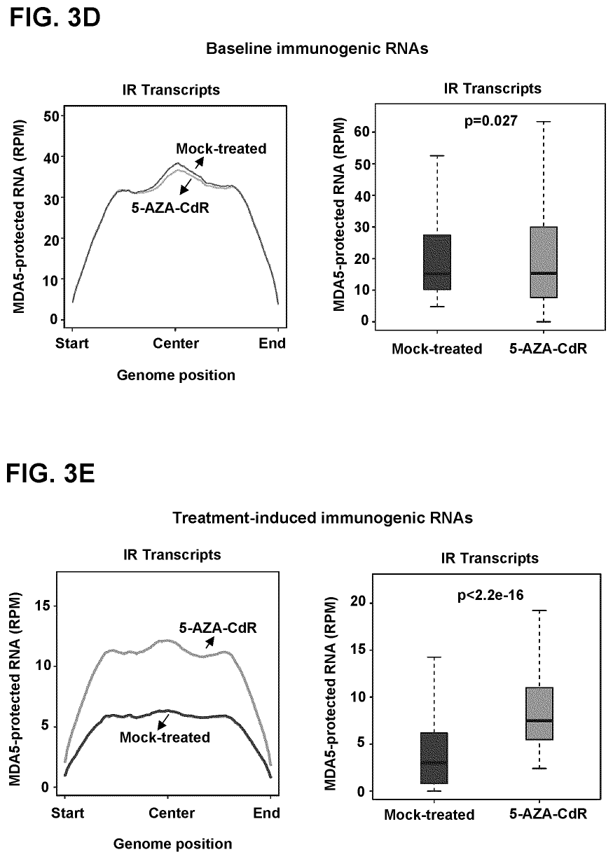

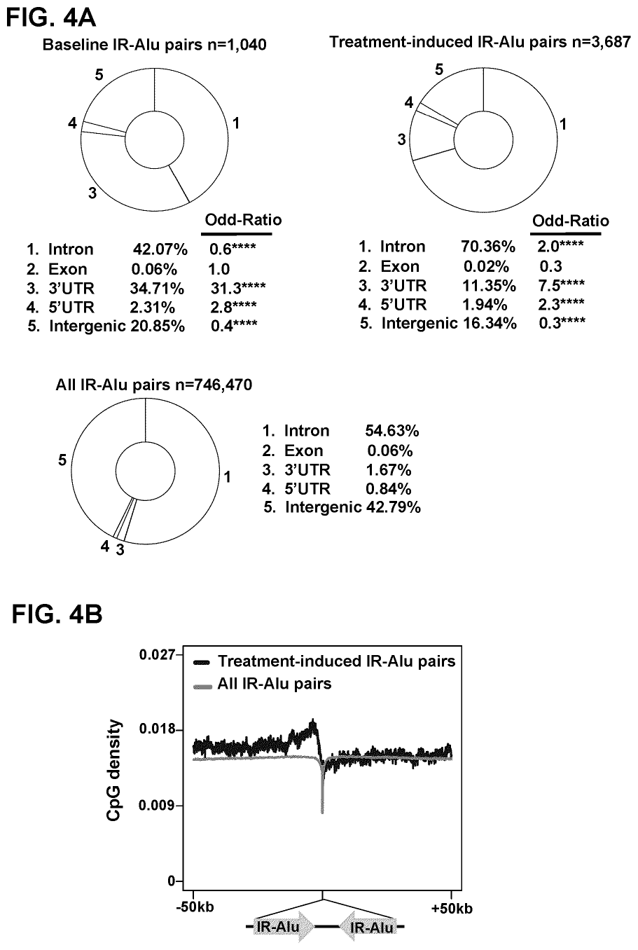

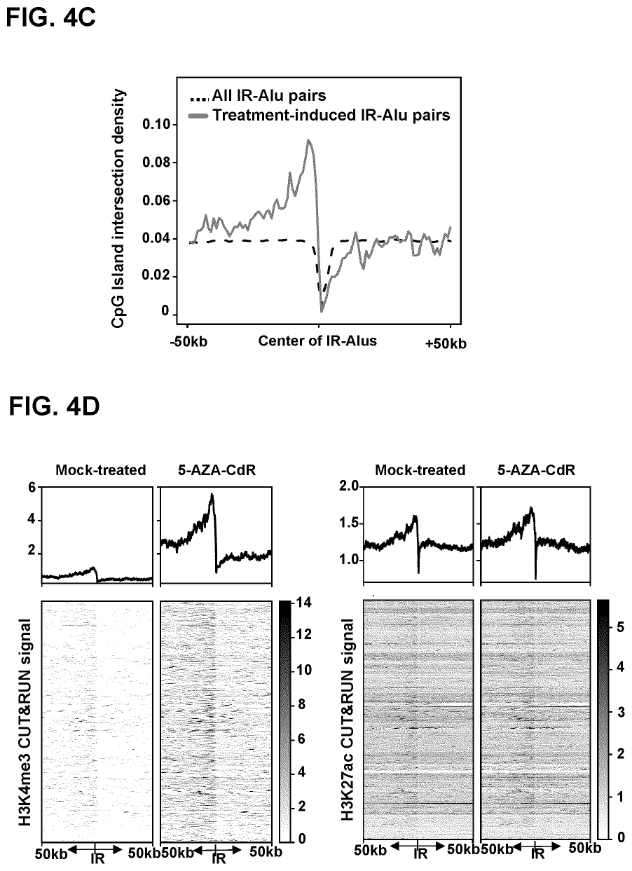

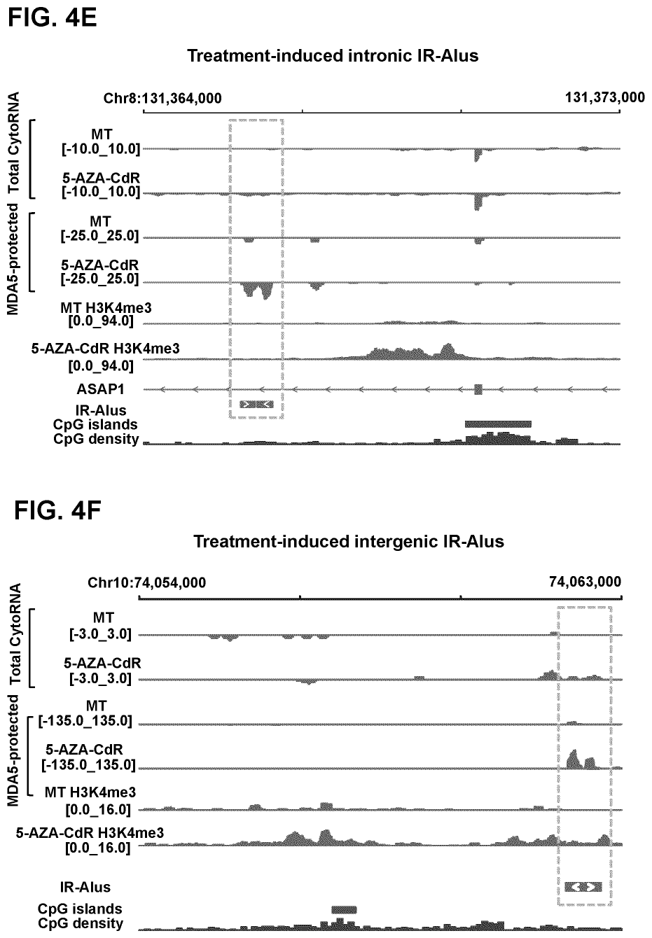

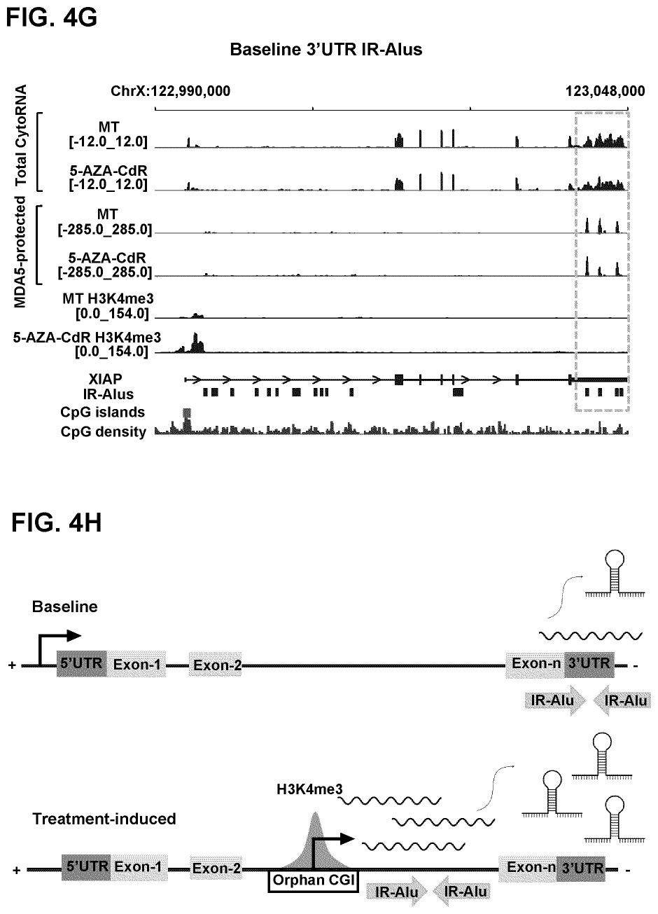

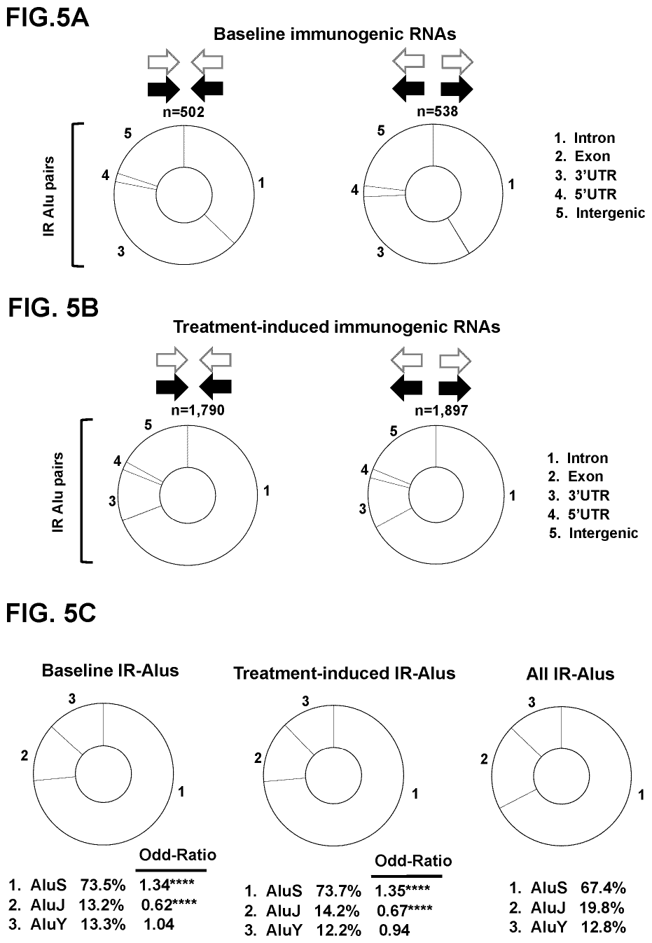

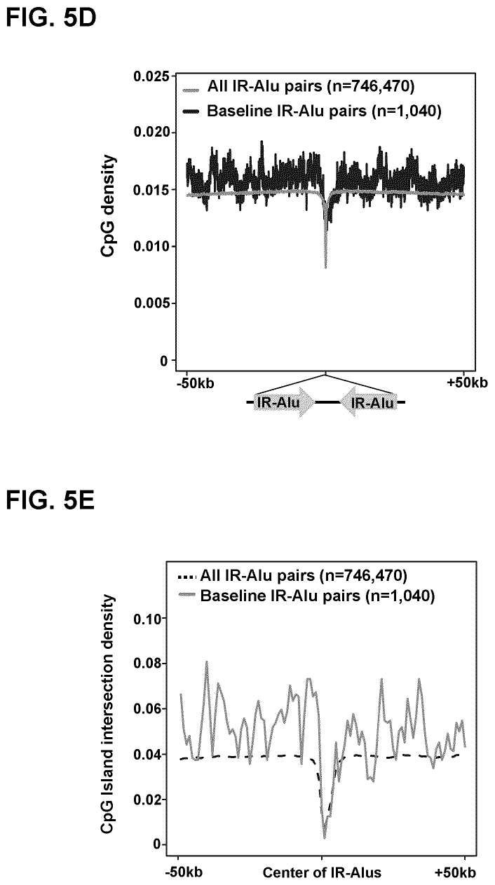

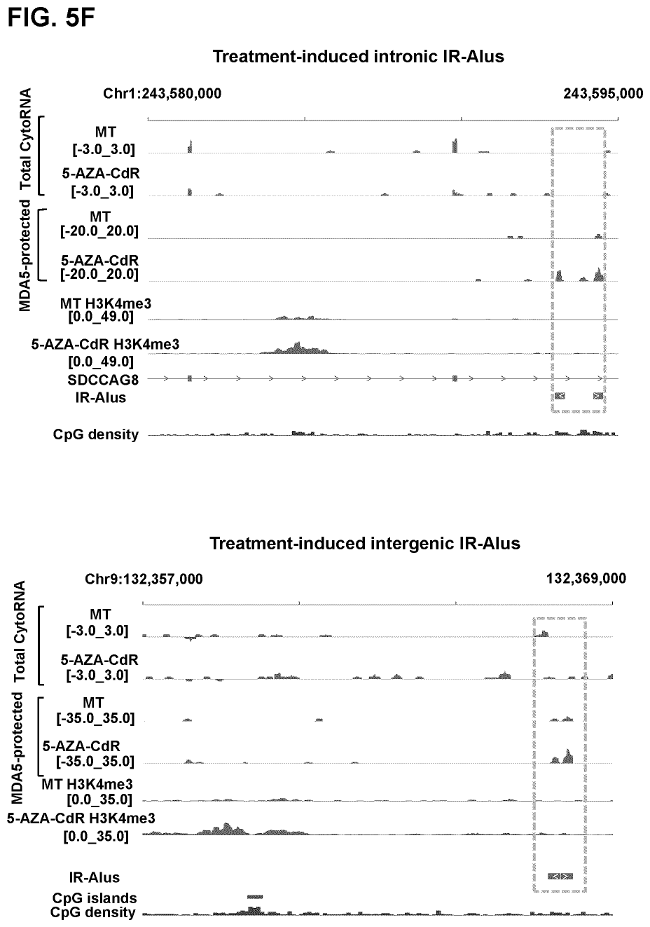

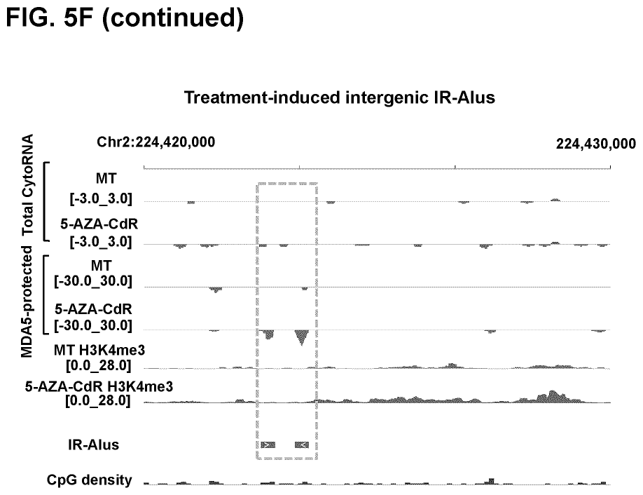

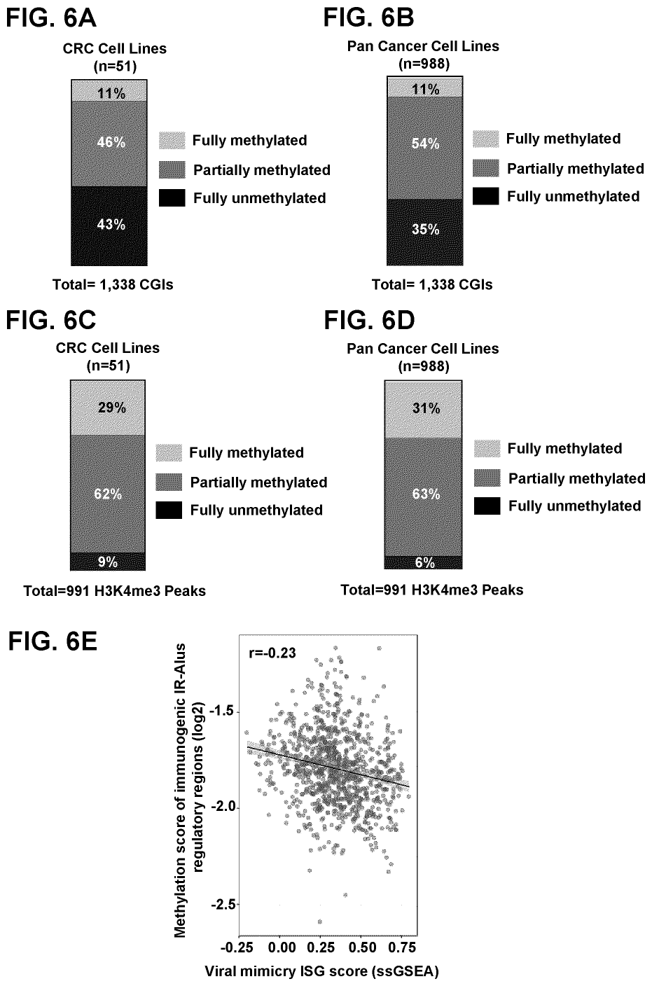

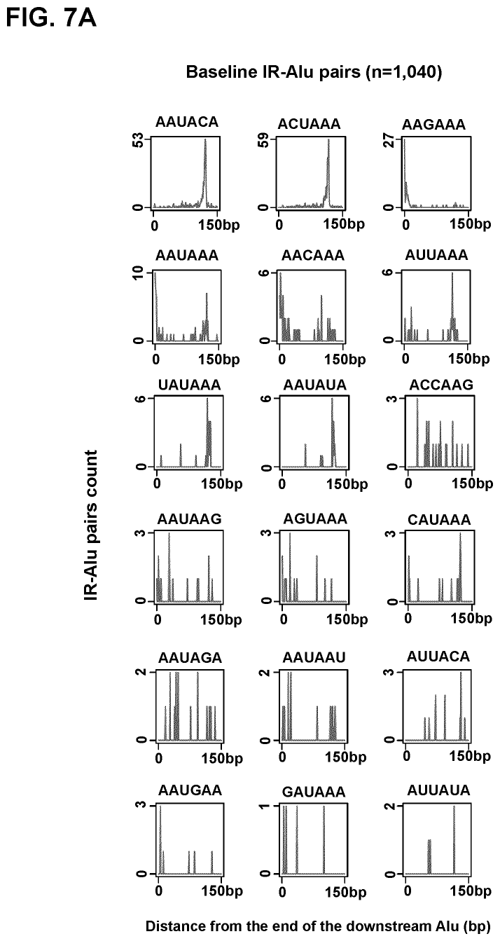

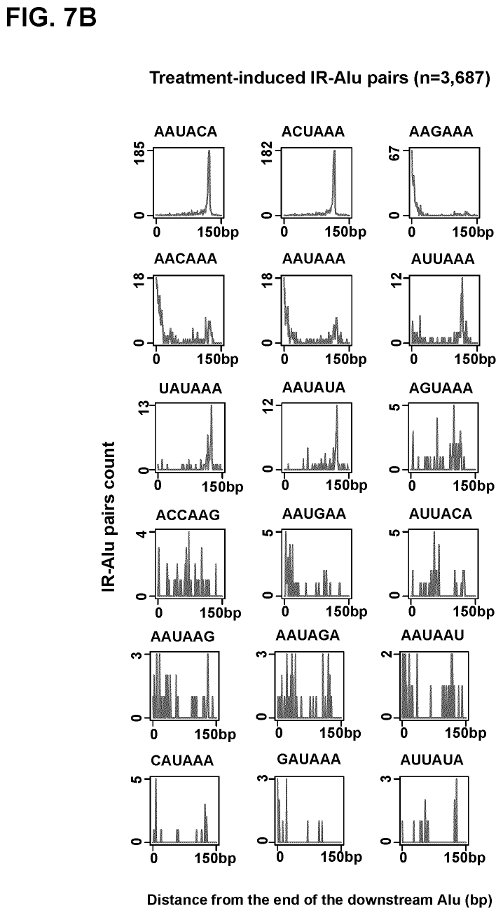

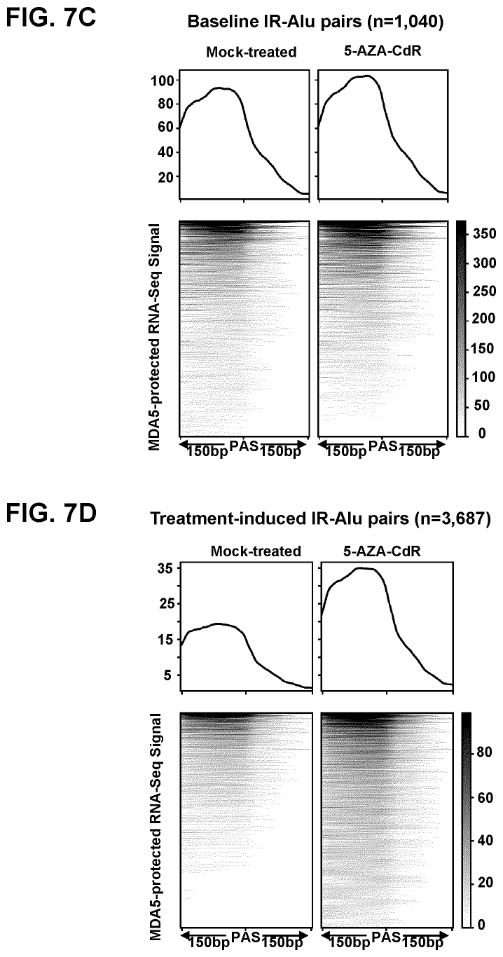

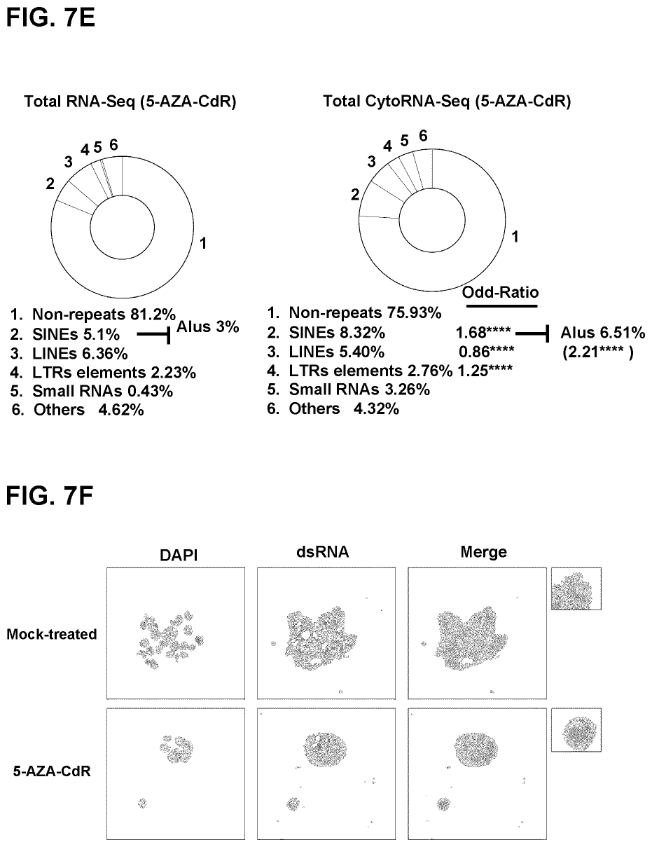

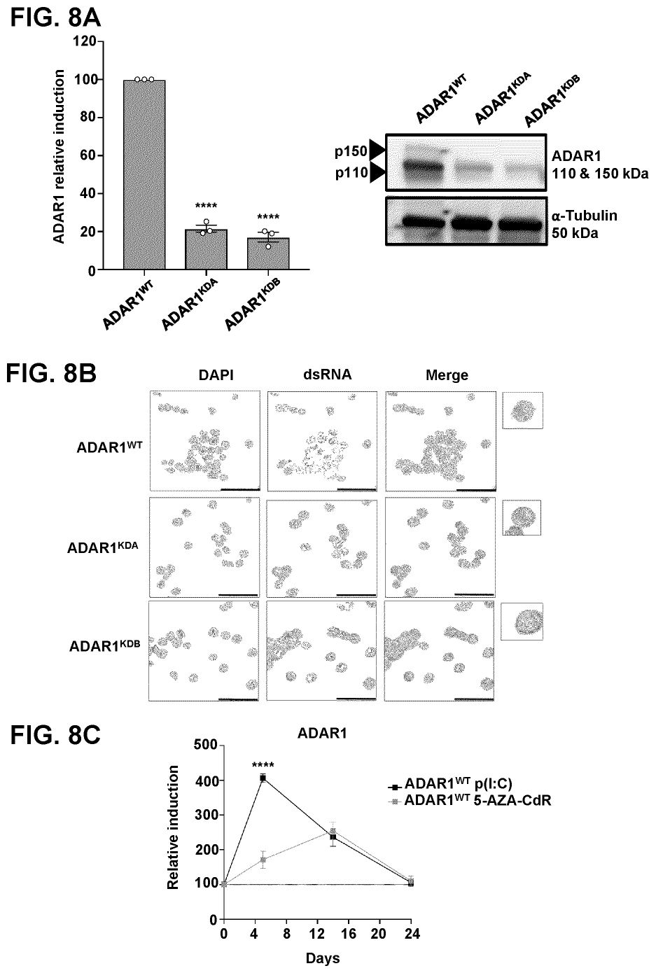

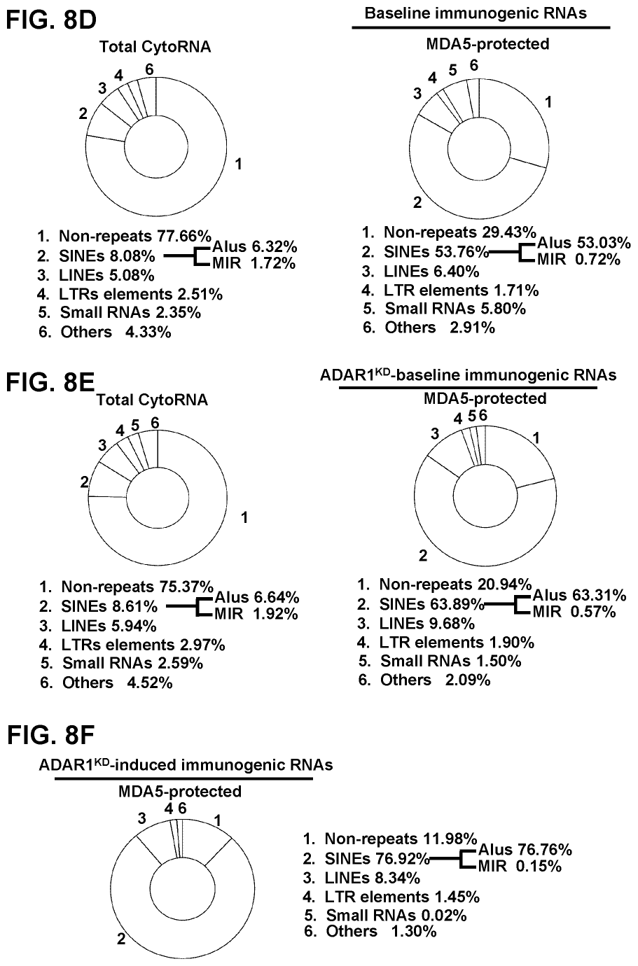

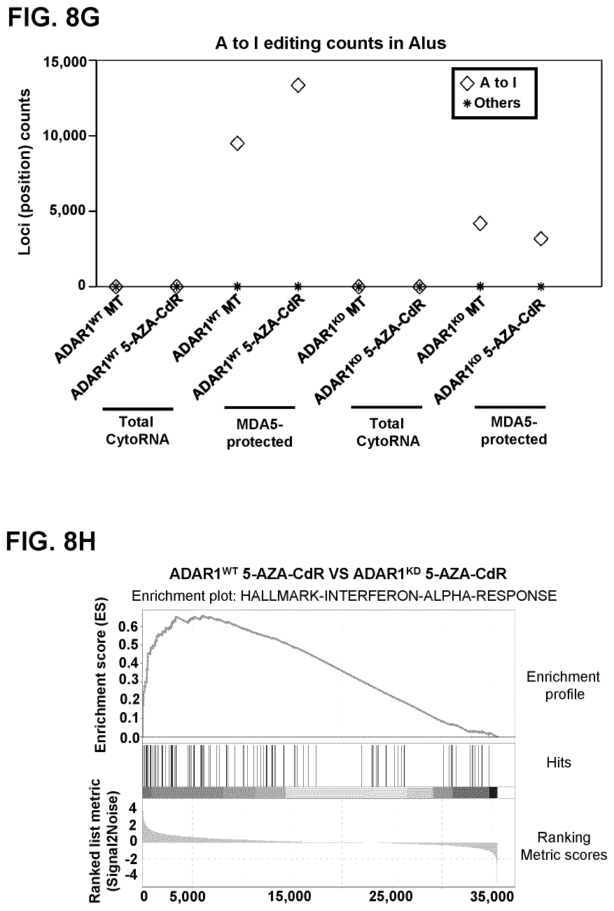

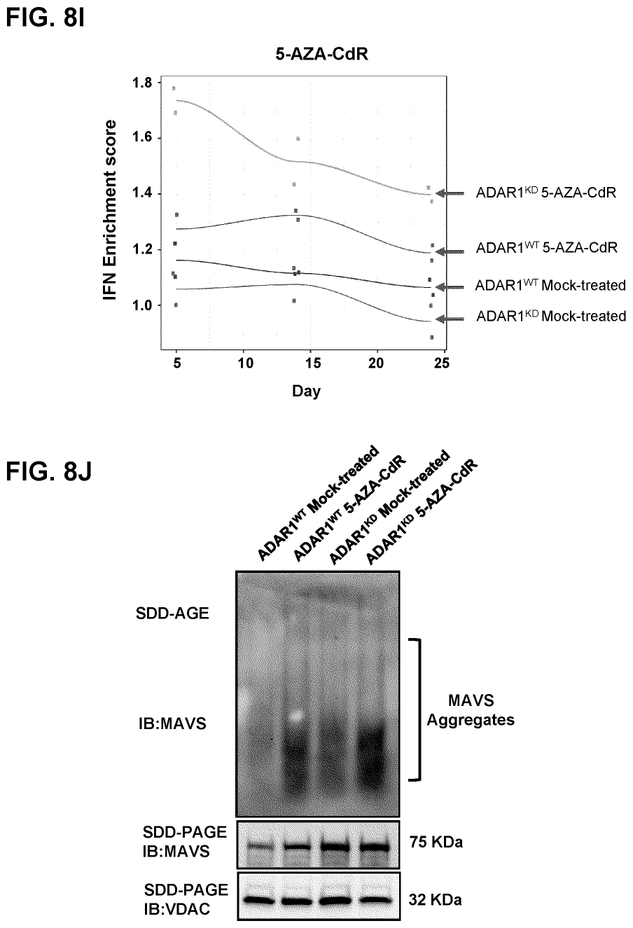

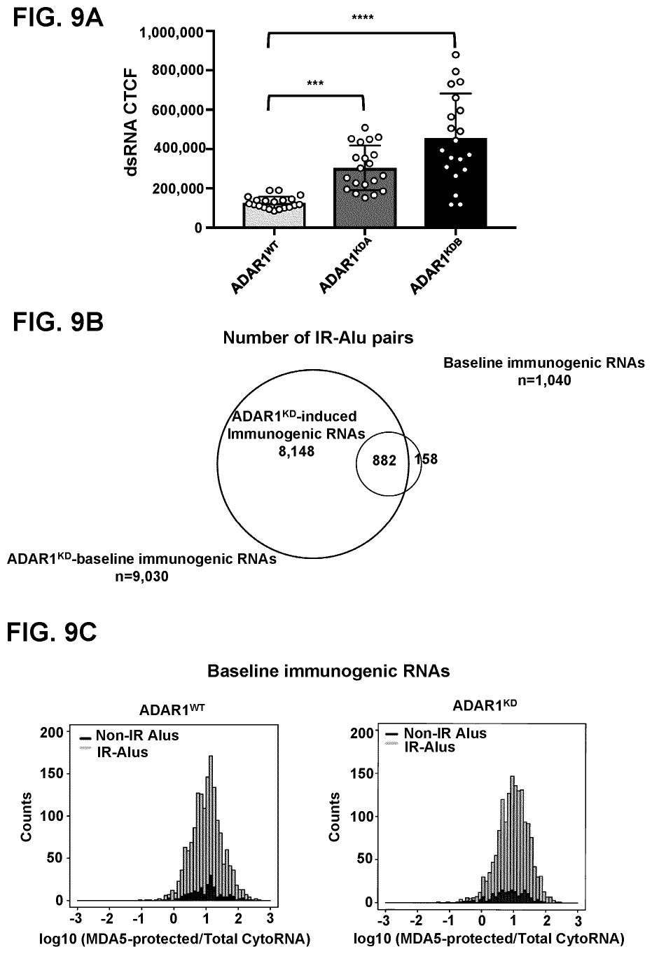

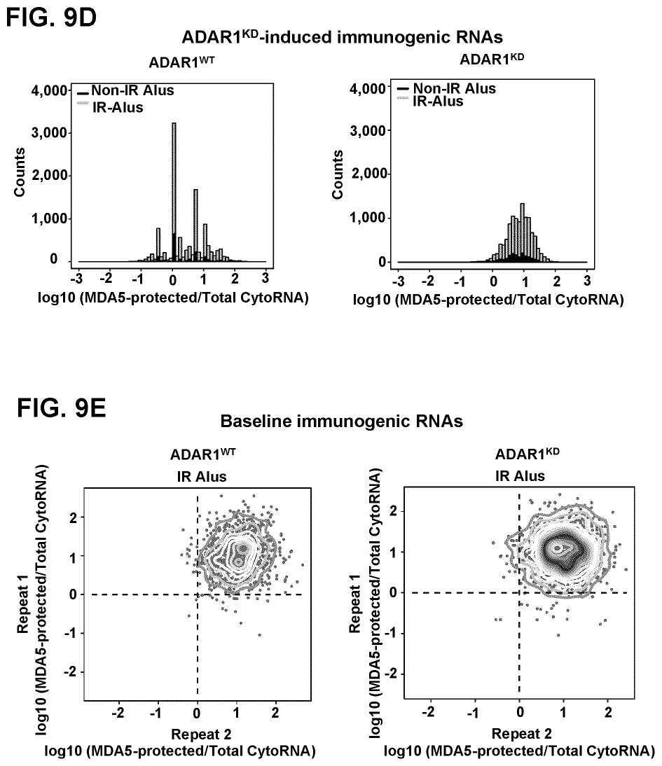

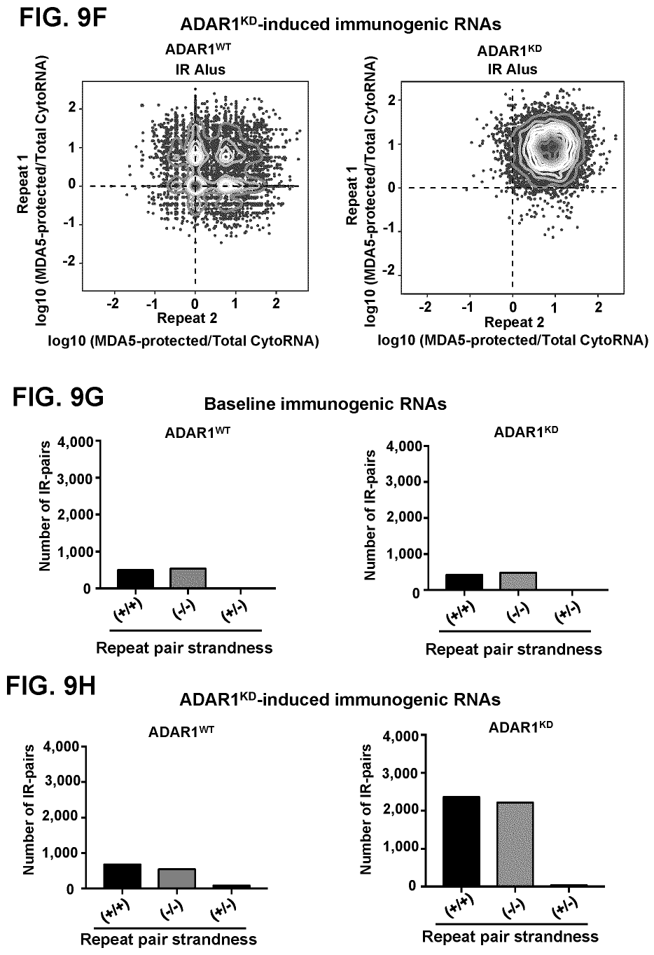

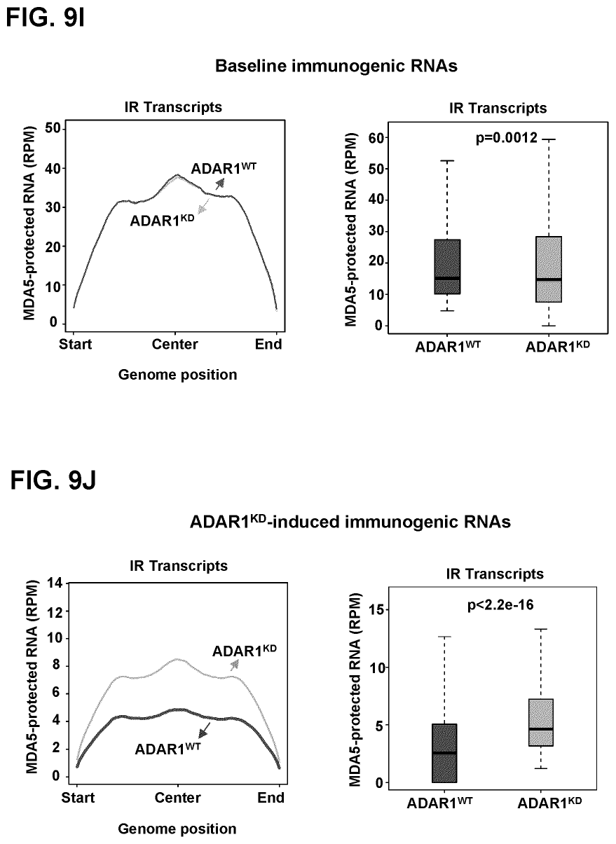

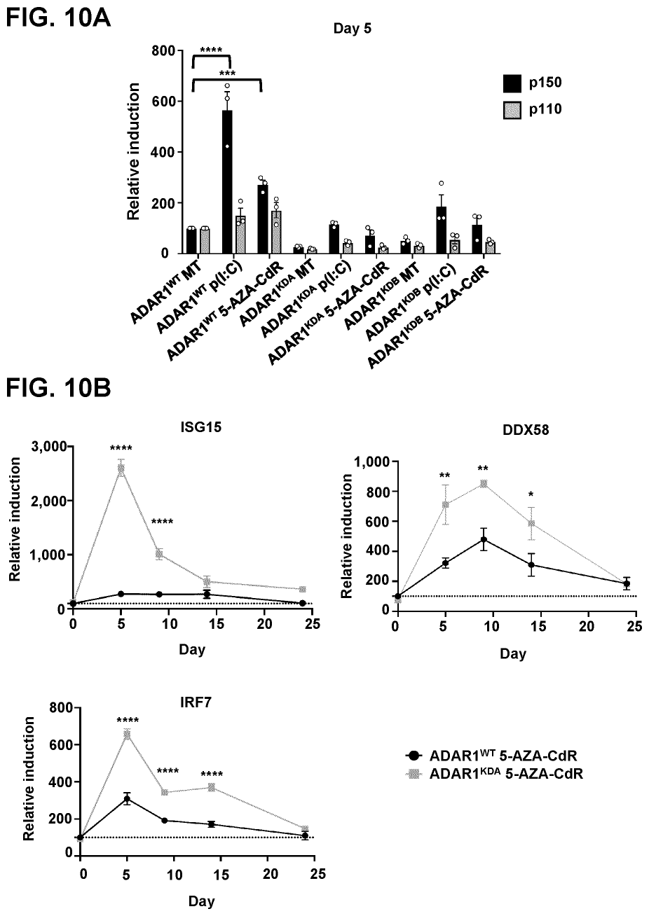

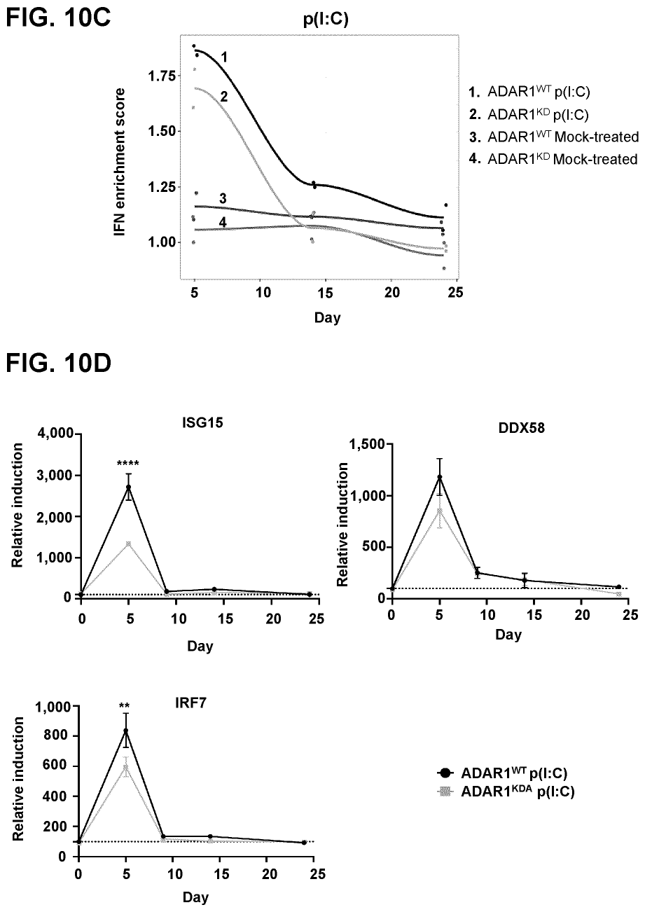

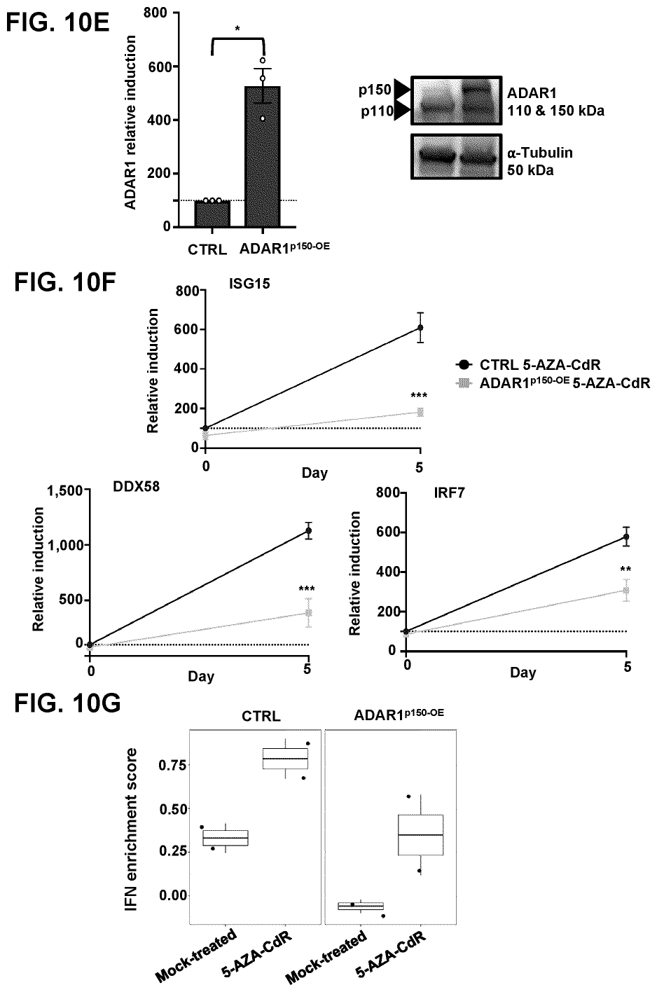

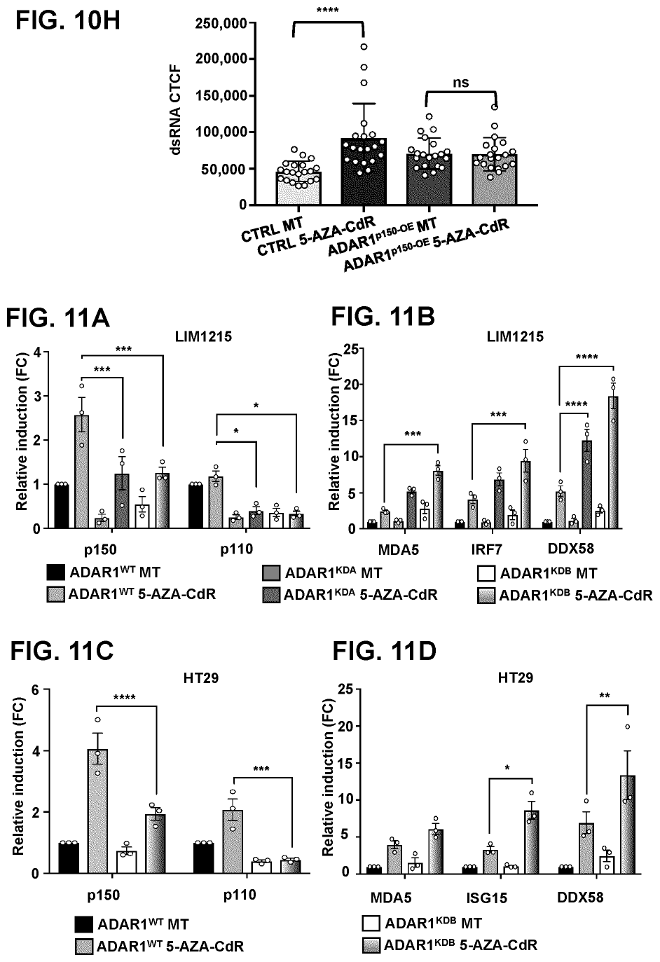

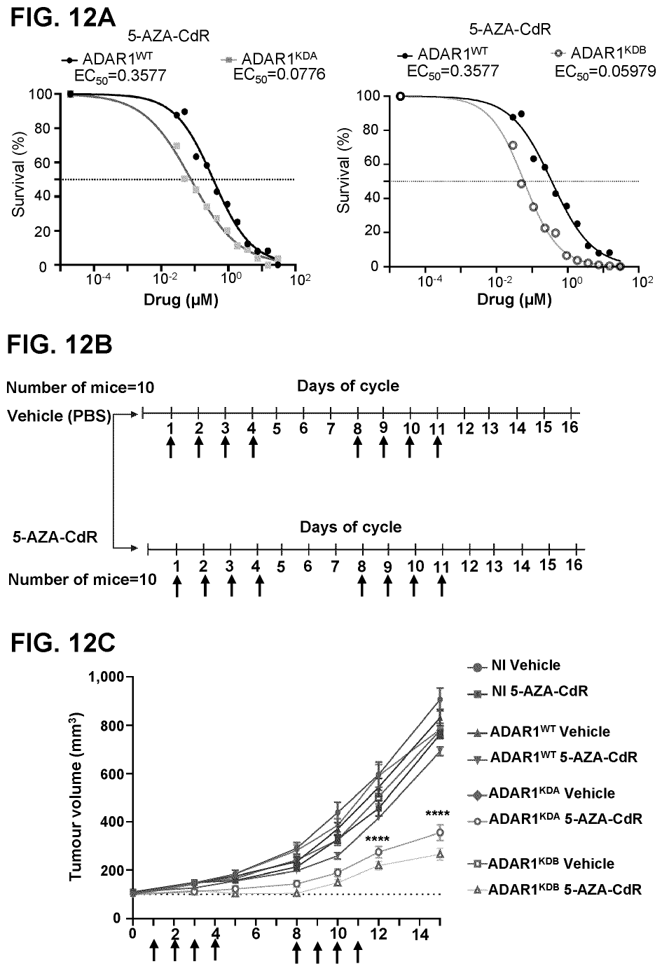

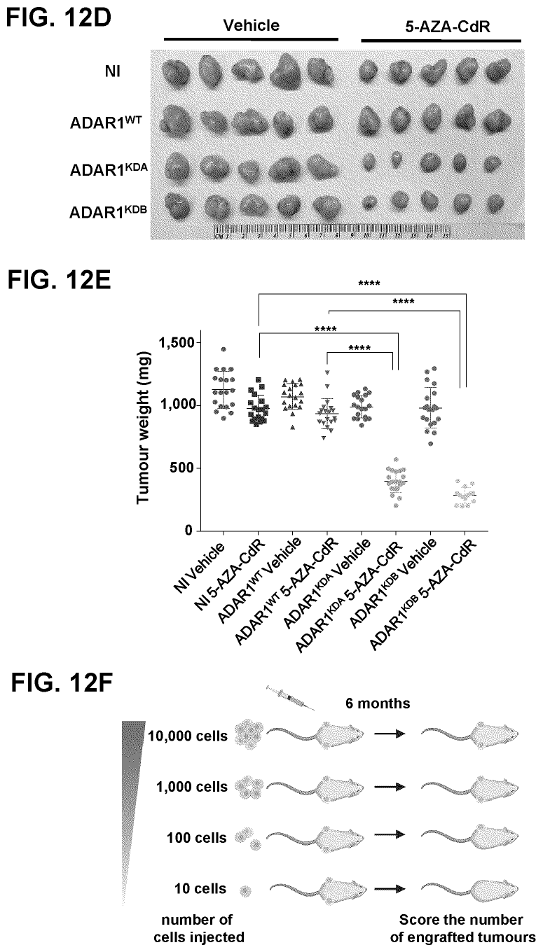

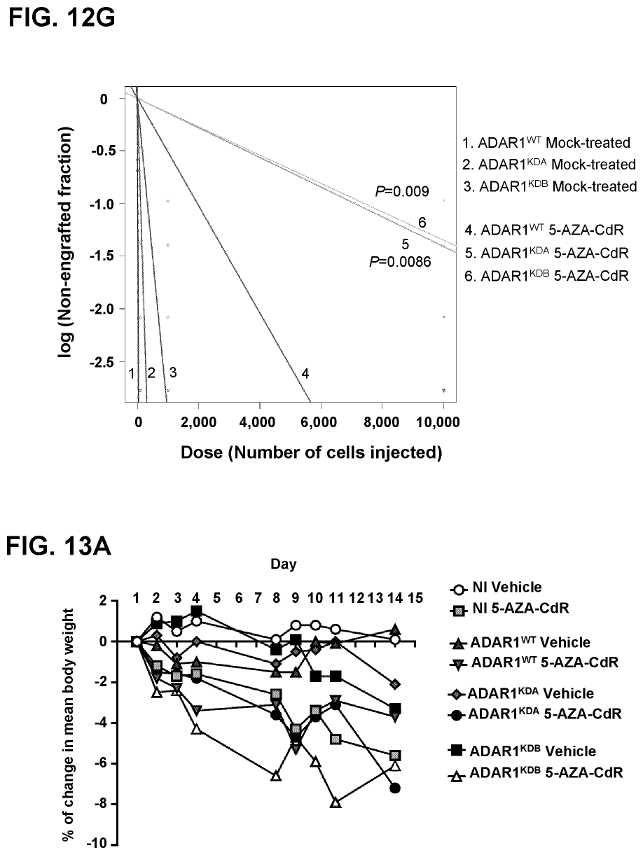

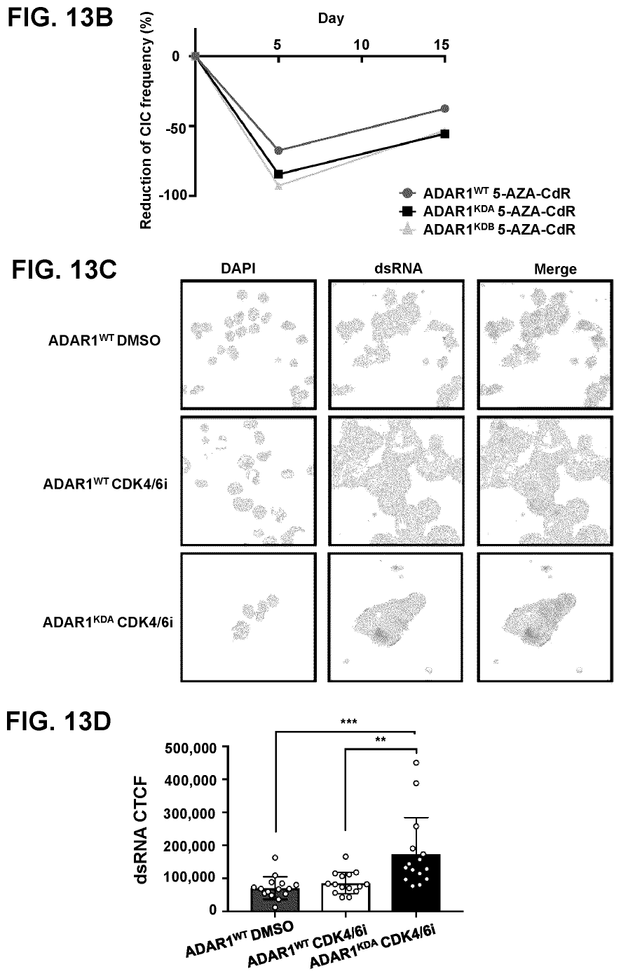

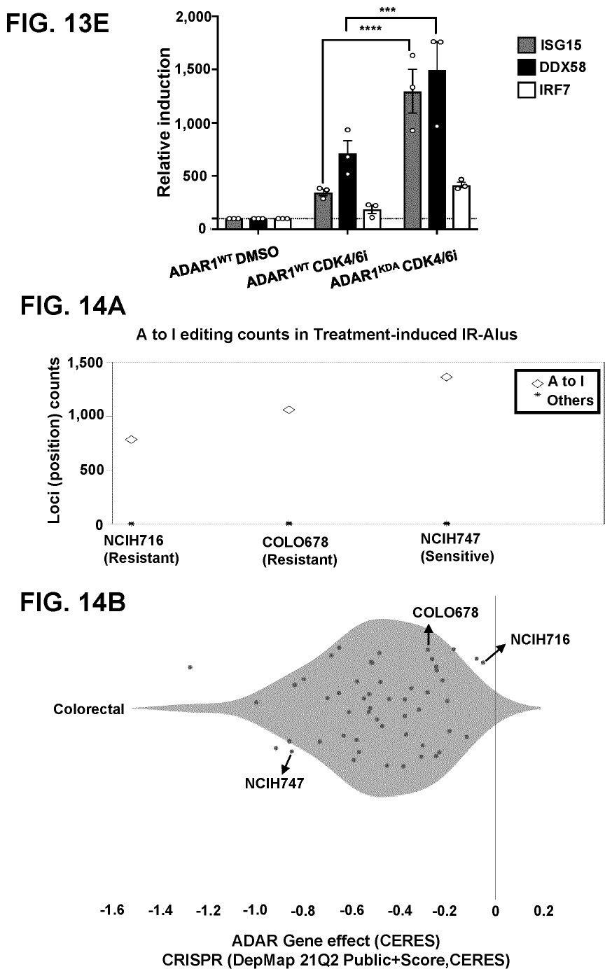

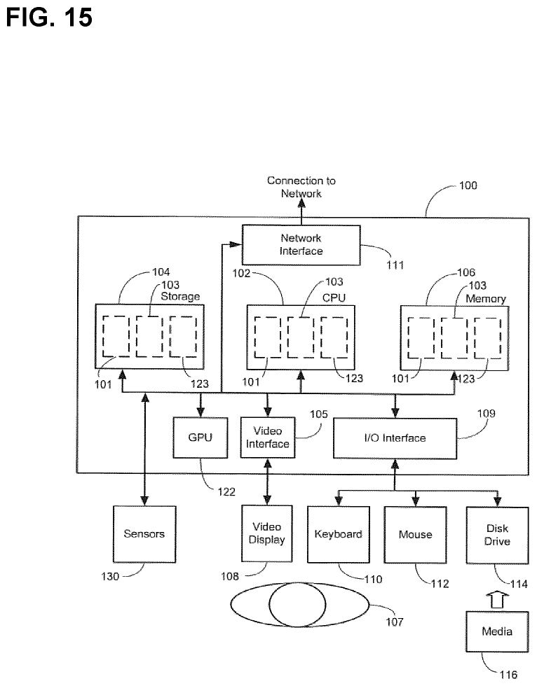

To identify drug-induced immunogenic retroelements on a global scale, we examined the endogenous dsRNA formation in patient-derived colorectal cancer cells (CRCs) that were treated with low doses of 5-AZA-CdR, an FDA-approved DNMTi, or mock-treated. To this end, we performed total stranded RNA-seq and assessed repetitive elements transcribed in both strands, therefore likely to produce dsRNA species. We observed increased sense and/or antisense transcription of long interspersed nuclear elements (LINEs) and ERVs, but not short interspersed nuclear elements (SINEs), after DNMTi treatment (FIG. 1A), consistent with previous studies showing disruption of repressive epigenetic marks can lead to formation of dsRNA from LINEs and ERVs through bidirectional transcription^{1-3,6,8,17,18}.

Viral mimicry response to epigenetic therapy is dependent on activation of the MDA5 pattern recognition receptor by endogenous dsRNAs^{1,6,8}. Therefore, to directly identify drug-induced immunogenic retroelements, we performed an MDA5-protection assay¹⁹, followed by RNA sequencing. Surprisingly, SINEs, specifically Alu retroelements, were remarkably enriched in the MDA5-protected RNA compared to total cytosolic RNA (FIG. 2A-C). Importantly, Alus enrichment in the MDA5-protected RNA increased from 53.03% in mock-treated samples (“Baseline immunogenic RNAs”) (FIG. 2A) to 72.33% in DNMTi-treated samples (“Immunogenic RNAs (5-AZA-CdR)”) (FIG. 2B). In contrast, only 1.71% of MDA5-protected RNAs in mock-treated samples were LTRs and 6.40% were LINEs (FIG. 2A). For DNMTi-treated samples, only 1.81% were LTRs and 7.56% were LINEs (FIG. 2B).

Then, to identify the immunogenic retroelements induced only by DNMTi treatment (“Treatment-Induced immunogenic RNAs”), we excluded those that were expressed in both the mock- and DNMTi-treated samples (FIG. 3A). Alu retroelements represented 88.72% of all MDA5-protected RNAs present only in DNMTi treated samples (“Treatment-Induced immunogenic RNAs”) (FIG. 2C). Treatment-induced immunogenic RNAs also included non-repeats (5.15%), LINEs (3.86%) and LTRs elements (1.37%) (FIG. 2C). Overall, our results reveal that while many dsRNA species are induced after DNMTi, Alu elements, but not LTRs/ERVs as previously assumed^{1,2,4-6,20}, are the major source of immunogenic dsRNA induced upon epigenetic therapy.

To uncover the molecular mechanisms of how repetitive elements generate immunogenic dsRNA, we examined all repeats in MDA5-protected transcripts and searched for repeat pairs within a 3 Kb window. Interestingly, we found that the majority of aligned pairs in both baseline and treatment-induced RNA were inverted repeat (IR) Alus (FIG. 3B-C). Moreover, MDA5-protected RNA contained higher levels of IR-Alus compared to non-IR (direct repeat) Alus (FIG. 2D-E). The expression of baseline IR-Alus was maintained but not enhanced after 5-AZA-CdR treatment (FIG. 2D, FIG. 3D) while treatment-induced IR-Alu hybrids became highly expressed in the MDA5-protected RNA only after treatment with 5-AZA-CdR (FIG. 2E, FIG. 3E). For the baseline immunogenic IR-Alu pairs, we found that each repeat within the pair were concordantly highly enriched in MDA5-protected RNA before and after treatment with DNMTi (FIG. 2F). Conversely, for the treatment-induced immunogenic IR-Alu pairs, we found that both repeats within the pair were either not expressed or only one repeat was expressed before DNMTi treatment, while both repeats were concordantly expressed in MDA5-protected RNA after treatment with DNMTi (FIG. 2G). Moreover, the majority of IR-Alu pairs were unidirectionally transcribed either in the sense (both repeats in the positive strand: “+/+”) or antisense (both repeats in the negative strand: “−/−”) direction in both baseline (FIG. 2H) and treatment-induced dsRNA (FIG. 2I). Taken together, these observations suggest that IR-Alu elements induced by DNMTi treatment give rise to immunogenic dsRNA via intramolecular pairing of IR-Alus, forming RNA stem-loops, rather than the proposed model of sense/anti-sense transcription⁶. These results also raise the question of whether murine cells will respond differently to epigenetic therapy, as murine SINEs (B1 & B2) are structurally distinct from human SINEs²¹.

DNMTi Induces Cryptic Transcription of Intronic and Intergenic Inverted Alus

Next, we investigated how DNMTi treatment induces expression of IR-Alu elements. We detected 746,470 IR-Alu pairs in the human genome, with about 55% originating from introns and 43% from intergenic regions (FIG. 4A). Consistent with previous studies^{19,22}, most of the baseline immunogenic IR-Alu pairs (MDA5-protected) were within 3′UTRs (35%) and introns (42%) (FIG. 4A). Unexpectedly, the vast majority of treatment-induced IR-Alu pairs were within introns (70%), followed by intergenic (16%) regions (FIG. 4A). These distributions were broadly independent of genomic orientation of each repeat within the pair (FIG. 5A-B). We also investigated the distribution of Alu subfamilies of the immunogenic IR-Alus. While the evolutionary younger AluY subfamilies²³ were observed at the same rate as IR-Alu pairs in the human genome, we observed an enrichment of the more active AluS subfamilies and a depletion of the more ancient AluJ subfamilies²³ among the immunogenic IR-Alus. (FIG. 5C).

In addition, we mapped the average CpG density flanking IR-Alu pairs. Notably, CpG density was high directly upstream of IR-Alu pairs induced by DNMTi treatment (FIG. 4B), but not upstream of baseline IR-Alus (FIG. 5D). Indeed, we detected a high density of bona-fide CpG islands (CGI) upstream of treatment-induced IR-Alu pairs compared to baseline and all other IR-Alu pairs in the human genome (FIG. 4C, FIG. 5E). Most, if not all, CGIs are sites of transcriptional initiation¹³. Of the roughly 25,000 CGIs present in the human genome, nearly half reside outside of promoters of annotated genes and are generally found in intergenic and intronic regions¹³. These “orphan” CGIs can act as transcriptional start sites for noncoding RNA^{13,24} or as alternative promoters of nearby genes^{25,26}. In contrast to CGIs at the promoters of annotated genes, orphan CGIs are much more frequently methylated¹³. We therefore assessed the methylation status of CGIs directly upstream of treatment-induced immunogenic IR-Alus repeats. Analysis of a panel of 51 CRC cancer cell lines from the Sanger's GDSC project²⁷ revealed that the majority (57%) of CGIs adjacent to immunogenic IR-Alus are either partially or fully methylated in untreated cells (FIG. 6A. These results were validated across a larger panel of 988 cancer cell lines from 33 different cancer types²⁷ (FIG. 6B).

Active promoters and enhancers are often marked by H3K4me3 and H3K27ac, respectively. We performed genome-wide CUT&RUN (cleavage under targets and release using nuclease)²⁸ analysis to assess these modifications at treatment-induced IR-Alus, and found that DNMTi treatment led to elevated levels of H3K4me3, but not H3K27ac, directly upstream of treatment-induced immunogenic IR-Alus repeats (FIG. 4D). Importantly, we assessed the DNA methylation status of these specific regulatory regions that became H3K4me3 marked upstream of the induced IR-Alus following epigenetic therapy and found the majority of these regions to be frequently methylated before treatment. We observed that 91% of these regions were either partially or fully methylated in the panel of 51 CRC cell lines (FIG. 6C) and 94% in the panel of 988 pan-cancer cell lines (FIG. 6D). Moreover, we observed that the DNA methylation status of these regulatory regions upstream of induced IR-Alus were inversely correlated (r=−0.23, p=7.58-13) with the expression of a panel of Interferon-Stimulated Genes (n=22 ISGs) from our viral mimicry signature¹ across the 988 pan-cancer cell lines (FIG. 6E).

Overall, our data suggest that orphan CGIs located upstream of treatment-induced IR-Alu regions in intronic and intergenic regions are frequently methylated and act as sites of cryptic transcription initiation upon treatment with DNA demethylating drugs, mediating the formation of immunogenic dsRNA (FIG. 4E-F and FIG. 5F). In contrast to treatment-induced IR-Alu regions, IR-Alus transcribed basally are mainly located at 3′-UTR (FIG. 4A and FIG. 4G) and thus likely originate from annotated transcriptional start sites (TSS) as previously suggested^{19,22}. Thus, DNMT inhibitors selectivity activate cryptic transcription of intronic and intergenic IR-Alus, leading to RNA stem-loops that stimulate MDA5 (FIG. 4H). These results highlight a previously unappreciated role of intronic and intergenic orphan CGIs as cryptic promoters of immunogenic IR-Alus. Moreover, the fact that immunogenic IR-Alus are directly regulated by CGIs implies a putative physiological function for these repeats, as CGIs are evolutionary conserved in mammals¹³.

To further characterize how these immunogenic IR-Alus can reach the cytoplasm, where they can be recognized by MDA5, we assessed the presence of poly(A) signals (PAS) within immunogenic IR-Alu pairs. It is well established that PAS play an important role in pre-mRNA cleavage and polyadenylation, leading to RNA stabilization and nuclear export²⁹. We searched for known poly(A) signal motifs²⁹ in the second half (3′ end) of the downstream Alu for each pair using the MDA5-protected RNA-seq data and observed that most baseline and treatment-induced IR-Alu pairs have at least one PAS, with motifs AAUACA and ACUAAA being the most frequently represented (FIG. 7A-B). Interestingly, MDA5-protected RNA-seq signal peaked immediately upstream of PAS motifs and finished abruptly at the 3′ end of these PAS (FIG. 7C-D). These results suggest that cleavage and polyadenylation occur at the 3′ end of the dsRNA stem and suggest a mechanism of how immunogenic dsRNAs are exported to the cytoplasm. Consistent with these findings, analysis of cytoplasmic RNA-seq revealed an enrichment of SINEs (odd-ratio 1.68, p<2.2e-16) compared to total RNA-seq (nuclear and cytoplasmic), among which Alus were particularly enriched (odd-ratio 2.21, p<2.2e-16) in 5-AZA-CdR treated cells (FIG. 7E). Altogether, these results help to explain the accumulation of cytoplasmic dsRNAs observed following epigenetic therapy^{1,6,8} (FIG. 7F).

ADAR1 Restricts Viral Mimicry Response to DNMTi

After identifying the precise nature of immunogenic IR-Alus induced by epigenetic therapy, we sought to investigate whether they create vulnerabilities that could act in synergy with epigenetic therapies. Interestingly, the RNA stem-loops formed by IR-Alu elements are a main substrate for the enzyme ADAR1 (Adenosine Deaminases Acting on RNA)¹⁴. ADAR1 regulates dsRNA through adenosine-to-inosine (A-to-I) editing and subsequent destabilization of RNA duplexes^{15,30}. This mechanism normally prevents baseline Alu sequences from activating MDA5^{15,22} and the downstream interferon response in mammals. Moreover, ADAR1 is itself an interferon-stimulated gene^{22,31,32}. Therefore, we hypothesized that ADAR1 acts as a negative feedback loop after epigenetic therapies, where the interferon response would increase ADAR1 expression and then limit the formation of immunogenic dsRNA and, in turn, the downstream anti-tumour response.

To test this hypothesis, we first determined if ADAR1 depletion affects dsRNA formation by IR-Alus in untreated patient-derived CRCs. We generated ADAR1 knock-down CRCs using two different hairpin RNAs (ADAR1^{KDA/B}) and confirmed depletion of both the constitutively expressed (p110) and interferon-inducible (p150) ADAR1 isoforms (FIG. 8A). Immunostaining with a monoclonal antibody against dsRNA greater than 40 bp revealed a significant increase in cytosolic dsRNA in ADAR1-depleted cells compared to wild-type cells, as expected (FIG. 8B, FIG. 9A). Importantly, cells treated with 5-AZA-CdR up-regulated ADAR1 expression, which was sustained for about fourteen days after drug withdrawal (FIG. 8C). To determine if MDA5 ligands also increase upon ADAR1 depletion, we performed the MDA5 protection assay followed by RNA-Seq, as above. Notably, SINE elements were enriched in MDA5-protected RNA (63.9%) compared to Total CytoRNA (8.6%) isolated from ADAR1-depleted cells (FIG. 8D-E). To examine the MDA5 ligands that are specifically induced by ADAR1 depletion, we excluded those that were present in both wild-type and ADAR-depleted cells (ADAR1^{KDA}-induced immunogenic RNAs) (FIG. 9B). This analysis revealed that the immunogenic dsRNA induced by ADAR1 depletion are enriched for Alus (76.76%, FIG. 8F). Again, these immunogenic Alus were primarily IR-Alu pairs unidirectionally transcribed in either the sense (+/+) or antisense (−/−) direction and induced upon ADAR1 depletion (FIG. 9C-J). These results suggest that ADAR1 can indeed destabilize immunogenic IR-Alus and could act as a negative feedback loop restricting the epigenetic therapy-mediated viral mimicry response.

Next, we tested whether epigenetic therapy can induce and activate ADAR1 editing activity. As a positive control, we verified that CRC cells transiently transfected with synthetic dsRNA (Poly(I:C)) displayed acute up-regulation of the interferon-inducible isoform of ADAR1 (p150) (FIG. 10A). Moreover, we found that treatment with 5-AZA-CdR resulted in genome-wide increased A-to-I editing of MDA5-protected Alus, and that depletion of ADAR1 abrogated this effect (FIG. 8G). Taken together, these results suggest that DNMTi treatment stimulates ADAR1 transcription and ADAR1-mediated A-to-I editing of immunogenic IR-Alu dsRNA.

Based on these findings, we hypothesized that ADAR1 depletion would synergize with the viral mimicry response induced by DNMTi treatment. To assess how ADAR1 depletion affects 5-AZA-CdR treated samples, we performed RNA-seq analyses in patient-derived CRC cells. Gene Set Enrichment Analysis (GSEA) revealed that the type I interferon response was the most enriched pathway between wild-type and ADAR1-depleted samples treated with 5-AZA-CdR (FIG. 8H). Expression of IFN stimulated genes (ISGs) including MDA5, ISG15 and DDX58 was also enhanced upon ADAR1 depletion in two other human CRC cell lines treated with 5-AZA-CdR (FIG. 11). Strikingly, these effects persisted as long as twenty-four days after initial treatment (FIG. 8I, FIG. 10B). In contrast, transfection of Poly(I:C) in both wild-type and ADAR1-depleted cells lead to comparable activation of ISGs across time-points (FIG. 10C-D). Since Poly(I:C) has no adenosine, ADAR1 cannot mediate A-to-I editing of this dsRNA substrate, suggesting that the synergistic effect of ADAR1 depletion on DNMTi is due to loss of its catalytic activity as opposed to other functions such as RNA-binding activity³³. As a molecular read-out of dsRNA pattern recognition activation, we measured MAVS aggregation in the mitochondrial fraction of cell lysates³⁴. Depletion of ADAR1 in 5-AZA-CdR-treated cells resulted in enhanced MAVS activation (FIG. 8J). In addition, we generated CRCs cells that overexpress the interferon inducible isoform of ADAR1 (ADAR1p150) (FIG. 10E-F) and observed it rescues the ability of 5-AZA-CdR to induce viral mimicry, as measured by expression of ISGs and dsRNA formation (FIG. 10G-H). Taken together, our findings establish that ADAR1 acts as a negative feedback loop, restricting the viral mimicry response induced by DNMTi treatment in cancer cells.

ADAR1 Depletion Synergizes with DNMTi Treatment

Finally, we sought to investigate whether ADAR1 inhibition would synergize with epigenetic therapy to increase the anti-tumour response. Indeed, ADAR1 knock-down causes four to six-fold reduction on 5-AZA-CdR EC50 in vitro (FIG. 12A). To examine the potential synergy in vivo, we injected ADAR1^{KDA/B} or wildtype patient-derived xenograft CRC cells subcutaneously into immune-deficient NOD.Cg-Prkdc (NSG) mice, and treated the mice with 5-AZA-CdR or vehicle (PBS) (FIG. 12B and FIG. 13A). Low dose 5-AZA-CdR treatment had very limited anti-tumour activity in mice, similar to observations in clinical trials for human solid tumours³⁵ (FIG. 12C-E). However, low dose 5-AZA-CdR treatments showed a remarkable anti-tumour activity in mice injected with ADAR1^{KDA/B} cells (FIG. 12C-E). Of note, ADAR1^{KDA/B} and wild-type cells grew equivalently in PBS-treated mice (FIG. 12C-E), consistent with recent reports showing that ADAR1 depletion alone does not have a significant anti-tumour effect in most cancer cells^{36,37}. Thus, ADAR1 inhibition in human cancer cells profoundly sensitizes tumours to 5-AZA-CdR treatment in vivo.

One of the major challenges facing conventional chemotherapy is the persistence of cancer initiating cells (CICs), often resulting in relapse³⁸⁻⁴⁰. We and others have previously shown that 5-AZA-CdR treatment can target colorectal cancer CICs by inducing the viral mimicry response^{1,41}. We therefore tested whether ADAR1^{KD} by itself or in combination with 5-AZA-CdR can decrease tumour initiation. To this end, we first employed an in vitro sphere initiation assay followed by limiting dilution analysis (LDA) using patient-derived CRC cells. Depletion of ADAR1 alone had minimal effects, but reduced the spheroid forming potential of CRC cells treated with 5-AZA-CdR (FIG. 13B). In addition, we performed in vivo tumour initiation assays by injecting serially diluted patient-derived CRC cells into NSG immunodeficient mice and inspected tumour engraftment for up to 6 months after injection (FIG. 12F). Relative to wild-type, ADAR1^{KDA/B} cells showed a substantially reduced tumour engraftment potential in mice treated with 5-AZA-CdR, but not in mock-treated mice (Table 1, FIG. 12G).

Finally, to investigate the potential of ADAR1 inhibition to synergize with other anti-cancer therapies by enhancing viral mimicry response, we combined ADAR1 depletion to CDK4/6 inhibition. CDK4/6 inhibitors were recently shown to up-regulate dsRNA expression, by reducing DNMT1 levels, leading to an interferon response³. Indeed, we observed a small accumulation of dsRNA in wild-type CRC cells treated with the CDK4/6 inhibitor plabociclib (FIG. 13C-D). However, depletion of ADAR1 in combination with palbociclib treatment resulted in stronger accumulation of dsRNA in the cytosol (FIG. 13C-D). Likewise, we observed synergistic up-regulation of interferon-stimulated genes by ADAR1 knockdown and palbociclib treatment (FIG. 13E).

Further, we analyzed the A to I editing in the RNAseq of the cell lines that are different in the response to ADAR1 inhibition. We quantified the A to I editing in the treatment-induced IR Aus in the RNA-seq of three cell lines which are NCIH716, COLO678 and NCIH747. Cell lines that have lower level of A to I editing in treatment-induced IR-Alus are resistant to ADAR1 inhibition, while cell lines that have higher level of A to I editing are sensitive to the ADAR1 inhibition (FIG. 14A-B). This analysis shows that A to I editing of IR Alus can predict the level of ADAR1 dependency and sensitivity to putative ADAR1 inhibitors.

In summary, our study identifies IR-Alus, rather than bi-directional ERVs transcripts, as the main immunogenic dsRNA induced by DNA hypomethylating agents. Molecularly, our data suggest that these treatment-induced IR pairs form stem-loops to act as endogenous, immunogenic dsRNA. We provide evidence that most of the immunogenic dsRNA are induced from cryptic promoters within intronic or intergenic regions, which are enriched for orphan CpG islands and acquire active epigenetic marks upon DNMTi treatment. The presence of CpG Islands suggest that treatment-induced immunogenic repeats have physiological roles, in line with our previous hypothesis that viral mimicry represents an ‘epigenetic checkpoint’ to reduce cell fitness in response to a genome-wide loss of heterochromatin, for instance due to environmental or genetic factors^{42,43}. Moreover, our study reveals that ADAR1 induction establishes a negative feedback loop that limits the ability of epigenetic therapy to induce a robust and sustained immunogenic dsRNA response. Indeed, inhibition of ADAR1 in combination with DNMTi treatment profoundly restrained colorectal cancer tumour growth in vitro and in vivo and reduced the frequency of cancer initiating cells that have self-renewal potential. Thus, our findings point to the potential use of immunogenic IR-Alus as biomarkers for epigenetic therapy response and suggest that combination of ADAR1 inhibition with epigenetic therapy represents a novel and potent therapeutic strategy. Finally, our findings show that measuring A to I editing in IR-Alus is predictive of ADAR1 dependency and, therefore, provide prognostic information regarding sensitivity to ADAR1 inhibitors.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1 Roulois, D. et al. DNA-Demethylating Agents Target Colorectal Cancer Cells by Inducing Viral Mimicry by Endogenous Transcripts. Cell 162, 961-973, doi:10.1016/j.cell.2015.07.056 (2015).
2 Chiappinelli, K. B. et al. Inhibiting DNA Methylation Causes an Interferon Response in Cancer via dsRNA Including Endogenous Retroviruses. Cell 162, 974-986, doi:10.1016/j.cell.2015.07.011 (2015).
3 Goel, S. et al. CDK4/6 inhibition triggers anti-tumour immunity. Nature 548, 471-475, doi:10.1038/nature23465 (2017).
4 Brocks, D. et al. DNMT and HDAC inhibitors induce cryptic transcription start sites encoded in long terminal repeats. Nat Genet 49, 1052-1060, doi:10.1038/ng.3889 (2017).
5 Zhang, H. et al. Targeting CDK9 Reactivates Epigenetically Silenced Genes in Cancer. Cell 175, 1244-1258 e1226, doi:10.1016/j.cell.2018.09.051 (2018).
6 Sheng, W. et al. LSD1 Ablation Stimulates Anti-tumor Immunity and Enables Checkpoint Blockade. Cell 174, 549-563 e519, doi:10.1016/j.cell.2018.05.052 (2018).
7 Liu, M. et al. Dual Inhibition of DNA and Histone Methyltransferases Increases Viral Mimicry in Ovarian Cancer Cells. Cancer Res 78, 5754-5766, doi:10.1158/0008-5472.CAN-17-3953 (2018).
8 Cuellar, T. L. et al. Silencing of retrotransposons by SETDB1 inhibits the interferon response in acute myeloid leukemia. J Cell Biol 216, 3535-3549, doi:10.1083/jcb.201612160 (2017).
9 Hirukawa, A. et al. Reduction of Global H3K27me(3) Enhances HER2/ErbB2 Targeted Therapy. Cell Rep 29, 249-257 e248, doi:10.1016/j.celrep.2019.08.105 (2019).
10 Guler, G. D. et al. Repression of Stress-Induced LINE-1 Expression Protects Cancer Cell Subpopulations from Lethal Drug Exposure. Cancer Cell 32, 221-237 e213, doi:10.1016/j.ccell.2017.07.002 (2017).
11 Loo Yau, H., Ettayebi, I. & De Carvalho, D. D. The Cancer Epigenome: Exploiting Its Vulnerabilities for Immunotherapy. Trends Cell Biol 29, 31-43, doi:10.1016/j.tcb.2018.07.006 (2019).
12 Jones, P. A., Ohtani, H., Chakravarthy, A. & De Carvalho, D. D. Epigenetic therapy in immune-oncology. Nat Rev Cancer 19, 151-161, doi:10.1038/541568-019-0109-9 (2019).
13 Deaton, A. M. & Bird, A. CpG islands and the regulation of transcription. Genes Dev 25, 1010-1022, doi:10.1101/gad.2037511 (2011).
14 Levanon, E. Y. et al. Systematic identification of abundant A-to-I editing sites in the human transcriptome. Nat Biotechnol 22, 1001-1005, doi:10.1038/nbt996 (2004).
15 Liddicoat, B. J. et al. RNA editing by ADAR1 prevents MDA5 sensing of endogenous dsRNA as nonself. Science 349, 1115-1120, doi:10.1126/science.aac7049 (2015).
16 Mehdipour, P., Murphy, T. & De Carvalho, D. D. The role of DNA-demethylating agents in cancer therapy. Pharmacol Ther, 107416, doi:10.1016/j.pharmthera.2019.107416 (2019).
17 Leruste, A. et al. Clonally Expanded T Cells Reveal Immunogenicity of Rhabdoid Tumors. Cancer Cell 36, 597-612 e598, doi:10.1016/j.ccell.2019.10.008 (2019).
18 Krug, B. et al. Pervasive H3K27 Acetylation Leads to ERV Expression and a Therapeutic Vulnerability in H3K27M Gliomas. Cancer Cell 35, 782-797 e788, doi:10.1016/j.ccell.2019.04.004 (2019).
19 Ahmad, S. et al. Breaching Self-Tolerance to Alu Duplex RNA Underlies MDA5-Mediated Inflammation. Cell 172, 797-810 e713, doi:10.1016/j.cell.2017.12.016 (2018).
20 Canadas, I. et al. Tumor innate immunity primed by specific interferon-stimulated endogenous retroviruses. Nat Med 24, 1143-1150, doi:10.1038/s41591-018-0116-5 (2018).
21 Kriegs, J. O., Churakov, G., Jurka, J., Brosius, J. & Schmitz, J. Evolutionary history of 7SL RNA-derived SINEs in Supraprimates. Trends Genet 23, 158-161, doi:10.1016/j.tig.2007.02.002 (2007).
22 Chung, H. et al. Human ADAR1 Prevents Endogenous RNA from Triggering Translational Shutdown. Cell 172, 811-824 e814, doi:10.1016/j.cell.2017.12.038 (2018).
23 Deininger, P. Alu elements: know the SINEs. Genome Biol 12, 236, doi:10.1186/gb-2011-12-12-236 (2011).
24 Sleutels, F., Zwart, R. & Barlow, D. P. The non-coding Air RNA is required for silencing autosomal imprinted genes. Nature 415, 810-813, doi:10.1038/415810a (2002).
25 Maunakea, A. K. et al. Conserved role of intragenic DNA methylation in regulating alternative promoters. Nature 466, 253-257, doi:10.1038/nature09165 (2010).
26 Sarda, S. & Hannenhalli, S. Orphan CpG islands as alternative promoters. Transcription 9, 171-176, doi:10.1080/21541264.2017.1373209 (2018).
27 Iorio, F. et al. A Landscape of Pharmacogenomic Interactions in Cancer. Cell 166, 740-754, doi:10.1016/j.cell.2016.06.017 (2016).
28 Skene, P. J. & Henikoff, S. An efficient targeted nuclease strategy for high-resolution mapping of DNA binding sites. Elife 6, doi:10.7554/eLife.21856 (2017).
29 Gruber, A. J. et al. A comprehensive analysis of 3′ end sequencing data sets reveals novel polyadenylation signals and the repressive role of heterogeneous ribonucleoprotein C on cleavage and polyadenylation. Genome Res 26, 1145-1159, doi:10.1101/gr.202432.115 (2016).
30 Samuel, C. E. Adenosine deaminases acting on RNA (ADARs) are both antiviral and proviral. Virology 411, 180-193, doi:10.1016/j.virol.2010.12.004 (2011).
31 Pestal, K. et al. Isoforms of RNA-Editing Enzyme ADAR1 Independently Control Nucleic Acid Sensor MDA5-Driven Autoimmunity and Multi-organ Development. Immunity 43, 933-944, doi:10.1016/j.immuni.2015.11.001 (2015).
32 Mannion, N. M. et al. The RNA-editing enzyme ADAR1 controls innate immune responses to RNA. Cell Rep 9, 1482-1494, doi:10.1016/j.celrep.2014.10.041 (2014).
33 Nishikura, K. A-to-I editing of coding and non-coding RNAs by ADARs. Nat Rev Mol Cell Biol 17, 83-96, doi:10.1038/nrm.2015.4 (2016).
34 Hou, F. et al. MAVS forms functional prion-like aggregates to activate and propagate antiviral innate immune response. Cell 146, 448-461, doi:10.1016/j.cell.2011.06.041 (2011).
35 Issa, J. P. & Kantarjian, H. M. Targeting DNA methylation. Clin Cancer Res 15, 3938-3946, doi:10.1158/1078-0432.CCR-08-2783 (2009).
36 Ishizuka, J. J. et al. Loss of ADAR1 in tumours overcomes resistance to immune checkpoint blockade. Nature 565, 43-48, doi:10.1038/s41586-018-0768-9 (2019).
37 Gannon, H. S. et al. Identification of ADAR1 adenosine deaminase dependency in a subset of cancer cells. Nat Commun 9, 5450, doi:10.1038/s41467-018-07824-4 (2018).
38 Kreso, A. et al. Self-renewal as a therapeutic target in human colorectal cancer. Nat Med 20, 29-36, doi:10.1038/nm.3418 (2014).
39 Merlos-Suarez, A. et al. The intestinal stem cell signature identifies colorectal cancer stem cells and predicts disease relapse. Cell Stem Cell 8, 511-524, doi:10.1016/j.stem.2011.02.020 (2011).
40 Phi, L. T. H. et al. Cancer Stem Cells (CSCs) in Drug Resistance and their Therapeutic Implications in Cancer Treatment. Stem Cells Int 2018, 5416923, doi:10.1155/2018/5416923 (2018).
41 Saito, Y. et al. Inhibition of DNA Methylation Suppresses Intestinal Tumor Organoids by Inducing an Anti-Viral Response. Sci Rep 6, 25311, doi:10.1038/srep25311 (2016).
42 Ishak, C. A., Classon, M. & De Carvalho, D. D. Deregulation of Retroelements as an Emerging Therapeutic Opportunity in Cancer. Trends Cancer 4, 583-597, doi:10.1016/j.trecan.2018.05.008 (2018).
43 Ishak, C. A. & De Carvalho, D. D. Reactivation of Endogenous Retroelements in Cancer Development and Therapy. Annual Review of Cancer Biology 4 (2019).

TABLE 1
Number of engrafted tumours compared to the number
of injected tumours in each cohort for in vivo LDA.

| | Number of Engrafted | |
|---|---|---|
| Number of cells | tumours/number of | |
| injected | injected tumours | Conditions |
| 10,000 | 8/8 | ADAR1^{WT} Mock-treated |
| 1,000 | 8/8 | ADAR1^{WT} Mock-treated |
| 100 | 8/8 | ADAR1^{WT} Mock-treated |
| 10 | 8/16 | ADAR1^{WT} Mock-treated |
| 10,000 | 7/8 | ADAR1^{WT} 5-AZA-CdR |
| 1,000 | 5/8 | ADAR1^{WT} 5-AZA-CdR |
| 100 | 3/8 | ADAR1^{WT} 5-AZA-CdR |
| 10 | 0/16 | ADAR1^{WT} 5-AZA-CdR |
| 10,000 | 8/8 | ADAR1^{KDA} Mock-treated |
| 1,000 | 7/8 | ADAR1^{KDA} Mock-treated |
| 100 | 7/8 | ADAR1^{KDA} Mock-treated |
| 10 | 7/16 | ADAR1^{KDA} Mock-treated |
| 10,000 | 5/8 | ADAR1^{KDA} 5-AZA-CdR |
| 1,000 | 3/8 | ADAR1^{KDA} 5-AZA-CdR |
| 100 | 0/8 | ADAR1^{KDA} 5-AZA-CdR |
| 10 | 0/16 | ADAR1^{KDA} 5-AZA-CdR |
| 10,000 | 8/8 | ADAR1^{KDB} Mock-treated |
| 1,000 | 6/8 | ADAR1^{KDB} Mock-treated |
| 100 | 6/8 | ADAR1^{KDB} Mock-treated |
| 10 | 1/16 | ADAR1^{KDB} Mock-treated |
| 10,000 | 6/8 | ADAR1^{KDB} 5-AZA-CdR |
| 1,000 | 1/8 | ADAR1^{KDB} 5-AZA-CdR |
| 100 | 0/8 | ADAR1^{KDB} 5-AZA-CdR |
| 10 | 0/16 | ADAR1^{KDB} 5-AZA-CdR |

TABLE A

| | | |
|---|---|---|
| chr1 | 1342004 | 1342952 |
| chr1 | 1509482 | 1510747 |
| chr1 | 1670756 | 1671101 |
| chr1 | 1821405 | 1823272 |
| chr1 | 6452814 | 6454185 |
| chr1 | 6614136 | 6614835 |
| chr1 | 8002408 | 8002699 |
| chr1 | 9188503 | 9189743 |
| chr1 | 9294401 | 9295118 |
| chr1 | 10003165 | 10003585 |
| chr1 | 10532177 | 10532800 |
| chr1 | 11072319 | 11073245 |
| chr1 | 11119030 | 11120634 |
| chr1 | 15911088 | 15911608 |
| chr1 | 16161260 | 16162880 |
| chr1 | 19638207 | 19639309 |
| chr1 | 20987516 | 20988238 |
| chr1 | 21503115 | 21503836 |
| chr1 | 22351917 | 22352194 |
| chr1 | 23036823 | 23038362 |
| chr1 | 24306363 | 24307017 |
| chr1 | 26737246 | 26738148 |
| chr1 | 27216161 | 27217045 |
| chr1 | 27286065 | 27287101 |
| chr1 | 28844653 | 28845293 |
| chr1 | 28857037 | 28857311 |
| chr1 | 28969553 | 28969898 |
| chr1 | 29507976 | 29508686 |
| chr1 | 31538412 | 31538938 |
| chr1 | 32666231 | 32666460 |
| chr1 | 33116543 | 33117363 |
| chr1 | 35544927 | 35545303 |
| chr1 | 39456655 | 39457271 |
| chr1 | 40505411 | 40506703 |
| chr1 | 41119852 | 41120136 |
| chr1 | 44412073 | 44412804 |
| chr1 | 45987163 | 45987803 |
| chr1 | 46049590 | 46050582 |
| chr1 | 46767425 | 46769036 |
| chr1 | 51425447 | 51426070 |
| chr1 | 52831511 | 52832181 |
| chr1 | 62190647 | 62190942 |
| chr1 | 70671183 | 70671442 |
| chr1 | 93811218 | 93811940 |
| chr1 | 100598130 | 100598754 |
| chr1 | 109618579 | 109618832 |
| chr1 | 110091039 | 110091590 |
| chr1 | 110880394 | 110880624 |
| chr1 | 112938495 | 112939113 |
| chr1 | 113161591 | 113162571 |
| chr1 | 114447454 | 114448024 |
| chr1 | 145610679 | 145611143 |
| chr1 | 147834674 | 147835013 |
| chr1 | 150039324 | 150039815 |
| chr1 | 150241047 | 150241654 |
| chr1 | 150266476 | 150266689 |
| chr1 | 150601710 | 150602201 |
| chr1 | 150980735 | 150981092 |
| chr1 | 151042870 | 151043537 |
| chr1 | 151170847 | 151171532 |
| chr1 | 151431540 | 151432032 |
| chr1 | 155197116 | 155197534 |
| chr1 | 155531914 | 155533280 |
| chr1 | 156611639 | 156612447 |
| chr1 | 160990718 | 160991225 |
| chr1 | 178693968 | 178695075 |
| chr1 | 201924336 | 201925053 |
| chr1 | 202776003 | 202777727 |
| chr1 | 202830061 | 202830923 |
| chr1 | 203764568 | 203765293 |
| chr1 | 204485473 | 204485831 |
| chr1 | 211500151 | 211500636 |
| chr1 | 212457967 | 212459694 |
| chr1 | 217804267 | 217804477 |
| chr1 | 225965330 | 225965734 |
| chr1 | 226069605 | 226070898 |
| chr1 | 231473012 | 231474382 |
| chr1 | 235291740 | 235292387 |
| chr1 | 235490338 | 235492160 |
| chr1 | 235667089 | 235668619 |
| chr1 | 246670148 | 246670768 |
| chr1 | 247170868 | 247171434 |
| chr1 | 247463848 | 247464710 |
| chr2 | 9346383 | 9347944 |
| chr2 | 9514723 | 9514995 |
| chr2 | 9770189 | 9771562 |
| chr2 | 10829724 | 10830241 |
| chr2 | 25015879 | 25016627 |
| chr2 | 26256301 | 26257567 |
| chr2 | 27341567 | 27341923 |
| chr2 | 28974212 | 28975388 |
| chr2 | 29117366 | 29118025 |
| chr2 | 32502965 | 32503471 |
| chr2 | 38603569 | 38604510 |
| chr2 | 38977535 | 38978684 |
| chr2 | 39663897 | 39664667 |
| chr2 | 43822928 | 43823370 |
| chr2 | 44222741 | 44223732 |
| chr2 | 44588954 | 44589358 |
| chr2 | 47596629 | 47597256 |
| chr2 | 54197776 | 54198490 |
| chr2 | 55746675 | 55746999 |
| chr2 | 55844267 | 55845065 |
| chr2 | 61292868 | 61293585 |
| chr2 | 61765406 | 61766091 |
| chr2 | 62115445 | 62115990 |
| chr2 | 64245934 | 64246893 |
| chr2 | 65356735 | 65357614 |
| chr2 | 65454553 | 65455444 |
| chr2 | 69614119 | 69614616 |
| chr2 | 69968749 | 69969592 |
| chr2 | 70475427 | 70476151 |
| chr2 | 71558723 | 71559525 |
| chr2 | 75937657 | 75938139 |
| chr2 | 85197445 | 85199321 |
| chr2 | 86422068 | 86422858 |
| chr2 | 86946401 | 86948451 |
| chr2 | 87157803 | 87158009 |
| chr2 | 88125153 | 88125434 |
| chr2 | 88354840 | 88355574 |
| chr2 | 91873047 | 91874525 |
| chr2 | 96930859 | 96932291 |
| chr2 | 97303912 | 97304387 |
| chr2 | 101924364 | 101925357 |
| chr2 | 102313549 | 102314478 |
| chr2 | 105946164 | 105946773 |
| chr2 | 109150330 | 109151301 |
| chr2 | 109335768 | 109336670 |
| chr2 | 109402907 | 109403566 |
| chr2 | 110370906 | 110373301 |
| chr2 | 113032793 | 113033496 |
| chr2 | 113190115 | 113191274 |
| chr2 | 113914681 | 113915396 |
| chr2 | 114195165 | 114195563 |
| chr2 | 114513712 | 114514621 |
| chr2 | 114647669 | 114648312 |
| chr2 | 120517066 | 120518010 |
| chr2 | 122406398 | 122407824 |
| chr2 | 128283578 | 128284524 |
| chr2 | 128568321 | 128569064 |
| chr2 | 128642787 | 128644407 |
| chr2 | 130938840 | 130940428 |
| chr2 | 131099469 | 131100863 |
| chr2 | 131555096 | 131555929 |
| chr2 | 134949067 | 134949727 |
| chr2 | 135675772 | 135677088 |
| chr2 | 153191492 | 153192974 |
| chr2 | 159313172 | 159313416 |
| chr2 | 170655289 | 170655757 |
| chr2 | 170681219 | 170681587 |
| chr2 | 172543901 | 172544690 |
| chr2 | 173292099 | 173293183 |
| chr2 | 175351364 | 175352154 |
| chr2 | 178077185 | 178078215 |
| chr2 | 178128273 | 178129847 |
| chr2 | 201981062 | 201981689 |
| chr2 | 203102615 | 203103328 |
| chr2 | 203130313 | 203130760 |
| chr2 | 203239865 | 203242418 |
| chr2 | 204399490 | 204400179 |
| chr2 | 207023910 | 207024981 |
| chr2 | 208546082 | 208546562 |
| chr2 | 216176549 | 216177132 |
| chr2 | 219081653 | 219082032 |
| chr2 | 219252421 | 219252631 |
| chr2 | 219263098 | 219265556 |
| chr2 | 219432767 | 219434061 |
| chr2 | 220252008 | 220252638 |
| chr2 | 224701962 | 224702790 |
| chr2 | 224808923 | 224810219 |
| chr2 | 230785912 | 230787665 |
| chr2 | 231577309 | 231578504 |
| chr2 | 231902026 | 231903122 |
| chr2 | 233562005 | 233562547 |
| chr2 | 234160100 | 234160735 |
| chr2 | 238969080 | 238969919 |
| chr2 | 239228874 | 239229436 |
| chr2 | 240964645 | 240965271 |
| chr2 | 242211289 | 242212308 |
| chr2 | 242254299 | 242256026 |
| chr2 | 242447017 | 242448558 |
| chr2 | 242663547 | 242663872 |
| chr3 | 9437945 | 9439760 |
| chr3 | 9811466 | 9811736 |
| chr3 | 9993730 | 9994136 |
| chr3 | 10149750 | 10150066 |
| chr3 | 10276025 | 10276415 |
| chr3 | 11746577 | 11746824 |
| chr3 | 15900497 | 15901990 |
| chr3 | 17783646 | 17784455 |
| chr3 | 19988333 | 19989321 |
| chr3 | 23244050 | 23245071 |
| chr3 | 23847871 | 23848633 |
| chr3 | 27524934 | 27526180 |
| chr3 | 28282826 | 28283457 |
| chr3 | 30647987 | 30648881 |
| chr3 | 32279423 | 32280742 |
| chr3 | 40494414 | 40494899 |
| chr3 | 44518838 | 44519368 |
| chr3 | 44802852 | 44803618 |
| chr3 | 47204789 | 47206336 |
| chr3 | 47516997 | 47517650 |
| chr3 | 48252071 | 48252370 |
| chr3 | 48754577 | 48754904 |
| chr3 | 49027116 | 49028356 |
| chr3 | 49130677 | 49131845 |
| chr3 | 49466478 | 49466843 |
| chr3 | 50374264 | 50375629 |
| chr3 | 56716492 | 56717658 |
| chr3 | 57583020 | 57583611 |
| chr3 | 57993838 | 57994704 |
| chr3 | 75483802 | 75485289 |
| chr3 | 88107550 | 88108509 |
| chr3 | 101280545 | 101280952 |
| chr3 | 113775147 | 113775582 |
| chr3 | 115502725 | 115503581 |
| chr3 | 124605059 | 124606729 |
| chr3 | 125093668 | 125094365 |
| chr3 | 128902134 | 128902978 |
| chr3 | 129829971 | 129831426 |
| chr3 | 130612285 | 130613485 |
| chr3 | 131100341 | 131101151 |
| chr3 | 136470433 | 136471923 |
| chr3 | 138553159 | 138554078 |
| chr3 | 139108249 | 139108985 |
| chr3 | 141943946 | 141944573 |
| chr3 | 142297219 | 142297849 |
| chr3 | 149374709 | 149376300 |
| chr3 | 155572358 | 155572604 |
| chr3 | 169379437 | 169381056 |
| chr3 | 169684394 | 169685006 |
| chr3 | 170074786 | 170076070 |
| chr3 | 172428416 | 172428950 |
| chr3 | 172468372 | 172468845 |
| chr3 | 176915511 | 176915854 |
| chr3 | 179040466 | 179041632 |
| chr3 | 179065560 | 179065979 |
| chr3 | 180630152 | 180630996 |
| chr3 | 182697749 | 182698731 |
| chr3 | 183353234 | 183354816 |
| chr3 | 183415602 | 183416069 |
| chr3 | 183852778 | 183853386 |
| chr3 | 183872925 | 183873712 |
| chr3 | 185303822 | 185304497 |
| chr3 | 185541048 | 185544146 |
| chr3 | 194207385 | 194208785 |
| chr3 | 194350336 | 194351847 |
| chr3 | 195452004 | 195452645 |
| chr3 | 195808278 | 195809219 |
| chr3 | 196438874 | 196439734 |
| chr3 | 196594576 | 196595486 |
| chr3 | 197024427 | 197025506 |
| chr4 | 330162 | 332068 |
| chr4 | 467471 | 467839 |
| chr4 | 492839 | 493379 |
| chr4 | 775071 | 775963 |
| chr4 | 1828588 | 1828802 |
| chr4 | 1843080 | 1843352 |
| chr4 | 2401507 | 2401759 |
| chr4 | 2537098 | 2539421 |
| chr4 | 3043018 | 3043547 |
| chr4 | 3956663 | 3958275 |
| chr4 | 6783872 | 6785283 |
| chr4 | 17811954 | 17813227 |
| chr4 | 39528728 | 39529723 |
| chr4 | 39640156 | 39640923 |
| chr4 | 39699359 | 39700214 |
| chr4 | 39978066 | 39979792 |
| chr4 | 48832807 | 48833569 |
| chr4 | 56261697 | 56262503 |
| chr4 | 56719704 | 56720228 |
| chr4 | 71553854 | 71554473 |
| chr4 | 71767793 | 71768663 |
| chr4 | 73934944 | 73935650 |
| chr4 | 74124405 | 74125130 |
| chr4 | 76649538 | 76650457 |
| chr4 | 76861089 | 76862426 |
| chr4 | 77869440 | 77872122 |
| chr4 | 84405696 | 84406521 |
| chr4 | 87855873 | 87857204 |
| chr4 | 89204952 | 89206069 |
| chr4 | 128801886 | 128802696 |
| chr4 | 128886004 | 128886971 |
| chr4 | 139936443 | 139937428 |
| chr4 | 140097251 | 140099836 |
| chr4 | 140201064 | 140201449 |
| chr4 | 151936653 | 151937519 |
| chr4 | 152020470 | 152021110 |
| chr4 | 152246191 | 152246940 |
| chr4 | 166248454 | 166249063 |
| chr5 | 14143017 | 14145350 |
| chr5 | 16935555 | 16936408 |
| chr5 | 21459469 | 21459852 |
| chr5 | 32173598 | 32174837 |
| chr5 | 33440635 | 33441365 |
| chr5 | 34007796 | 34008105 |
| chr5 | 34656123 | 34657330 |
| chr5 | 34915293 | 34916240 |
| chr5 | 43120926 | 43122005 |
| chr5 | 43313191 | 43313924 |
| chr5 | 43396897 | 43397364 |
| chr5 | 43483519 | 43484555 |
| chr5 | 60626505 | 60629809 |
| chr5 | 64858775 | 64859283 |
| chr5 | 65220759 | 65222416 |
| chr5 | 68665087 | 68665866 |
| chr5 | 69321024 | 69321534 |
| chr5 | 78809347 | 78810468 |
| chr5 | 79703436 | 79704444 |
| chr5 | 79783635 | 79784145 |
| chr5 | 112196849 | 112197247 |
| chr5 | 112312387 | 112313006 |
| chr5 | 125759047 | 125759298 |
| chr5 | 125930601 | 125931049 |
| chr5 | 130500226 | 130501095 |
| chr5 | 131705169 | 131705971 |
| chr5 | 132298789 | 132299740 |
| chr5 | 132387100 | 132388369 |
| chr5 | 133511971 | 133512902 |
| chr5 | 133702629 | 133702905 |
| chr5 | 133747366 | 133747799 |
| chr5 | 134074083 | 134074494 |
| chr5 | 134181483 | 134182250 |
| chr5 | 134209887 | 134210504 |
| chr5 | 137368105 | 137368722 |
| chr5 | 137878264 | 137879425 |
| chr5 | 138609320 | 138609851 |
| chr5 | 138677333 | 138678248 |
| chr5 | 138940584 | 138941792 |
| chr5 | 138897460 | 138897975 |
| chr5 | 141071624 | 141071967 |
| chr5 | 141703749 | 141705023 |
| chr5 | 141736249 | 141736980 |
| chr5 | 145582725 | 145583511 |
| chr5 | 148930249 | 148931244 |
| chr5 | 151137992 | 151138418 |
| chr5 | 154134139 | 154137009 |
| chr5 | 159545606 | 159546534 |
| chr5 | 167689227 | 167689738 |
| chr5 | 172483115 | 172483862 |
| chr5 | 175874629 | 175875428 |
| chr5 | 176432854 | 176433908 |
| chr5 | 176543900 | 176544228 |
| chr5 | 176873349 | 176874737 |
| chr5 | 177659197 | 177659927 |
| chr5 | 178286874 | 178287942 |
| chr5 | 179105169 | 179106412 |
| chr5 | 179334344 | 179335115 |
| chr5 | 179921201 | 179922179 |
| chr5 | 180670373 | 180670996 |
| chr6 | 3063811 | 3064553 |
| chr6 | 15244877 | 15246160 |
| chr6 | 27100174 | 27101124 |
| chr6 | 28890951 | 28892013 |
| chr6 | 30180933 | 30182187 |
| chr6 | 30584885 | 30585677 |
| chr6 | 30710307 | 30712440 |
| chr6 | 31125894 | 31126561 |
| chr6 | 31599519 | 31599761 |
| chr6 | 31670594 | 31671274 |
| chr6 | 31830299 | 31830948 |
| chr6 | 32097820 | 32098086 |
| chr6 | 33281112 | 33281827 |
| chr6 | 33588617 | 33589909 |
| chr6 | 34393325 | 34394014 |
| chr6 | 34725063 | 34725306 |
| chr6 | 35310150 | 35310686 |
| chr6 | 35888479 | 35889060 |
| chr6 | 35995245 | 35996372 |
| chr6 | 36514995 | 36515308 |
| chr6 | 37786707 | 37788114 |
| chr6 | 42016243 | 42016731 |
| chr6 | 42072032 | 42072701 |
| chr6 | 42227081 | 42227488 |
| chr6 | 42419510 | 42419743 |
| chr6 | 42531984 | 42532292 |
| chr6 | 42713166 | 42713986 |
| chr6 | 42981408 | 42982077 |
| chr6 | 43543124 | 43544076 |
| chr6 | 43596790 | 43597690 |
| chr6 | 44094708 | 44096134 |
| chr6 | 52226753 | 52228006 |
| chr6 | 52441269 | 52441793 |
| chr6 | 52528939 | 52529651 |
| chr6 | 52859565 | 52859767 |
| chr6 | 56911202 | 56911988 |
| chr6 | 58287429 | 58287828 |
| chr6 | 71122892 | 71123820 |
| chr6 | 71377385 | 71378178 |
| chr6 | 76311330 | 76312563 |
| chr6 | 90529137 | 90529698 |
| chr6 | 99872726 | 99873206 |
| chr6 | 107349276 | 107349720 |
| chr6 | 107955163 | 107956693 |
| chr6 | 111195383 | 111197576 |
| chr6 | 111580363 | 111580887 |
| chr6 | 131383431 | 131384371 |
| chr6 | 133119483 | 133119802 |
| chr6 | 138428188 | 138428816 |
| chr6 | 139013563 | 139014881 |
| chr6 | 139094449 | 139095009 |
| chr6 | 139308498 | 139309559 |
| chr6 | 143771610 | 143772084 |
| chr6 | 149638018 | 149639518 |
| chr6 | 149867031 | 149867574 |
| chr6 | 150067323 | 150067766 |
| chr6 | 150038664 | 150039440 |
| chr6 | 150184344 | 150185505 |
| chr6 | 151711094 | 151712829 |
| chr6 | 159420465 | 159421293 |
| chr6 | 160113557 | 160114863 |
| chr6 | 166755812 | 166756510 |
| chr6 | 168812524 | 168812738 |
| chr7 | 766085 | 766952 |
| chr7 | 2250016 | 2250275 |
| chr7 | 2393628 | 2395174 |
| chr7 | 5372280 | 5372717 |
| chr7 | 5539957 | 5541567 |
| chr7 | 6388210 | 6388743 |
| chr7 | 23338797 | 23339264 |
| chr7 | 23454792 | 23455197 |
| chr7 | 23508184 | 23509712 |
| chr7 | 23570757 | 23571819 |
| chr7 | 25164548 | 25165236 |
| chr7 | 32535086 | 32535540 |
| chr7 | 32930730 | 32931956 |
| chr7 | 43945583 | 43946456 |
| chr7 | 44058608 | 44058988 |
| chr7 | 44621293 | 44622377 |
| chr7 | 45002111 | 45002845 |
| chr7 | 56119045 | 56119963 |
| chr7 | 64029902 | 64030972 |
| chr7 | 64254700 | 64255067 |
| chr7 | 64498577 | 64498930 |
| chr7 | 64541192 | 64541752 |
| chr7 | 64535252 | 64535630 |
| chr7 | 65338006 | 65338212 |
| chr7 | 65579764 | 65580200 |
| chr7 | 66021169 | 66022278 |
| chr7 | 66093348 | 66094174 |
| chr7 | 66119075 | 66120404 |
| chr7 | 66146725 | 66148011 |
| chr7 | 66205245 | 66206565 |
| chr7 | 66309503 | 66310040 |
| chr7 | 66460112 | 66460803 |
| chr7 | 66751106 | 66751333 |
| chr7 | 72349774 | 72350765 |
| chr7 | 72395367 | 72396135 |
| chr7 | 72935612 | 72937090 |
| chr7 | 72971527 | 72972220 |
| chr7 | 73021486 | 73021720 |
| chr7 | 73037528 | 73038957 |
| chr7 | 73152564 | 73153716 |
| chr7 | 73867787 | 73868061 |
| chr7 | 74070951 | 74071154 |
| chr7 | 74306265 | 74307187 |
| chr7 | 75114831 | 75115715 |
| chr7 | 75156588 | 75157524 |
| chr7 | 75508166 | 75508759 |
| chr7 | 75677041 | 75677767 |
| chr7 | 76588998 | 76589608 |
| chr7 | 77165936 | 77166255 |
| chr7 | 77325410 | 77326409 |
| chr7 | 87505100 | 87506493 |
| chr7 | 97834643 | 97835193 |
| chr7 | 98923235 | 98923975 |
| chr7 | 99036099 | 99036892 |
| chr7 | 99140766 | 99141151 |
| chr7 | 99678768 | 99679702 |
| chr7 | 99724482 | 99725808 |
| chr7 | 99933238 | 99934087 |
| chr7 | 100025804 | 100028002 |
| chr7 | 100091180 | 100091598 |
| chr7 | 100449772 | 100450617 |
| chr7 | 100860297 | 100861029 |
| chr7 | 101457382 | 101461030 |
| chr7 | 102212867 | 102213262 |
| chr7 | 104653119 | 104654966 |
| chr7 | 104624337 | 104624848 |
| chr7 | 104941752 | 104941978 |
| chr7 | 105028260 | 105029774 |
| chr7 | 106809461 | 106809815 |
| chr7 | 121035820 | 121036670 |
| chr7 | 128095678 | 128096197 |
| chr7 | 129592032 | 129592891 |
| chr7 | 141401432 | 141402207 |
| chr7 | 148844254 | 148845556 |
| chr7 | 150725173 | 150726018 |
| chr7 | 150746002 | 150746381 |
| chr7 | 152372980 | 152373421 |
| chr7 | 158596444 | 158597251 |
| chr7 | 158712559 | 158713094 |
| chr8 | 179001 | 179288 |
| chr8 | 8243559 | 8243984 |
| chr8 | 8748652 | 8751478 |
| chr8 | 11725059 | 11725600 |
| chr8 | 23081956 | 23082975 |
| chr8 | 25041795 | 25042820 |
| chr8 | 28479635 | 28480798 |
| chr8 | 33371897 | 33372526 |
| chr8 | 38033408 | 38034643 |
| chr8 | 41386448 | 41386925 |
| chr8 | 42128689 | 42129141 |
| chr8 | 42396235 | 42397195 |
| chr8 | 42911215 | 42912042 |
| chr8 | 42995248 | 42995519 |
| chr8 | 48872458 | 48873907 |
| chr8 | 54934665 | 54935160 |
| chr8 | 55014024 | 55014681 |
| chr8 | 56792115 | 56793325 |
| chr8 | 56986556 | 56987308 |
| chr8 | 57123572 | 57124997 |
| chr8 | 64080674 | 64081766 |
| chr8 | 66556764 | 66557513 |
| chr8 | 67578694 | 67579509 |
| chr8 | 74197853 | 74198211 |
| chr8 | 74658490 | 74659828 |
| chr8 | 74790342 | 74791412 |
| chr8 | 81083320 | 81084057 |
| chr8 | 82598267 | 82598684 |
| chr8 | 82632973 | 82633672 |
| chr8 | 95651206 | 95651760 |
| chr8 | 95731798 | 95732449 |
| chr8 | 95835249 | 95835932 |
| chr8 | 95906788 | 95909088 |
| chr8 | 96037018 | 96037600 |
| chr8 | 98636694 | 98636904 |
| chr8 | 101733213 | 101735222 |
| chr8 | 103250753 | 103251209 |
| chr8 | 103424045 | 103425154 |
| chr8 | 103666157 | 103668861 |
| chr8 | 120844798 | 120845115 |
| chr8 | 124253054 | 124253707 |
| chr8 | 124408092 | 124409038 |
| chr8 | 125551211 | 125551521 |
| chr8 | 126103760 | 126104122 |
| chr8 | 128806081 | 128806899 |
| chr8 | 130951372 | 130952651 |
| chr8 | 131370087 | 131371317 |
| chr8 | 134308328 | 134310145 |
| chr8 | 141644429 | 141645598 |
| chr8 | 142010440 | 142011907 |
| chr8 | 143750759 | 143751448 |
| chr8 | 144798487 | 144799038 |
| chr8 | 145170253 | 145171437 |
| chr8 | 145235202 | 145235442 |
| chr8 | 145980532 | 145981234 |
| chr8 | 146012190 | 146012913 |
| chr9 | 178851 | 179249 |
| chr9 | 2843965 | 2844308 |
| chr9 | 4740694 | 4741868 |
| chr9 | 6412353 | 6414097 |
| chr9 | 19102470 | 19103110 |
| chr9 | 21989773 | 21990108 |
| chr9 | 33001216 | 33001668 |
| chr9 | 33816798 | 33818300 |
| chr9 | 35732124 | 35732757 |
| chr9 | 36487107 | 36488083 |
| chr9 | 37464814 | 37466569 |
| chr9 | 68377476 | 68377826 |
| chr9 | 70645539 | 70645863 |
| chr9 | 71736074 | 71737367 |
| chr9 | 74525249 | 74526679 |
| chr9 | 91002741 | 91004330 |
| chr9 | 97021464 | 97021967 |
| chr9 | 97766649 | 97767955 |
| chr9 | 98546650 | 98547028 |
| chr9 | 99145524 | 99145849 |
| chr9 | 99179479 | 99181723 |
| chr9 | 99212164 | 99213507 |
| chr9 | 99380814 | 99382236 |
| chr9 | 99687155 | 99687587 |
| chr9 | 100395340 | 100396414 |
| chr9 | 102668916 | 102669473 |
| chr9 | 102860775 | 102861677 |
| chr9 | 114658618 | 114659773 |
| chr9 | 115095149 | 115095919 |
| chr9 | 123555399 | 123555899 |
| chr9 | 125667114 | 125667804 |
| chr9 | 127177366 | 127177818 |
| chr9 | 127532040 | 127533691 |
| chr9 | 128508321 | 128510670 |
| chr9 | 129567144 | 129567803 |
| chr9 | 130158774 | 130159057 |
| chr9 | 130516407 | 130517849 |
| chr9 | 130652926 | 130653160 |
| chr9 | 130674546 | 130674958 |
| chr9 | 130829535 | 130830949 |
| chr9 | 130889698 | 130891144 |
| chr9 | 130953519 | 130954251 |
| chr9 | 131012454 | 131013429 |
| chr9 | 131084541 | 131085471 |
| chr9 | 131154346 | 131155923 |
| chr9 | 131266838 | 131267277 |
| chr9 | 131450972 | 131452682 |
| chr9 | 131643911 | 131644749 |
| chr9 | 132199361 | 132199984 |
| chr9 | 132244401 | 132245732 |
| chr9 | 132331218 | 132331458 |
| chr9 | 132312156 | 132312892 |
| chr9 | 132565316 | 132566033 |
| chr9 | 132815766 | 132816615 |
| chr9 | 133454629 | 133455496 |
| chr9 | 133534534 | 133542394 |
| chr9 | 133556516 | 133557645 |
| chr9 | 133587865 | 133588901 |
| chr9 | 134248688 | 134249160 |
| chr9 | 135282052 | 135282315 |
| chr9 | 135905412 | 135906550 |
| chr9 | 138798462 | 138800325 |
| chr9 | 140093882 | 140095686 |
| chr9 | 140473002 | 140473681 |
| chr9 | 140484219 | 140485721 |
| chr10 | 977260 | 978072 |
| chr10 | 1021641 | 1022509 |
| chr10 | 5726455 | 5727697 |
| chr10 | 5854706 | 5855965 |
| chr10 | 5930914 | 5932389 |
| chr10 | 6186520 | 6187855 |
| chr10 | 12237832 | 12238377 |
| chr10 | 14645752 | 14646705 |
| chr10 | 14920320 | 14921688 |
| chr10 | 14995680 | 14996431 |
| chr10 | 15130386 | 15130913 |
| chr10 | 15412419 | 15413040 |
| chr10 | 18940528 | 18940765 |
| chr10 | 21783198 | 21786420 |
| chr10 | 26986142 | 26987088 |
| chr10 | 27443030 | 27443405 |
| chr10 | 28591622 | 28592117 |
| chr10 | 28821189 | 28823092 |
| chr10 | 29214051 | 29214362 |
| chr10 | 32216901 | 32217102 |
| chr10 | 35625049 | 35626540 |
| chr10 | 38383200 | 38383962 |
| chr10 | 43891546 | 43892917 |
| chr10 | 63662886 | 63663299 |
| chr10 | 65224870 | 65225887 |
| chr10 | 65280870 | 65282030 |
| chr10 | 69834413 | 69835233 |
| chr10 | 70231396 | 70231797 |
| chr10 | 70286960 | 70287396 |
| chr10 | 70480704 | 70481598 |
| chr10 | 70660525 | 70661473 |
| chr10 | 72200065 | 72201368 |
| chr10 | 72575513 | 72576302 |
| chr10 | 73975671 | 73976115 |
| chr10 | 74020773 | 74021023 |
| chr10 | 74451828 | 74452597 |
| chr10 | 75006629 | 75007184 |
| chr10 | 75385082 | 75385994 |
| chr10 | 75489947 | 75490554 |
| chr10 | 75503973 | 75504222 |
| chr10 | 75757484 | 75758227 |
| chr10 | 79685719 | 79686507 |
| chr10 | 82168064 | 82168917 |
| chr10 | 88280635 | 88282089 |
| chr10 | 88853757 | 88855498 |
| chr10 | 93557970 | 93558699 |
| chr10 | 96305116 | 96305893 |
| chr10 | 98591707 | 98593273 |
| chr10 | 99257940 | 99259520 |
| chr10 | 102027099 | 102027490 |
| chr10 | 102672367 | 102673159 |
| chr10 | 103577394 | 103578256 |
| chr10 | 103990213 | 103991829 |
| chr10 | 104473399 | 104474711 |
| chr10 | 104952242 | 104953232 |
| chr10 | 105726687 | 105727255 |
| chr10 | 112402953 | 112404328 |
| chr10 | 114206604 | 114207320 |
| chr10 | 115999008 | 115999603 |
| chr10 | 116527524 | 116528474 |
| chr10 | 120839592 | 120840480 |
| chr10 | 123686972 | 123688109 |
| chr10 | 123734027 | 123734865 |
| chr10 | 123922850 | 123923542 |
| chr10 | 124134088 | 124134933 |
| chr10 | 126480092 | 126480810 |
| chr10 | 126605120 | 126606212 |
| chr10 | 131908731 | 131909886 |
| chr10 | 131927304 | 131927519 |
| chr11 | 459088 | 459345 |
| chr11 | 541896 | 542227 |
| chr11 | 660454 | 660681 |
| chr11 | 746877 | 748032 |
| chr11 | 881368 | 881666 |
| chr11 | 3012799 | 3014181 |
| chr11 | 3078142 | 3078984 |
| chr11 | 3818447 | 3819307 |
| chr11 | 4115689 | 4116489 |
| chr11 | 8703783 | 8704616 |
| chr11 | 9335730 | 9336477 |
| chr11 | 9406108 | 9406702 |
| chr11 | 13030330 | 13032584 |
| chr11 | 19733936 | 19735320 |
| chr11 | 20385161 | 20385673 |
| chr11 | 32851160 | 32851951 |
| chr11 | 33060699 | 33061846 |
| chr11 | 46615197 | 46615664 |
| chr11 | 47198027 | 47198885 |
| chr11 | 47573911 | 47574899 |
| chr11 | 47869793 | 47870356 |
| chr11 | 57508730 | 57509569 |
| chr11 | 59382687 | 59382929 |
| chr11 | 61196921 | 61197697 |
| chr11 | 62413632 | 62414293 |
| chr11 | 62521370 | 62521962 |
| chr11 | 62598679 | 62600064 |
| chr11 | 62606541 | 62607120 |
| chr11 | 63580537 | 63581740 |
| chr11 | 63952677 | 63954228 |
| chr11 | 64025913 | 64026736 |
| chr11 | 64034860 | 64035066 |
| chr11 | 64993280 | 64993484 |
| chr11 | 65081937 | 65083333 |
| chr11 | 65153726 | 65154606 |
| chr11 | 65547499 | 65549261 |
| chr11 | 66079092 | 66080920 |
| chr11 | 66383571 | 66385047 |
| chr11 | 66468372 | 66468575 |
| chr11 | 66511803 | 66512719 |
| chr11 | 66529656 | 66529896 |
| chr11 | 66885275 | 66887330 |
| chr11 | 67017607 | 67017822 |
| chr11 | 67887977 | 67889768 |
| chr11 | 68201118 | 68201321 |
| chr11 | 74178175 | 74178801 |
| chr11 | 76091524 | 76092638 |
| chr11 | 82904478 | 82905660 |
| chr11 | 101980632 | 101982330 |
| chr11 | 102962274 | 102963049 |
| chr11 | 116968849 | 116969512 |
| chr11 | 118868641 | 118869204 |
| chr11 | 118842379 | 118842933 |
| chr11 | 119039429 | 119039944 |
| chr11 | 126138359 | 126139166 |
| chr11 | 129871690 | 129873423 |
| chr11 | 130029658 | 130030262 |
| chr12 | 498098 | 498920 |
| chr12 | 1099557 | 1100785 |
| chr12 | 2921849 | 2922127 |
| chr12 | 2985978 | 2986639 |
| chr12 | 4430043 | 4430747 |
| chr12 | 6387654 | 6388168 |
| chr12 | 6961005 | 6961873 |
| chr12 | 9800543 | 9800852 |
| chr12 | 12419679 | 12420541 |
| chr12 | 12502942 | 12503465 |
| chr12 | 13044042 | 13044667 |
| chr12 | 14922877 | 14923974 |
| chr12 | 27932789 | 27933738 |
| chr12 | 31476717 | 31479466 |
| chr12 | 31742760 | 31744324 |
| chr12 | 31881630 | 31882461 |
| chr12 | 31944806 | 31945230 |
| chr12 | 32112161 | 32112833 |
| chr12 | 32259249 | 32260165 |
| chr12 | 32552025 | 32553038 |
| chr12 | 32908374 | 32908981 |
| chr12 | 39836211 | 39837499 |
| chr12 | 42631224 | 42632619 |
| chr12 | 42719761 | 42720119 |
| chr12 | 46661054 | 46663408 |
| chr12 | 49075489 | 49076301 |
| chr12 | 49688873 | 49691360 |
| chr12 | 49716630 | 49717018 |
| chr12 | 49726591 | 49727114 |
| chr12 | 49735719 | 49736875 |
| chr12 | 50560726 | 50561386 |
| chr12 | 50898621 | 50899164 |
| chr12 | 51610892 | 51611724 |
| chr12 | 53574025 | 53575150 |
| chr12 | 53645719 | 53646071 |
| chr12 | 53835295 | 53835568 |
| chr12 | 54069625 | 54070177 |
| chr12 | 54673322 | 54673550 |
| chr12 | 56039988 | 56040337 |
| chr12 | 56223263 | 56223598 |
| chr12 | 56211325 | 56211553 |
| chr12 | 56693680 | 56694345 |
| chr12 | 56709458 | 56710110 |
| chr12 | 57081290 | 57082445 |
| chr12 | 57824155 | 57824395 |
| chr12 | 58129828 | 58130361 |
| chr12 | 62860383 | 62860855 |
| chr12 | 64237403 | 64238401 |
| chr12 | 64845599 | 64846314 |
| chr12 | 65003793 | 65004899 |
| chr12 | 65152869 | 65153489 |
| chr12 | 66217627 | 66220181 |
| chr12 | 66524464 | 66524792 |
| chr12 | 66563202 | 66563945 |
| chr12 | 69004220 | 69005377 |
| chr12 | 69139815 | 69140206 |
| chr12 | 69201622 | 69202663 |
| chr12 | 72056768 | 72057787 |
| chr12 | 74564752 | 74565178 |
| chr12 | 76952707 | 76953830 |
| chr12 | 92538710 | 92540333 |
| chr12 | 93771465 | 93772289 |
| chr12 | 96640797 | 96641167 |
| chr12 | 98897604 | 98897849 |
| chr12 | 102513668 | 102514076 |
| chr12 | 104234273 | 104235327 |
| chr12 | 104659057 | 104659583 |
| chr12 | 104682601 | 104682993 |
| chr12 | 109124224 | 109125964 |
| chr12 | 110718392 | 110720062 |
| chr12 | 110841021 | 110841548 |
| chr12 | 110939456 | 110940173 |
| chr12 | 111020507 | 111021192 |
| chr12 | 112035612 | 112037558 |
| chr12 | 112279692 | 112280714 |
| chr12 | 112546147 | 112546706 |
| chr12 | 112846955 | 112847200 |
| chr12 | 112856457 | 112857243 |
| chr12 | 113622761 | 113623931 |
| chr12 | 117175623 | 117176436 |
| chr12 | 117257031 | 117257502 |
| chr12 | 117476810 | 117477106 |
| chr12 | 118809324 | 118811021 |
| chr12 | 120632222 | 120632784 |
| chr12 | 120875654 | 120876366 |
| chr12 | 120966788 | 120967038 |
| chr12 | 121018728 | 121019369 |
| chr12 | 121341414 | 121342436 |
| chr12 | 121416485 | 121416817 |
| chr12 | 121711846 | 121712162 |
| chr12 | 121789970 | 121790274 |
| chr12 | 121837380 | 121838396 |
| chr12 | 121880053 | 121880718 |
| chr12 | 122326482 | 122327044 |
| chr12 | 122984995 | 122985644 |
| chr12 | 123236994 | 123237664 |
| chr12 | 123920582 | 123921462 |
| chr12 | 124017593 | 124018994 |
| chr12 | 132379542 | 132380052 |
| chr12 | 133335487 | 133335851 |
| chr12 | 133532642 | 133532979 |
| chr12 | 133706528 | 133707594 |
| chr13 | 20175966 | 20176766 |
| chr13 | 28194183 | 28196249 |
| chr13 | 45149970 | 45152288 |
| chr13 | 45563288 | 45564002 |
| chr13 | 45914617 | 45915656 |
| chr13 | 50265224 | 50265598 |
| chr13 | 50570375 | 50571643 |
| chr13 | 53024155 | 53024938 |
| chr13 | 53226382 | 53226960 |
| chr13 | 73355703 | 73356612 |
| chr13 | 73632860 | 73634370 |
| chr13 | 95248288 | 95248672 |
| chr13 | 98794398 | 98796241 |
| chr13 | 99127039 | 99127344 |
| chr13 | 100150692 | 100151020 |
| chr13 | 111464738 | 111465988 |
| chr13 | 113410004 | 113410362 |
| chr14 | 21737406 | 21738094 |
| chr14 | 21924242 | 21924568 |
| chr14 | 21944914 | 21945812 |
| chr14 | 23291078 | 23291337 |
| chr14 | 23388092 | 23388857 |
| chr14 | 23517223 | 23517741 |
| chr14 | 31494794 | 31496056 |
| chr14 | 35098694 | 35099478 |
| chr14 | 35451634 | 35452544 |
| chr14 | 45431628 | 45432115 |
| chr14 | 45603373 | 45603906 |
| chr14 | 50052650 | 50053033 |
| chr14 | 50319426 | 50319632 |
| chr14 | 50697800 | 50698733 |
| chr14 | 50778680 | 50779544 |
| chr14 | 51134480 | 51135304 |
| chr14 | 51296727 | 51298477 |
| chr14 | 55031826 | 55035030 |
| chr14 | 55493614 | 55494032 |
| chr14 | 56046634 | 56047986 |
| chr14 | 58764507 | 58764961 |
| chr14 | 58893618 | 58893953 |
| chr14 | 60558340 | 60559176 |
| chr14 | 61188116 | 61190772 |
| chr14 | 64009645 | 64010525 |
| chr14 | 64969967 | 64972180 |
| chr14 | 69444639 | 69446826 |
| chr14 | 71373964 | 71375402 |
| chr14 | 73924588 | 73924980 |
| chr14 | 75179629 | 75179875 |
| chr14 | 75535825 | 75536376 |
| chr14 | 92572554 | 92573103 |
| chr14 | 92587549 | 92589022 |
| chr14 | 100704362 | 100707067 |
| chr14 | 102605597 | 102606977 |
| chr14 | 102973317 | 102976666 |
| chr14 | 103057926 | 103060044 |
| chr14 | 103851217 | 103852708 |
| chr14 | 104094551 | 104096361 |
| chr14 | 104313161 | 104314990 |
| chr14 | 105486611 | 105487915 |
| chr14 | 105911709 | 105912069 |
| chr15 | 23378183 | 23378615 |
| chr15 | 25683348 | 25684415 |
| chr15 | 28834219 | 28834690 |
| chr15 | 35261686 | 35262124 |
| chr15 | 38544002 | 38545532 |
| chr15 | 40650055 | 40651102 |
| chr15 | 40886396 | 40886918 |
| chr15 | 40986871 | 40987772 |
| chr15 | 41576041 | 41576536 |
| chr15 | 41836145 | 41836612 |
| chr15 | 42840462 | 42841116 |
| chr15 | 43981924 | 43982358 |
| chr15 | 44038504 | 44039122 |
| chr15 | 44486741 | 44487860 |
| chr15 | 44719186 | 44720138 |
| chr15 | 44828637 | 44829949 |
| chr15 | 45003460 | 45004345 |
| chr15 | 45814335 | 45815344 |
| chr15 | 49447339 | 49447576 |
| chr15 | 50646437 | 50647742 |
| chr15 | 57210226 | 57212005 |
| chr15 | 59279741 | 59280720 |
| chr15 | 59663863 | 59665081 |
| chr15 | 64647914 | 64648523 |
| chr15 | 65321431 | 65321996 |
| chr15 | 65476794 | 65478055 |
| chr15 | 65809685 | 65810168 |
| chr15 | 68346303 | 68347408 |
| chr15 | 68521418 | 68522237 |
| chr15 | 72489478 | 72490119 |
| chr15 | 72766260 | 72767693 |
| chr15 | 74284252 | 74284821 |
| chr15 | 74833241 | 74833961 |
| chr15 | 75165373 | 75165903 |
| chr15 | 75870802 | 75871814 |
| chr15 | 75917646 | 75918212 |
| chr15 | 85197232 | 85197695 |
| chr15 | 85258839 | 85259825 |
| chr15 | 85923604 | 85924680 |
| chr15 | 90644998 | 90646164 |
| chr15 | 90727879 | 90728570 |
| chr15 | 90776699 | 90777636 |
| chr16 | 278838 | 279495 |
| chr16 | 1358729 | 1360232 |
| chr16 | 1363813 | 1364496 |
| chr16 | 1524520 | 1525534 |
| chr16 | 1574515 | 1574986 |
| chr16 | 2867245 | 2868002 |
| chr16 | 3085222 | 3085469 |
| chr16 | 3493098 | 3493569 |
| chr16 | 4357552 | 4357955 |
| chr16 | 4400955 | 4401572 |
| chr16 | 4524138 | 4524712 |
| chr16 | 4659348 | 4659613 |
| chr16 | 5121749 | 5122192 |
| chr16 | 8768256 | 8768619 |
| chr16 | 8891322 | 8891928 |
| chr16 | 11890324 | 11891521 |
| chr16 | 11945065 | 11945517 |
| chr16 | 12008880 | 12010114 |
| chr16 | 15743759 | 15744423 |
| chr16 | 19097731 | 19098229 |
| chr16 | 19729194 | 19729896 |
| chr16 | 22448200 | 22449028 |
| chr16 | 23568398 | 23569488 |
| chr16 | 28223083 | 28223638 |
| chr16 | 28936053 | 28937036 |
| chr16 | 29465353 | 29467143 |
| chr16 | 29605964 | 29606945 |
| chr16 | 29675845 | 29676120 |
| chr16 | 30593965 | 30594525 |
| chr16 | 30759403 | 30760320 |
| chr16 | 30905549 | 30905783 |
| chr16 | 31075084 | 31075756 |
| chr16 | 31084343 | 31086048 |
| chr16 | 47494697 | 47495207 |
| chr16 | 52579636 | 52580845 |
| chr16 | 53088716 | 53089279 |
| chr16 | 53164000 | 53165838 |
| chr16 | 53737617 | 53738235 |
| chr16 | 56484865 | 56485774 |
| chr16 | 57219486 | 57220583 |
| chr16 | 58662975 | 58664048 |
| chr16 | 66729825 | 66731262 |
| chr16 | 66864407 | 66864991 |
| chr16 | 67143282 | 67143827 |
| chr16 | 67562575 | 67563390 |
| chr16 | 67752849 | 67753446 |
| chr16 | 67840275 | 67841106 |
| chr16 | 68732058 | 68732306 |
| chr16 | 68771034 | 68772344 |
| chr16 | 69166352 | 69166908 |
| chr16 | 69143499 | 69143919 |
| chr16 | 69345137 | 69345905 |
| chr16 | 69419316 | 69420086 |
| chr16 | 69458264 | 69458626 |
| chr16 | 70323127 | 70323489 |
| chr16 | 70414429 | 70415865 |
| chr16 | 70472588 | 70473649 |
| chr16 | 71917738 | 71918542 |
| chr16 | 74401546 | 74402639 |
| chr16 | 74700305 | 74701005 |
| chr16 | 75497828 | 75498780 |
| chr16 | 75512545 | 75513716 |
| chr16 | 75656905 | 75657360 |
| chr16 | 78133174 | 78134066 |
| chr16 | 81110485 | 81111243 |
| chr16 | 84149912 | 84151110 |
| chr16 | 85044730 | 85045436 |
| chr16 | 87416583 | 87418238 |
| chr16 | 87799123 | 87799747 |
| chr16 | 89487760 | 89488158 |
| chr16 | 89555775 | 89557753 |
| chr16 | 89723192 | 89724636 |
| chr16 | 89713911 | 89714850 |
| chr16 | 89882392 | 89883458 |
| chr16 | 90014250 | 90014613 |
| chr17 | 1302606 | 1303889 |
| chr17 | 1613226 | 1614110 |
| chr17 | 2206517 | 2207248 |
| chr17 | 2496493 | 2498023 |
| chr17 | 4268625 | 4270155 |
| chr17 | 4401959 | 4403323 |
| chr17 | 4842858 | 4843636 |
| chr17 | 4890282 | 4890991 |
| chr17 | 8124704 | 8125188 |
| chr17 | 8126224 | 8126486 |
| chr17 | 12920956 | 12921566 |
| chr17 | 16255618 | 16257148 |
| chr17 | 17739511 | 17740525 |
| chr17 | 17875167 | 17875933 |
| chr17 | 18217821 | 18219056 |
| chr17 | 19118213 | 19118802 |
| chr17 | 20755763 | 20756133 |
| chr17 | 20945386 | 20945633 |
| chr17 | 26971782 | 26972481 |
| chr17 | 27139185 | 27139671 |
| chr17 | 27276219 | 27278877 |
| chr17 | 27503598 | 27504014 |
| chr17 | 27619491 | 27620393 |
| chr17 | 27717135 | 27718037 |
| chr17 | 28088027 | 28088607 |
| chr17 | 29033009 | 29033351 |
| chr17 | 29058408 | 29058957 |
| chr17 | 29151481 | 29151901 |
| chr17 | 29421595 | 29422067 |
| chr17 | 30228604 | 30228962 |
| chr17 | 30243859 | 30244564 |
| chr17 | 30669000 | 30669287 |
| chr17 | 30770960 | 30772137 |
| chr17 | 33416129 | 33416578 |
| chr17 | 33894866 | 33895142 |
| chr17 | 34890466 | 34891480 |
| chr17 | 35289388 | 35289623 |
| chr17 | 35715796 | 35716627 |
| chr17 | 36003250 | 36003528 |
| chr17 | 36412765 | 36413545 |
| chr17 | 36858278 | 36861697 |
| chr17 | 37557366 | 37558982 |
| chr17 | 37607393 | 37607718 |
| chr17 | 39942175 | 39943176 |
| chr17 | 39992021 | 39992627 |
| chr17 | 40020980 | 40021924 |
| chr17 | 40074291 | 40075380 |
| chr17 | 40168999 | 40169694 |
| chr17 | 40573740 | 40575526 |
| chr17 | 41278134 | 41278460 |
| chr17 | 41363727 | 41364273 |
| chr17 | 42257043 | 42257402 |
| chr17 | 42580377 | 42581046 |
| chr17 | 43128789 | 43129038 |
| chr17 | 43198277 | 43198773 |
| chr17 | 45177258 | 45177755 |
| chr17 | 46178149 | 46178943 |
| chr17 | 47022062 | 47022344 |
| chr17 | 47438906 | 47439938 |
| chr17 | 48680336 | 48680555 |
| chr17 | 48784561 | 48785331 |
| chr17 | 49197522 | 49198474 |
| chr17 | 53045603 | 53046368 |
| chr17 | 54910496 | 54912470 |
| chr17 | 55162173 | 55163908 |
| chr17 | 55333068 | 55334245 |
| chr17 | 55927212 | 55928098 |
| chr17 | 56064830 | 56066051 |
| chr17 | 57231855 | 57232655 |
| chr17 | 57696654 | 57697280 |
| chr17 | 58041846 | 58042463 |
| chr17 | 58603022 | 58603710 |
| chr17 | 59940825 | 59941067 |
| chr17 | 60142247 | 60143516 |
| chr17 | 60555507 | 60557168 |
| chr17 | 62492882 | 62493323 |
| chr17 | 62657409 | 62658858 |
| chr17 | 65362343 | 65362837 |
| chr17 | 66097528 | 66098631 |
| chr17 | 70536483 | 70536687 |
| chr17 | 73073313 | 73073941 |
| chr17 | 73149830 | 73150885 |
| chr17 | 73498099 | 73498390 |
| chr17 | 73974971 | 73975731 |
| chr17 | 74533281 | 74534566 |
| chr17 | 74706465 | 74707067 |
| chr17 | 74722031 | 74723420 |
| chr17 | 74732345 | 74734222 |
| chr17 | 74965089 | 74965303 |
| chr17 | 75447477 | 75447821 |
| chr17 | 76183077 | 76183449 |
| chr17 | 76164395 | 76165272 |
| chr17 | 76354818 | 76357038 |
| chr17 | 77965805 | 77966296 |
| chr17 | 79235486 | 79235751 |
| chr17 | 79603627 | 79603886 |
| chr17 | 79650194 | 79651447 |
| chr17 | 79846204 | 79846486 |
| chr17 | 79804259 | 79804488 |
| chr17 | 80454652 | 80455632 |
| chr17 | 80605301 | 80606324 |
| chr18 | 712232 | 713086 |
| chr18 | 9333896 | 9334244 |
| chr18 | 12947924 | 12948654 |
| chr18 | 19180049 | 19181026 |
| chr18 | 19744936 | 19752363 |
| chr18 | 23669971 | 23671275 |
| chr18 | 43677956 | 43678352 |
| chr18 | 61089262 | 61090357 |
| chr19 | 984165 | 984866 |
| chr19 | 1591427 | 1591628 |
| chr19 | 1734221 | 1734627 |
| chr19 | 1826829 | 1828546 |
| chr19 | 2135931 | 2136164 |
| chr19 | 2424005 | 2427983 |
| chr19 | 3546144 | 3546382 |
| chr19 | 3761480 | 3761826 |
| chr19 | 4986182 | 4986405 |
| chr19 | 7069396 | 7069921 |
| chr19 | 9648934 | 9649410 |
| chr19 | 9694921 | 9695433 |
| chr19 | 9938325 | 9938815 |
| chr19 | 10691835 | 10692057 |
| chr19 | 10735999 | 10736396 |
| chr19 | 11031933 | 11032195 |
| chr19 | 11170550 | 11170837 |
| chr19 | 11909402 | 11909841 |
| chr19 | 11998804 | 11999131 |
| chr19 | 12146030 | 12146649 |
| chr19 | 12444032 | 12444548 |
| chr19 | 12661804 | 12662302 |
| chr19 | 12707696 | 12708114 |
| chr19 | 12844998 | 12845664 |
| chr19 | 13813261 | 13813599 |
| chr19 | 13858446 | 13858904 |
| chr19 | 13869717 | 13870085 |
| chr19 | 14628406 | 14629689 |
| chr19 | 16682657 | 16683792 |
| chr19 | 16770636 | 16772632 |
| chr19 | 17355181 | 17356471 |
| chr19 | 17487895 | 17488125 |
| chr19 | 17877468 | 17877777 |
| chr19 | 18433642 | 18434178 |
| chr19 | 18632298 | 18633453 |
| chr19 | 19383703 | 19384283 |
| chr19 | 19495952 | 19497038 |
| chr19 | 19729127 | 19729814 |
| chr19 | 19745475 | 19745729 |
| chr19 | 21933434 | 21933853 |
| chr19 | 33555246 | 33556431 |
| chr19 | 34662931 | 34664060 |
| chr19 | 36207084 | 36209691 |
| chr19 | 36822395 | 36822690 |
| chr19 | 37769016 | 37769434 |
| chr19 | 38270279 | 38270617 |
| chr19 | 38572648 | 38573752 |
| chr19 | 39138062 | 39138570 |
| chr19 | 39281367 | 39283662 |
| chr19 | 39935931 | 39936638 |
| chr19 | 44808897 | 44809229 |
| chr19 | 45147074 | 45147721 |
| chr19 | 45251975 | 45252330 |
| chr19 | 45954065 | 45954504 |
| chr19 | 46366202 | 46366605 |
| chr19 | 47248749 | 47249948 |
| chr19 | 47290585 | 47291983 |
| chr19 | 47353614 | 47354150 |
| chr19 | 47747363 | 47747715 |
| chr19 | 48075968 | 48076195 |
| chr19 | 48918115 | 48918340 |
| chr19 | 49106882 | 49107178 |
| chr19 | 49217245 | 49217869 |
| chr19 | 49652212 | 49654260 |
| chr19 | 51307439 | 51308380 |
| chr19 | 52598480 | 52599238 |
| chr19 | 52674327 | 52674855 |
| chr19 | 53030824 | 53031359 |
| chr19 | 53193139 | 53193945 |
| chr19 | 53228973 | 53229195 |
| chr19 | 53445344 | 53445933 |
| chr19 | 53696029 | 53696650 |
| chr19 | 54960191 | 54960948 |
| chr19 | 57683596 | 57683850 |
| chr19 | 58325855 | 58326330 |
| chr19 | 58331004 | 58331434 |
| chr19 | 58715220 | 58716033 |
| chr19 | 58728277 | 58728729 |
| chr19 | 58739943 | 58740554 |
| chr19 | 58789914 | 58790972 |
| chr19 | 59084201 | 59085059 |
| chr20 | 304826 | 308323 |
| chr20 | 3758206 | 3759184 |
| chr20 | 3766343 | 3767636 |
| chr20 | 3800761 | 3801546 |
| chr20 | 3869453 | 3870570 |
| chr20 | 5093360 | 5094145 |
| chr20 | 5591490 | 5591875 |
| chr20 | 5930962 | 5931623 |
| chr20 | 6103436 | 6103970 |
| chr20 | 10414276 | 10414993 |
| chr20 | 17549628 | 17550051 |
| chr20 | 17948755 | 17949921 |
| chr20 | 21283636 | 21284446 |
| chr20 | 25370335 | 25371823 |
| chr20 | 25565437 | 25566547 |
| chr20 | 30192006 | 30192273 |
| chr20 | 32077657 | 32078469 |
| chr20 | 32232170 | 32232403 |
| chr20 | 32890559 | 32891378 |
| chr20 | 32950984 | 32951969 |
| chr20 | 33412857 | 33413602 |
| chr20 | 33871956 | 33872739 |
| chr20 | 34022007 | 34022502 |
| chr20 | 34188719 | 34190267 |
| chr20 | 34203678 | 34204301 |
| chr20 | 34252556 | 34252927 |
| chr20 | 34286821 | 34287701 |
| chr20 | 34329490 | 34330563 |
| chr20 | 34680003 | 34681001 |
| chr20 | 35491001 | 35492805 |
| chr20 | 37590617 | 37591184 |
| chr20 | 42939450 | 42940043 |
| chr20 | 42955433 | 42955728 |
| chr20 | 44420497 | 44420868 |
| chr20 | 44518897 | 44520361 |
| chr20 | 46130428 | 46131057 |
| chr20 | 47538163 | 47538672 |
| chr20 | 47804353 | 47805160 |
| chr20 | 48329645 | 48330682 |
| chr20 | 48429172 | 48429645 |
| chr20 | 48522602 | 48523134 |
| chr20 | 48769945 | 48770709 |
| chr20 | 49347365 | 49348659 |
| chr20 | 49574816 | 49576287 |
| chr20 | 50383675 | 50384904 |
| chr20 | 52198058 | 52198400 |
| chr20 | 56283891 | 56285395 |
| chr20 | 60696561 | 60698342 |
| chr20 | 60860732 | 60860999 |
| chr20 | 61339858 | 61341220 |
| chr20 | 61492119 | 61493626 |
| chr20 | 62257589 | 62259476 |
| chr20 | 62317505 | 62317775 |
| chr20 | 62461266 | 62462037 |
| chr20 | 62492849 | 62494249 |
| chr20 | 62509163 | 62509369 |
| chr20 | 62569797 | 62570776 |
| chr20 | 62605261 | 62605463 |
| chr21 | 9968264 | 9968620 |
| chr21 | 11106718 | 11107132 |
| chr21 | 33103456 | 33104975 |
| chr21 | 34602241 | 34603265 |
| chr21 | 34696532 | 34697750 |
| chr21 | 36258952 | 36259472 |
| chr21 | 37528205 | 37529561 |
| chr21 | 38352856 | 38353274 |
| chr21 | 40684370 | 40686324 |
| chr21 | 44299111 | 44299808 |
| chr21 | 44394084 | 44395850 |
| chr21 | 46221388 | 46222456 |
| chr21 | 47648191 | 47649622 |
| chr21 | 47705627 | 47706428 |
| chr22 | 18111132 | 18111672 |
| chr22 | 18121280 | 18122009 |
| chr22 | 18531215 | 18531504 |
| chr22 | 21311336 | 21312201 |
| chr22 | 21903231 | 21905437 |
| chr22 | 21921936 | 21922715 |
| chr22 | 24110029 | 24110995 |
| chr22 | 24384134 | 24384405 |
| chr22 | 25507309 | 25507537 |
| chr22 | 25816912 | 25817539 |
| chr22 | 28314588 | 28315958 |
| chr22 | 29196082 | 29196962 |
| chr22 | 29279412 | 29280287 |
| chr22 | 29445638 | 29446113 |
| chr22 | 29949478 | 29949824 |
| chr22 | 29976920 | 29977216 |
| chr22 | 30278978 | 30279858 |
| chr22 | 30901242 | 30901744 |
| chr22 | 31063462 | 31064384 |
| chr22 | 31364564 | 31365575 |
| chr22 | 32145635 | 32146200 |
| chr22 | 35776988 | 35777221 |
| chr22 | 36424219 | 36425303 |
| chr22 | 37414319 | 37416111 |
| chr22 | 38239792 | 38240409 |
| chr22 | 38301954 | 38302871 |
| chr22 | 38712684 | 38713333 |
| chr22 | 38793180 | 38793451 |
| chr22 | 39052054 | 39053054 |
| chr22 | 41346636 | 41347759 |
| chr22 | 41777430 | 41778281 |
| chr22 | 42195953 | 42196961 |
| chr22 | 42228507 | 42229984 |
| chr22 | 42971800 | 42972206 |
| chr22 | 42977728 | 42978214 |
| chr22 | 43010440 | 43011063 |
| chr22 | 43252653 | 43253667 |
| chr22 | 44319578 | 44320513 |
| chr22 | 45124981 | 45125855 |
| chr22 | 45182016 | 45182223 |
| chr22 | 45559298 | 45560172 |
| chr22 | 45705511 | 45706616 |
| chr22 | 46041368 | 46041910 |
| chr22 | 46518454 | 46518729 |
| chr22 | 50241735 | 50243918 |
| chr22 | 50845158 | 50845437 |
| chrX | 2825438 | 2825646 |
| chrX | 16737618 | 16738099 |
| chrX | 23761070 | 23761610 |
| chrX | 24072558 | 24073135 |
| chrX | 45710269 | 45710979 |
| chrX | 47003750 | 47004987 |
| chrX | 48595526 | 48595997 |
| chrX | 48900510 | 48901469 |
| chrX | 54556428 | 54556959 |
| chrX | 69353104 | 69353904 |
| chrX | 70473812 | 70474287 |
| chrX | 70752638 | 70752965 |
| chrX | 73164003 | 73164247 |
| chrX | 73512193 | 73512569 |
| chrX | 77041003 | 77041725 |
| chrX | 77150837 | 77151247 |
| chrX | 100603622 | 100603950 |
| chrX | 103401400 | 103401778 |
| chrX | 109245900 | 109246673 |
| chrX | 110924384 | 110925275 |
| chrX | 114795414 | 114796228 |
| chrX | 118533163 | 118533787 |
| chrX | 118986684 | 118987252 |
| chrX | 122866199 | 122867097 |
| chrX | 122993547 | 122994335 |
| chrX | 128977325 | 128977998 |
| chrX | 129243674 | 129245575 |
| chrX | 129299432 | 129300105 |
| chrX | 132090363 | 132092617 |
| chrX | 133930787 | 133931355 |
| chrX | 135962102 | 135962951 |
| chrX | 153284685 | 153285655 |
| chrX | 153774893 | 153776136 |

TABLE B

| Repeat1 | Repeat1_Chr | Start1 | End1 | Repeat1_Sign | Repeat2 | Repeat2_Chr |
|---|---|---|---|---|---|---|
| AluSz | chr14 | 20786926 | 20787221 | − | AluSp | chr14 |
| AluYc | chr14 | 20791806 | 20792099 | + | AluSx | chr14 |
| AluSg | chr14 | 21696547 | 21696849 | − | AluSx | chr14 |
| AluSp | chr14 | 21888925 | 21889219 | − | AluSx | chr14 |
| AluSx | chr14 | 21890218 | 21890518 | + | AluY | chr14 |
| AluJb | chr14 | 21939494 | 21939800 | + | AluSx1 | chr14 |
| AluY | chr14 | 23301544 | 23301848 | − | AluSp | chr14 |
| AluY | chr14 | 23301544 | 23301848 | − | AluSc | chr14 |
| AluSx4 | chr14 | 23382840 | 23383083 | + | AluSx1 | chr14 |
| AluSx1 | chr14 | 23384706 | 23385002 | − | AluJr | chr14 |
| AluSq2 | chr14 | 23501372 | 23501683 | + | AluY | chr14 |
| AluSz | chr14 | 31432626 | 31432913 | − | AluSz | chr14 |
| AluSx3 | chr14 | 31484583 | 31484874 | − | AluJo | chr14 |
| AluSc8 | chr14 | 34902888 | 34903189 | + | AluSq2 | chr14 |
| AluSq2 | chr14 | 34912757 | 34913040 | − | AluSg | chr14 |
| AluSx3 | chr14 | 34927700 | 34927930 | − | AluSq2 | chr14 |
| AluSz | chr14 | 35042499 | 35042794 | + | AluSg4 | chr14 |
| AluSg4 | chr14 | 35042830 | 35043144 | − | AluSg | chr14 |
| AluSq2 | chr14 | 35091153 | 35091467 | − | AluY | chr14 |
| AluY | chr14 | 35091505 | 35091795 | + | AluSx3 | chr14 |
| AluSx3 | chr14 | 35094131 | 35094420 | − | AluYk4 | chr14 |
| AluSg4 | chr14 | 35422726 | 35423022 | − | AluSz | chr14 |
| AluY | chr14 | 35422413 | 35422721 | − | AluSz | chr14 |
| AluY | chr14 | 35422413 | 35422721 | − | AluSz | chr14 |
| AluSg4 | chr14 | 35422726 | 35423022 | − | AluSz | chr14 |
| AluSx | chr14 | 35427726 | 35428022 | − | AluSc8 | chr14 |
| AluJb | chr14 | 35495416 | 35495737 | − | AluSc | chr14 |
| AluSc | chr14 | 35495869 | 35496172 | + | AluSz | chr14 |
| AluSz6 | chr14 | 36012838 | 36013147 | − | AluSx | chr14 |
| AluSx1 | chr14 | 45435073 | 45435367 | − | AluSx | chr14 |
| AluSx | chr14 | 45585777 | 45586078 | + | AluSx | chr14 |
| AluSx1 | chr14 | 50038662 | 50038967 | − | AluSz | chr14 |
| AluSz | chr14 | 50039905 | 50040210 | + | AluSx1 | chr14 |
| AluSz | chr14 | 50299702 | 50300000 | + | AluSq2 | chr14 |
| AluSz | chr14 | 50680134 | 50680388 | + | AluY | chr14 |
| AluSx | chr14 | 50746497 | 50746802 | + | AluSx | chr14 |
| AluSq2 | chr14 | 50762446 | 50762756 | − | AluSz | chr14 |
| AluJb | chr14 | 50762121 | 50762439 | − | AluSz | chr14 |
| AluSg | chr14 | 50791488 | 50791791 | − | AluJb | chr14 |
| AluJb | chr14 | 50793784 | 50794076 | + | AluSg7 | chr14 |
| AluSx1 | chr14 | 51122148 | 51122443 | − | AluSp | chr14 |
| AluSx1 | chr14 | 51267806 | 51268108 | − | AluSg7 | chr14 |
| AluSg7 | chr14 | 51269791 | 51270089 | + | AluSx1 | chr14 |
| AluSx | chr14 | 55063021 | 55063326 | + | AluSp | chr14 |
| AluSx | chr14 | 55063021 | 55063326 | + | AluJb | chr14 |
| AluY | chr14 | 55074176 | 55074471 | − | AluSz | chr14 |
| AluSz | chr14 | 55522369 | 55522664 | + | AluY | chr14 |
| AluSx3 | chr14 | 56021129 | 56021419 | − | AluY | chr14 |
| AluSq2 | chr14 | 57942172 | 57942441 | + | AluSz6 | chr14 |
| AluSz | chr14 | 58778936 | 58779231 | − | AluSz6 | chr14 |
| AluSx1 | chr14 | 58892555 | 58892850 | + | AluSx3 | chr14 |
| AluSq2 | chr14 | 60565376 | 60565684 | + | AluSq2 | chr14 |
| AluSq2 | chr14 | 61183783 | 61184089 | − | AluSg | chr14 |
| AluSx3 | chr14 | 61212952 | 61213255 | + | AluJb | chr14 |
| AluSc8 | chr14 | 61214633 | 61214937 | + | AluJb | chr14 |
| AluSx | chr14 | 63946894 | 63947181 | + | AluJr | chr14 |
| AluSx1 | chr14 | 63992586 | 63992877 | − | AluSp | chr14 |
| AluJb | chr14 | 63993265 | 63993578 | − | AluSp | chr14 |
| AluSx1 | chr14 | 64985857 | 64986155 | − | AluJo | chr14 |
| AluSg | chr14 | 69416731 | 69417039 | + | AluY | chr14 |
| AluJb | chr14 | 71390666 | 71390978 | + | AluJo | chr14 |
| AluJb | chr14 | 71390666 | 71390978 | + | AluSz6 | chr14 |
| AluY | chr14 | 71937531 | 71937860 | − | AluSg | chr14 |
| AluSx1 | chr14 | 71946791 | 71947105 | − | AluSx1 | chr14 |
| AluJb | chr14 | 73659737 | 73660025 | + | AluJo | chr14 |
| AluSz | chr14 | 73917220 | 73917511 | + | AluSx | chr14 |
| AluSc5 | chr14 | 75202274 | 75202569 | − | AluSx | chr14 |
| AluY | chr14 | 75521783 | 75522089 | − | AluSg4 | chr14 |
| AluSx | chr14 | 78024962 | 78025233 | − | AluSz6 | chr14 |
| AluSx1 | chr14 | 92525883 | 92526191 | + | AluSz | chr14 |
| AluSx1 | chr14 | 92525883 | 92526191 | + | AluSg4 | chr14 |
| AluSg | chr14 | 92586994 | 92587195 | − | AluSq2 | chr14 |
| AluSp | chr14 | 92586682 | 92586993 | − | AluSq2 | chr14 |
| AluSz | chr14 | 92633346 | 92633655 | − | AluSx1 | chr14 |
| AluSq | chr14 | 93736442 | 93736741 | − | AluSx | chr14 |
| AluSz | chr14 | 1.01E+08 | 1.01E+08 | − | AluSq2 | chr14 |
| AluSq2 | chr14 | 1.01E+08 | 1.01E+08 | + | AluSc | chr14 |
| AluSg | chr14 | 1.03E+08 | 1.03E+08 | − | AluSx | chr14 |
| AluSg | chr14 | 1.03E+08 | 1.03E+08 | − | AluSp | chr14 |
| AluSz | chr14 | 1.03E+08 | 1.03E+08 | − | AluSg | chr14 |
| AluSz | chr14 | 1.03E+08 | 1.03E+08 | − | AluSx | chr14 |
| AluSq2 | chr14 | 1.03E+08 | 1.03E+08 | − | AluSq2 | chr14 |
| AluSq2 | chr14 | 1.03E+08 | 1.03E+08 | + | AluSx1 | chr14 |
| AluSx3 | chr14 | 1.03E+08 | 1.03E+08 | − | AluSz | chr14 |
| AluSz | chr14 | 1.04E+08 | 1.04E+08 | + | AluSx | chr14 |
| AluY | chr14 | 1.04E+08 | 1.04E+08 | − | AluSp | chr14 |
| AluSz | chr14 | 1.04E+08 | 1.04E+08 | − | AluSp | chr14 |
| AluSp | chr14 | 1.04E+08 | 1.04E+08 | + | AluJr | chr14 |
| AluSz | chr14 | 1.04E+08 | 1.04E+08 | − | AluSx | chr14 |
| AluJr | chr14 | 1.04E+08 | 1.04E+08 | − | AluSx | chr14 |
| AluSp | chr14 | 1.04E+08 | 1.04E+08 | + | AluSz6 | chr14 |
| AluSx | chr14 | 1.04E+08 | 1.04E+08 | + | AluSz6 | chr14 |
| AluSq10 | chr14 | 1.04E+08 | 1.04E+08 | − | AluSx | chr14 |
| AluSg | chr14 | 1.04E+08 | 1.04E+08 | − | AluY | chr14 |
| AluSg | chr14 | 1.04E+08 | 1.04E+08 | − | AluSx1 | chr14 |
| AluY | chr14 | 1.04E+08 | 1.04E+08 | + | AluSx3 | chr14 |
| AluSp | chr14 | 1.04E+08 | 1.04E+08 | + | AluSx1 | chr14 |
| AluSx1 | chr14 | 1.04E+08 | 1.04E+08 | − | AluSz6 | chr14 |
| AluY | chr14 | 1.04E+08 | 1.04E+08 | + | AluJb | chr14 |
| AluSz | chr14 | 1.05E+08 | 1.05E+08 | − | AluSc8 | chr14 |
| AluJb | chr14 | 1.06E+08 | 1.06E+08 | + | AluJb | chr14 |
| AluSc8 | chr14 | 1.07E+08 | 1.07E+08 | + | AluSp | chr14 |
| AluSx | chr8 | 176871 | 177164 | − | AluSx1 | chr8 |
| AluSz | chr8 | 8199367 | 8199659 | + | AluY | chr8 |
| AluSx1 | chr8 | 8224745 | 8225004 | + | AluSc8 | chr8 |
| AluSc8 | chr8 | 8225158 | 8225456 | − | AluSz | chr8 |
| AluSz | chr8 | 8228383 | 8228667 | + | AluY | chr8 |
| AluSx1 | chr8 | 8632023 | 8632321 | − | AluSz6 | chr8 |
| AluSz6 | chr8 | 8632435 | 8632726 | + | AluSx1 | chr8 |
| AluSx | chr8 | 8698703 | 8698990 | + | AluSx1 | chr8 |
| AluSq | chr8 | 8708070 | 8708344 | + | AluSz | chr8 |
| AluSg | chr8 | 11700446 | 11700754 | − | AluSx1 | chr8 |
| AluSq2 | chr8 | 11700139 | 11700445 | − | AluSx1 | chr8 |
| AluSx1 | chr8 | 23044840 | 23045148 | − | AluY | chr8 |
| AluSc | chr8 | 25055092 | 25055266 | − | AluSx | chr8 |
| AluJb | chr8 | 28499678 | 28499983 | + | AluYk4 | chr8 |
| AluSx | chr8 | 33355019 | 33355311 | − | AluSx | chr8 |
| AluSx1 | chr8 | 38074062 | 38074363 | − | AluY | chr8 |
| AluSx | chr8 | 38399673 | 38399983 | − | AluJr | chr8 |
| AluYc | chr8 | 38829194 | 38829475 | + | AluJr4 | chr8 |
| AluSx3 | chr8 | 41399622 | 41399929 | + | AluSq | chr8 |
| AluSz | chr8 | 42152635 | 42152936 | − | AluSg7 | chr8 |
| AluY | chr8 | 42169397 | 42169702 | − | AluSx | chr8 |
| AluY | chr8 | 42381532 | 42381830 | + | AluSp | chr8 |
| AluY | chr8 | 42381532 | 42381830 | + | AluJo | chr8 |
| AluSg | chr8 | 42937909 | 42938203 | + | AluJr | chr8 |
| AluY | chr8 | 42998673 | 42998988 | − | AluJb | chr8 |
| AluSx1 | chr8 | 48692467 | 48692775 | − | AluSc | chr8 |
| AluSx | chr8 | 48797716 | 48798013 | − | AluSx3 | chr8 |
| AluSg4 | chr8 | 48858291 | 48858581 | − | AluSx | chr8 |
| AluSg4 | chr8 | 48858291 | 48858581 | − | AluSz6 | chr8 |
| AluJb | chr8 | 54902913 | 54903223 | − | AluSx | chr8 |
| AluY | chr8 | 54968206 | 54968500 | − | AluSz | chr8 |
| AluSz | chr8 | 54970821 | 54971122 | + | AluSq2 | chr8 |
| AluSc8 | chr8 | 56738431 | 56738707 | − | AluSx | chr8 |
| AluJb | chr8 | 56737485 | 56737780 | − | AluSx | chr8 |
| AluJb | chr8 | 56803287 | 56803587 | − | AluSp | chr8 |
| AluJb | chr8 | 56803287 | 56803587 | − | AluSx3 | chr8 |
| AluSz6 | chr8 | 56829454 | 56829764 | + | AluJo | chr8 |
| AluSg7 | chr8 | 56977544 | 56977854 | − | AluSg | chr8 |
| AluSg7 | chr8 | 56977544 | 56977854 | − | AluY | chr8 |
| AluSg | chr8 | 56979211 | 56979520 | + | AluYb9 | chr8 |
| AluY | chr8 | 57131519 | 57131819 | + | AluSx1 | chr8 |
| AluSq2 | chr8 | 64115021 | 64115320 | − | AluSq2 | chr8 |
| AluSp | chr8 | 66596625 | 66596919 | − | AluSp | chr8 |
| AluSp | chr8 | 66596969 | 66597252 | + | AluY | chr8 |
| AluY | chr8 | 66599583 | 66599879 | − | AluSx | chr8 |
| AluY | chr8 | 66599583 | 66599879 | − | AluSx1 | chr8 |
| AluSp | chr8 | 66642404 | 66642714 | − | AluSz | chr8 |
| AluSq2 | chr8 | 67606501 | 67606793 | − | AluSc | chr8 |
| AluJb | chr8 | 74228267 | 74228553 | − | AluJo | chr8 |
| AluSx | chr8 | 74654487 | 74654791 | + | AluSp | chr8 |
| AluSx | chr8 | 74785125 | 74785442 | − | AluJb | chr8 |
| AluSp | chr8 | 80888321 | 80888617 | − | AluSz6 | chr8 |
| AluSp | chr8 | 81078542 | 81078766 | − | AluSx | chr8 |
| AluJb | chr8 | 82588940 | 82589238 | + | AluSp | chr8 |
| AluSx1 | chr8 | 82612336 | 82612607 | − | AluSp | chr8 |
| AluSp | chr8 | 82612893 | 82613202 | + | AluSq2 | chr8 |
| AluSq2 | chr8 | 82615652 | 82615957 | − | AluJb | chr8 |
| AluSz | chr8 | 95661167 | 95661442 | + | AluSx1 | chr8 |
| AluSx | chr8 | 95688320 | 95688593 | + | AluSc8 | chr8 |
| AluSg | chr8 | 95707573 | 95707890 | − | AluSx | chr8 |
| AluY | chr8 | 95754305 | 95754598 | + | AluSx | chr8 |
| AluSp | chr8 | 95864818 | 95865135 | − | AluSz6 | chr8 |
| AluSq2 | chr8 | 95872528 | 95872832 | + | AluSp | chr8 |
| AluSq2 | chr8 | 95872528 | 95872832 | + | AluSz6 | chr8 |
| AluSx1 | chr8 | 95914922 | 95915229 | − | AluJo | chr8 |
| AluJb | chr8 | 96051199 | 96051400 | − | AluSc | chr8 |
| AluSq2 | chr8 | 98676987 | 98677289 | − | AluY | chr8 |
| AluY | chr8 | 98678073 | 98678374 | + | AluSx1 | chr8 |
| AluSx1 | chr8 | 98681386 | 98681674 | − | AluSz | chr8 |
| AluSx1 | chr8 | 98678925 | 98679224 | − | AluSz | chr8 |
| AluSp | chr8 | 98706429 | 98706714 | − | AluSx | chr8 |
| AluSx1 | chr8 | 1.02E+08 | 1.02E+08 | − | AluSz | chr8 |
| AluSx1 | chr8 | 1.03E+08 | 1.03E+08 | + | AluSc5 | chr8 |
| AluSz6 | chr8 | 1.03E+08 | 1.03E+08 | + | AluSg | chr8 |
| AluSx3 | chr8 | 1.03E+08 | 1.03E+08 | + | AluSg | chr8 |
| AluSx1 | chr8 | 1.03E+08 | 1.03E+08 | − | AluSx3 | chr8 |
| AluSx | chr8 | 1.03E+08 | 1.03E+08 | − | AluSx | chr8 |
| AluSc | chr8 | 1.04E+08 | 1.04E+08 | − | AluYc | chr8 |
| AluSq | chr8 | 1.21E+08 | 1.21E+08 | − | AluJb | chr8 |
| AluSz | chr8 | 1.21E+08 | 1.21E+08 | + | AluSx4 | chr8 |
| AluSx3 | chr8 | 1.24E+08 | 1.24E+08 | − | AluY | chr8 |
| AluSq2 | chr8 | 1.24E+08 | 1.24E+08 | + | AluJo | chr8 |
| AluSq2 | chr8 | 1.26E+08 | 1.26E+08 | − | AluSx | chr8 |
| AluSg | chr8 | 1.26E+08 | 1.26E+08 | − | AluSx | chr8 |
| AluJb | chr8 | 1.26E+08 | 1.26E+08 | + | AluJb | chr8 |
| AluSx | chr8 | 1.26E+08 | 1.26E+08 | + | AluSp | chr8 |
| AluSp | chr8 | 1.26E+08 | 1.26E+08 | − | AluJb | chr8 |
| AluY | chr8 | 1.26E+08 | 1.26E+08 | + | AluSp | chr8 |
| AluY | chr8 | 1.26E+08 | 1.26E+08 | + | AluSz6 | chr8 |
| AluSq2 | chr8 | 1.28E+08 | 1.28E+08 | − | AluJr | chr8 |
| AluSq2 | chr8 | 1.28E+08 | 1.28E+08 | − | AluY | chr8 |
| AluSg | chr8 | 1.28E+08 | 1.28E+08 | − | AluSx1 | chr8 |
| AluSg | chr8 | 1.29E+08 | 1.29E+08 | + | AluJr | chr8 |
| AluSx | chr8 | 1.29E+08 | 1.29E+08 | − | AluSg | chr8 |
| AluSz6 | chr8 | 1.29E+08 | 1.29E+08 | − | AluY | chr8 |
| AluSp | chr8 | 1.29E+08 | 1.29E+08 | − | AluSx1 | chr8 |
| AluSp | chr8 | 1.29E+08 | 1.29E+08 | − | AluJb | chr8 |
| AluSq | chr8 | 1.29E+08 | 1.29E+08 | − | AluSp | chr8 |
| AluSz | chr8 | 1.29E+08 | 1.29E+08 | + | AluSx | chr8 |
| AluY | chr8 | 1.29E+08 | 1.29E+08 | − | AluSz6 | chr8 |
| AluSq2 | chr8 | 1.29E+08 | 1.29E+08 | + | AluSg | chr8 |
| AluY | chr8 | 1.31E+08 | 1.31E+08 | + | AluJo | chr8 |
| AluSq2 | chr8 | 1.31E+08 | 1.31E+08 | − | AluSz6 | chr8 |
| AluSz | chr8 | 1.31E+08 | 1.31E+08 | + | AluY | chr8 |
| AluSz6 | chr8 | 1.34E+08 | 1.34E+08 | + | AluSq2 | chr8 |
| AluSg | chr8 | 1.35E+08 | 1.35E+08 | − | AluSq2 | chr8 |
| AluSx1 | chr8 | 1.42E+08 | 1.42E+08 | − | AluSq | chr8 |
| AluSx1 | chr8 | 1.42E+08 | 1.42E+08 | − | AluY | chr8 |
| AluY | chr8 | 1.42E+08 | 1.42E+08 | − | AluJb | chr8 |
| AluY | chr8 | 1.42E+08 | 1.42E+08 | + | AluSx3 | chr8 |
| AluSx1 | chr8 | 1.42E+08 | 1.42E+08 | − | AluJb | chr8 |
| AluSx1 | chr8 | 1.42E+08 | 1.42E+08 | − | AluY | chr8 |
| AluSx1 | chr8 | 1.42E+08 | 1.42E+08 | − | AluSp | chr8 |
| AluJr | chr8 | 1.44E+08 | 1.44E+08 | − | AluJr | chr8 |
| AluY | chr8 | 1.45E+08 | 1.45E+08 | − | AluSp | chr8 |
| AluJo | chr8 | 1.45E+08 | 1.45E+08 | − | AluSq2 | chr8 |
| AluSg | chr8 | 1.45E+08 | 1.45E+08 | + | AluSz | chr8 |
| AluJb | chr8 | 1.46E+08 | 1.46E+08 | + | AluSx1 | chr8 |
| AluY | chr8 | 1.46E+08 | 1.46E+08 | − | AluSx1 | chr8 |
| AluSp | chr8 | 1.46E+08 | 1.46E+08 | + | AluSg4 | chr8 |
| AluSx | chr17 | 1273733 | 1273975 | − | AluY | chr17 |
| AluY | chr17 | 1274008 | 1274313 | + | AluSx3 | chr17 |
| AluY | chr17 | 1606723 | 1606910 | − | AluSq | chr17 |
| AluY | chr17 | 1606723 | 1606910 | − | AluJb | chr17 |
| AluSq | chr17 | 2208989 | 2209292 | − | AluSx1 | chr17 |
| AluSg | chr17 | 2500698 | 2500998 | − | AluSq2 | chr17 |
| AluSq2 | chr17 | 2501259 | 2501567 | + | AluSz6 | chr17 |
| AluSq | chr17 | 4266144 | 4266430 | + | AluSx1 | chr17 |
| AluSg7 | chr17 | 4432225 | 4432528 | − | AluSx1 | chr17 |
| AluSx | chr17 | 4830358 | 4830664 | − | AluSp | chr17 |
| AluSx | chr17 | 4830358 | 4830664 | − | AluSc | chr17 |
| AluSp | chr17 | 4831979 | 4832251 | + | AluSx1 | chr17 |
| AluSc | chr17 | 4832253 | 4832454 | + | AluSx1 | chr17 |
| AluSx1 | chr17 | 4833045 | 4833344 | − | AluSx1 | chr17 |
| AluSp | chr17 | 4831979 | 4832251 | + | AluSp | chr17 |
| AluSp | chr17 | 4835057 | 4835351 | − | AluSx | chr17 |
| AluJb | chr17 | 4929763 | 4930056 | + | AluSx1 | chr17 |
| AluSx1 | chr17 | 4930410 | 4930724 | − | AluJr | chr17 |
| AluY | chr17 | 6220108 | 6220379 | − | AluSq2 | chr17 |
| AluSz | chr17 | 8173195 | 8173495 | − | AluSx | chr17 |
| AluSz | chr17 | 8173195 | 8173495 | − | AluY | chr17 |
| AluY | chr17 | 8175301 | 8175606 | + | AluSz | chr17 |
| AluSx | chr17 | 8174987 | 8175282 | + | AluSz | chr17 |
| AluSx | chr17 | 12913026 | 12913325 | + | AluSg | chr17 |
| AluSg4 | chr17 | 15886379 | 15886682 | + | AluSx | chr17 |
| AluSx1 | chr17 | 16249783 | 16250071 | + | AluJb | chr17 |
| AluSx | chr17 | 17737015 | 17737296 | + | AluJr | chr17 |
| AluSx | chr17 | 17870913 | 17871220 | − | AluSg4 | chr17 |
| AluSg4 | chr17 | 17871699 | 17871998 | + | AluSx1 | chr17 |
| AluSq | chr17 | 18175094 | 18175409 | + | AluSx3 | chr17 |
| AluJb | chr17 | 19156724 | 19157011 | − | AluSz | chr17 |
| AluY | chr17 | 20509730 | 20510030 | + | AluSz | chr17 |
| AluSq | chr17 | 20803462 | 20803776 | + | AluJb | chr17 |
| AluSq | chr17 | 20803462 | 20803776 | + | AluSg | chr17 |
| AluSp | chr17 | 20942609 | 20942884 | + | AluSg | chr17 |
| AluSg | chr17 | 20944201 | 20944495 | − | AluSx1 | chr17 |
| AluJb | chr17 | 26479655 | 26479949 | − | AluSp | chr17 |
| AluSx | chr17 | 26935378 | 26935672 | − | AluSz | chr17 |
| AluSg7 | chr17 | 27116219 | 27116510 | − | AluY | chr17 |
| AluY | chr17 | 27119048 | 27119349 | + | AluSz | chr17 |
| AluSz | chr17 | 27121235 | 27121537 | − | AluSz | chr17 |
| AluYf4 | chr17 | 27128190 | 27128499 | + | AluSc | chr17 |
| AluSx3 | chr17 | 27274003 | 27274297 | − | AluSx1 | chr17 |
| AluY | chr17 | 27496124 | 27496367 | − | AluJb | chr17 |
| AluJb | chr17 | 27498631 | 27498937 | + | AluYk4 | chr17 |
| AluJb | chr17 | 27498631 | 27498937 | + | AluJr | chr17 |
| AluYk4 | chr17 | 27500411 | 27500703 | − | AluSz | chr17 |
| AluJr | chr17 | 27500799 | 27501089 | − | AluSz | chr17 |
| AluSz | chr17 | 27501552 | 27501842 | + | AluJb | chr17 |
| AluJb | chr17 | 27502371 | 27502681 | − | AluSg7 | chr17 |
| AluYk4 | chr17 | 27500411 | 27500703 | − | AluSg7 | chr17 |
| AluJr | chr17 | 27500799 | 27501089 | − | AluSg7 | chr17 |
| AluSg | chr17 | 27598275 | 27598584 | + | AluY | chr17 |
| AluSx1 | chr17 | 27598807 | 27599083 | + | AluY | chr17 |
| AluSq | chr17 | 27604749 | 27605056 | + | AluSx | chr17 |
| AluSx | chr17 | 27606833 | 27607132 | − | AluSz | chr17 |
| AluSz | chr17 | 27607723 | 27608026 | + | AluY | chr17 |
| AluY | chr17 | 27608791 | 27609078 | − | AluSg | chr17 |
| AluSx | chr17 | 27606833 | 27607132 | − | AluSg | chr17 |
| AluJb | chr17 | 27731222 | 27731479 | − | AluSx | chr17 |
| AluSg | chr17 | 27749779 | 27750059 | + | AluSz | chr17 |
| AluSx1 | chr17 | 28080152 | 28080447 | − | AluSg7 | chr17 |
| AluSx1 | chr17 | 28777076 | 28777377 | − | AluJr | chr17 |
| AluSz6 | chr17 | 29062356 | 29062655 | + | AluSq10 | chr17 |
| AluSz | chr17 | 29063490 | 29063741 | + | AluSq10 | chr17 |
| AluSz6 | chr17 | 29062356 | 29062655 | + | AluSc8 | chr17 |
| AluSz | chr17 | 29063490 | 29063741 | + | AluSc8 | chr17 |
| AluSg | chr17 | 29078174 | 29078459 | + | AluSc | chr17 |
| AluSc | chr17 | 29078929 | 29079193 | − | AluSc | chr17 |
| AluY | chr17 | 29078623 | 29078918 | − | AluSc | chr17 |
| AluSc | chr17 | 29081226 | 29081529 | + | AluSg | chr17 |
| AluSc8 | chr17 | 29097638 | 29097937 | − | AluSg4 | chr17 |
| AluSx | chr17 | 29107989 | 29108296 | − | AluSq2 | chr17 |
| AluSq2 | chr17 | 29110361 | 29110651 | + | AluSc | chr17 |
| AluSc | chr17 | 29111777 | 29112065 | − | AluSx | chr17 |
| AluSc | chr17 | 29111777 | 29112065 | − | AluSc8 | chr17 |
| AluSx | chr17 | 29142553 | 29142861 | − | AluSx | chr17 |
| AluSx1 | chr17 | 29142092 | 29142403 | − | AluSx | chr17 |
| AluSx | chr17 | 29144174 | 29144464 | + | AluSq | chr17 |
| AluSx | chr17 | 29193204 | 29193513 | − | AluSz6 | chr17 |
| AluJb | chr17 | 29458954 | 29459249 | − | AluSx1 | chr17 |
| AluSg4 | chr17 | 30189033 | 30189324 | + | AluSq2 | chr17 |
| AluSc | chr17 | 30285585 | 30285885 | + | AluSz | chr17 |
| AluSz | chr17 | 30287621 | 30287896 | − | AluSx | chr17 |
| AluSc | chr17 | 30544495 | 30544798 | + | AluSx | chr17 |
| AluSc8 | chr17 | 30655628 | 30655937 | + | AluSx | chr17 |
| AluSx1 | chr17 | 30799560 | 30799854 | − | AluSx1 | chr17 |
| AluY | chr17 | 33381940 | 33382236 | − | AluSq2 | chr17 |
| AluSp | chr17 | 33403419 | 33403698 | + | AluJo | chr17 |
| AluSx | chr17 | 33897907 | 33898192 | − | AluSz6 | chr17 |
| AluSx1 | chr17 | 33937157 | 33937465 | − | AluY | chr17 |
| AluSx1 | chr17 | 34873098 | 34873399 | − | AluSq2 | chr17 |
| AluSq2 | chr17 | 34873651 | 34873946 | + | AluSx1 | chr17 |
| AluSc5 | chr17 | 34924596 | 34924891 | − | AluSx | chr17 |
| AluJr | chr17 | 35313067 | 35313354 | − | AluSq2 | chr17 |
| AluJr | chr17 | 35313067 | 35313354 | − | AluSq2 | chr17 |
| AluSp | chr17 | 35682510 | 35682812 | − | AluSx4 | chr17 |
| AluSp | chr17 | 35682510 | 35682812 | − | AluSx1 | chr17 |
| AluJb | chr17 | 35993483 | 35993707 | + | AluSx1 | chr17 |
| AluSg7 | chr17 | 36394552 | 36394835 | − | AluSg | chr17 |
| AluSx1 | chr17 | 36394103 | 36394400 | − | AluSg | chr17 |
| AluSq2 | chr17 | 36879996 | 36880294 | − | AluSx1 | chr17 |
| AluSx1 | chr17 | 37412292 | 37412597 | + | AluSg7 | chr17 |
| AluSz | chr17 | 37550810 | 37551111 | − | AluSx | chr17 |
| AluSp | chr17 | 37598886 | 37599183 | + | AluSg | chr17 |
| AluSz | chr17 | 38439157 | 38439444 | + | AluJr | chr17 |
| AluJo | chr17 | 38631317 | 38631628 | − | AluSx | chr17 |
| AluSx | chr17 | 39930580 | 39930884 | − | AluSg | chr17 |
| AluSq4 | chr17 | 39935207 | 39935511 | + | AluY | chr17 |
| AluSz6 | chr17 | 39989574 | 39989870 | − | AluSx | chr17 |
| AluJo | chr17 | 40008275 | 40008568 | − | AluSq | chr17 |
| AluY | chr17 | 40030825 | 40031125 | − | AluSq2 | chr17 |
| AluY | chr17 | 40158839 | 40159143 | + | AluY | chr17 |
| AluSg | chr17 | 40158535 | 40158836 | + | AluY | chr17 |
| AluSq2 | chr17 | 40160049 | 40160347 | + | AluY | chr17 |
| AluSx1 | chr17 | 40470137 | 40470443 | − | AluSz | chr17 |
| AluJb | chr17 | 40623470 | 40623759 | + | AluSz | chr17 |
| AluY | chr17 | 40623157 | 40623457 | + | AluSz | chr17 |
| AluSx1 | chr17 | 40622859 | 40623152 | + | AluSz | chr17 |
| AluY | chr17 | 41283560 | 41283750 | + | AluSz | chr17 |
| AluSz6 | chr17 | 41379533 | 41379831 | − | AluSp | chr17 |
| AluSx | chr17 | 42255842 | 42256134 | − | AluSg | chr17 |
| AluSc8 | chr17 | 42518228 | 42518515 | − | AluSq2 | chr17 |
| AluSq2 | chr17 | 42521859 | 42522143 | + | AluSq | chr17 |
| AluSp | chr17 | 42532588 | 42532895 | + | AluSx | chr17 |
| AluJr | chr17 | 43126524 | 43126820 | + | AluSq2 | chr17 |
| AluSx1 | chr17 | 43219478 | 43219786 | + | AluSg7 | chr17 |
| AluSq2 | chr17 | 44182183 | 44182478 | − | AluSq2 | chr17 |
| AluSq2 | chr17 | 44182183 | 44182478 | − | AluJb | chr17 |
| AluSx | chr17 | 44189769 | 44190048 | + | AluSp | chr17 |
| AluSq2 | chr17 | 44189454 | 44189761 | + | AluSp | chr17 |
| AluSq2 | chr17 | 44189454 | 44189761 | + | AluSq2 | chr17 |
| AluSx | chr17 | 44189769 | 44190048 | + | AluSq2 | chr17 |
| AluJb | chr17 | 45173274 | 45173580 | + | AluJr4 | chr17 |
| AluSg7 | chr17 | 46193349 | 46193644 | + | AluJb | chr17 |
| AluSc8 | chr17 | 47015951 | 47016249 | + | AluSc8 | chr17 |
| AluSz | chr17 | 47422560 | 47422863 | − | AluYk4 | chr17 |
| AluSz | chr17 | 48717122 | 48717424 | − | AluJb | chr17 |
| AluSz6 | chr17 | 48805912 | 48806213 | − | AluSz | chr17 |
| AluSp | chr17 | 49104025 | 49104336 | − | AluSz | chr17 |
| AluSc8 | chr17 | 49188200 | 49188497 | + | AluSx1 | chr17 |
| AluSq2 | chr17 | 53035810 | 53036109 | + | AluSz6 | chr17 |
| AluSz6 | chr17 | 53036978 | 53037277 | − | AluSx | chr17 |
| AluY | chr17 | 54907423 | 54907731 | + | AluSg7 | chr17 |
| AluSc8 | chr17 | 55179837 | 55180132 | − | AluSx1 | chr17 |
| AluSc8 | chr17 | 55381438 | 55381747 | + | AluSx1 | chr17 |
| AluSg7 | chr17 | 55533689 | 55533972 | + | AluY | chr17 |
| AluSz | chr17 | 55538895 | 55539181 | − | AluJb | chr17 |
| AluY | chr17 | 55545606 | 55545902 | + | AluSx | chr17 |
| AluSx | chr17 | 55724508 | 55724839 | + | AluSx | chr17 |
| AluSx | chr17 | 55914036 | 55914339 | − | AluSz | chr17 |
| AluSx1 | chr17 | 56085768 | 56086053 | + | AluSz | chr17 |
| AluSz | chr17 | 56088198 | 56088496 | − | AluSc | chr17 |
| AluSz | chr17 | 56461012 | 56461277 | − | AluJr | chr17 |
| AluSz | chr17 | 56461012 | 56461277 | − | AluSg | chr17 |
| AluSq2 | chr17 | 56474740 | 56475040 | + | AluSg | chr17 |
| AluSx | chr17 | 57280570 | 57280885 | − | AluJb | chr17 |
| AluSx | chr17 | 57280570 | 57280885 | − | AluSz | chr17 |
| AluY | chr17 | 57351898 | 57352162 | + | AluSx1 | chr17 |
| AluSx1 | chr17 | 57706044 | 57706322 | − | AluJb | chr17 |
| AluSx3 | chr17 | 57770003 | 57770298 | − | AluSz6 | chr17 |
| AluY | chr17 | 57884546 | 57884857 | − | AluSq2 | chr17 |
| AluJb | chr17 | 57921435 | 57921733 | − | AluSz | chr17 |
| AluSx1 | chr17 | 57920204 | 57920506 | − | AluSz | chr17 |
| AluSz | chr17 | 58037890 | 58038183 | + | AluSx | chr17 |
| AluSz | chr17 | 58037890 | 58038183 | + | AluSc8 | chr17 |
| AluSc | chr17 | 58584119 | 58584408 | + | AluJb | chr17 |
| AluY | chr17 | 59923304 | 59923552 | + | AluJb | chr17 |
| AluJr4 | chr17 | 59921665 | 59921971 | + | AluJb | chr17 |
| AluSp | chr17 | 60046435 | 60046733 | + | AluSz | chr17 |
| AluSx | chr17 | 60082259 | 60082554 | − | AluSz | chr17 |
| AluSx | chr17 | 60082259 | 60082554 | − | AluSc8 | chr17 |
| AluJo | chr17 | 60118940 | 60119243 | − | AluSc | chr17 |
| AluSg | chr17 | 60552714 | 60553005 | − | AluSx3 | chr17 |
| AluSc8 | chr17 | 60553028 | 60553320 | − | AluSx3 | chr17 |
| AluSg | chr17 | 60552714 | 60553005 | − | AluSx | chr17 |
| AluSc8 | chr17 | 60553028 | 60553320 | − | AluSx | chr17 |
| AluSx1 | chr17 | 62464413 | 62464705 | − | AluSx1 | chr17 |
| AluJb | chr17 | 62479349 | 62479649 | + | AluJb | chr17 |
| AluSz6 | chr17 | 62631163 | 62631468 | + | AluSz | chr17 |
| AluSq2 | chr17 | 64384415 | 64384712 | − | AluSq2 | chr17 |
| AluSz | chr17 | 65354033 | 65354336 | − | AluJb | chr17 |
| AluSx | chr17 | 65404069 | 65404369 | − | AluSg7 | chr17 |
| AluJo | chr17 | 65406500 | 65406791 | − | AluSg7 | chr17 |
| AluSz | chr17 | 65433902 | 65434211 | + | AluSx1 | chr17 |
| AluSx | chr17 | 65872640 | 65872949 | + | AluSq4 | chr17 |
| AluJb | chr17 | 66116713 | 66116943 | + | AluSp | chr17 |
| AluSx1 | chr17 | 66438335 | 66438631 | + | AluSx1 | chr17 |
| AluSz6 | chr17 | 70364184 | 70364469 | + | AluSx | chr17 |
| AluY | chr17 | 70380443 | 70380740 | − | AluJb | chr17 |
| AluSc | chr17 | 70491592 | 70491893 | − | AluSx | chr17 |
| AluSx3 | chr17 | 73097374 | 73097680 | + | AluJb | chr17 |
| AluSx3 | chr17 | 73097374 | 73097680 | + | AluSg4 | chr17 |
| AluSx3 | chr17 | 73097374 | 73097680 | + | AluSp | chr17 |
| AluSc | chr17 | 73136034 | 73136310 | − | AluSq | chr17 |
| AluSx1 | chr17 | 73533493 | 73533790 | + | AluSz6 | chr17 |
| AluSx1 | chr17 | 73939052 | 73939316 | + | AluSx | chr17 |
| AluSg | chr17 | 73973421 | 73973671 | + | AluSx1 | chr17 |
| AluSz | chr17 | 74388753 | 74389045 | + | AluSx1 | chr17 |
| AluSp | chr17 | 74555297 | 74555577 | − | AluSq2 | chr17 |
| AluSx3 | chr17 | 74750483 | 74750781 | + | AluSp | chr17 |
| AluSx3 | chr17 | 74750483 | 74750781 | + | AluSz | chr17 |
| AluJb | chr17 | 74761535 | 74761836 | − | AluSx | chr17 |
| AluJo | chr17 | 74781412 | 74781720 | − | AluSx1 | chr17 |
| AluSx1 | chr17 | 74781881 | 74782174 | + | AluSz | chr17 |
| AluJb | chr17 | 74956334 | 74956632 | − | AluJb | chr17 |
| AluJb | chr17 | 74956334 | 74956632 | − | AluSp | chr17 |
| AluSx1 | chr17 | 75459644 | 75459957 | + | AluSx1 | chr17 |
| AluJb | chr17 | 76177848 | 76178136 | − | AluSp | chr17 |
| AluY | chr17 | 76189777 | 76190081 | + | AluSz6 | chr17 |
| AluY | chr17 | 76189777 | 76190081 | + | AluSp | chr17 |
| AluSp | chr17 | 76191587 | 76191876 | − | AluSq2 | chr17 |
| AluSz6 | chr17 | 76191266 | 76191575 | − | AluSq2 | chr17 |
| AluSz6 | chr17 | 76191266 | 76191575 | − | AluJb | chr17 |
| AluSp | chr17 | 76191587 | 76191876 | − | AluJb | chr17 |
| AluJb | chr17 | 76194066 | 76194320 | + | AluSz | chr17 |
| AluSz | chr17 | 76400361 | 76400621 | − | AluY | chr17 |
| AluSc | chr17 | 78013216 | 78013474 | − | AluSx | chr17 |
| AluSx | chr17 | 78014196 | 78014503 | + | AluSz | chr17 |
| AluJo | chr17 | 79221346 | 79221665 | + | AluY | chr17 |
| AluSx1 | chr17 | 79270123 | 79270425 | − | AluSx3 | chr17 |
| AluSc | chr17 | 79527453 | 79527689 | + | AluSz | chr17 |
| AluSq2 | chr17 | 79599339 | 79599639 | − | AluY | chr17 |
| AluSz | chr17 | 79645594 | 79645889 | − | AluJr | chr17 |
| AluJr | chr17 | 79645925 | 79646213 | + | AluSx3 | chr17 |
| AluJr | chr17 | 79645925 | 79646213 | + | AluSp | chr17 |
| AluY | chr17 | 79839444 | 79839739 | − | AluSg | chr17 |
| AluY | chr17 | 79839444 | 79839739 | − | AluY | chr17 |
| AluY | chr17 | 79839444 | 79839739 | − | AluSc8 | chr17 |
| AluSc8 | chr17 | 79841746 | 79842055 | + | AluSq2 | chr17 |
| AluSp | chr17 | 79852582 | 79852893 | − | AluSc5 | chr17 |
| AluSp | chr17 | 80488085 | 80488342 | + | AluSx | chr17 |
| AluSz | chr17 | 80503457 | 80503757 | − | AluSq | chr17 |
| AluSc | chr17 | 80584132 | 80584434 | + | AluJb | chr17 |
| AluY | chr18 | 683985 | 684294 | − | AluJb | chr18 |
| AluSz | chr18 | 704160 | 704457 | + | AluSz | chr18 |
| AluSx1 | chr18 | 3601097 | 3601383 | − | AluSz | chr18 |
| AluSc | chr18 | 3599614 | 3599914 | − | AluSz | chr18 |
| AluSq | chr18 | 3606841 | 3607130 | − | AluSp | chr18 |
| AluSp | chr18 | 3608049 | 3608334 | + | AluSz | chr18 |
| AluSg7 | chr18 | 9368190 | 9368498 | − | AluSx | chr18 |
| AluSg7 | chr18 | 9368190 | 9368498 | − | AluSc | chr18 |
| AluSx1 | chr18 | 12964633 | 12964936 | − | AluSz | chr18 |
| AluY | chr18 | 19169794 | 19170087 | − | AluSc | chr18 |
| AluSg | chr18 | 19170182 | 19170480 | − | AluSc | chr18 |
| AluSx | chr18 | 19786365 | 19786673 | + | AluSz | chr18 |
| AluSc8 | chr18 | 21690959 | 21691211 | − | AluSg | chr18 |
| AluSz | chr18 | 23634708 | 23635001 | − | AluSq2 | chr18 |
| AluSg | chr18 | 29648877 | 29649183 | + | AluSz | chr18 |
| AluSg | chr18 | 29648877 | 29649183 | + | AluSp | chr18 |
| AluSg | chr18 | 29648877 | 29649183 | + | AluSz | chr18 |
| AluSx1 | chr18 | 43675857 | 43676154 | − | AluSp | chr18 |
| AluJb | chr18 | 61063492 | 61063782 | − | AluSx | chr18 |
| AluJb | chr18 | 61063492 | 61063782 | − | AluSc | chr18 |
| AluSc | chr4 | 372062 | 372356 | − | AluSq2 | chr4 |
| AluSq2 | chr4 | 374161 | 374460 | + | AluSz | chr4 |
| AluSq2 | chr4 | 374161 | 374460 | + | AluY | chr4 |
| AluSc8 | chr4 | 458587 | 458885 | + | AluY | chr4 |
| AluSc | chr4 | 479495 | 479761 | + | AluY | chr4 |
| AluSx | chr4 | 487186 | 487490 | + | AluY | chr4 |
| AluSx1 | chr4 | 764561 | 764876 | + | AluSx | chr4 |
| AluJr | chr4 | 768978 | 769261 | − | AluJr | chr4 |
| AluJr | chr4 | 771314 | 771597 | + | AluSg | chr4 |
| AluJr | chr4 | 771314 | 771597 | + | AluSq2 | chr4 |
| AluSg | chr4 | 772487 | 772730 | − | AluJo | chr4 |
| AluSc5 | chr4 | 1860274 | 1860563 | + | AluJo | chr4 |
| AluSz6 | chr4 | 1885077 | 1885376 | + | AluSc5 | chr4 |
| AluSz6 | chr4 | 1885077 | 1885376 | + | AluSz6 | chr4 |
| AluSx | chr4 | 2393488 | 2393660 | − | AluSx1 | chr4 |
| AluSx | chr4 | 2573274 | 2573566 | − | AluSx3 | chr4 |
| AluY | chr4 | 3086459 | 3086764 | + | AluSx1 | chr4 |
| AluSx1 | chr4 | 3945441 | 3945753 | + | AluJr | chr4 |
| AluSx1 | chr4 | 3945441 | 3945753 | + | AluSz | chr4 |
| AluSz | chr4 | 3946069 | 3946378 | − | AluSz6 | chr4 |
| AluSx3 | chr4 | 6790998 | 6791298 | − | AluJb | chr4 |
| AluY | chr4 | 6790686 | 6790997 | − | AluJb | chr4 |
| AluSx | chr4 | 17804654 | 17804947 | − | AluJr4 | chr4 |
| AluSx1 | chr4 | 39522412 | 39522715 | + | AluSc | chr4 |
| AluSx1 | chr4 | 39522412 | 39522715 | + | AluSz6 | chr4 |
| AluSc | chr4 | 39524385 | 39524673 | − | AluSp | chr4 |
| AluSz6 | chr4 | 39524677 | 39524971 | − | AluSp | chr4 |
| AluSp | chr4 | 39549526 | 39549824 | − | AluSx1 | chr4 |
| AluSx1 | chr4 | 39550533 | 39550833 | + | AluSz | chr4 |
| AluSx1 | chr4 | 39550533 | 39550833 | + | AluSx | chr4 |
| AluSx1 | chr4 | 39550533 | 39550833 | + | AluSz | chr4 |
| AluSz | chr4 | 39605876 | 39606158 | + | AluSz6 | chr4 |
| AluY | chr4 | 39712776 | 39713080 | − | AluSp | chr4 |
| AluSx | chr4 | 39718336 | 39718638 | + | AluSg | chr4 |
| AluJo | chr4 | 39725734 | 39726024 | − | AluJb | chr4 |
| AluJb | chr4 | 39724660 | 39724950 | − | AluJb | chr4 |
| AluSq | chr4 | 39758633 | 39758945 | − | AluSz | chr4 |
| AluJb | chr4 | 39758068 | 39758284 | − | AluSz | chr4 |
| AluSp | chr4 | 39833220 | 39833524 | + | AluY | chr4 |
| AluSx4 | chr4 | 39830989 | 39831284 | + | AluY | chr4 |
| AluY | chr4 | 39831290 | 39831600 | + | AluY | chr4 |
| AluSx4 | chr4 | 39833542 | 39833758 | + | AluY | chr4 |
| AluJb | chr4 | 39895749 | 39896045 | + | AluJr | chr4 |
| AluJr | chr4 | 39897647 | 39897953 | − | AluJb | chr4 |
| AluSp | chr4 | 39976157 | 39976426 | − | AluSx1 | chr4 |
| AluSx | chr4 | 48836939 | 48837250 | − | AluSx1 | chr4 |
| AluSq | chr4 | 56268002 | 56268302 | + | AluSz6 | chr4 |
| AluSz | chr4 | 56279470 | 56279781 | − | AluY | chr4 |
| AluJo | chr4 | 56279165 | 56279429 | − | AluY | chr4 |
| AluSq2 | chr4 | 56760209 | 56760509 | + | AluSp | chr4 |
| AluSz | chr4 | 71581862 | 71582162 | − | AluSq | chr4 |
| AluSq | chr4 | 71582279 | 71582592 | + | AluSp | chr4 |
| AluSz | chr4 | 71769689 | 71769999 | − | AluSg | chr4 |
| AluSz | chr4 | 73922557 | 73922861 | + | AluSq2 | chr4 |
| AluJo | chr4 | 74106059 | 74106349 | + | AluSx | chr4 |
| AluSc | chr4 | 76683318 | 76683614 | − | AluY | chr4 |
| AluSx3 | chr4 | 76693399 | 76693689 | − | AluSq2 | chr4 |
| AluSg | chr4 | 76858296 | 76858598 | + | AluJb | chr4 |
| AluSx | chr4 | 77863997 | 77864298 | + | AluSx1 | chr4 |
| AluSx | chr4 | 84381317 | 84381607 | − | AluSx | chr4 |
| AluSq2 | chr4 | 87844629 | 87844935 | + | AluSz | chr4 |
| AluJo | chr4 | 87852550 | 87852849 | − | AluY | chr4 |
| AluJo | chr4 | 87852550 | 87852849 | − | AluSp | chr4 |
| AluY | chr4 | 87904909 | 87905214 | + | AluSx | chr4 |
| AluSx1 | chr4 | 87918070 | 87918363 | − | AluY | chr4 |
| AluSx1 | chr4 | 89179955 | 89180268 | + | AluSq | chr4 |
| AluSx | chr4 | 89180269 | 89180443 | + | AluSq | chr4 |
| AluSx | chr4 | 99461495 | 99461788 | − | AluSz6 | chr4 |
| AluSp | chr4 | 99731043 | 99731323 | − | AluSx | chr4 |
| AluSp | chr4 | 1.1E+08 | 1.1E+08 | + | AluSc8 | chr4 |
| AluJb | chr4 | 1.29E+08 | 1.29E+08 | + | AluSx | chr4 |
| AluJb | chr4 | 1.29E+08 | 1.29E+08 | + | AluSx1 | chr4 |
| AluSz6 | chr4 | 1.29E+08 | 1.29E+08 | − | AluSz6 | chr4 |
| AluSx | chr4 | 1.29E+08 | 1.29E+08 | + | AluYf5 | chr4 |
| AluSp | chr4 | 1.29E+08 | 1.29E+08 | + | AluSx1 | chr4 |
| AluSx3 | chr4 | 1.29E+08 | 1.29E+08 | + | AluSx1 | chr4 |
| AluY | chr4 | 1.4E+08 | 1.4E+08 | − | AluSq2 | chr4 |
| AluY | chr4 | 1.4E+08 | 1.4E+08 | − | AluSq2 | chr4 |
| AluY | chr4 | 1.4E+08 | 1.4E+08 | + | AluSx | chr4 |
| AluSz | chr4 | 1.4E+08 | 1.4E+08 | + | AluY | chr4 |
| AluSx | chr4 | 1.41E+08 | 1.41E+08 | + | AluSx | chr4 |
| AluSx1 | chr4 | 1.41E+08 | 1.41E+08 | − | AluSc | chr4 |
| AluSx3 | chr4 | 1.52E+08 | 1.52E+08 | + | AluSp | chr4 |
| AluSx3 | chr4 | 1.52E+08 | 1.52E+08 | + | AluSq2 | chr4 |
| AluSq2 | chr4 | 1.52E+08 | 1.52E+08 | − | AluSz6 | chr4 |
| AluSz | chr4 | 1.52E+08 | 1.52E+08 | − | AluY | chr4 |
| AluJb | chr4 | 1.52E+08 | 1.52E+08 | + | AluSz | chr4 |
| AluSc | chr4 | 1.53E+08 | 1.53E+08 | − | AluJo | chr4 |
| AluSx | chr4 | 1.54E+08 | 1.54E+08 | − | AluSz6 | chr4 |
| AluSx | chr4 | 1.54E+08 | 1.54E+08 | − | AluJb | chr4 |
| AluJb | chr4 | 1.6E+08 | 1.6E+08 | + | AluJb | chr4 |
| AluSx3 | chr4 | 1.66E+08 | 1.66E+08 | − | AluJb | chr4 |
| AluSx1 | chr4 | 1.7E+08 | 1.7E+08 | + | AluSq2 | chr4 |
| AluY | chr4 | 1.86E+08 | 1.86E+08 | − | AluSc | chr4 |
| AluY | chr4 | 1.86E+08 | 1.86E+08 | − | AluY | chr4 |
| AluSc | chr4 | 1.86E+08 | 1.86E+08 | + | AluSx | chr4 |
| AluJb | chr16 | 262782 | 263063 | − | AluSx3 | chr16 |
| AluSx3 | chr16 | 265338 | 265652 | + | AluSg | chr16 |
| AluSx3 | chr16 | 265338 | 265652 | + | AluSx | chr16 |
| AluSg | chr16 | 266377 | 266678 | − | AluSq | chr16 |
| AluSx | chr16 | 267019 | 267315 | − | AluSq | chr16 |
| AluSz | chr16 | 1342081 | 1342388 | − | AluSx3 | chr16 |
| AluSg | chr16 | 1408862 | 1409136 | − | AluSx1 | chr16 |
| AluSg | chr16 | 1408862 | 1409136 | − | AluSx1 | chr16 |
| AluY | chr16 | 1523372 | 1523673 | − | AluSp | chr16 |
| AluSz | chr16 | 1567090 | 1567368 | + | AluJb | chr16 |
| AluSc | chr16 | 2911818 | 2912142 | − | AluY | chr16 |
| AluSx | chr16 | 2912143 | 2912411 | − | AluY | chr16 |
| AluJb | chr16 | 3083500 | 3083816 | + | AluSz | chr16 |
| AluJb | chr16 | 3083500 | 3083816 | + | AluJb | chr16 |
| AluJo | chr16 | 3511836 | 3512105 | + | AluSz | chr16 |
| AluSx | chr16 | 4331716 | 4332011 | + | AluYk4 | chr16 |
| AluY | chr16 | 4398951 | 4399255 | − | AluJb | chr16 |
| AluSz6 | chr16 | 4509726 | 4510024 | − | AluSc | chr16 |
| AluSz6 | chr16 | 4509726 | 4510024 | − | AluSq | chr16 |
| AluSc | chr16 | 4510597 | 4510899 | + | AluSx1 | chr16 |
| AluSx1 | chr16 | 4517234 | 4517534 | − | AluSp | chr16 |
| AluSx1 | chr16 | 4517234 | 4517534 | − | AluSx | chr16 |
| AluSq2 | chr16 | 4530653 | 4530954 | − | AluY | chr16 |
| AluY | chr16 | 4531068 | 4531379 | + | AluSz | chr16 |
| AluY | chr16 | 4678110 | 4678404 | + | AluSx | chr16 |
| AluY | chr16 | 4677800 | 4678101 | + | AluSx | chr16 |
| AluSc8 | chr16 | 4677507 | 4677799 | + | AluSx | chr16 |
| AluSz | chr16 | 5136055 | 5136353 | + | AluJr | chr16 |
| AluSq2 | chr16 | 8792192 | 8792490 | − | AluSz | chr16 |
| AluSx1 | chr16 | 8909982 | 8910282 | − | AluSp | chr16 |
| AluSx1 | chr16 | 11863281 | 11863585 | + | AluJb | chr16 |
| AluJb | chr16 | 11928694 | 11928957 | + | AluSx1 | chr16 |
| AluJb | chr16 | 11928694 | 11928957 | + | AluSc | chr16 |
| AluSx | chr16 | 11957785 | 11958082 | − | AluSx | chr16 |
| AluSq2 | chr16 | 11989006 | 11989305 | + | AluSp | chr16 |
| AluSp | chr16 | 11992299 | 11992606 | − | AluSq | chr16 |
| AluSq | chr16 | 11993910 | 11994200 | + | AluSx | chr16 |
| AluSx1 | chr16 | 12003500 | 12003804 | + | AluSx | chr16 |
| AluSx | chr16 | 12005409 | 12005707 | − | AluSx1 | chr16 |
| AluSx | chr16 | 12127456 | 12127743 | − | AluSx1 | chr16 |
| AluSz6 | chr16 | 15792445 | 15792733 | + | AluJb | chr16 |
| AluSz6 | chr16 | 15792445 | 15792733 | + | AluSz6 | chr16 |
| AluSz6 | chr16 | 15792445 | 15792733 | + | AluSz | chr16 |
| AluSz6 | chr16 | 15793964 | 15794251 | − | AluSx | chr16 |
| AluJb | chr16 | 15793121 | 15793335 | − | AluSx | chr16 |
| AluSq | chr16 | 19065756 | 19066049 | + | AluSz | chr16 |
| AluSx1 | chr16 | 19129505 | 19129803 | + | AluJo | chr16 |
| AluSx | chr16 | 19473940 | 19474249 | + | AluJr | chr16 |
| AluSc | chr16 | 19714032 | 19714347 | + | AluSg | chr16 |
| AluSx1 | chr16 | 21966083 | 21966396 | + | AluSp | chr16 |
| AluSp | chr16 | 21967139 | 21967444 | − | AluSx | chr16 |
| AluSz | chr16 | 22407539 | 22407827 | − | AluJb | chr16 |
| AluY | chr16 | 22504693 | 22504990 | + | AluY | chr16 |
| AluSx | chr16 | 22517933 | 22518222 | + | AluY | chr16 |
| AluSx1 | chr16 | 23537040 | 23537339 | + | AluSx | chr16 |
| AluSq2 | chr16 | 27488622 | 27488925 | − | AluSz6 | chr16 |
| AluSg | chr16 | 28210898 | 28211164 | − | AluSx1 | chr16 |
| AluSx1 | chr16 | 28968343 | 28968653 | − | AluJr | chr16 |
| AluSx | chr16 | 29417757 | 29418049 | + | AluSz6 | chr16 |
| AluSc8 | chr16 | 29594521 | 29594818 | + | AluSc | chr16 |
| AluSx1 | chr16 | 29706799 | 29707110 | − | AluJr | chr16 |
| AluSx1 | chr16 | 29706799 | 29707110 | − | AluSg | chr16 |
| AluSx1 | chr16 | 29706799 | 29707110 | − | AluSz | chr16 |
| AluJb | chr16 | 30589378 | 30589676 | + | AluY | chr16 |
| AluY | chr16 | 30591676 | 30591975 | − | AluSq2 | chr16 |
| AluY | chr16 | 30591676 | 30591975 | − | AluY | chr16 |
| AluSx | chr16 | 30591508 | 30591675 | − | AluY | chr16 |
| AluY | chr16 | 30591676 | 30591975 | − | AluSg7 | chr16 |
| AluSg | chr16 | 30781441 | 30781728 | − | AluY | chr16 |
| AluSz | chr16 | 30894858 | 30895137 | − | AluY | chr16 |
| AluSp | chr16 | 30945078 | 30945371 | − | AluSq2 | chr16 |
| AluY | chr16 | 31073820 | 31074114 | + | AluJo | chr16 |
| AluSx1 | chr16 | 31132438 | 31132741 | − | AluY | chr16 |
| AluSx1 | chr16 | 31132438 | 31132741 | − | AluSz | chr16 |
| AluJb | chr16 | 47447324 | 47447613 | + | AluSq10 | chr16 |
| AluSz | chr16 | 52538738 | 52539045 | − | AluJr | chr16 |
| AluJr4 | chr16 | 53136480 | 53136761 | + | AluY | chr16 |
| AluJr | chr16 | 53198204 | 53198503 | + | AluSz6 | chr16 |
| AluSq | chr16 | 53728064 | 53728376 | + | AluSq2 | chr16 |
| AluSx1 | chr16 | 56477085 | 56477385 | − | AluSq2 | chr16 |
| AluSz | chr16 | 57203994 | 57204295 | − | AluSq | chr16 |
| AluJr | chr16 | 58652816 | 58653111 | + | AluSg | chr16 |
| AluJr | chr16 | 58652816 | 58653111 | + | AluSp | chr16 |
| AluSx3 | chr16 | 66719544 | 66719847 | − | AluSc | chr16 |
| AluSx3 | chr16 | 66719544 | 66719847 | − | AluSz | chr16 |
| AluJr | chr16 | 66857637 | 66857939 | − | AluY | chr16 |
| AluJr | chr16 | 67170472 | 67170746 | − | AluY | chr16 |
| AluSc | chr16 | 67607654 | 67607945 | + | AluSx1 | chr16 |
| AluSg | chr16 | 67715927 | 67716238 | − | AluSx | chr16 |
| AluSz | chr16 | 67715330 | 67715611 | − | AluSx | chr16 |
| AluSx1 | chr16 | 67884713 | 67885003 | − | AluSc5 | chr16 |
| AluSx | chr16 | 68779726 | 68780019 | − | AluSg | chr16 |
| AluSx | chr16 | 68798198 | 68798495 | + | AluSx1 | chr16 |
| AluSx1 | chr16 | 69158327 | 69158577 | − | AluSq | chr16 |
| AluSc | chr16 | 69193316 | 69193606 | + | AluJr | chr16 |
| AluSz | chr16 | 69376216 | 69376497 | + | AluJb | chr16 |
| AluSx | chr16 | 69410822 | 69411126 | + | AluSc | chr16 |
| AluSc | chr16 | 69411235 | 69411497 | − | AluSg7 | chr16 |
| AluSc5 | chr16 | 69462281 | 69462578 | + | AluJb | chr16 |
| AluSc5 | chr16 | 69462281 | 69462578 | + | AluSq2 | chr16 |
| AluJb | chr16 | 69464121 | 69464422 | − | AluJb | chr16 |
| AluSq2 | chr16 | 69464577 | 69464875 | − | AluJb | chr16 |
| AluY | chr16 | 69485573 | 69485866 | + | AluSx1 | chr16 |
| AluSz6 | chr16 | 69486302 | 69486580 | + | AluSx1 | chr16 |
| AluSc | chr16 | 69486630 | 69486914 | + | AluSx1 | chr16 |
| AluY | chr16 | 69485573 | 69485866 | + | AluSc8 | chr16 |
| AluSc | chr16 | 69486630 | 69486914 | + | AluSc8 | chr16 |
| AluSz6 | chr16 | 69486302 | 69486580 | + | AluSc8 | chr16 |
| AluSc | chr16 | 69486630 | 69486914 | + | AluSc5 | chr16 |
| AluSz6 | chr16 | 69486302 | 69486580 | + | AluSc5 | chr16 |
| AluSx1 | chr16 | 69667899 | 69668202 | + | AluSq2 | chr16 |
| AluY | chr16 | 69698175 | 69698487 | − | AluSp | chr16 |
| AluSq2 | chr16 | 69709531 | 69709843 | − | AluSx1 | chr16 |
| AluSx1 | chr16 | 69711774 | 69712081 | + | AluSg | chr16 |
| AluJr | chr16 | 69722895 | 69723197 | + | AluSz6 | chr16 |
| AluSx | chr16 | 69721063 | 69721358 | + | AluSz6 | chr16 |
| AluSg | chr16 | 70297869 | 70298163 | + | AluSq2 | chr16 |
| AluY | chr16 | 70296591 | 70296900 | + | AluSq2 | chr16 |
| AluSg7 | chr16 | 70410652 | 70410943 | − | AluSx | chr16 |
| AluSg7 | chr16 | 70410652 | 70410943 | − | AluSx | chr16 |
| AluY | chr16 | 70463215 | 70463513 | + | AluSq2 | chr16 |
| AluSz | chr16 | 70608391 | 70608695 | − | AluJo | chr16 |
| AluY | chr16 | 71958915 | 71959216 | − | AluSx1 | chr16 |
| AluSx1 | chr16 | 71959903 | 71960191 | + | AluY | chr16 |
| AluY | chr16 | 71960254 | 71960547 | − | AluSz | chr16 |
| AluY | chr16 | 71958915 | 71959216 | − | AluSz | chr16 |
| AluSq4 | chr16 | 74361633 | 74361922 | − | AluSc5 | chr16 |
| AluSc5 | chr16 | 74363548 | 74363843 | + | AluSp | chr16 |
| AluSc | chr16 | 74361311 | 74361613 | + | AluSp | chr16 |
| AluSg | chr16 | 74674044 | 74674341 | − | AluSx3 | chr16 |
| AluSg | chr16 | 74674044 | 74674341 | − | AluSq2 | chr16 |
| AluJo | chr16 | 75484577 | 75484858 | − | AluJb | chr16 |
| AluSx | chr16 | 75507789 | 75508092 | + | AluJb | chr16 |
| AluJb | chr16 | 75508182 | 75508472 | − | AluSz | chr16 |
| AluSp | chr16 | 75626098 | 75626389 | − | AluY | chr16 |
| AluY | chr16 | 75626839 | 75627147 | + | AluSz6 | chr16 |
| AluYb8 | chr16 | 78154187 | 78154491 | + | AluSg7 | chr16 |
| AluSz6 | chr16 | 81088266 | 81088552 | − | AluJb | chr16 |
| AluJb | chr16 | 81089021 | 81089308 | + | AluSz | chr16 |
| AluSq2 | chr16 | 84107333 | 84107632 | + | AluJb | chr16 |
| AluSg7 | chr16 | 85017326 | 85017570 | + | AluSx | chr16 |
| AluSx1 | chr16 | 87389612 | 87389862 | + | AluSq2 | chr16 |
| AluSz | chr16 | 87770912 | 87771166 | + | AluSx1 | chr16 |
| AluJo | chr16 | 87776734 | 87777000 | − | AluY | chr16 |
| AluJo | chr16 | 89471745 | 89472040 | − | AluSx | chr16 |
| AluJr | chr16 | 89558166 | 89558450 | − | AluSz | chr16 |
| AluY | chr16 | 89581344 | 89581643 | − | AluSz6 | chr16 |
| AluSg | chr16 | 89581666 | 89581973 | − | AluSz6 | chr16 |
| AluSg | chr16 | 89581666 | 89581973 | − | AluY | chr16 |
| AluY | chr16 | 89581344 | 89581643 | − | AluY | chr16 |
| AluYk4 | chr16 | 89594517 | 89594827 | + | AluJb | chr16 |
| AluYk4 | chr16 | 89594517 | 89594827 | + | AluSg | chr16 |
| AluYc | chr16 | 89719418 | 89719716 | − | AluSc | chr16 |
| AluSx | chr16 | 89716580 | 89716750 | − | AluSc | chr16 |
| AluY | chr16 | 89754486 | 89754788 | − | AluSz | chr16 |
| AluY | chr16 | 89878462 | 89878767 | + | AluSq2 | chr16 |
| AluSp | chr16 | 89878773 | 89879078 | + | AluSq2 | chr16 |
| AluY | chr16 | 89931404 | 89931713 | − | AluSc8 | chr16 |
| AluJr4 | chr16 | 90054277 | 90054564 | + | AluJb | chr16 |
| AluYk4 | chr3 | 9487691 | 9487992 | + | AluSc | chr3 |
| AluSq2 | chr3 | 9842113 | 9842408 | − | AluSg7 | chr3 |
| AluY | chr3 | 10039769 | 10040050 | + | AluSx1 | chr3 |
| AluSx | chr3 | 10192564 | 10192864 | − | AluJr | chr3 |
| AluJr | chr3 | 10193472 | 10193766 | + | AluSc8 | chr3 |
| AluYk4 | chr3 | 10323003 | 10323311 | − | AluSz | chr3 |
| AluSz | chr3 | 11735813 | 11736112 | + | AluSg7 | chr3 |
| AluJb | chr3 | 12444510 | 12444806 | + | AluJr | chr3 |
| AluSq | chr3 | 12577047 | 12577304 | − | AluSq | chr3 |
| AluSc | chr3 | 15918687 | 15918990 | − | AluSx1 | chr3 |
| AluSq2 | chr3 | 17751775 | 17752086 | + | AluSg | chr3 |
| AluSc | chr3 | 20015655 | 20015944 | − | AluSx | chr3 |
| AluJb | chr3 | 23290480 | 23290779 | + | AluY | chr3 |
| AluY | chr3 | 23897039 | 23897349 | + | AluSx3 | chr3 |
| AluSz | chr3 | 27516331 | 27516609 | + | AluSx | chr3 |
| AluSx1 | chr3 | 28284549 | 28284846 | − | AluSx3 | chr3 |
| AluY | chr3 | 30661399 | 30661702 | + | AluSq2 | chr3 |
| AluSx1 | chr3 | 31680381 | 31680667 | + | AluSx1 | chr3 |
| AluSx1 | chr3 | 31735581 | 31735889 | + | AluSp | chr3 |
| AluSg | chr3 | 31770917 | 31771210 | − | AluSc5 | chr3 |
| AluSc | chr3 | 31951475 | 31951770 | − | AluSx | chr3 |
| AluSz6 | chr3 | 32284353 | 32284684 | − | AluJb | chr3 |
| AluSx | chr3 | 32796001 | 32796313 | − | AluSq2 | chr3 |
| AluSc8 | chr3 | 34272384 | 34272635 | + | AluSx3 | chr3 |
| AluSz | chr3 | 40432446 | 40432753 | − | AluSx1 | chr3 |
| AluSx | chr3 | 40504700 | 40504996 | − | AluSx | chr3 |
| AluSg | chr3 | 44479652 | 44479951 | − | AluSz | chr3 |
| AluSz6 | chr3 | 44537658 | 44537960 | − | AluY | chr3 |
| AluJb | chr3 | 44801140 | 44801407 | + | AluY | chr3 |
| AluJb | chr3 | 44801140 | 44801407 | + | AluSx | chr3 |
| AluJb | chr3 | 47176783 | 47177053 | − | AluSx1 | chr3 |
| AluSx3 | chr3 | 47487667 | 47487960 | − | AluSg | chr3 |
| AluSz | chr3 | 47510556 | 47510844 | + | AluSz | chr3 |
| AluSp | chr3 | 47947890 | 47948186 | + | AluSp | chr3 |
| AluSz | chr3 | 48287521 | 48287792 | + | AluSq2 | chr3 |
| AluSq2 | chr3 | 48290588 | 48290892 | − | AluSx1 | chr3 |
| AluSx1 | chr3 | 48291336 | 48291631 | + | AluSx | chr3 |
| AluSx | chr3 | 48291967 | 48292261 | − | AluSx3 | chr3 |
| AluSq2 | chr3 | 48290588 | 48290892 | − | AluSx3 | chr3 |
| AluSx | chr3 | 48291967 | 48292261 | − | AluJb | chr3 |
| AluSq2 | chr3 | 48290588 | 48290892 | − | AluJb | chr3 |
| AluSx3 | chr3 | 48292855 | 48293064 | + | AluSc8 | chr3 |
| AluSx1 | chr3 | 48291336 | 48291631 | + | AluSc8 | chr3 |
| AluJb | chr3 | 48293251 | 48293543 | + | AluSc8 | chr3 |
| AluSx3 | chr3 | 48292855 | 48293064 | + | AluYf4 | chr3 |
| AluJb | chr3 | 48293251 | 48293543 | + | AluYf4 | chr3 |
| AluSx1 | chr3 | 48291336 | 48291631 | + | AluYf4 | chr3 |
| AluSx3 | chr3 | 48292855 | 48293064 | + | AluY | chr3 |
| AluJb | chr3 | 48293251 | 48293543 | + | AluY | chr3 |
| AluJb | chr3 | 48745473 | 48745707 | + | AluY | chr3 |
| AluY | chr3 | 48748051 | 48748341 | − | AluSx | chr3 |
| AluSx | chr3 | 48751273 | 48751550 | + | AluSz | chr3 |
| AluSx | chr3 | 48751273 | 48751550 | + | AluSg4 | chr3 |
| AluSq2 | chr3 | 49030094 | 49030398 | − | AluSp | chr3 |
| AluSx1 | chr3 | 49046297 | 49046614 | − | AluY | chr3 |
| AluSg | chr3 | 49099489 | 49099792 | + | AluSz | chr3 |
| AluY | chr3 | 49122313 | 49122588 | + | AluY | chr3 |
| AluSx1 | chr3 | 49121995 | 49122214 | + | AluY | chr3 |
| AluSq2 | chr3 | 49397901 | 49398190 | − | AluSc | chr3 |
| AluSp | chr3 | 49460908 | 49461207 | + | AluSp | chr3 |
| AluSx | chr3 | 50058791 | 50059097 | + | AluSp | chr3 |
| AluSp | chr3 | 50070138 | 50070438 | + | AluSc | chr3 |
| AluSp | chr3 | 50070138 | 50070438 | + | AluSg7 | chr3 |
| AluSp | chr3 | 50370244 | 50370539 | + | AluSg7 | chr3 |
| AluJo | chr3 | 50369936 | 50370241 | + | AluSg7 | chr3 |
| AluSx | chr3 | 56699403 | 56699686 | − | AluY | chr3 |
| AluSg | chr3 | 57577184 | 57577491 | − | AluSx1 | chr3 |
| AluSx | chr3 | 57829495 | 57829804 | − | AluSz6 | chr3 |
| AluSc | chr3 | 58010429 | 58010737 | − | AluSz | chr3 |
| AluSc8 | chr3 | 73102780 | 73103075 | + | AluSx | chr3 |
| AluY | chr3 | 75461599 | 75461801 | + | AluSx | chr3 |
| AluSx | chr3 | 75463010 | 75463313 | − | AluYc | chr3 |
| AluSx | chr3 | 75463010 | 75463313 | − | AluSx1 | chr3 |
| AluSx1 | chr3 | 75465732 | 75466018 | + | AluSx | chr3 |
| AluYc | chr3 | 75463358 | 75463617 | + | AluSx | chr3 |
| AluSx1 | chr3 | 75465732 | 75466018 | + | AluSx1 | chr3 |
| AluSz | chr3 | 75474019 | 75474324 | + | AluJb | chr3 |
| AluY | chr3 | 75782648 | 75782951 | + | AluY | chr3 |
| AluY | chr3 | 75782648 | 75782951 | + | AluSc8 | chr3 |
| AluJr | chr3 | 78964826 | 78965100 | − | AluY | chr3 |
| AluSx1 | chr3 | 88137949 | 88138222 | − | AluSc8 | chr3 |
| AluSq2 | chr3 | 93846785 | 93847075 | + | AluSg7 | chr3 |
| AluSc | chr3 | 1.01E+08 | 1.01E+08 | + | AluSx3 | chr3 |
| AluSx | chr3 | 1.13E+08 | 1.13E+08 | − | AluSx | chr3 |
| AluJb | chr3 | 1.14E+08 | 1.14E+08 | − | AluJb | chr3 |
| AluSx3 | chr3 | 1.16E+08 | 1.16E+08 | + | AluY | chr3 |
| AluSx | chr3 | 1.25E+08 | 1.25E+08 | + | AluSx | chr3 |
| AluSz | chr3 | 1.25E+08 | 1.25E+08 | − | AluSz6 | chr3 |
| AluSc8 | chr3 | 1.29E+08 | 1.29E+08 | + | AluSc | chr3 |
| AluSx | chr3 | 1.29E+08 | 1.29E+08 | + | AluSc | chr3 |
| AluSg | chr3 | 1.29E+08 | 1.29E+08 | + | AluSx3 | chr3 |
| AluJr | chr3 | 1.29E+08 | 1.29E+08 | + | AluSx1 | chr3 |
| AluJb | chr3 | 1.29E+08 | 1.29E+08 | + | AluSg | chr3 |
| AluSx | chr3 | 1.29E+08 | 1.29E+08 | + | AluJb | chr3 |
| AluJb | chr3 | 1.3E+08 | 1.3E+08 | − | AluJr | chr3 |
| AluSz | chr3 | 1.3E+08 | 1.3E+08 | + | AluJb | chr3 |
| AluSx3 | chr3 | 1.31E+08 | 1.31E+08 | − | AluSz | chr3 |
| AluSc8 | chr3 | 1.31E+08 | 1.31E+08 | − | AluSz6 | chr3 |
| AluSx1 | chr3 | 1.36E+08 | 1.36E+08 | − | AluSc | chr3 |
| AluSc | chr3 | 1.36E+08 | 1.36E+08 | + | AluSx | chr3 |
| AluSx | chr3 | 1.36E+08 | 1.36E+08 | − | AluJb | chr3 |
| AluSx1 | chr3 | 1.36E+08 | 1.36E+08 | − | AluJb | chr3 |
| AluSx1 | chr3 | 1.37E+08 | 1.37E+08 | + | AluSx1 | chr3 |
| AluSq | chr3 | 1.38E+08 | 1.38E+08 | − | AluSg | chr3 |
| AluJb | chr3 | 1.38E+08 | 1.38E+08 | − | AluSx4 | chr3 |
| AluSc8 | chr3 | 1.39E+08 | 1.39E+08 | + | AluSx | chr3 |
| AluSg4 | chr3 | 1.39E+08 | 1.39E+08 | − | AluSx | chr3 |
| AluSp | chr3 | 1.42E+08 | 1.42E+08 | − | AluSg | chr3 |
| AluSg | chr3 | 1.42E+08 | 1.42E+08 | + | AluSc8 | chr3 |
| AluSp | chr3 | 1.42E+08 | 1.42E+08 | − | AluSx | chr3 |
| AluSc8 | chr3 | 1.42E+08 | 1.42E+08 | − | AluSx | chr3 |
| AluSg | chr3 | 1.42E+08 | 1.42E+08 | + | AluJb | chr3 |
| AluSx4 | chr3 | 1.42E+08 | 1.42E+08 | − | AluSx | chr3 |
| AluSx4 | chr3 | 1.42E+08 | 1.42E+08 | − | AluSx | chr3 |
| AluSx | chr3 | 1.42E+08 | 1.42E+08 | − | AluSg | chr3 |
| AluSc | chr3 | 1.42E+08 | 1.42E+08 | − | AluY | chr3 |
| AluSc | chr3 | 1.42E+08 | 1.42E+08 | − | AluJo | chr3 |
| AluJo | chr3 | 1.42E+08 | 1.42E+08 | − | AluJr | chr3 |
| AluJr4 | chr3 | 1.42E+08 | 1.42E+08 | + | AluSx | chr3 |
| AluSq2 | chr3 | 1.49E+08 | 1.49E+08 | + | AluSp | chr3 |
| AluSq | chr3 | 1.56E+08 | 1.56E+08 | − | AluSx3 | chr3 |
| AluSx1 | chr3 | 1.56E+08 | 1.56E+08 | − | AluSx | chr3 |
| AluSp | chr3 | 1.56E+08 | 1.56E+08 | − | AluSx | chr3 |
| AluSp | chr3 | 1.56E+08 | 1.56E+08 | − | AluSz | chr3 |
| AluSp | chr3 | 1.56E+08 | 1.56E+08 | − | AluSz | chr3 |
| AluSz | chr3 | 1.56E+08 | 1.56E+08 | + | AluSc8 | chr3 |
| AluSz | chr3 | 1.56E+08 | 1.56E+08 | + | AluSc8 | chr3 |
| AluSc8 | chr3 | 1.56E+08 | 1.56E+08 | − | AluSc5 | chr3 |
| AluSp | chr3 | 1.56E+08 | 1.56E+08 | − | AluSx | chr3 |
| AluYk4 | chr3 | 1.56E+08 | 1.56E+08 | + | AluY | chr3 |
| AluSx | chr3 | 1.69E+08 | 1.69E+08 | + | AluSq | chr3 |
| AluJb | chr3 | 1.7E+08 | 1.7E+08 | − | AluSx1 | chr3 |
| AluJb | chr3 | 1.7E+08 | 1.7E+08 | − | AluSx1 | chr3 |
| AluSx3 | chr3 | 1.7E+08 | 1.7E+08 | + | AluSc | chr3 |
| AluSz | chr3 | 1.7E+08 | 1.7E+08 | + | AluSq2 | chr3 |
| AluSp | chr3 | 1.72E+08 | 1.72E+08 | − | AluSz6 | chr3 |
| AluY | chr3 | 1.72E+08 | 1.72E+08 | − | AluSg | chr3 |
| AluSx1 | chr3 | 1.72E+08 | 1.72E+08 | + | AluSx | chr3 |
| AluSg4 | chr3 | 1.73E+08 | 1.73E+08 | + | AluJb | chr3 |
| AluJr | chr3 | 1.77E+08 | 1.77E+08 | − | AluJb | chr3 |
| AluJb | chr3 | 1.77E+08 | 1.77E+08 | + | AluSz | chr3 |
| AluSz | chr3 | 1.77E+08 | 1.77E+08 | + | AluJb | chr3 |
| AluSx1 | chr3 | 1.77E+08 | 1.77E+08 | + | AluSg | chr3 |
| AluSg | chr3 | 1.77E+08 | 1.77E+08 | − | AluY | chr3 |
| AluSg | chr3 | 1.77E+08 | 1.77E+08 | − | AluSx1 | chr3 |
| AluSp | chr3 | 1.77E+08 | 1.77E+08 | + | AluJb | chr3 |
| AluY | chr3 | 1.77E+08 | 1.77E+08 | + | AluSx | chr3 |
| AluSq2 | chr3 | 1.77E+08 | 1.77E+08 | − | AluSz | chr3 |
| AluY | chr3 | 1.77E+08 | 1.77E+08 | + | AluSg | chr3 |
| AluSx4 | chr3 | 1.79E+08 | 1.79E+08 | − | AluSx1 | chr3 |
| AluY | chr3 | 1.79E+08 | 1.79E+08 | − | AluSz | chr3 |
| AluY | chr3 | 1.81E+08 | 1.81E+08 | + | AluSp | chr3 |
| AluY | chr3 | 1.81E+08 | 1.81E+08 | + | AluJo | chr3 |
| AluSc5 | chr3 | 1.83E+08 | 1.83E+08 | − | AluSq2 | chr3 |
| AluSg | chr3 | 1.83E+08 | 1.83E+08 | − | AluSc | chr3 |
| AluSz6 | chr3 | 1.83E+08 | 1.83E+08 | − | AluSc5 | chr3 |
| AluSc5 | chr3 | 1.83E+08 | 1.83E+08 | + | AluY | chr3 |
| AluSx1 | chr3 | 1.83E+08 | 1.83E+08 | − | AluJo | chr3 |
| AluJr | chr3 | 1.84E+08 | 1.84E+08 | + | AluSz | chr3 |
| AluJr | chr3 | 1.84E+08 | 1.84E+08 | − | AluJb | chr3 |
| AluSx1 | chr3 | 1.84E+08 | 1.84E+08 | − | AluJb | chr3 |
| AluSx | chr3 | 1.84E+08 | 1.84E+08 | − | AluJb | chr3 |
| AluY | chr3 | 1.84E+08 | 1.84E+08 | − | AluJb | chr3 |
| AluSx | chr3 | 1.85E+08 | 1.85E+08 | − | AluSc | chr3 |
| AluSz | chr3 | 1.85E+08 | 1.85E+08 | − | AluSx1 | chr3 |
| AluSz | chr3 | 1.85E+08 | 1.85E+08 | − | AluSz | chr3 |
| AluSz | chr3 | 1.85E+08 | 1.85E+08 | + | AluSx1 | chr3 |
| AluSx1 | chr3 | 1.85E+08 | 1.85E+08 | + | AluSx1 | chr3 |
| AluSq2 | chr3 | 1.85E+08 | 1.85E+08 | + | AluJr | chr3 |
| AluJr | chr3 | 1.85E+08 | 1.85E+08 | − | AluSz | chr3 |
| AluJo | chr3 | 1.85E+08 | 1.85E+08 | + | AluJb | chr3 |
| AluSx | chr3 | 1.85E+08 | 1.85E+08 | − | AluSx1 | chr3 |
| AluSg7 | chr3 | 1.86E+08 | 1.86E+08 | + | AluJo | chr3 |
| AluSg7 | chr3 | 1.86E+08 | 1.86E+08 | + | AluY | chr3 |
| AluY | chr3 | 1.86E+08 | 1.86E+08 | − | AluSx | chr3 |
| AluJo | chr3 | 1.86E+08 | 1.86E+08 | − | AluSx | chr3 |
| AluSx | chr3 | 1.86E+08 | 1.86E+08 | + | AluJb | chr3 |
| AluSz | chr3 | 1.91E+08 | 1.91E+08 | − | AluSc | chr3 |
| AluSg | chr3 | 1.94E+08 | 1.94E+08 | + | AluSx | chr3 |
| AluSz | chr3 | 1.94E+08 | 1.94E+08 | − | AluSx1 | chr3 |
| AluSx1 | chr3 | 1.94E+08 | 1.94E+08 | + | AluSz | chr3 |
| AluSx4 | chr3 | 1.94E+08 | 1.94E+08 | + | AluSx1 | chr3 |
| AluSz | chr3 | 1.94E+08 | 1.94E+08 | + | AluSx1 | chr3 |
| AluSg | chr3 | 1.95E+08 | 1.95E+08 | + | AluY | chr3 |
| AluSx | chr3 | 1.96E+08 | 1.96E+08 | − | AluSq | chr3 |
| AluSp | chr3 | 1.96E+08 | 1.96E+08 | + | AluSx | chr3 |
| AluSx | chr3 | 1.96E+08 | 1.96E+08 | − | AluSc5 | chr3 |
| AluJo | chr3 | 1.96E+08 | 1.96E+08 | − | AluSc5 | chr3 |
| AluSc5 | chr3 | 1.96E+08 | 1.96E+08 | + | AluSx | chr3 |
| AluSx | chr3 | 1.96E+08 | 1.96E+08 | − | AluSx3 | chr3 |
| AluSx | chr3 | 1.96E+08 | 1.96E+08 | − | AluSx3 | chr3 |
| AluJo | chr3 | 1.96E+08 | 1.96E+08 | − | AluSx3 | chr3 |
| AluSx | chr3 | 1.96E+08 | 1.96E+08 | − | AluSz | chr3 |
| AluSx1 | chr3 | 1.96E+08 | 1.96E+08 | + | AluSx | chr3 |
| AluSx3 | chr3 | 1.96E+08 | 1.96E+08 | + | AluSx | chr3 |
| AluSx3 | chr3 | 1.96E+08 | 1.96E+08 | + | AluSx | chr3 |
| AluSx1 | chr3 | 1.96E+08 | 1.96E+08 | − | AluSx3 | chr3 |
| AluSz6 | chr3 | 1.96E+08 | 1.96E+08 | + | AluSq2 | chr3 |
| AluSx | chr3 | 1.97E+08 | 1.97E+08 | − | AluSp | chr3 |
| AluSp | chr3 | 1.97E+08 | 1.97E+08 | + | AluSq2 | chr3 |
| AluSx | chr3 | 1.97E+08 | 1.97E+08 | − | AluSx3 | chr3 |
| AluSq2 | chr3 | 1.97E+08 | 1.97E+08 | − | AluSx3 | chr3 |
| AluSx1 | chr3 | 1.97E+08 | 1.97E+08 | + | AluSx1 | chr3 |
| AluSg7 | chr3 | 1.97E+08 | 1.97E+08 | + | AluSz6 | chr3 |
| AluSg7 | chr3 | 1.97E+08 | 1.97E+08 | + | AluSq2 | chr3 |
| AluSx | chr3 | 1.97E+08 | 1.97E+08 | − | AluSx1 | chr3 |
| AluJo | chr3 | 1.98E+08 | 1.98E+08 | + | AluSp | chr3 |
| AluSx1 | chr3 | 1.98E+08 | 1.98E+08 | − | AluSp | chr3 |
| AluJo | chr7 | 757012 | 757298 | + | AluSx | chr7 |
| AluSx | chr7 | 758002 | 758293 | − | AluSp | chr7 |
| AluSz | chr7 | 2231092 | 2231382 | + | AluSq2 | chr7 |
| AluSx1 | chr7 | 2409401 | 2409710 | − | AluSc | chr7 |
| AluSx3 | chr7 | 5368144 | 5368439 | − | AluSx1 | chr7 |
| AluSx1 | chr7 | 5538226 | 5538499 | − | AluSx | chr7 |
| AluY | chr7 | 5537698 | 5538016 | − | AluSx | chr7 |
| AluSq2 | chr7 | 6417896 | 6418195 | + | AluSq | chr7 |
| AluSx1 | chr7 | 6418439 | 6418736 | + | AluSq | chr7 |
| AluSq | chr7 | 6427096 | 6427377 | + | AluSx | chr7 |
| AluSc8 | chr7 | 11086692 | 11086992 | − | AluSx1 | chr7 |
| AluSp | chr7 | 20218221 | 20218522 | − | AluSc | chr7 |
| AluSp | chr7 | 20218221 | 20218522 | − | AluSz | chr7 |
| AluSp | chr7 | 23340877 | 23341177 | − | AluSc | chr7 |
| AluSc | chr7 | 23341309 | 23341601 | + | AluSx1 | chr7 |
| AluSz | chr7 | 23394833 | 23395149 | − | AluSz6 | chr7 |
| AluSz | chr7 | 23394833 | 23395149 | − | AluJb | chr7 |
| AluSz6 | chr7 | 23451598 | 23451901 | − | AluSc8 | chr7 |
| AluSx1 | chr7 | 23491286 | 23491572 | + | AluY | chr7 |
| AluY | chr7 | 23488855 | 23489139 | + | AluY | chr7 |
| AluY | chr7 | 23550709 | 23551049 | + | AluSp | chr7 |
| AluSx | chr7 | 23555512 | 23555770 | + | AluSx3 | chr7 |
| AluSx3 | chr7 | 23558289 | 23558606 | − | AluSc5 | chr7 |
| AluSz | chr7 | 24867643 | 24867907 | − | AluSx | chr7 |
| AluSq2 | chr7 | 25158900 | 25159192 | − | AluSp | chr7 |
| AluSx1 | chr7 | 25159710 | 25160003 | − | AluSq4 | chr7 |
| AluSq2 | chr7 | 25158900 | 25159192 | − | AluSq4 | chr7 |
| AluSx1 | chr7 | 25159710 | 25160003 | − | AluJb | chr7 |
| AluSq2 | chr7 | 25158900 | 25159192 | − | AluJb | chr7 |
| AluSc8 | chr7 | 27854363 | 27854655 | − | AluSg | chr7 |
| AluSc | chr7 | 32531856 | 32532155 | + | AluSz | chr7 |
| AluSx | chr7 | 32635048 | 32635343 | − | AluJb | chr7 |
| AluSq | chr7 | 32916285 | 32916599 | + | AluSc8 | chr7 |
| AluSx1 | chr7 | 36284551 | 36284856 | − | AluSx | chr7 |
| AluSx | chr7 | 36286453 | 36286761 | + | AluSz | chr7 |
| AluSx3 | chr7 | 38763043 | 38763352 | + | AluSp | chr7 |
| AluSx1 | chr7 | 40100386 | 40100679 | + | AluY | chr7 |
| AluSg | chr7 | 43931381 | 43931680 | − | AluJr | chr7 |
| AluSg | chr7 | 44018641 | 44018934 | − | AluJo | chr7 |
| AluSx1 | chr7 | 44041626 | 44041936 | + | AluSx | chr7 |
| AluSz | chr7 | 44041267 | 44041561 | + | AluSx | chr7 |
| AluSx | chr7 | 44616160 | 44616455 | − | AluSx1 | chr7 |
| AluSx | chr7 | 44727688 | 44727991 | + | AluY | chr7 |
| AluSx | chr7 | 45048620 | 45048921 | + | AluSx | chr7 |
| AluSz | chr7 | 56103317 | 56103612 | − | AluSp | chr7 |
| AluSx | chr7 | 56120472 | 56120778 | − | AluSz | chr7 |
| AluSq2 | chr7 | 56124138 | 56124445 | + | AluY | chr7 |
| AluSx1 | chr7 | 64012441 | 64012730 | − | AluSx | chr7 |
| AluSx | chr7 | 64020482 | 64020791 | − | AluSx1 | chr7 |
| AluSz | chr7 | 64278375 | 64278663 | − | AluSg | chr7 |
| AluSp | chr7 | 64283253 | 64283556 | − | AluSx4 | chr7 |
| AluY | chr7 | 64460599 | 64460900 | − | AluJb | chr7 |
| AluJb | chr7 | 64460901 | 64461187 | + | AluSz | chr7 |
| AluY | chr7 | 64460599 | 64460900 | − | AluSx | chr7 |
| AluSz | chr7 | 64462703 | 64463005 | − | AluSx | chr7 |
| AluSz | chr7 | 64535688 | 64535983 | + | AluY | chr7 |
| AluSx3 | chr7 | 64536591 | 64536901 | + | AluY | chr7 |
| AluY | chr7 | 64537352 | 64537652 | − | AluJb | chr7 |
| AluSx3 | chr7 | 64536591 | 64536901 | + | AluSg4 | chr7 |
| AluY | chr7 | 64554267 | 64554576 | − | AluSp | chr7 |
| AluSq2 | chr7 | 65189624 | 65189922 | − | AluSz | chr7 |
| AluY | chr7 | 65340407 | 65340703 | − | AluSx3 | chr7 |
| AluSg | chr7 | 65368157 | 65368451 | − | AluJb | chr7 |
| AluSp | chr7 | 65601206 | 65601516 | − | AluSx3 | chr7 |
| AluSp | chr7 | 65618144 | 65618438 | + | AluSz6 | chr7 |
| AluSx3 | chr7 | 65618766 | 65619057 | + | AluSz6 | chr7 |
| AluY | chr7 | 65619599 | 65619869 | − | AluSx1 | chr7 |
| AluSz6 | chr7 | 65619158 | 65619381 | − | AluSx1 | chr7 |
| AluSx1 | chr7 | 65620049 | 65620356 | + | AluJb | chr7 |
| AluSx3 | chr7 | 65618766 | 65619057 | + | AluJb | chr7 |
| AluSp | chr7 | 65618144 | 65618438 | + | AluJb | chr7 |
| AluSx3 | chr7 | 65618766 | 65619057 | + | AluSx | chr7 |
| AluSx1 | chr7 | 65620049 | 65620356 | + | AluSx | chr7 |
| AluSp | chr7 | 65618144 | 65618438 | + | AluSx | chr7 |
| AluSx1 | chr7 | 65620049 | 65620356 | + | AluSz | chr7 |
| AluSz | chr7 | 65622829 | 65623131 | − | AluYk4 | chr7 |
| AluSx | chr7 | 65621310 | 65621625 | − | AluYk4 | chr7 |
| AluJb | chr7 | 65620523 | 65620823 | − | AluYk4 | chr7 |
| AluYk4 | chr7 | 65623784 | 65624086 | + | AluSx | chr7 |
| AluJr4 | chr7 | 65991763 | 65992063 | + | AluSq | chr7 |
| AluSz | chr7 | 65989195 | 65989486 | + | AluSq | chr7 |
| AluSx | chr7 | 66000102 | 66000394 | + | AluSq | chr7 |
| AluSq | chr7 | 66001537 | 66001828 | − | AluSx1 | chr7 |
| AluSx1 | chr7 | 66004609 | 66004901 | + | AluSz | chr7 |
| AluSz | chr7 | 66005211 | 66005468 | − | AluSx1 | chr7 |
| AluSx1 | chr7 | 66005666 | 66005938 | + | AluSq2 | chr7 |
| AluSx1 | chr7 | 66005666 | 66005938 | + | AluSx1 | chr7 |
| AluSx1 | chr7 | 66008673 | 66008971 | − | AluJr | chr7 |
| AluSq2 | chr7 | 66008275 | 66008560 | − | AluSx | chr7 |
| AluSx1 | chr7 | 66008673 | 66008971 | − | AluSx | chr7 |
| AluJr | chr7 | 66009219 | 66009511 | + | AluJb | chr7 |
| AluSx | chr7 | 66009557 | 66009849 | + | AluJb | chr7 |
| AluSx | chr7 | 66009557 | 66009849 | + | AluSp | chr7 |
| AluJr | chr7 | 66009219 | 66009511 | + | AluSp | chr7 |
| AluSp | chr7 | 66012145 | 66012440 | − | AluSx1 | chr7 |
| AluSx1 | chr7 | 66013972 | 66014262 | + | AluSx1 | chr7 |
| AluSx1 | chr7 | 66013972 | 66014262 | + | AluSz | chr7 |
| AluJr | chr7 | 66121103 | 66121402 | − | AluSp | chr7 |
| AluJr | chr7 | 66121103 | 66121402 | − | AluSq2 | chr7 |
| AluSp | chr7 | 66121527 | 66121828 | + | AluY | chr7 |
| AluSq2 | chr7 | 66123240 | 66123548 | + | AluY | chr7 |
| AluJr | chr7 | 66121103 | 66121402 | − | AluSg4 | chr7 |
| AluY | chr7 | 66123720 | 66124023 | − | AluSg4 | chr7 |
| AluJr | chr7 | 66148715 | 66149026 | − | AluSx | chr7 |
| AluSx | chr7 | 66149151 | 66149466 | + | AluY | chr7 |
| AluSx | chr7 | 66149151 | 66149466 | + | AluY | chr7 |
| AluSx | chr7 | 66149151 | 66149466 | + | AluSx | chr7 |
| AluY | chr7 | 66151801 | 66152100 | − | AluSq2 | chr7 |
| AluSx | chr7 | 66152101 | 66152412 | − | AluSq2 | chr7 |
| AluSq2 | chr7 | 66154508 | 66154808 | + | AluSg7 | chr7 |
| AluSz | chr7 | 66158785 | 66159095 | − | AluSx1 | chr7 |
| AluJr | chr7 | 66161063 | 66161340 | − | AluSx1 | chr7 |
| AluJo | chr7 | 66166204 | 66166485 | + | AluY | chr7 |
| AluSx | chr7 | 66172398 | 66172688 | + | AluSz | chr7 |
| AluSz | chr7 | 66173263 | 66173575 | − | AluJr | chr7 |
| AluSx | chr7 | 66172398 | 66172688 | + | AluSq2 | chr7 |
| AluY | chr7 | 66179641 | 66179951 | + | AluSz6 | chr7 |
| AluSz6 | chr7 | 66181305 | 66181611 | + | AluSz6 | chr7 |
| AluSz6 | chr7 | 66181749 | 66182057 | − | AluSx | chr7 |
| AluSg | chr7 | 66187147 | 66187447 | + | AluSx | chr7 |
| AluJb | chr7 | 66203448 | 66203740 | + | AluSp | chr7 |
| AluSx | chr7 | 66216012 | 66216317 | − | AluSq4 | chr7 |
| AluSq4 | chr7 | 66217199 | 66217495 | + | AluSg | chr7 |
| AluSg | chr7 | 66218689 | 66218991 | − | AluY | chr7 |
| AluY | chr7 | 66250580 | 66250877 | + | AluSz | chr7 |
| AluY | chr7 | 66318559 | 66318857 | + | AluSp | chr7 |
| AluSx1 | chr7 | 66466373 | 66466706 | − | AluSz6 | chr7 |
| AluSz6 | chr7 | 66738968 | 66739290 | − | AluSx1 | chr7 |
| AluSz6 | chr7 | 66738968 | 66739290 | − | AluSz6 | chr7 |
| AluSz | chr7 | 66777360 | 66777658 | + | AluJo | chr7 |
| AluJo | chr7 | 66777801 | 66778109 | − | AluSz | chr7 |
| AluSz | chr7 | 66780903 | 66781207 | + | AluJb | chr7 |
| AluSz | chr7 | 66780903 | 66781207 | + | AluSq | chr7 |
| AluSx1 | chr7 | 72369053 | 72369355 | + | AluSx1 | chr7 |
| AluY | chr7 | 72406904 | 72407113 | − | AluSc5 | chr7 |
| AluSx | chr7 | 72870990 | 72871297 | + | AluSx | chr7 |
| AluSx | chr7 | 72871499 | 72871807 | − | AluSx | chr7 |
| AluYc | chr7 | 72895576 | 72895863 | + | AluJo | chr7 |
| AluJo | chr7 | 72952862 | 72953175 | + | AluSx | chr7 |
| AluJo | chr7 | 72952862 | 72953175 | + | AluSz6 | chr7 |
| AluSz6 | chr7 | 72953590 | 72953881 | − | AluY | chr7 |
| AluSx | chr7 | 72953292 | 72953589 | − | AluY | chr7 |
| AluSz6 | chr7 | 72953590 | 72953881 | − | AluSz | chr7 |
| AluSx | chr7 | 72953292 | 72953589 | − | AluSz | chr7 |
| AluJb | chr7 | 73019608 | 73019905 | + | AluSx | chr7 |
| AluSx | chr7 | 73020744 | 73021055 | − | AluSc | chr7 |
| AluSc | chr7 | 73022638 | 73022938 | + | AluSq | chr7 |
| AluSq | chr7 | 73023567 | 73023874 | − | AluSz | chr7 |
| AluY | chr7 | 73148019 | 73148316 | + | AluSz | chr7 |
| AluJo | chr7 | 73871314 | 73871619 | − | AluSq2 | chr7 |
| AluSq2 | chr7 | 73873500 | 73873806 | + | AluSz | chr7 |
| AluSq2 | chr7 | 74106102 | 74106407 | − | AluSc | chr7 |
| AluSx3 | chr7 | 74106408 | 74106701 | − | AluSc | chr7 |
| AluJb | chr7 | 74273942 | 74274254 | − | AluSx3 | chr7 |
| AluSx3 | chr7 | 74277036 | 74277329 | + | AluSz | chr7 |
| AluSx1 | chr7 | 74281621 | 74281920 | + | AluSx | chr7 |
| AluSx1 | chr7 | 74281621 | 74281920 | + | AluSx3 | chr7 |
| AluSz | chr7 | 74285831 | 74286040 | + | AluSp | chr7 |
| AluSz | chr7 | 74285831 | 74286040 | + | AluSc | chr7 |
| AluSp | chr7 | 74294675 | 74294974 | − | AluSx | chr7 |
| AluSp | chr7 | 74294675 | 74294974 | − | AluSx1 | chr7 |
| AluSp | chr7 | 74297544 | 74297844 | − | AluY | chr7 |
| AluSq2 | chr7 | 74296806 | 74297114 | − | AluY | chr7 |
| AluSx | chr7 | 74341689 | 74341992 | + | AluSq | chr7 |
| AluYa5 | chr7 | 75102945 | 75103253 | − | AluSz | chr7 |
| AluSc8 | chr7 | 75117791 | 75118089 | + | AluSx3 | chr7 |
| AluSx3 | chr7 | 75118235 | 75118538 | − | AluSx1 | chr7 |
| AluSx3 | chr7 | 75118235 | 75118538 | − | AluSz6 | chr7 |
| AluSx | chr7 | 75511650 | 75511939 | − | AluY | chr7 |
| AluY | chr7 | 75512557 | 75512851 | + | AluSx3 | chr7 |
| AluY | chr7 | 75512557 | 75512851 | + | AluJr | chr7 |
| AluSz | chr7 | 75674733 | 75675027 | + | AluSz | chr7 |
| AluSx1 | chr7 | 76237406 | 76237714 | − | AluSx | chr7 |
| AluSx1 | chr7 | 76241234 | 76241522 | + | AluSx1 | chr7 |
| AluSx | chr7 | 76239686 | 76239967 | + | AluSx1 | chr7 |
| AluSx | chr7 | 76636326 | 76636647 | + | AluSx1 | chr7 |
| AluSx1 | chr7 | 76636704 | 76637003 | + | AluSx1 | chr7 |
| AluSx1 | chr7 | 76638168 | 76638481 | − | AluSz | chr7 |
| AluSx1 | chr7 | 76637211 | 76637419 | − | AluSz | chr7 |
| AluSx1 | chr7 | 76638168 | 76638481 | − | AluSx1 | chr7 |
| AluSx1 | chr7 | 76637211 | 76637419 | − | AluSx1 | chr7 |
| AluSq2 | chr7 | 76693489 | 76693778 | + | AluSq2 | chr7 |
| AluSc | chr7 | 77185885 | 77186173 | + | AluJr | chr7 |
| AluY | chr7 | 77184950 | 77185243 | + | AluJr | chr7 |
| AluSx | chr7 | 77238578 | 77238886 | − | AluY | chr7 |
| AluSx3 | chr7 | 77253750 | 77254055 | − | AluSz6 | chr7 |
| AluSz6 | chr7 | 77254173 | 77254474 | + | AluJb | chr7 |
| AluSc8 | chr7 | 77262657 | 77262938 | + | AluY | chr7 |
| AluY | chr7 | 77267263 | 77267574 | − | AluSz | chr7 |
| AluY | chr7 | 77308362 | 77308638 | − | AluSx1 | chr7 |
| AluSx1 | chr7 | 77308673 | 77308981 | + | AluSc | chr7 |
| AluSc | chr7 | 77309343 | 77309606 | − | AluJb | chr7 |
| AluSp | chr7 | 77319001 | 77319301 | + | AluSx3 | chr7 |
| AluSx3 | chr7 | 77319371 | 77319671 | − | AluY | chr7 |
| AluSz | chr7 | 77368748 | 77369050 | + | AluSx | chr7 |
| AluSz | chr7 | 77368748 | 77369050 | + | AluSz | chr7 |
| AluSx3 | chr7 | 77385444 | 77385745 | − | AluSg | chr7 |
| AluSx1 | chr7 | 83596790 | 83597089 | + | AluSx1 | chr7 |
| AluSg | chr7 | 83608450 | 83608746 | − | AluSc | chr7 |
| AluSz | chr7 | 87527102 | 87527392 | − | AluY | chr7 |
| AluY | chr7 | 97883061 | 97883364 | + | AluSx | chr7 |
| AluSz | chr7 | 97966548 | 97966800 | − | AluSp | chr7 |
| AluY | chr7 | 98940340 | 98940625 | − | AluSq2 | chr7 |
| AluSx | chr7 | 98957950 | 98958249 | − | AluSz | chr7 |
| AluJo | chr7 | 99072413 | 99072619 | + | AluSc | chr7 |
| AluJb | chr7 | 99074968 | 99075261 | − | AluSq2 | chr7 |
| AluSc | chr7 | 99074664 | 99074963 | − | AluSq2 | chr7 |
| AluY | chr7 | 99162797 | 99163092 | − | AluSp | chr7 |
| AluSz6 | chr7 | 99164552 | 99164787 | − | AluSp | chr7 |
| AluSp | chr7 | 99164875 | 99165169 | + | AluSc8 | chr7 |
| AluSz | chr7 | 99719061 | 99719362 | − | AluY | chr7 |
| AluY | chr7 | 99719392 | 99719699 | + | AluSq | chr7 |
| AluY | chr7 | 99748327 | 99748635 | + | AluSg | chr7 |
| AluY | chr7 | 99748327 | 99748635 | + | AluY | chr7 |
| AluY | chr7 | 99941012 | 99941322 | − | AluSc | chr7 |
| AluSc | chr7 | 1E+08 | 1E+08 | + | AluSg | chr7 |
| AluSx1 | chr7 | 1E+08 | 1E+08 | + | AluSz | chr7 |
| AluSx1 | chr7 | 1E+08 | 1E+08 | + | AluSx1 | chr7 |
| AluSx1 | chr7 | 1.01E+08 | 1.01E+08 | − | AluSx | chr7 |
| AluSz6 | chr7 | 1.01E+08 | 1.01E+08 | + | AluSq2 | chr7 |
| AluSg | chr7 | 1.02E+08 | 1.02E+08 | − | AluSz | chr7 |
| AluSx | chr7 | 1.05E+08 | 1.05E+08 | + | AluSx | chr7 |
| AluSx1 | chr7 | 1.05E+08 | 1.05E+08 | + | AluJb | chr7 |
| AluSg | chr7 | 1.05E+08 | 1.05E+08 | − | AluY | chr7 |
| AluSg | chr7 | 1.05E+08 | 1.05E+08 | − | AluSx1 | chr7 |
| AluSx | chr7 | 1.05E+08 | 1.05E+08 | + | AluY | chr7 |
| AluSz | chr7 | 1.05E+08 | 1.05E+08 | + | AluSz | chr7 |
| AluSx | chr7 | 1.05E+08 | 1.05E+08 | + | AluSx | chr7 |
| AluSx | chr7 | 1.05E+08 | 1.05E+08 | − | AluSx3 | chr7 |
| AluSp | chr7 | 1.05E+08 | 1.05E+08 | + | AluSx1 | chr7 |
| AluSq2 | chr7 | 1.05E+08 | 1.05E+08 | + | AluSz6 | chr7 |
| AluSq2 | chr7 | 1.05E+08 | 1.05E+08 | + | AluSz | chr7 |
| AluJo | chr7 | 1.05E+08 | 1.05E+08 | − | AluSx1 | chr7 |
| AluSc | chr7 | 1.07E+08 | 1.07E+08 | − | AluSx | chr7 |
| AluSz | chr7 | 1.16E+08 | 1.16E+08 | − | AluSq | chr7 |
| AluSq | chr7 | 1.16E+08 | 1.16E+08 | + | AluJb | chr7 |
| AluJr4 | chr7 | 1.21E+08 | 1.21E+08 | + | AluSc8 | chr7 |
| AluSq | chr7 | 1.27E+08 | 1.27E+08 | − | AluSq | chr7 |
| AluJb | chr7 | 1.28E+08 | 1.28E+08 | − | AluSz | chr7 |
| AluSx1 | chr7 | 1.28E+08 | 1.28E+08 | − | AluJb | chr7 |
| AluSq2 | chr7 | 1.29E+08 | 1.29E+08 | + | AluY | chr7 |
| AluSq2 | chr7 | 1.3E+08 | 1.3E+08 | − | AluSx1 | chr7 |
| AluSx1 | chr7 | 1.3E+08 | 1.3E+08 | + | AluSp | chr7 |
| AluSz | chr7 | 1.39E+08 | 1.39E+08 | + | AluSx | chr7 |
| AluJo | chr7 | 1.4E+08 | 1.4E+08 | + | AluJb | chr7 |
| AluSx | chr7 | 1.4E+08 | 1.4E+08 | − | AluSg | chr7 |
| AluSg | chr7 | 1.41E+08 | 1.41E+08 | − | AluSz | chr7 |
| AluY | chr7 | 1.46E+08 | 1.46E+08 | + | AluSp | chr7 |
| AluY | chr7 | 1.49E+08 | 1.49E+08 | + | AluSx1 | chr7 |
| AluJb | chr7 | 1.51E+08 | 1.51E+08 | − | AluY | chr7 |
| AluSx1 | chr7 | 1.51E+08 | 1.51E+08 | + | AluSx | chr7 |
| AluSg7 | chr7 | 1.52E+08 | 1.52E+08 | + | AluSx1 | chr7 |
| AluSg7 | chr7 | 1.52E+08 | 1.52E+08 | + | AluSg | chr7 |
| AluJb | chr7 | 1.52E+08 | 1.52E+08 | − | AluY | chr7 |
| AluSx | chr7 | 1.52E+08 | 1.52E+08 | − | AluSc | chr7 |
| AluSc | chr7 | 1.52E+08 | 1.52E+08 | + | AluSc | chr7 |
| AluSx | chr7 | 1.52E+08 | 1.52E+08 | − | AluSz | chr7 |
| AluSz | chr7 | 1.52E+08 | 1.52E+08 | + | AluSx3 | chr7 |
| AluSx3 | chr7 | 1.52E+08 | 1.52E+08 | − | AluSx1 | chr7 |
| AluSx | chr7 | 1.52E+08 | 1.52E+08 | + | AluSx1 | chr7 |
| AluSx1 | chr7 | 1.58E+08 | 1.58E+08 | + | AluSx | chr7 |
| AluJb | chr7 | 1.59E+08 | 1.59E+08 | + | AluY | chr7 |
| AluJo | chr7 | 1.59E+08 | 1.59E+08 | + | AluSx1 | chr7 |
| AluSx1 | chr22 | 18109925 | 18110194 | − | AluSx | chr22 |
| AluSx1 | chr22 | 18119892 | 18120189 | + | AluSz | chr22 |
| AluY | chr22 | 18567004 | 18567308 | − | AluSq2 | chr22 |
| AluSz | chr22 | 18578589 | 18578887 | − | AluSz6 | chr22 |
| AluJr | chr22 | 20926927 | 20927229 | + | AluSz6 | chr22 |
| AluSx | chr22 | 21309033 | 21309316 | − | AluSg7 | chr22 |
| AluSq2 | chr22 | 21925720 | 21925990 | − | AluSg | chr22 |
| AluSc | chr22 | 21970658 | 21970964 | + | AluSx3 | chr22 |
| AluSz6 | chr22 | 24148827 | 24149131 | + | AluY | chr22 |
| AluSz6 | chr22 | 24148827 | 24149131 | + | AluSp | chr22 |
| AluSx | chr22 | 24377466 | 24377771 | − | AluSx1 | chr22 |
| AluY | chr22 | 24377087 | 24377392 | − | AluSx1 | chr22 |
| AluY | chr22 | 24375705 | 24375998 | − | AluSx1 | chr22 |
| AluY | chr22 | 25518660 | 25518957 | + | AluSc8 | chr22 |
| AluJb | chr22 | 25866499 | 25866759 | − | AluSz6 | chr22 |
| AluJb | chr22 | 28294083 | 28294378 | + | AluY | chr22 |
| AluY | chr22 | 28295314 | 28295610 | − | AluSx1 | chr22 |
| AluY | chr22 | 28295314 | 28295610 | − | AluSx1 | chr22 |
| AluSx1 | chr22 | 29193487 | 29193772 | + | AluSx1 | chr22 |
| AluJr4 | chr22 | 29286747 | 29287044 | − | AluY | chr22 |
| AluY | chr22 | 29288879 | 29289182 | + | AluJb | chr22 |
| AluJb | chr22 | 29289967 | 29290241 | − | AluYc | chr22 |
| AluSc | chr22 | 29495528 | 29495834 | − | AluSz6 | chr22 |
| AluY | chr22 | 29900374 | 29900675 | + | AluSx | chr22 |
| AluSx | chr22 | 29901855 | 29902146 | − | AluJb | chr22 |
| AluSc | chr22 | 29969015 | 29969323 | − | AluSp | chr22 |
| AluSc8 | chr22 | 30285894 | 30286194 | − | AluSx1 | chr22 |
| AluSx1 | chr22 | 30286226 | 30286521 | + | AluSz6 | chr22 |
| AluY | chr22 | 30883451 | 30883750 | − | AluSz | chr22 |
| AluSz | chr22 | 30883904 | 30884204 | + | AluSx | chr22 |
| AluJo | chr22 | 31051162 | 31051448 | + | AluJo | chr22 |
| AluJo | chr22 | 31051983 | 31052261 | − | AluY | chr22 |
| AluSx | chr22 | 31053295 | 31053459 | + | AluSq2 | chr22 |
| AluSz | chr22 | 31359375 | 31359671 | − | AluSc8 | chr22 |
| AluY | chr22 | 31358963 | 31359261 | − | AluSc8 | chr22 |
| AluSx1 | chr22 | 31356852 | 31357155 | − | AluSc8 | chr22 |
| AluSz | chr22 | 31359375 | 31359671 | − | AluSx | chr22 |
| AluSc8 | chr22 | 32139046 | 32139295 | + | AluSp | chr22 |
| AluSg | chr22 | 33918537 | 33918844 | + | AluSq | chr22 |
| AluSx1 | chr22 | 34149765 | 34150067 | + | AluY | chr22 |
| AluY | chr22 | 35804959 | 35805254 | − | AluJr | chr22 |
| AluSx | chr22 | 36158551 | 36158852 | − | AluSz6 | chr22 |
| AluSx | chr22 | 36420508 | 36420804 | + | AluSq2 | chr22 |
| AluSx | chr22 | 37423010 | 37423293 | − | AluSq | chr22 |
| AluSx1 | chr22 | 38274948 | 38275198 | − | AluSc | chr22 |
| AluSq | chr22 | 38340626 | 38340931 | + | AluJb | chr22 |
| AluSq | chr22 | 38340626 | 38340931 | + | AluSz | chr22 |
| AluSg | chr22 | 38703304 | 38703567 | + | AluJb | chr22 |
| AluSz | chr22 | 38750534 | 38750831 | + | AluSc8 | chr22 |
| AluJb | chr22 | 38750065 | 38750351 | + | AluSc8 | chr22 |
| AluSz | chr22 | 38750534 | 38750831 | + | AluSq2 | chr22 |
| AluJb | chr22 | 38750065 | 38750351 | + | AluSq2 | chr22 |
| AluSq2 | chr22 | 38751633 | 38751896 | − | AluSc | chr22 |
| AluSc8 | chr22 | 38751016 | 38751322 | − | AluSc | chr22 |
| AluSc8 | chr22 | 38751016 | 38751322 | − | AluSq2 | chr22 |
| AluSq2 | chr22 | 38751633 | 38751896 | − | AluSq2 | chr22 |
| AluJb | chr22 | 39058516 | 39058801 | − | AluSc | chr22 |
| AluSc | chr22 | 39059180 | 39059406 | + | AluJr | chr22 |
| AluSz | chr22 | 40910479 | 40910783 | − | AluSz6 | chr22 |
| AluSz6 | chr22 | 41323197 | 41323501 | + | AluSc8 | chr22 |
| AluY | chr22 | 41323597 | 41323907 | + | AluSc8 | chr22 |
| AluSc8 | chr22 | 41326130 | 41326432 | − | AluJr | chr22 |
| AluSx3 | chr22 | 41352400 | 41352642 | − | AluSc8 | chr22 |
| AluSx3 | chr22 | 41352400 | 41352642 | − | AluJb | chr22 |
| AluJb | chr22 | 41353368 | 41353652 | + | AluSx3 | chr22 |
| AluSc8 | chr22 | 41353070 | 41353358 | + | AluSx3 | chr22 |
| AluSx1 | chr22 | 41357814 | 41358087 | − | AluSp | chr22 |
| AluJb | chr22 | 41539172 | 41539467 | + | AluSz6 | chr22 |
| AluSq | chr22 | 41819499 | 41819796 | − | AluY | chr22 |
| AluSz6 | chr22 | 42207432 | 42207734 | − | AluSq2 | chr22 |
| AluSc | chr22 | 42246172 | 42246468 | + | AluSx | chr22 |
| AluSc8 | chr22 | 42245861 | 42246159 | + | AluSx | chr22 |
| AluSx | chr22 | 42247479 | 42247798 | − | AluY | chr22 |
| AluY | chr22 | 42247843 | 42248143 | + | AluSq | chr22 |
| AluY | chr22 | 42249758 | 42250061 | + | AluSq | chr22 |
| AluJb | chr22 | 42285136 | 42285430 | − | AluSx | chr22 |
| AluSp | chr22 | 42959798 | 42960102 | + | AluSq2 | chr22 |
| AluSp | chr22 | 42966146 | 42966434 | + | AluSc | chr22 |
| AluSx1 | chr22 | 42974166 | 42974466 | − | AluSx1 | chr22 |
| AluSp | chr22 | 42984623 | 42984912 | − | AluSz6 | chr22 |
| AluSq2 | chr22 | 43224283 | 43224575 | + | AluSz6 | chr22 |
| AluSx1 | chr22 | 43255406 | 43255701 | + | AluSx1 | chr22 |
| AluY | chr22 | 43320757 | 43321049 | − | AluSz | chr22 |
| AluSp | chr22 | 44342957 | 44343268 | + | AluSp | chr22 |
| AluSg | chr22 | 45165849 | 45166152 | + | AluSx1 | chr22 |
| AluSq2 | chr22 | 45171325 | 45171629 | − | AluSq2 | chr22 |
| AluSq2 | chr22 | 45186286 | 45186584 | + | AluSx3 | chr22 |
| AluSx3 | chr22 | 45186613 | 45186910 | − | AluSq2 | chr22 |
| AluSq2 | chr22 | 45187303 | 45187604 | + | AluSg | chr22 |
| AluSq2 | chr22 | 45186286 | 45186584 | + | AluSg | chr22 |
| AluSx1 | chr22 | 45247789 | 45248097 | + | AluSc8 | chr22 |
| AluSx1 | chr22 | 45247789 | 45248097 | + | AluSz | chr22 |
| AluSx1 | chr22 | 45528961 | 45529265 | − | AluSx1 | chr22 |
| AluJb | chr22 | 45536132 | 45536421 | − | AluSx1 | chr22 |
| AluSx1 | chr22 | 45736690 | 45736982 | − | AluSz | chr22 |
| AluSx1 | chr22 | 45736690 | 45736982 | − | AluSx1 | chr22 |
| AluY | chr22 | 46090929 | 46091221 | + | AluSg4 | chr22 |
| AluJb | chr22 | 46550241 | 46550533 | + | AluJo | chr22 |
| AluSc | chr22 | 50272511 | 50272818 | + | AluSx | chr22 |
| AluSq10 | chr22 | 50849219 | 50849519 | − | AluSz6 | chr22 |
| AluSq10 | chr22 | 50849219 | 50849519 | − | AluSx1 | chr22 |
| AluSz | chr13 | 19708499 | 19708769 | − | AluSz | chr13 |
| AluJr | chr13 | 19707884 | 19708174 | − | AluSz | chr13 |
| AluSz | chr13 | 19710320 | 19710600 | + | AluSx | chr13 |
| AluSx | chr13 | 20194148 | 20194450 | − | AluY | chr13 |
| AluY | chr13 | 20194717 | 20195018 | + | AluJr | chr13 |
| AluJr | chr13 | 20195025 | 20195330 | − | AluJb | chr13 |
| AluSx | chr13 | 20194148 | 20194450 | − | AluJb | chr13 |
| AluJo | chr13 | 20284983 | 20285280 | − | AluY | chr13 |
| AluSz | chr13 | 28226726 | 28227021 | + | AluSg7 | chr13 |
| AluY | chr13 | 43657300 | 43657602 | − | AluSg | chr13 |
| AluSz6 | chr13 | 45125571 | 45125856 | − | AluY | chr13 |
| AluSx | chr13 | 45604502 | 45604818 | − | AluSq2 | chr13 |
| AluSz | chr13 | 45898928 | 45899217 | − | AluJb | chr13 |
| AluSz | chr13 | 45898928 | 45899217 | − | AluJo | chr13 |
| AluSx1 | chr13 | 45944643 | 45944934 | − | AluSx | chr13 |
| AluSx1 | chr13 | 50239425 | 50239706 | − | AluYk12 | chr13 |
| AluSq2 | chr13 | 50575144 | 50575445 | + | AluSg4 | chr13 |
| AluSx | chr13 | 50596990 | 50597291 | + | AluJo | chr13 |
| AluY | chr13 | 52850141 | 52850444 | + | AluSx3 | chr13 |
| AluSx3 | chr13 | 53041570 | 53041869 | + | AluY | chr13 |
| AluSx | chr13 | 53263822 | 53264126 | + | AluJb | chr13 |
| AluSz6 | chr13 | 73353262 | 73353554 | − | AluSg | chr13 |
| AluY | chr13 | 73384996 | 73385297 | − | AluSx1 | chr13 |
| AluSq2 | chr13 | 73641978 | 73642273 | − | AluSx | chr13 |
| AluJo | chr13 | 80110885 | 80111184 | + | AluSz | chr13 |
| AluSx1 | chr13 | 95237317 | 95237600 | − | AluSz6 | chr13 |
| AluSz | chr13 | 96120709 | 96121003 | + | AluSx1 | chr13 |
| AluSz | chr13 | 96147300 | 96147590 | − | AluY | chr13 |
| AluSp | chr13 | 98807319 | 98807621 | + | AluJb | chr13 |
| AluSz6 | chr13 | 98817188 | 98817492 | + | AluSx1 | chr13 |
| AluSg | chr13 | 98909745 | 98910043 | + | AluY | chr13 |
| AluY | chr13 | 99116390 | 99116680 | + | AluSq | chr13 |
| AluSg4 | chr13 | 1E+08 | 1E+08 | + | AluSc | chr13 |
| AluSc | chr13 | 1.11E+08 | 1.11E+08 | − | AluSx1 | chr13 |
| AluSx1 | chr13 | 1.13E+08 | 1.13E+08 | − | AluSx3 | chr13 |
| AluJb | chr12 | 468637 | 468920 | + | AluY | chr12 |
| AluSc | chr12 | 975543 | 975824 | − | AluSx | chr12 |
| AluSx1 | chr12 | 1119365 | 1119671 | + | AluSq2 | chr12 |
| AluJr | chr12 | 2961536 | 2961829 | − | AluSx1 | chr12 |
| AluSx1 | chr12 | 3027278 | 3027577 | − | AluJb | chr12 |
| AluSx | chr12 | 4436293 | 4436601 | + | AluSx4 | chr12 |
| AluSg | chr12 | 4462721 | 4462985 | + | AluSz6 | chr12 |
| AluJo | chr12 | 6430185 | 6430481 | − | AluYc | chr12 |
| AluSg | chr12 | 6994519 | 6994811 | − | AluSx3 | chr12 |
| AluSx3 | chr12 | 6996055 | 6996377 | + | AluSz | chr12 |
| AluSc | chr12 | 7247715 | 7248009 | − | AluY | chr12 |
| AluSq | chr12 | 7249880 | 7250177 | − | AluY | chr12 |
| AluYk4 | chr12 | 8948738 | 8949027 | − | AluSx | chr12 |
| AluSz6 | chr12 | 9811202 | 9811497 | + | AluSx | chr12 |
| AluJb | chr12 | 12350696 | 12350926 | + | AluSx1 | chr12 |
| AluSx1 | chr12 | 12350932 | 12351150 | − | AluSg | chr12 |
| AluSx | chr12 | 12372940 | 12373237 | + | AluJb | chr12 |
| AluY | chr12 | 12526913 | 12527224 | − | AluJb | chr12 |
| AluSz6 | chr12 | 13049881 | 13050181 | + | AluSx | chr12 |
| AluSx | chr12 | 13050541 | 13050843 | − | AluJo | chr12 |
| AluSp | chr12 | 13055720 | 13056028 | + | AluJo | chr12 |
| AluJr | chr12 | 13055415 | 13055718 | + | AluJo | chr12 |
| AluSp | chr12 | 13055720 | 13056028 | + | AluSx | chr12 |
| AluJr | chr12 | 13055415 | 13055718 | + | AluSx | chr12 |
| AluSx | chr12 | 14323624 | 14323849 | + | AluSp | chr12 |
| AluSq2 | chr12 | 14911593 | 14911907 | − | AluSg7 | chr12 |
| AluSc8 | chr12 | 15816578 | 15816880 | + | AluSq2 | chr12 |
| AluY | chr12 | 19398910 | 19399210 | − | AluSg7 | chr12 |
| AluSx1 | chr12 | 26615599 | 26615901 | − | AluSp | chr12 |
| AluY | chr12 | 27939653 | 27939957 | − | AluSx1 | chr12 |
| AluSx | chr12 | 27945439 | 27945755 | − | AluJo | chr12 |
| AluSq2 | chr12 | 31430806 | 31431107 | + | AluSz | chr12 |
| AluSz6 | chr12 | 31733415 | 31733728 | − | AluJb | chr12 |
| AluSx3 | chr12 | 31883252 | 31883559 | + | AluSx3 | chr12 |
| AluY | chr12 | 31901177 | 31901368 | + | AluSx4 | chr12 |
| AluSx | chr12 | 32141633 | 32141923 | − | AluY | chr12 |
| AluSz | chr12 | 32147816 | 32148106 | − | AluSq2 | chr12 |
| AluSx1 | chr12 | 32146863 | 32147162 | − | AluSq2 | chr12 |
| AluSz | chr12 | 32147816 | 32148106 | − | AluJb | chr12 |
| AluSx | chr12 | 32286140 | 32286435 | + | AluY | chr12 |
| AluSp | chr12 | 32558868 | 32559175 | − | AluSx1 | chr12 |
| AluSx1 | chr12 | 32637524 | 32637833 | + | AluY | chr12 |
| AluY | chr12 | 32895823 | 32896128 | − | AluSp | chr12 |
| AluSz6 | chr12 | 32895041 | 32895337 | − | AluSp | chr12 |
| AluSx | chr12 | 32894233 | 32894517 | − | AluSp | chr12 |
| AluSx1 | chr12 | 39821463 | 39821761 | − | AluSc5 | chr12 |
| AluJr | chr12 | 42627320 | 42627609 | − | AluSx1 | chr12 |
| AluSx1 | chr12 | 42753628 | 42753931 | + | AluSg | chr12 |
| AluSp | chr12 | 46669287 | 46669596 | + | AluSx | chr12 |
| AluSq2 | chr12 | 49051418 | 49051699 | − | AluSz | chr12 |
| AluSp | chr12 | 49721998 | 49722304 | − | AluY | chr12 |
| AluJb | chr12 | 49747150 | 49747458 | + | AluJo | chr12 |
| AluSq2 | chr12 | 49771744 | 49772026 | − | AluY | chr12 |
| AluSx | chr12 | 49781138 | 49781445 | + | AluJb | chr12 |
| AluSz | chr12 | 49863392 | 49863691 | + | AluSq2 | chr12 |
| AluSz | chr12 | 49885991 | 49886303 | − | AluSq | chr12 |
| AluSz | chr12 | 49903054 | 49903352 | − | AluSz | chr12 |
| AluSx1 | chr12 | 50545451 | 50545728 | − | AluSx1 | chr12 |
| AluY | chr12 | 50552823 | 50553119 | + | AluSx1 | chr12 |
| AluSx1 | chr12 | 50553379 | 50553673 | − | AluSx | chr12 |
| AluSx1 | chr12 | 50553379 | 50553673 | − | AluSz6 | chr12 |
| AluSx | chr12 | 50553806 | 50554117 | + | AluSx | chr12 |
| AluSz6 | chr12 | 50555278 | 50555583 | + | AluSx | chr12 |
| AluSq | chr12 | 50595400 | 50595712 | + | AluSz | chr12 |
| AluSz | chr12 | 50851325 | 50851609 | + | AluJb | chr12 |
| AluJb | chr12 | 50851719 | 50852017 | − | AluSc | chr12 |
| AluSx1 | chr12 | 50910476 | 50910782 | + | AluY | chr12 |
| AluJb | chr12 | 50928730 | 50929033 | + | AluSx1 | chr12 |
| AluSc | chr12 | 50937731 | 50938035 | + | AluSx3 | chr12 |
| AluSz | chr12 | 50937424 | 50937727 | + | AluSx3 | chr12 |
| AluSp | chr12 | 51324366 | 51324664 | − | AluSq2 | chr12 |
| AluSq | chr12 | 51607309 | 51607615 | − | AluSx1 | chr12 |
| AluY | chr12 | 53570222 | 53570516 | + | AluJr | chr12 |
| AluSx4 | chr12 | 53690189 | 53690496 | − | AluSx | chr12 |
| AluYk11 | chr12 | 53838114 | 53838412 | − | AluSp | chr12 |
| AluSp | chr12 | 53841147 | 53841388 | + | AluSc | chr12 |
| AluSp | chr12 | 53841147 | 53841388 | + | AluSc | chr12 |
| AluSp | chr12 | 53863223 | 53863526 | + | AluSz | chr12 |
| AluSz | chr12 | 53863545 | 53863829 | − | AluSg | chr12 |
| AluSg | chr12 | 53863888 | 53864186 | + | AluJb | chr12 |
| AluSq2 | chr12 | 54065654 | 54065889 | + | AluSz6 | chr12 |
| AluSz6 | chr12 | 54065995 | 54066264 | − | AluSp | chr12 |
| AluSz | chr12 | 54720753 | 54721070 | − | AluSz | chr12 |
| AluSq2 | chr12 | 54726239 | 54726547 | + | AluSz6 | chr12 |
| AluSz | chr12 | 56081272 | 56081573 | − | AluSx | chr12 |
| AluY | chr12 | 56219327 | 56219627 | − | AluSx3 | chr12 |
| AluSz | chr12 | 56217486 | 56217777 | − | AluSx3 | chr12 |
| AluY | chr12 | 56227595 | 56227906 | + | AluSz | chr12 |
| AluSx3 | chr12 | 56691288 | 56691501 | + | AluSp | chr12 |
| AluSp | chr12 | 56706993 | 56707290 | + | AluY | chr12 |
| AluSx1 | chr12 | 57071957 | 57072260 | − | AluJr4 | chr12 |
| AluSx1 | chr12 | 57071957 | 57072260 | − | AluSz6 | chr12 |
| AluSz6 | chr12 | 57072914 | 57073215 | + | AluSq2 | chr12 |
| AluSz6 | chr12 | 57072914 | 57073215 | + | AluSx1 | chr12 |
| AluJr4 | chr12 | 57072619 | 57072906 | + | AluSx1 | chr12 |
| AluSq2 | chr12 | 57648003 | 57648312 | + | AluSx | chr12 |
| AluSx1 | chr12 | 57792268 | 57792571 | − | AluSg | chr12 |
| AluSx1 | chr12 | 57807237 | 57807527 | + | AluSz | chr12 |
| AluSg | chr12 | 57820794 | 57821100 | + | AluSx | chr12 |
| AluSg | chr12 | 57820794 | 57821100 | + | AluSx1 | chr12 |
| AluSp | chr12 | 58178380 | 58178680 | + | AluY | chr12 |
| AluSc5 | chr12 | 62791357 | 62791649 | + | AluJb | chr12 |
| AluSz | chr12 | 62907024 | 62907305 | − | AluY | chr12 |
| AluSq | chr12 | 64267029 | 64267343 | + | AluSc8 | chr12 |
| AluSz | chr12 | 64857197 | 64857497 | + | AluSc | chr12 |
| AluY | chr12 | 64886669 | 64886972 | − | AluSx1 | chr12 |
| AluSp | chr12 | 64886078 | 64886385 | − | AluSx1 | chr12 |
| AluJb | chr12 | 65022882 | 65023187 | − | AluJo | chr12 |
| AluSx | chr12 | 65029901 | 65030193 | − | AluY | chr12 |
| AluSz6 | chr12 | 65031196 | 65031509 | − | AluY | chr12 |
| AluSz | chr12 | 65053447 | 65053726 | + | AluSx | chr12 |
| AluJo | chr12 | 65055005 | 65055271 | + | AluSx | chr12 |
| AluSx | chr12 | 65055452 | 65055760 | − | AluJb | chr12 |
| AluY | chr12 | 65192586 | 65192870 | − | AluSx3 | chr12 |
| AluSz | chr12 | 66237547 | 66237853 | + | AluSx | chr12 |
| AluSx3 | chr12 | 66254866 | 66255174 | + | AluSq2 | chr12 |
| AluSp | chr12 | 66511862 | 66512161 | + | AluSq2 | chr12 |
| AluJb | chr12 | 66515383 | 66515683 | + | AluSx3 | chr12 |
| AluJo | chr12 | 66542939 | 66543252 | + | AluJb | chr12 |
| AluSx | chr12 | 69030529 | 69030733 | − | AluSq2 | chr12 |
| AluSq2 | chr12 | 69031096 | 69031408 | + | AluSz | chr12 |
| AluSz | chr12 | 69158078 | 69158367 | − | AluSp | chr12 |
| AluSp | chr12 | 69159994 | 69160284 | + | AluJb | chr12 |
| AluJb | chr12 | 69160372 | 69160664 | − | AluJb | chr12 |
| AluSz | chr12 | 69158078 | 69158367 | − | AluJb | chr12 |
| AluSg | chr12 | 69214825 | 69215125 | − | AluJb | chr12 |
| AluJb | chr12 | 69215221 | 69215528 | + | AluJb | chr12 |
| AluJr | chr12 | 70814991 | 70815273 | + | AluY | chr12 |
| AluSx | chr12 | 72052063 | 72052368 | − | AluY | chr12 |
| AluY | chr12 | 72054802 | 72055104 | + | AluSx1 | chr12 |
| AluSz | chr12 | 74523072 | 74523380 | + | AluJb | chr12 |
| AluSx3 | chr12 | 76950439 | 76950743 | − | AluSz | chr12 |
| AluJb | chr12 | 80193508 | 80193789 | + | AluY | chr12 |
| AluY | chr12 | 80194514 | 80194811 | − | AluSp | chr12 |
| AluSz | chr12 | 81220312 | 81220600 | − | AluY | chr12 |
| AluSx1 | chr12 | 83162970 | 83163278 | + | AluSx | chr12 |
| AluSc | chr12 | 83258273 | 83258579 | − | AluSq | chr12 |
| AluSx | chr12 | 89828954 | 89829257 | − | AluSx | chr12 |
| AluSx1 | chr12 | 92496707 | 92497015 | − | AluY | chr12 |
| AluY | chr12 | 93776451 | 93776735 | − | AluSx | chr12 |
| AluJb | chr12 | 93779753 | 93780048 | − | AluSp | chr12 |
| AluSp | chr12 | 93782967 | 93783201 | + | AluSx1 | chr12 |
| AluSx1 | chr12 | 93785059 | 93785363 | + | AluSx1 | chr12 |
| AluSg7 | chr12 | 93783397 | 93783688 | + | AluSx1 | chr12 |
| AluSz | chr12 | 94724789 | 94725092 | + | AluY | chr12 |
| AluJo | chr12 | 96607502 | 96607784 | − | AluSx | chr12 |
| AluSx1 | chr12 | 98885456 | 98885764 | − | AluSg | chr12 |
| AluSx | chr12 | 98917448 | 98917748 | − | AluSg | chr12 |
| AluSg | chr12 | 1.03E+08 | 1.03E+08 | − | AluSp | chr12 |
| AluSx1 | chr12 | 1.04E+08 | 1.04E+08 | − | AluSx1 | chr12 |
| AluSq2 | chr12 | 1.05E+08 | 1.05E+08 | + | AluSx | chr12 |
| AluSp | chr12 | 1.05E+08 | 1.05E+08 | + | AluY | chr12 |
| AluSx | chr12 | 1.09E+08 | 1.09E+08 | − | AluSx | chr12 |
| AluSq2 | chr12 | 1.09E+08 | 1.09E+08 | − | AluY | chr12 |
| AluJb | chr12 | 1.09E+08 | 1.09E+08 | − | AluSp | chr12 |
| AluSc | chr12 | 1.11E+08 | 1.11E+08 | + | AluJb | chr12 |
| AluJb | chr12 | 1.11E+08 | 1.11E+08 | − | AluSc | chr12 |
| AluY | chr12 | 1.11E+08 | 1.11E+08 | + | AluSc | chr12 |
| AluSg | chr12 | 1.11E+08 | 1.11E+08 | + | AluSc | chr12 |
| AluSz6 | chr12 | 1.11E+08 | 1.11E+08 | + | AluSc | chr12 |
| AluSz | chr12 | 1.11E+08 | 1.11E+08 | − | AluY | chr12 |
| AluSz | chr12 | 1.11E+08 | 1.11E+08 | − | AluSc | chr12 |
| AluSz | chr12 | 1.11E+08 | 1.11E+08 | − | AluSz | chr12 |
| AluSz | chr12 | 1.11E+08 | 1.11E+08 | − | AluSp | chr12 |
| AluSc | chr12 | 1.11E+08 | 1.11E+08 | + | AluSg4 | chr12 |
| AluY | chr12 | 1.11E+08 | 1.11E+08 | + | AluSg4 | chr12 |
| AluSz | chr12 | 1.11E+08 | 1.11E+08 | + | AluSg4 | chr12 |
| AluSg4 | chr12 | 1.11E+08 | 1.11E+08 | − | AluJb | chr12 |
| AluSz | chr12 | 1.11E+08 | 1.11E+08 | − | AluJb | chr12 |
| AluSg4 | chr12 | 1.11E+08 | 1.11E+08 | − | AluSx1 | chr12 |
| AluSz | chr12 | 1.11E+08 | 1.11E+08 | − | AluSx1 | chr12 |
| AluSg4 | chr12 | 1.11E+08 | 1.11E+08 | − | AluSz | chr12 |
| AluSx | chr12 | 1.11E+08 | 1.11E+08 | + | AluSq2 | chr12 |
| AluY | chr12 | 1.11E+08 | 1.11E+08 | − | AluSx | chr12 |
| AluY | chr12 | 1.11E+08 | 1.11E+08 | − | AluSq2 | chr12 |
| AluSx | chr12 | 1.11E+08 | 1.11E+08 | − | AluSg | chr12 |
| AluSg | chr12 | 1.11E+08 | 1.11E+08 | − | AluSg | chr12 |
| AluSq2 | chr12 | 1.12E+08 | 1.12E+08 | + | AluSx | chr12 |
| AluSg7 | chr12 | 1.12E+08 | 1.12E+08 | + | AluSx | chr12 |
| AluSx | chr12 | 1.12E+08 | 1.12E+08 | − | AluSx4 | chr12 |
| AluSx3 | chr12 | 1.12E+08 | 1.12E+08 | + | AluY | chr12 |
| AluSx3 | chr12 | 1.12E+08 | 1.12E+08 | + | AluSx3 | chr12 |
| AluSx3 | chr12 | 1.12E+08 | 1.12E+08 | − | AluSx1 | chr12 |
| AluY | chr12 | 1.12E+08 | 1.12E+08 | − | AluSx1 | chr12 |
| AluSx3 | chr12 | 1.12E+08 | 1.12E+08 | − | AluSq2 | chr12 |
| AluY | chr12 | 1.12E+08 | 1.12E+08 | − | AluSq2 | chr12 |
| AluSx1 | chr12 | 1.12E+08 | 1.12E+08 | + | AluSc | chr12 |
| AluSq2 | chr12 | 1.12E+08 | 1.12E+08 | + | AluSc | chr12 |
| AluSc | chr12 | 1.12E+08 | 1.12E+08 | − | AluSz6 | chr12 |
| AluSc | chr12 | 1.12E+08 | 1.12E+08 | + | AluY | chr12 |
| AluSz6 | chr12 | 1.12E+08 | 1.12E+08 | + | AluY | chr12 |
| AluY | chr12 | 1.12E+08 | 1.12E+08 | − | AluSx1 | chr12 |
| AluSx1 | chr12 | 1.12E+08 | 1.12E+08 | + | AluSx1 | chr12 |
| AluSc | chr12 | 1.12E+08 | 1.12E+08 | + | AluSx1 | chr12 |
| AluSq | chr12 | 1.12E+08 | 1.12E+08 | + | AluSg | chr12 |
| AluSg | chr12 | 1.12E+08 | 1.12E+08 | − | AluSx | chr12 |
| AluY | chr12 | 1.12E+08 | 1.12E+08 | − | AluSx | chr12 |
| AluY | chr12 | 1.12E+08 | 1.12E+08 | − | AluSg | chr12 |
| AluSz6 | chr12 | 1.13E+08 | 1.13E+08 | + | AluSx1 | chr12 |
| AluSx4 | chr12 | 1.13E+08 | 1.13E+08 | − | AluSx1 | chr12 |
| AluSc | chr12 | 1.13E+08 | 1.13E+08 | + | AluSz6 | chr12 |
| AluSx | chr12 | 1.13E+08 | 1.13E+08 | − | AluSc | chr12 |
| AluJb | chr12 | 1.14E+08 | 1.14E+08 | + | AluSx | chr12 |
| AluSx1 | chr12 | 1.14E+08 | 1.14E+08 | − | AluJb | chr12 |
| AluSx1 | chr12 | 1.14E+08 | 1.14E+08 | − | AluJb | chr12 |
| AluJb | chr12 | 1.14E+08 | 1.14E+08 | + | AluJo | chr12 |
| AluJb | chr12 | 1.14E+08 | 1.14E+08 | + | AluJo | chr12 |
| AluSz | chr12 | 1.17E+08 | 1.17E+08 | − | AluJr | chr12 |
| AluSz6 | chr12 | 1.17E+08 | 1.17E+08 | − | AluSp | chr12 |
| AluY | chr12 | 1.17E+08 | 1.17E+08 | + | AluJo | chr12 |
| AluJo | chr12 | 1.17E+08 | 1.17E+08 | − | AluSx | chr12 |
| AluSx | chr12 | 1.17E+08 | 1.17E+08 | + | AluSx | chr12 |
| AluSz | chr12 | 1.17E+08 | 1.17E+08 | − | AluSx1 | chr12 |
| AluSx1 | chr12 | 1.17E+08 | 1.17E+08 | − | AluSx1 | chr12 |
| AluY | chr12 | 1.19E+08 | 1.19E+08 | + | AluSq2 | chr12 |
| AluSx1 | chr12 | 1.21E+08 | 1.21E+08 | − | AluSx | chr12 |
| AluSx1 | chr12 | 1.21E+08 | 1.21E+08 | − | AluY | chr12 |
| AluSg | chr12 | 1.21E+08 | 1.21E+08 | − | AluY | chr12 |
| AluY | chr12 | 1.21E+08 | 1.21E+08 | + | AluSg | chr12 |
| AluY | chr12 | 1.21E+08 | 1.21E+08 | + | AluSg | chr12 |
| AluSg | chr12 | 1.21E+08 | 1.21E+08 | − | AluSx1 | chr12 |
| AluSg | chr12 | 1.21E+08 | 1.21E+08 | − | AluSx1 | chr12 |
| AluJb | chr12 | 1.21E+08 | 1.21E+08 | − | AluSc8 | chr12 |
| AluSc8 | chr12 | 1.21E+08 | 1.21E+08 | + | AluSp | chr12 |
| AluSx4 | chr12 | 1.21E+08 | 1.21E+08 | + | AluSx1 | chr12 |
| AluSx1 | chr12 | 1.21E+08 | 1.21E+08 | − | AluSx | chr12 |
| AluSx | chr12 | 1.21E+08 | 1.21E+08 | + | AluSc8 | chr12 |
| AluSg | chr12 | 1.21E+08 | 1.21E+08 | − | AluY | chr12 |
| AluSc8 | chr12 | 1.21E+08 | 1.21E+08 | − | AluY | chr12 |
| AluY | chr12 | 1.21E+08 | 1.21E+08 | − | AluSp | chr12 |
| AluY | chr12 | 1.21E+08 | 1.21E+08 | − | AluSq2 | chr12 |
| AluSq2 | chr12 | 1.21E+08 | 1.21E+08 | + | AluY | chr12 |
| AluY | chr12 | 1.21E+08 | 1.21E+08 | − | AluSz6 | chr12 |
| AluJo | chr12 | 1.21E+08 | 1.21E+08 | − | AluSz | chr12 |
| AluSc8 | chr12 | 1.21E+08 | 1.21E+08 | + | AluSc | chr12 |
| AluSx1 | chr12 | 1.21E+08 | 1.21E+08 | + | AluSx1 | chr12 |
| AluSx | chr12 | 1.22E+08 | 1.22E+08 | + | AluY | chr12 |
| AluSx | chr12 | 1.22E+08 | 1.22E+08 | + | AluSq2 | chr12 |
| AluJr | chr12 | 1.22E+08 | 1.22E+08 | + | AluSq2 | chr12 |
| AluSz | chr12 | 1.22E+08 | 1.22E+08 | + | AluSx | chr12 |
| AluSx | chr12 | 1.22E+08 | 1.22E+08 | − | AluSg | chr12 |
| AluSg | chr12 | 1.22E+08 | 1.22E+08 | + | AluSx1 | chr12 |
| AluSq4 | chr12 | 1.22E+08 | 1.22E+08 | + | AluY | chr12 |
| AluJb | chr12 | 1.22E+08 | 1.22E+08 | + | AluY | chr12 |
| AluSq4 | chr12 | 1.22E+08 | 1.22E+08 | + | AluSx | chr12 |
| AluY | chr12 | 1.22E+08 | 1.22E+08 | − | AluSq | chr12 |
| AluSx | chr12 | 1.22E+08 | 1.22E+08 | − | AluSq | chr12 |
| AluSq4 | chr12 | 1.22E+08 | 1.22E+08 | + | AluSx | chr12 |
| AluSq | chr12 | 1.22E+08 | 1.22E+08 | + | AluSx | chr12 |
| AluJb | chr12 | 1.22E+08 | 1.22E+08 | + | AluY | chr12 |
| AluY | chr12 | 1.22E+08 | 1.22E+08 | − | AluSx1 | chr12 |
| AluSq | chr12 | 1.23E+08 | 1.23E+08 | + | AluSx | chr12 |
| AluSx | chr12 | 1.23E+08 | 1.23E+08 | + | AluSx | chr12 |
| AluSq | chr12 | 1.23E+08 | 1.23E+08 | + | AluJo | chr12 |
| AluSq | chr12 | 1.23E+08 | 1.23E+08 | + | AluSz | chr12 |
| AluSz | chr12 | 1.23E+08 | 1.23E+08 | − | AluSz | chr12 |
| AluJo | chr12 | 1.23E+08 | 1.23E+08 | − | AluSz | chr12 |
| AluSx | chr12 | 1.23E+08 | 1.23E+08 | − | AluSz | chr12 |
| AluSz | chr12 | 1.23E+08 | 1.23E+08 | + | AluSc5 | chr12 |
| AluSz | chr12 | 1.23E+08 | 1.23E+08 | + | AluSz | chr12 |
| AluSz | chr12 | 1.23E+08 | 1.23E+08 | − | AluY | chr12 |
| AluSz | chr12 | 1.23E+08 | 1.23E+08 | − | AluY | chr12 |
| AluSc5 | chr12 | 1.23E+08 | 1.23E+08 | − | AluY | chr12 |
| AluJo | chr12 | 1.23E+08 | 1.23E+08 | − | AluY | chr12 |
| AluSx | chr12 | 1.23E+08 | 1.23E+08 | − | AluY | chr12 |
| AluY | chr12 | 1.23E+08 | 1.23E+08 | + | AluSx | chr12 |
| AluSz | chr12 | 1.23E+08 | 1.23E+08 | + | AluSx | chr12 |
| AluSc5 | chr12 | 1.23E+08 | 1.23E+08 | − | AluJb | chr12 |
| AluSz | chr12 | 1.23E+08 | 1.23E+08 | − | AluJb | chr12 |
| AluSx | chr12 | 1.23E+08 | 1.23E+08 | − | AluJb | chr12 |
| AluSz | chr12 | 1.23E+08 | 1.23E+08 | − | AluSx1 | chr12 |
| AluSx | chr12 | 1.23E+08 | 1.23E+08 | − | AluSx1 | chr12 |
| AluSx1 | chr12 | 1.23E+08 | 1.23E+08 | + | AluSp | chr12 |
| AluJb | chr12 | 1.23E+08 | 1.23E+08 | + | AluSp | chr12 |
| AluY | chr12 | 1.23E+08 | 1.23E+08 | + | AluSp | chr12 |
| AluSp | chr12 | 1.23E+08 | 1.23E+08 | − | AluJb | chr12 |
| AluSx | chr12 | 1.23E+08 | 1.23E+08 | − | AluJb | chr12 |
| AluSp | chr12 | 1.23E+08 | 1.23E+08 | − | AluJr | chr12 |
| AluSg | chr12 | 1.23E+08 | 1.23E+08 | + | AluSx1 | chr12 |
| AluSx1 | chr12 | 1.23E+08 | 1.23E+08 | − | AluSg | chr12 |
| AluJo | chr12 | 1.23E+08 | 1.23E+08 | + | AluSx | chr12 |
| AluSx | chr12 | 1.23E+08 | 1.23E+08 | − | AluSz6 | chr12 |
| AluSg | chr12 | 1.24E+08 | 1.24E+08 | − | AluSg | chr12 |
| AluSc | chr12 | 1.24E+08 | 1.24E+08 | − | AluSx | chr12 |
| AluJb | chr12 | 1.24E+08 | 1.24E+08 | + | AluSz6 | chr12 |
| AluSp | chr12 | 1.32E+08 | 1.32E+08 | − | AluSz6 | chr12 |
| AluSz6 | chr12 | 1.32E+08 | 1.32E+08 | + | AluSq2 | chr12 |
| AluSx1 | chr12 | 1.33E+08 | 1.33E+08 | + | AluJr | chr12 |
| AluSq | chr12 | 1.33E+08 | 1.33E+08 | + | AluSx1 | chr12 |
| AluSq | chr12 | 1.33E+08 | 1.33E+08 | + | AluSg | chr12 |
| AluSx1 | chr12 | 1.33E+08 | 1.33E+08 | − | AluSx | chr12 |
| AluSg | chr12 | 1.33E+08 | 1.33E+08 | − | AluSx | chr12 |
| AluSg | chr12 | 1.33E+08 | 1.33E+08 | − | AluSz | chr12 |
| AluSx1 | chr12 | 1.33E+08 | 1.33E+08 | − | AluSz | chr12 |
| AluSz | chr12 | 1.33E+08 | 1.33E+08 | + | AluSg7 | chr12 |
| AluSq | chr12 | 1.33E+08 | 1.33E+08 | + | AluSg7 | chr12 |
| AluSx | chr12 | 1.33E+08 | 1.33E+08 | + | AluSg7 | chr12 |
| AluSc8 | chr12 | 1.34E+08 | 1.34E+08 | − | AluJb | chr12 |
| AluJb | chr12 | 1.34E+08 | 1.34E+08 | + | AluSz | chr12 |
| AluSc | chr12 | 1.34E+08 | 1.34E+08 | − | AluSx | chr12 |
| AluSq | chr21 | 9945466 | 9945733 | + | AluY | chr21 |
| AluSx3 | chr21 | 9943628 | 9943928 | + | AluY | chr21 |
| AluY | chr21 | 11088477 | 11088787 | − | AluSx | chr21 |
| AluSx | chr21 | 11089237 | 11089543 | + | AluSp | chr21 |
| AluSp | chr21 | 11089714 | 11090007 | − | AluSg7 | chr21 |
| AluSx | chr21 | 11089237 | 11089543 | + | AluSx1 | chr21 |
| AluSx | chr21 | 11089237 | 11089543 | + | AluSg | chr21 |
| AluSg | chr21 | 11092484 | 11092737 | − | AluSp | chr21 |
| AluSx1 | chr21 | 11092171 | 11092483 | − | AluSp | chr21 |
| AluSp | chr21 | 11094648 | 11094933 | + | AluSx | chr21 |
| AluJb | chr21 | 11096205 | 11096503 | − | AluSx1 | chr21 |
| AluJb | chr21 | 11096205 | 11096503 | − | AluSp | chr21 |
| AluSp | chr21 | 11177846 | 11178117 | + | AluY | chr21 |
| AluSc | chr21 | 11177369 | 11177658 | + | AluY | chr21 |
| AluSq2 | chr21 | 20364878 | 20365185 | − | AluJb | chr21 |
| AluSx3 | chr21 | 33099384 | 33099688 | − | AluSc | chr21 |
| AluSx1 | chr21 | 34636149 | 34636462 | − | AluSz6 | chr21 |
| AluSc | chr21 | 34728044 | 34728328 | + | AluSg7 | chr21 |
| AluSx | chr21 | 36244144 | 36244439 | − | AluSq | chr21 |
| AluSq | chr21 | 36246625 | 36246932 | + | AluSq | chr21 |
| AluSz | chr21 | 37526092 | 37526403 | + | AluSx | chr21 |
| AluY | chr21 | 38254301 | 38254593 | + | AluSc8 | chr21 |
| AluSq2 | chr21 | 38391006 | 38391293 | − | AluSp | chr21 |
| AluSx | chr21 | 40673905 | 40674209 | − | AluSz6 | chr21 |
| AluSx1 | chr21 | 44329240 | 44329551 | − | AluSp | chr21 |
| AluSx3 | chr21 | 44329796 | 44330094 | − | AluSp | chr21 |
| AluSz | chr21 | 44398584 | 44398893 | − | AluY | chr21 |
| AluSq2 | chr21 | 44397648 | 44397953 | − | AluY | chr21 |
| AluY | chr21 | 46214442 | 46214747 | − | AluSx | chr21 |
| AluY | chr21 | 46214442 | 46214747 | − | AluJb | chr21 |
| AluSx | chr21 | 47638261 | 47638555 | + | AluSc | chr21 |
| AluY | chr21 | 47698595 | 47698885 | − | AluSx3 | chr21 |
| AluSc8 | chr20 | 296400 | 296673 | − | AluSq2 | chr20 |
| AluJo | chr20 | 296683 | 296971 | − | AluSq2 | chr20 |
| AluSc8 | chr20 | 296400 | 296673 | − | AluJb | chr20 |
| AluJo | chr20 | 296683 | 296971 | − | AluJb | chr20 |
| AluSc8 | chr20 | 296400 | 296673 | − | AluSg4 | chr20 |
| AluJo | chr20 | 296683 | 296971 | − | AluSg4 | chr20 |
| AluSx | chr20 | 3805298 | 3805594 | + | AluY | chr20 |
| AluSx | chr20 | 3805298 | 3805594 | + | AluSx | chr20 |
| AluSx4 | chr20 | 3809902 | 3810201 | − | AluJo | chr20 |
| AluSx | chr20 | 3808397 | 3808708 | − | AluJo | chr20 |
| AluY | chr20 | 3807911 | 3808200 | − | AluJo | chr20 |
| AluJo | chr20 | 3810557 | 3810856 | + | AluSp | chr20 |
| AluSp | chr20 | 3810874 | 3811173 | − | AluSx | chr20 |
| AluSx4 | chr20 | 3809902 | 3810201 | − | AluSx | chr20 |
| AluSp | chr20 | 3810874 | 3811173 | − | AluY | chr20 |
| AluSx4 | chr20 | 3809902 | 3810201 | − | AluY | chr20 |
| AluJo | chr20 | 3810557 | 3810856 | + | AluSz | chr20 |
| AluY | chr20 | 3812904 | 3813214 | + | AluSz | chr20 |
| AluSx | chr20 | 3812604 | 3812903 | + | AluSz | chr20 |
| AluY | chr20 | 3812904 | 3813214 | + | AluSc | chr20 |
| AluJo | chr20 | 3810557 | 3810856 | + | AluSc | chr20 |
| AluSx | chr20 | 3812604 | 3812903 | + | AluSc | chr20 |
| AluSx1 | chr20 | 3849000 | 3849295 | + | AluY | chr20 |
| AluJr | chr20 | 3848054 | 3848256 | + | AluY | chr20 |
| AluSx1 | chr20 | 3849000 | 3849295 | + | AluSx1 | chr20 |
| AluJr | chr20 | 3848054 | 3848256 | + | AluSx1 | chr20 |
| AluSx1 | chr20 | 3849000 | 3849295 | + | AluSx1 | chr20 |
| AluSz | chr20 | 3878374 | 3878662 | + | AluSx1 | chr20 |
| AluJb | chr20 | 3878066 | 3878372 | + | AluSx1 | chr20 |
| AluY | chr20 | 3885998 | 3886293 | − | AluJb | chr20 |
| AluSz | chr20 | 3899548 | 3899855 | − | AluSp | chr20 |
| AluSp | chr20 | 3901212 | 3901508 | + | AluSz | chr20 |
| AluSx | chr20 | 3905897 | 3906179 | − | AluY | chr20 |
| AluSx | chr20 | 5091546 | 5091845 | + | AluSg | chr20 |
| AluSx4 | chr20 | 5091975 | 5092284 | + | AluSg | chr20 |
| AluY | chr20 | 5561090 | 5561399 | + | AluSz | chr20 |
| AluSg | chr20 | 5568022 | 5568311 | − | AluSp | chr20 |
| AluSg | chr20 | 5568022 | 5568311 | − | AluSx | chr20 |
| AluSx | chr20 | 5980640 | 5980937 | − | AluJr | chr20 |
| AluJr4 | chr20 | 6053951 | 6054240 | − | AluSp | chr20 |
| AluSx1 | chr20 | 10413159 | 10413463 | + | AluSz | chr20 |
| AluSx3 | chr20 | 10459997 | 10460293 | − | AluSc5 | chr20 |
| AluSg7 | chr20 | 17547309 | 17547596 | − | AluSp | chr20 |
| AluSp | chr20 | 17957288 | 17957603 | − | AluSx | chr20 |
| AluJb | chr20 | 17967015 | 17967303 | − | AluSz | chr20 |
| AluSq2 | chr20 | 17966714 | 17967010 | − | AluSz | chr20 |
| AluSq | chr20 | 21296140 | 21296435 | − | AluSq2 | chr20 |
| AluJr | chr20 | 21295149 | 21295362 | − | AluSq2 | chr20 |
| AluY | chr20 | 25366886 | 25367195 | − | AluSx1 | chr20 |
| AluSq2 | chr20 | 25391192 | 25391498 | − | AluJb | chr20 |
| AluSz | chr20 | 25558752 | 25559043 | − | AluY | chr20 |
| AluSp | chr20 | 25558457 | 25558751 | − | AluY | chr20 |
| AluSx1 | chr20 | 30188807 | 30189103 | + | AluSx | chr20 |
| AluSx | chr20 | 30189148 | 30189457 | − | AluSx1 | chr20 |
| AluSx1 | chr20 | 30190072 | 30190361 | + | AluSx | chr20 |
| AluSx1 | chr20 | 30188807 | 30189103 | + | AluSx | chr20 |
| AluSx3 | chr20 | 32083212 | 32083511 | + | AluSc | chr20 |
| AluSx | chr20 | 32092210 | 32092508 | − | AluSp | chr20 |
| AluSx1 | chr20 | 32097352 | 32097628 | + | AluY | chr20 |
| AluSx1 | chr20 | 32097352 | 32097628 | + | AluY | chr20 |
| AluSz | chr20 | 32234380 | 32234690 | + | AluSp | chr20 |
| AluSz | chr20 | 32859372 | 32859671 | − | AluJo | chr20 |
| AluJo | chr20 | 32862475 | 32862757 | + | AluSx | chr20 |
| AluSx | chr20 | 32862933 | 32863237 | − | AluYc | chr20 |
| AluSg | chr20 | 32885111 | 32885401 | − | AluSx1 | chr20 |
| AluY | chr20 | 32958693 | 32958992 | + | AluSx | chr20 |
| AluY | chr20 | 32958693 | 32958992 | + | AluSq2 | chr20 |
| AluY | chr20 | 32958693 | 32958992 | + | AluSx1 | chr20 |
| AluJb | chr20 | 33055052 | 33055351 | + | AluSp | chr20 |
| AluJb | chr20 | 33055052 | 33055351 | + | AluY | chr20 |
| AluY | chr20 | 33055840 | 33056138 | − | AluSx1 | chr20 |
| AluSp | chr20 | 33055478 | 33055686 | − | AluSx1 | chr20 |
| AluSx1 | chr20 | 33056314 | 33056624 | + | AluSx1 | chr20 |
| AluJb | chr20 | 33055052 | 33055351 | + | AluSx1 | chr20 |
| AluSx1 | chr20 | 33074131 | 33074430 | − | AluSq2 | chr20 |
| AluSp | chr20 | 33084329 | 33084638 | + | AluSg | chr20 |
| AluY | chr20 | 33395329 | 33395627 | − | AluSx1 | chr20 |
| AluY | chr20 | 33617428 | 33617738 | − | AluSp | chr20 |
| AluY | chr20 | 33868903 | 33869218 | + | AluSx | chr20 |
| AluSx | chr20 | 33987729 | 33988023 | − | AluY | chr20 |
| AluJb | chr20 | 34173304 | 34173582 | − | AluY | chr20 |
| AluJb | chr20 | 34173304 | 34173582 | − | AluSg | chr20 |
| AluSg | chr20 | 34196377 | 34196668 | − | AluSg | chr20 |
| AluSx1 | chr20 | 34195796 | 34196129 | − | AluSg | chr20 |
| AluSx | chr20 | 34196198 | 34196376 | − | AluSg | chr20 |
| AluSg | chr20 | 34198387 | 34198680 | + | AluSx | chr20 |
| AluSx | chr20 | 34199323 | 34199618 | − | AluY | chr20 |
| AluY | chr20 | 34199782 | 34200079 | + | AluSg | chr20 |
| AluSg | chr20 | 34198387 | 34198680 | + | AluSg | chr20 |
| AluSg | chr20 | 34198387 | 34198680 | + | AluSx4 | chr20 |
| AluY | chr20 | 34199782 | 34200079 | + | AluSx4 | chr20 |
| AluY | chr20 | 34199782 | 34200079 | + | AluSx | chr20 |
| AluSg | chr20 | 34198387 | 34198680 | + | AluSx | chr20 |
| AluSg | chr20 | 34200082 | 34200351 | − | AluSx | chr20 |
| AluSx | chr20 | 34199323 | 34199618 | − | AluSx | chr20 |
| AluSx | chr20 | 34201089 | 34201400 | − | AluSx | chr20 |
| AluSx4 | chr20 | 34200623 | 34200918 | − | AluSx | chr20 |
| AluSx | chr20 | 34199323 | 34199618 | − | AluJo | chr20 |
| AluSx | chr20 | 34201089 | 34201400 | − | AluJo | chr20 |
| AluSg | chr20 | 34200082 | 34200351 | − | AluJo | chr20 |
| AluSx4 | chr20 | 34200623 | 34200918 | − | AluJo | chr20 |
| AluSg | chr20 | 34200082 | 34200351 | − | AluSp | chr20 |
| AluJb | chr20 | 34217578 | 34217883 | − | AluSx1 | chr20 |
| AluSx1 | chr20 | 34222288 | 34222539 | + | AluJo | chr20 |
| AluJo | chr20 | 34222660 | 34222964 | − | AluSz | chr20 |
| AluSx1 | chr20 | 34264786 | 34265085 | − | AluJr | chr20 |
| AluSx1 | chr20 | 34272391 | 34272666 | + | AluSx | chr20 |
| AluSx1 | chr20 | 34308414 | 34308698 | + | AluSq2 | chr20 |
| AluSq2 | chr20 | 34311365 | 34311659 | − | AluJr4 | chr20 |
| AluSq2 | chr20 | 34311365 | 34311659 | − | AluSx1 | chr20 |
| AluSx1 | chr20 | 34311972 | 34312267 | + | AluSc8 | chr20 |
| AluJr4 | chr20 | 34311705 | 34311968 | + | AluSc8 | chr20 |
| AluSc | chr20 | 34324577 | 34324872 | − | AluY | chr20 |
| AluSx1 | chr20 | 34711355 | 34711651 | − | AluY | chr20 |
| AluJr | chr20 | 35526446 | 35526730 | − | AluSg | chr20 |
| AluSg | chr20 | 35527035 | 35527342 | + | AluSz | chr20 |
| AluSq2 | chr20 | 35534092 | 35534390 | + | AluSz6 | chr20 |
| AluSq2 | chr20 | 35534092 | 35534390 | + | AluJo | chr20 |
| AluSz6 | chr20 | 35535200 | 35535494 | − | AluSq2 | chr20 |
| AluJo | chr20 | 35535495 | 35535827 | − | AluSq2 | chr20 |
| AluSx1 | chr20 | 35542749 | 35543062 | − | AluJr | chr20 |
| AluSz6 | chr20 | 35542160 | 35542456 | − | AluJr | chr20 |
| AluSz | chr20 | 37640609 | 37640911 | + | AluSz6 | chr20 |
| AluY | chr20 | 40100181 | 40100474 | − | AluSx1 | chr20 |
| AluSq2 | chr20 | 42205299 | 42205601 | + | AluSc | chr20 |
| AluSq2 | chr20 | 42205299 | 42205601 | + | AluSx1 | chr20 |
| AluSx1 | chr20 | 42206289 | 42206584 | − | AluSx1 | chr20 |
| AluSc | chr20 | 42205988 | 42206288 | − | AluSx1 | chr20 |
| AluSx1 | chr20 | 42206843 | 42207139 | + | AluSx | chr20 |
| AluSq2 | chr20 | 42205299 | 42205601 | + | AluSx | chr20 |
| AluSx | chr20 | 42207652 | 42207955 | − | AluSz | chr20 |
| AluSc | chr20 | 42205988 | 42206288 | − | AluSz | chr20 |
| AluSx1 | chr20 | 42206289 | 42206584 | − | AluSz | chr20 |
| AluSx1 | chr20 | 42206843 | 42207139 | + | AluY | chr20 |
| AluSz | chr20 | 42209086 | 42209388 | + | AluY | chr20 |
| AluSz | chr20 | 42214676 | 42214970 | − | AluSx3 | chr20 |
| AluSp | chr20 | 42934182 | 42934478 | + | AluSx | chr20 |
| AluSx | chr20 | 42975500 | 42975782 | + | AluJb | chr20 |
| AluSx1 | chr20 | 42990766 | 42991081 | + | AluSg7 | chr20 |
| AluSz6 | chr20 | 42996801 | 42997106 | − | AluSq2 | chr20 |
| AluSq2 | chr20 | 42997606 | 42997918 | + | AluY | chr20 |
| AluSq2 | chr20 | 42997606 | 42997918 | + | AluSx | chr20 |
| AluSz | chr20 | 43051353 | 43051618 | − | AluSz | chr20 |
| AluSz | chr20 | 43051675 | 43051979 | + | AluSx1 | chr20 |
| AluSx1 | chr20 | 43053628 | 43053918 | − | AluSz6 | chr20 |
| AluSx | chr20 | 43674436 | 43674747 | − | AluJr | chr20 |
| AluSg | chr20 | 43706836 | 43707145 | − | AluJb | chr20 |
| AluSp | chr20 | 44467534 | 44467837 | − | AluY | chr20 |
| AluY | chr20 | 44512990 | 44513300 | − | AluSx1 | chr20 |
| AluY | chr20 | 44512990 | 44513300 | − | AluJb | chr20 |
| AluSq | chr20 | 44524552 | 44524850 | + | AluSg | chr20 |
| AluSx3 | chr20 | 45869758 | 45870058 | + | AluY | chr20 |
| AluY | chr20 | 45870553 | 45870860 | − | AluSq2 | chr20 |
| AluJb | chr20 | 45950601 | 45950904 | − | AluSq | chr20 |
| AluSc | chr20 | 46141033 | 46141340 | + | AluSg7 | chr20 |
| AluSc | chr20 | 46141033 | 46141340 | + | AluSz6 | chr20 |
| AluSg7 | chr20 | 46143915 | 46144199 | − | AluSz | chr20 |
| AluSz6 | chr20 | 46144214 | 46144523 | − | AluSz | chr20 |
| AluSg7 | chr20 | 46143915 | 46144199 | − | AluSz | chr20 |
| AluSz6 | chr20 | 46144214 | 46144523 | − | AluSz | chr20 |
| AluSz | chr20 | 46145383 | 46145682 | + | AluSx1 | chr20 |
| AluSz | chr20 | 46145686 | 46145959 | + | AluSx1 | chr20 |
| AluSx1 | chr20 | 46146183 | 46146494 | − | AluJr | chr20 |
| AluSz6 | chr20 | 46144214 | 46144523 | − | AluJr | chr20 |
| AluSg7 | chr20 | 46143915 | 46144199 | − | AluJr | chr20 |
| AluJr | chr20 | 46146809 | 46147126 | + | AluSq2 | chr20 |
| AluSz | chr20 | 46145383 | 46145682 | + | AluSq2 | chr20 |
| AluSz | chr20 | 46145686 | 46145959 | + | AluSq2 | chr20 |
| AluSx1 | chr20 | 46146183 | 46146494 | − | AluSx1 | chr20 |
| AluSq2 | chr20 | 46147152 | 46147468 | − | AluSx1 | chr20 |
| AluSx1 | chr20 | 46146183 | 46146494 | − | AluSz | chr20 |
| AluSq2 | chr20 | 46147152 | 46147468 | − | AluSz | chr20 |
| AluSx1 | chr20 | 46159206 | 46159508 | − | AluSx1 | chr20 |
| AluSq2 | chr20 | 47577021 | 47577319 | − | AluSx1 | chr20 |
| AluJb | chr20 | 47576161 | 47576433 | − | AluSx1 | chr20 |
| AluSx1 | chr20 | 47725831 | 47726125 | + | AluSx | chr20 |
| AluSc | chr20 | 47779449 | 47779742 | + | AluSx | chr20 |
| AluY | chr20 | 47779030 | 47779326 | + | AluSx | chr20 |
| AluSx | chr20 | 47779957 | 47780260 | − | AluSx1 | chr20 |
| AluSx1 | chr20 | 47780488 | 47780779 | + | AluJb | chr20 |
| AluY | chr20 | 47779030 | 47779326 | + | AluJb | chr20 |
| AluSc | chr20 | 47779449 | 47779742 | + | AluJb | chr20 |
| AluY | chr20 | 47799095 | 47799392 | + | AluSq2 | chr20 |
| AluJb | chr20 | 48324166 | 48324450 | − | AluSq2 | chr20 |
| AluSq2 | chr20 | 48326027 | 48326321 | + | AluSg4 | chr20 |
| AluY | chr20 | 48433268 | 48433564 | − | AluSq2 | chr20 |
| AluSx | chr20 | 48556310 | 48556620 | + | AluY | chr20 |
| AluY | chr20 | 48556950 | 48557248 | − | AluJb | chr20 |
| AluJb | chr20 | 48559078 | 48559373 | + | AluSz6 | chr20 |
| AluSz6 | chr20 | 48560597 | 48560899 | − | AluSz | chr20 |
| AluSz | chr20 | 48787798 | 48788093 | + | AluYc3 | chr20 |
| AluYc3 | chr20 | 48790144 | 48790442 | − | AluSx1 | chr20 |
| AluSg | chr20 | 49353227 | 49353534 | − | AluSx | chr20 |
| AluSx | chr20 | 49355702 | 49355981 | + | AluSz6 | chr20 |
| AluSx | chr20 | 49355702 | 49355981 | + | AluSx | chr20 |
| AluSx | chr20 | 49358889 | 49359202 | − | AluY | chr20 |
| AluSz6 | chr20 | 49358615 | 49358880 | − | AluY | chr20 |
| AluY | chr20 | 49360164 | 49360446 | + | AluSp | chr20 |
| AluY | chr20 | 49554926 | 49555216 | − | AluSp | chr20 |
| AluSg7 | chr20 | 49558763 | 49559056 | + | AluSc | chr20 |
| AluSx1 | chr20 | 50354569 | 50354869 | − | AluSx | chr20 |
| AluSx3 | chr20 | 51712699 | 51712981 | − | AluSq2 | chr20 |
| AluSz | chr20 | 52085801 | 52086076 | + | AluSp | chr20 |
| AluY | chr20 | 52112822 | 52113114 | + | AluSx1 | chr20 |
| AluSg | chr20 | 52118611 | 52118903 | − | AluJb | chr20 |
| AluSz6 | chr20 | 52189997 | 52190296 | − | AluSx1 | chr20 |
| AluJb | chr20 | 56301405 | 56301686 | + | AluSz6 | chr20 |
| AluSq | chr20 | 60741710 | 60742011 | + | AluSx1 | chr20 |
| AluSx1 | chr20 | 60742091 | 60742398 | − | AluJb | chr20 |
| AluSz6 | chr20 | 60864678 | 60864985 | + | AluY | chr20 |
| AluSz6 | chr20 | 60864678 | 60864985 | + | AluSq | chr20 |
| AluY | chr20 | 61381761 | 61382067 | + | AluJr | chr20 |
| AluJo | chr20 | 61486510 | 61486797 | − | AluSp | chr20 |
| AluJo | chr20 | 61486510 | 61486797 | − | AluSg | chr20 |
| AluSx1 | chr20 | 62228089 | 62228397 | + | AluJr | chr20 |
| AluSx | chr20 | 62340708 | 62340981 | + | AluY | chr20 |
| AluY | chr20 | 62344590 | 62344881 | − | AluSq10 | chr20 |
| AluSx1 | chr20 | 62344417 | 62344589 | − | AluSq10 | chr20 |
| AluY | chr20 | 62344590 | 62344881 | − | AluSz6 | chr20 |
| AluSx1 | chr20 | 62344417 | 62344589 | − | AluSz6 | chr20 |
| AluSz6 | chr20 | 62345496 | 62345798 | + | AluJo | chr20 |
| AluSq10 | chr20 | 62344942 | 62345242 | + | AluJo | chr20 |
| AluY | chr20 | 62497804 | 62498111 | − | AluSz | chr20 |
| AluSp | chr20 | 62498299 | 62498606 | − | AluSz | chr20 |
| AluJb | chr20 | 62501196 | 62501495 | + | AluSz6 | chr20 |
| AluJb | chr20 | 62501196 | 62501495 | + | AluSx | chr20 |
| AluSx1 | chr20 | 62529915 | 62530213 | + | AluSc8 | chr20 |
| AluSc8 | chr20 | 62530931 | 62531231 | − | AluJb | chr20 |
| AluSz | chr20 | 62552019 | 62552296 | − | AluY | chr20 |
| AluSp | chr20 | 62552302 | 62552611 | − | AluY | chr20 |
| AluY | chr20 | 62552663 | 62552960 | + | AluSx | chr20 |
| AluSx | chr20 | 62553139 | 62553431 | − | AluSx | chr20 |
| AluSp | chr20 | 62552302 | 62552611 | − | AluSx | chr20 |
| AluSz | chr20 | 62552019 | 62552296 | − | AluSx | chr20 |
| AluSc8 | chr20 | 62617841 | 62618140 | − | AluJb | chr20 |
| AluSx1 | chr20 | 62651486 | 62651778 | + | AluSx | chr20 |
| AluJb | chr11 | 508175 | 508458 | − | AluSx | chr11 |
| AluJb | chr11 | 508175 | 508458 | − | AluSq | chr11 |
| AluSq | chr11 | 508762 | 509062 | + | AluSg | chr11 |
| AluSp | chr11 | 588373 | 588678 | − | AluSc | chr11 |
| AluSx1 | chr11 | 700245 | 700546 | + | AluY | chr11 |
| AluSz | chr11 | 745814 | 746100 | − | AluSq | chr11 |
| AluJb | chr11 | 927065 | 927360 | − | AluJb | chr11 |
| AluSx | chr11 | 3007322 | 3007633 | + | AluSq | chr11 |
| AluSp | chr11 | 3034391 | 3034675 | − | AluSx3 | chr11 |
| AluY | chr11 | 3812129 | 3812424 | − | AluSq10 | chr11 |
| AluSq10 | chr11 | 3812481 | 3812792 | + | AluSc8 | chr11 |
| AluY | chr11 | 3812129 | 3812424 | − | AluSq2 | chr11 |
| AluSc8 | chr11 | 3814528 | 3814814 | − | AluSq2 | chr11 |
| AluSq | chr11 | 4135502 | 4135796 | + | AluSz | chr11 |
| AluSq | chr11 | 8707789 | 8708076 | − | AluSp | chr11 |
| AluSc8 | chr11 | 9312959 | 9313245 | − | AluJo | chr11 |
| AluSg7 | chr11 | 9313252 | 9313552 | − | AluJo | chr11 |
| AluSc8 | chr11 | 9312959 | 9313245 | − | AluSq | chr11 |
| AluSg7 | chr11 | 9313252 | 9313552 | − | AluSq | chr11 |
| AluSx | chr11 | 9317889 | 9318178 | + | AluY | chr11 |
| AluSq | chr11 | 9315761 | 9316052 | + | AluY | chr11 |
| AluJo | chr11 | 9315471 | 9315757 | + | AluY | chr11 |
| AluY | chr11 | 9318643 | 9318931 | − | AluSp | chr11 |
| AluSg | chr11 | 9436167 | 9436470 | − | AluSp | chr11 |
| AluSx | chr11 | 9831092 | 9831392 | − | AluJb | chr11 |
| AluYf4 | chr11 | 11921702 | 11922006 | − | AluSz | chr11 |
| AluSz | chr11 | 12891423 | 12891726 | − | AluSx3 | chr11 |
| AluSx1 | chr11 | 13019740 | 13020043 | − | AluSz | chr11 |
| AluY | chr11 | 13039314 | 13039616 | − | AluJb | chr11 |
| AluSg7 | chr11 | 14298255 | 14298523 | + | AluSx1 | chr11 |
| AluSz | chr11 | 14607696 | 14607993 | + | AluSc8 | chr11 |
| AluSx | chr11 | 17152070 | 17152383 | + | AluY | chr11 |
| AluSq | chr11 | 17152452 | 17152748 | + | AluY | chr11 |
| AluSx | chr11 | 19783507 | 19783809 | + | AluSc | chr11 |
| AluSz | chr11 | 20387236 | 20387544 | + | AluY | chr11 |
| AluSq2 | chr11 | 27231586 | 27231887 | − | AluJo | chr11 |
| AluSg | chr11 | 32875116 | 32875403 | + | AluJb | chr11 |
| AluJo | chr11 | 33096195 | 33096495 | + | AluY | chr11 |
| AluSx | chr11 | 43822407 | 43822704 | + | AluY | chr11 |
| AluJr4 | chr11 | 43844826 | 43845118 | − | AluJb | chr11 |
| AluSg | chr11 | 43878975 | 43879285 | + | AluSx3 | chr11 |
| AluSq2 | chr11 | 46490412 | 46490709 | + | AluJr | chr11 |
| AluJr | chr11 | 46491208 | 46491527 | − | AluY | chr11 |
| AluSp | chr11 | 46596729 | 46597020 | + | AluSg | chr11 |
| AluSp | chr11 | 46607358 | 46607653 | − | AluSz6 | chr11 |
| AluSp | chr11 | 46640923 | 46641237 | − | AluSq2 | chr11 |
| AluSc8 | chr11 | 47246862 | 47247167 | + | AluSx | chr11 |
| AluSz6 | chr11 | 47502307 | 47502547 | + | AluSx1 | chr11 |
| AluSx1 | chr11 | 47502652 | 47502981 | − | AluSx | chr11 |
| AluJb | chr11 | 47532049 | 47532339 | − | AluJb | chr11 |
| AluJb | chr11 | 47532453 | 47532754 | + | AluY | chr11 |
| AluSq | chr11 | 47555570 | 47555881 | − | AluY | chr11 |
| AluSx1 | chr11 | 47553582 | 47553875 | − | AluY | chr11 |
| AluY | chr11 | 47555997 | 47556306 | + | AluSc8 | chr11 |
| AluSc8 | chr11 | 47557309 | 47557613 | − | AluSx | chr11 |
| AluSq | chr11 | 47555570 | 47555881 | − | AluSx | chr11 |
| AluSq | chr11 | 47567839 | 47568082 | − | AluSg4 | chr11 |
| AluSg | chr11 | 47860276 | 47860581 | + | AluJr | chr11 |
| AluJr | chr11 | 47863234 | 47863548 | − | AluJo | chr11 |
| AluSx1 | chr11 | 57535309 | 57535606 | − | AluSz | chr11 |
| AluSx3 | chr11 | 57534437 | 57534732 | − | AluSz | chr11 |
| AluSx1 | chr11 | 57535309 | 57535606 | − | AluSx1 | chr11 |
| AluSx3 | chr11 | 57534437 | 57534732 | − | AluSx1 | chr11 |
| AluSx1 | chr11 | 57535309 | 57535606 | − | AluSc | chr11 |
| AluSc | chr11 | 57537845 | 57538153 | + | AluJb | chr11 |
| AluSz | chr11 | 57536497 | 57536806 | + | AluJb | chr11 |
| AluSx1 | chr11 | 57537516 | 57537830 | + | AluJb | chr11 |
| AluJb | chr11 | 57539398 | 57539703 | − | AluSx1 | chr11 |
| AluSq2 | chr11 | 57548405 | 57548708 | − | AluSz | chr11 |
| AluJo | chr11 | 57547529 | 57547826 | − | AluSz | chr11 |
| AluSq2 | chr11 | 57548405 | 57548708 | − | AluY | chr11 |
| AluY | chr11 | 57550863 | 57551144 | + | AluJb | chr11 |
| AluSz | chr11 | 57550476 | 57550778 | + | AluJb | chr11 |
| AluY | chr11 | 57550863 | 57551144 | + | AluSz | chr11 |
| AluSz | chr11 | 57550476 | 57550778 | + | AluSz | chr11 |
| AluY | chr11 | 57550863 | 57551144 | + | AluSg | chr11 |
| AluSz | chr11 | 57550476 | 57550778 | + | AluSg | chr11 |
| AluSg | chr11 | 57553043 | 57553318 | − | AluSc8 | chr11 |
| AluSz | chr11 | 57551975 | 57552277 | − | AluSc8 | chr11 |
| AluJb | chr11 | 57551384 | 57551693 | − | AluSc8 | chr11 |
| AluSx3 | chr11 | 57577801 | 57578096 | − | AluJb | chr11 |
| AluSx1 | chr11 | 59357178 | 59357471 | + | AluSx | chr11 |
| AluSx4 | chr11 | 61177166 | 61177452 | + | AluJo | chr11 |
| AluJo | chr11 | 61177584 | 61177855 | − | AluSz | chr11 |
| AluY | chr11 | 62408024 | 62408325 | + | AluSx | chr11 |
| AluSx | chr11 | 62409056 | 62409350 | − | AluSg | chr11 |
| AluSq2 | chr11 | 62517261 | 62517555 | − | AluY | chr11 |
| AluSx3 | chr11 | 62516039 | 62516338 | − | AluSx | chr11 |
| AluSq2 | chr11 | 62517261 | 62517555 | − | AluSx | chr11 |
| AluSq2 | chr11 | 62587169 | 62587475 | − | AluY | chr11 |
| AluSq2 | chr11 | 62587169 | 62587475 | − | AluSc | chr11 |
| AluSz | chr11 | 62605624 | 62605926 | − | AluSq | chr11 |
| AluSz | chr11 | 62653480 | 62653790 | + | AluJr | chr11 |
| AluSx | chr11 | 63586674 | 63586949 | − | AluSx1 | chr11 |
| AluSg | chr11 | 63627098 | 63627401 | − | AluSc | chr11 |
| AluSp | chr11 | 63942770 | 63943069 | + | AluSx1 | chr11 |
| AluSx | chr11 | 63947645 | 63947956 | + | AluSx | chr11 |
| AluSz | chr11 | 64075364 | 64075626 | − | AluSx | chr11 |
| AluSz | chr11 | 64075364 | 64075626 | − | AluY | chr11 |
| AluSz | chr11 | 64075364 | 64075626 | − | AluSg | chr11 |
| AluY | chr11 | 64076011 | 64076304 | + | AluY | chr11 |
| AluSx | chr11 | 64075705 | 64076010 | + | AluY | chr11 |
| AluSg | chr11 | 64077124 | 64077426 | + | AluY | chr11 |
| AluSg | chr11 | 64077124 | 64077426 | + | AluSq2 | chr11 |
| AluY | chr11 | 64076011 | 64076304 | + | AluSq2 | chr11 |
| AluSx | chr11 | 64075705 | 64076010 | + | AluSq2 | chr11 |
| AluSx1 | chr11 | 65030692 | 65030994 | − | AluSp | chr11 |
| AluSc5 | chr11 | 65090401 | 65090704 | + | AluSx | chr11 |
| AluSc5 | chr11 | 65090401 | 65090704 | + | AluSx1 | chr11 |
| AluSc5 | chr11 | 65090401 | 65090704 | + | AluSc8 | chr11 |
| AluSx | chr11 | 65139388 | 65139682 | − | AluSz | chr11 |
| AluJr4 | chr11 | 65540237 | 65540512 | + | AluSx | chr11 |
| AluJb | chr11 | 66121422 | 66121672 | + | AluSp | chr11 |
| AluSp | chr11 | 66124418 | 66124687 | − | AluY | chr11 |
| AluSx1 | chr11 | 66416476 | 66416768 | − | AluSq2 | chr11 |
| AluSq2 | chr11 | 66417759 | 66418044 | + | AluY | chr11 |
| AluSq2 | chr11 | 66417759 | 66418044 | + | AluY | chr11 |
| AluSp | chr11 | 66513912 | 66514197 | + | AluSx | chr11 |
| AluSx | chr11 | 66517737 | 66518037 | + | AluSz | chr11 |
| AluSg7 | chr11 | 66560905 | 66561216 | − | AluJr4 | chr11 |
| AluSg7 | chr11 | 66560905 | 66561216 | − | AluSq | chr11 |
| AluSq | chr11 | 66562322 | 66562614 | + | AluSx | chr11 |
| AluJr4 | chr11 | 66561297 | 66561612 | + | AluSx | chr11 |
| AluY | chr11 | 66896924 | 66897233 | + | AluSq2 | chr11 |
| AluSx | chr11 | 66912681 | 66912974 | + | AluSx3 | chr11 |
| AluSx1 | chr11 | 67062001 | 67062280 | − | AluY | chr11 |
| AluJb | chr11 | 67852705 | 67852987 | − | AluSz | chr11 |
| AluJb | chr11 | 67860464 | 67860766 | − | AluJb | chr11 |
| AluJr | chr11 | 68244909 | 68245209 | + | AluSq4 | chr11 |
| AluSg | chr11 | 70907327 | 70907629 | − | AluSx1 | chr11 |
| AluSx | chr11 | 71657914 | 71658208 | − | AluJo | chr11 |
| AluSx | chr11 | 71997523 | 71997816 | − | AluSz6 | chr11 |
| AluSz6 | chr11 | 71998361 | 71998659 | − | AluSz6 | chr11 |
| AluSz6 | chr11 | 71999250 | 71999557 | + | AluSc | chr11 |
| AluSc | chr11 | 72000002 | 72000232 | − | AluSx1 | chr11 |
| AluSx1 | chr11 | 72001711 | 72002016 | + | AluSx | chr11 |
| AluSz6 | chr11 | 71999250 | 71999557 | + | AluSx | chr11 |
| AluSx | chr11 | 72002358 | 72002650 | − | AluY | chr11 |
| AluSc | chr11 | 72000002 | 72000232 | − | AluY | chr11 |
| AluSx1 | chr11 | 74166763 | 74167027 | − | AluSz | chr11 |
| AluY | chr11 | 75751280 | 75751543 | + | AluSc | chr11 |
| AluSz | chr11 | 76076044 | 76076355 | + | AluJb | chr11 |
| AluSq2 | chr11 | 76959761 | 76960005 | − | AluSc | chr11 |
| AluSg | chr11 | 76959241 | 76959544 | − | AluSc | chr11 |
| AluJr | chr11 | 78174119 | 78174405 | − | AluSq2 | chr11 |
| AluSq4 | chr11 | 82786287 | 82786584 | + | AluSg | chr11 |
| AluSg | chr11 | 82788106 | 82788395 | − | AluSx1 | chr11 |
| AluSz | chr11 | 82787500 | 82787802 | − | AluSx1 | chr11 |
| AluSx1 | chr11 | 82790670 | 82790964 | + | AluYc | chr11 |
| AluYc3 | chr11 | 82900385 | 82900671 | + | AluSc | chr11 |
| AluJb | chr11 | 1.02E+08 | 1.02E+08 | − | AluSz | chr11 |
| AluSz | chr11 | 1.02E+08 | 1.02E+08 | − | AluSc5 | chr11 |
| AluSc5 | chr11 | 1.02E+08 | 1.02E+08 | + | AluJb | chr11 |
| AluSp | chr11 | 1.02E+08 | 1.02E+08 | − | AluJb | chr11 |
| AluSc8 | chr11 | 1.02E+08 | 1.02E+08 | − | AluSq2 | chr11 |
| AluSp | chr11 | 1.02E+08 | 1.02E+08 | − | AluSq2 | chr11 |
| AluSc8 | chr11 | 1.02E+08 | 1.02E+08 | − | AluSx | chr11 |
| AluSp | chr11 | 1.02E+08 | 1.02E+08 | − | AluSx | chr11 |
| AluSz | chr11 | 1.03E+08 | 1.03E+08 | + | AluSg | chr11 |
| AluSx | chr11 | 1.17E+08 | 1.17E+08 | − | AluSq4 | chr11 |
| AluSx1 | chr11 | 1.19E+08 | 1.19E+08 | + | AluSc8 | chr11 |
| AluSq2 | chr11 | 1.19E+08 | 1.19E+08 | + | AluSz | chr11 |
| AluSq | chr11 | 1.19E+08 | 1.19E+08 | + | AluSz | chr11 |
| AluSq | chr11 | 1.19E+08 | 1.19E+08 | + | AluSx | chr11 |
| AluSq2 | chr11 | 1.19E+08 | 1.19E+08 | + | AluSx | chr11 |
| AluSq2 | chr11 | 1.19E+08 | 1.19E+08 | + | AluSx4 | chr11 |
| AluSz | chr11 | 1.19E+08 | 1.19E+08 | − | AluJb | chr11 |
| AluSx | chr11 | 1.19E+08 | 1.19E+08 | − | AluJb | chr11 |
| AluSx4 | chr11 | 1.19E+08 | 1.19E+08 | − | AluJb | chr11 |
| AluJb | chr11 | 1.19E+08 | 1.19E+08 | + | AluY | chr11 |
| AluJb | chr11 | 1.19E+08 | 1.19E+08 | + | AluSg | chr11 |
| AluSg | chr11 | 1.19E+08 | 1.19E+08 | − | AluJb | chr11 |
| AluSp | chr11 | 1.19E+08 | 1.19E+08 | − | AluSp | chr11 |
| AluSc | chr11 | 1.23E+08 | 1.23E+08 | + | AluSc | chr11 |
| AluSx1 | chr11 | 1.26E+08 | 1.26E+08 | + | AluSq2 | chr11 |
| AluSx1 | chr11 | 1.3E+08 | 1.3E+08 | + | AluY | chr11 |
| AluSx | chr11 | 1.3E+08 | 1.3E+08 | + | AluSg | chr11 |
| AluSx | chr11 | 1.3E+08 | 1.3E+08 | + | AluSx1 | chr11 |
| AluY | chr5 | 14176299 | 14176588 | + | AluJb | chr5 |
| AluSx1 | chr5 | 16925576 | 16925820 | − | AluSz | chr5 |
| AluSz | chr5 | 20743201 | 20743491 | + | AluSx1 | chr5 |
| AluSz | chr5 | 20743201 | 20743491 | + | AluY | chr5 |
| AluSc | chr5 | 21462690 | 21462995 | − | AluSq2 | chr5 |
| AluSx3 | chr5 | 21462393 | 21462688 | − | AluSq2 | chr5 |
| AluSx | chr5 | 32165004 | 32165302 | − | AluSx1 | chr5 |
| AluSx | chr5 | 32165004 | 32165302 | − | AluSq2 | chr5 |
| AluSx1 | chr5 | 32165407 | 32165707 | + | AluSq2 | chr5 |
| AluSq2 | chr5 | 32166640 | 32166928 | + | AluSq2 | chr5 |
| AluSx | chr5 | 33450939 | 33451235 | + | AluY | chr5 |
| AluJb | chr5 | 33992532 | 33992826 | + | AluSz | chr5 |
| AluSz | chr5 | 34668226 | 34668514 | + | AluSc | chr5 |
| AluJo | chr5 | 34906462 | 34906763 | + | AluSp | chr5 |
| AluY | chr5 | 37037208 | 37037494 | + | AluJr | chr5 |
| AluSp | chr5 | 37287011 | 37287304 | − | AluSp | chr5 |
| AluSp | chr5 | 37287011 | 37287304 | − | AluSz | chr5 |
| AluY | chr5 | 43128478 | 43128779 | + | AluSx1 | chr5 |
| AluY | chr5 | 43128478 | 43128779 | + | AluSg | chr5 |
| AluSg | chr5 | 43129757 | 43130057 | − | AluSx | chr5 |
| AluSx1 | chr5 | 43129047 | 43129337 | − | AluSx | chr5 |
| AluJb | chr5 | 43309341 | 43309639 | − | AluSz | chr5 |
| AluJo | chr5 | 43380621 | 43380940 | + | AluJr | chr5 |
| AluSx1 | chr5 | 43520011 | 43520316 | + | AluY | chr5 |
| AluY | chr5 | 60189806 | 60190109 | − | AluSg7 | chr5 |
| AluSx3 | chr5 | 60673298 | 60673555 | + | AluSp | chr5 |
| AluSx1 | chr5 | 64842938 | 64843235 | + | AluY | chr5 |
| AluSx1 | chr5 | 64963775 | 64964079 | − | AluSc | chr5 |
| AluSc8 | chr5 | 65243072 | 65243243 | − | AluSq | chr5 |
| AluSq | chr5 | 65243554 | 65243854 | + | AluSx1 | chr5 |
| AluSq2 | chr5 | 68662593 | 68662885 | − | AluSc5 | chr5 |
| AluSp | chr5 | 69370189 | 69370483 | + | AluSx | chr5 |
| AluSp | chr5 | 69370539 | 69370839 | + | AluSx1 | chr5 |
| AluSp | chr5 | 69370189 | 69370483 | + | AluSx1 | chr5 |
| AluSp | chr5 | 70816734 | 70817029 | + | AluSp | chr5 |
| AluY | chr5 | 77429788 | 77429994 | + | AluSq2 | chr5 |
| AluSq2 | chr5 | 78717427 | 78717729 | − | AluSz | chr5 |
| AluY | chr5 | 78798633 | 78798909 | − | AluSz | chr5 |
| AluSx1 | chr5 | 79449000 | 79449223 | + | AluSp | chr5 |
| AluSq | chr5 | 79741721 | 79742015 | − | AluSx | chr5 |
| AluJo | chr5 | 79776462 | 79776740 | + | AluSx3 | chr5 |
| AluJo | chr5 | 79776462 | 79776740 | + | AluSx | chr5 |
| AluSz | chr5 | 1.03E+08 | 1.03E+08 | − | AluY | chr5 |
| AluSz | chr5 | 1.04E+08 | 1.04E+08 | − | AluSx | chr5 |
| AluSz6 | chr5 | 1.04E+08 | 1.04E+08 | − | AluY | chr5 |
| AluSx1 | chr5 | 1.12E+08 | 1.12E+08 | + | AluSc | chr5 |
| AluSc | chr5 | 1.12E+08 | 1.12E+08 | − | AluSx3 | chr5 |
| AluSp | chr5 | 1.12E+08 | 1.12E+08 | − | AluSq | chr5 |
| AluY | chr5 | 1.26E+08 | 1.26E+08 | + | AluSz | chr5 |
| AluSz6 | chr5 | 1.26E+08 | 1.26E+08 | − | AluSc | chr5 |
| AluJb | chr5 | 1.3E+08 | 1.3E+08 | − | AluSp | chr5 |
| AluSg | chr5 | 1.31E+08 | 1.31E+08 | + | AluJb | chr5 |
| AluJb | chr5 | 1.31E+08 | 1.31E+08 | − | AluSp | chr5 |
| AluSz6 | chr5 | 1.31E+08 | 1.31E+08 | − | AluSc5 | chr5 |
| AluJb | chr5 | 1.32E+08 | 1.32E+08 | + | AluSx | chr5 |
| AluY | chr5 | 1.32E+08 | 1.32E+08 | + | AluSq10 | chr5 |
| AluSz6 | chr5 | 1.32E+08 | 1.32E+08 | + | AluSq10 | chr5 |
| AluSq10 | chr5 | 1.32E+08 | 1.32E+08 | − | AluSx1 | chr5 |
| AluY | chr5 | 1.32E+08 | 1.32E+08 | + | AluJb | chr5 |
| AluY | chr5 | 1.34E+08 | 1.34E+08 | − | AluSx3 | chr5 |
| AluY | chr5 | 1.34E+08 | 1.34E+08 | − | AluSx1 | chr5 |
| AluSq2 | chr5 | 1.34E+08 | 1.34E+08 | − | AluSx1 | chr5 |
| AluSc | chr5 | 1.34E+08 | 1.34E+08 | + | AluSg | chr5 |
| AluSx1 | chr5 | 1.34E+08 | 1.34E+08 | + | AluSp | chr5 |
| AluSc8 | chr5 | 1.34E+08 | 1.34E+08 | − | AluSg | chr5 |
| AluSx1 | chr5 | 1.34E+08 | 1.34E+08 | − | AluSx1 | chr5 |
| AluSq | chr5 | 1.34E+08 | 1.34E+08 | − | AluSx1 | chr5 |
| AluSx1 | chr5 | 1.34E+08 | 1.34E+08 | − | AluSx1 | chr5 |
| AluSx1 | chr5 | 1.34E+08 | 1.34E+08 | − | AluY | chr5 |
| AluSx1 | chr5 | 1.34E+08 | 1.34E+08 | − | AluSp | chr5 |
| AluSx1 | chr5 | 1.34E+08 | 1.34E+08 | − | AluSp | chr5 |
| AluSx1 | chr5 | 1.34E+08 | 1.34E+08 | − | AluSg | chr5 |
| AluSq2 | chr5 | 1.34E+08 | 1.34E+08 | − | AluSg | chr5 |
| AluJb | chr5 | 1.37E+08 | 1.37E+08 | − | AluSp | chr5 |
| AluSx1 | chr5 | 1.37E+08 | 1.37E+08 | + | AluY | chr5 |
| AluSx1 | chr5 | 1.38E+08 | 1.38E+08 | + | AluSz | chr5 |
| AluSx1 | chr5 | 1.38E+08 | 1.38E+08 | − | AluSx | chr5 |
| AluSx3 | chr5 | 1.39E+08 | 1.39E+08 | + | AluSq2 | chr5 |
| AluSx | chr5 | 1.39E+08 | 1.39E+08 | − | AluSc | chr5 |
| AluSz | chr5 | 1.39E+08 | 1.39E+08 | + | AluSx4 | chr5 |
| AluSx | chr5 | 1.39E+08 | 1.39E+08 | + | AluJb | chr5 |
| AluJb | chr5 | 1.39E+08 | 1.39E+08 | − | AluY | chr5 |
| AluSx1 | chr5 | 1.39E+08 | 1.39E+08 | − | AluJb | chr5 |
| AluSx1 | chr5 | 1.4E+08 | 1.4E+08 | − | AluSz | chr5 |
| AluSz | chr5 | 1.4E+08 | 1.4E+08 | + | AluSx3 | chr5 |
| AluSx3 | chr5 | 1.41E+08 | 1.41E+08 | + | AluSx1 | chr5 |
| AluSz6 | chr5 | 1.42E+08 | 1.42E+08 | − | AluSx | chr5 |
| AluY | chr5 | 1.42E+08 | 1.42E+08 | + | AluJb | chr5 |
| AluJb | chr5 | 1.42E+08 | 1.42E+08 | − | AluJb | chr5 |
| AluJb | chr5 | 1.42E+08 | 1.42E+08 | + | AluSc | chr5 |
| AluSz | chr5 | 1.46E+08 | 1.46E+08 | − | AluSp | chr5 |
| AluSx | chr5 | 1.46E+08 | 1.46E+08 | − | AluSq2 | chr5 |
| AluSx | chr5 | 1.49E+08 | 1.49E+08 | − | AluSc | chr5 |
| AluSx | chr5 | 1.49E+08 | 1.49E+08 | − | AluSq2 | chr5 |
| AluSq | chr5 | 1.51E+08 | 1.51E+08 | − | AluSz | chr5 |
| AluSx3 | chr5 | 1.54E+08 | 1.54E+08 | + | AluSp | chr5 |
| AluSx | chr5 | 1.57E+08 | 1.57E+08 | − | AluY | chr5 |
| AluSq2 | chr5 | 1.6E+08 | 1.6E+08 | + | AluSz6 | chr5 |
| AluSq2 | chr5 | 1.6E+08 | 1.6E+08 | + | AluSz | chr5 |
| AluSz6 | chr5 | 1.6E+08 | 1.6E+08 | − | AluSx | chr5 |
| AluSz | chr5 | 1.6E+08 | 1.6E+08 | − | AluSx | chr5 |
| AluSx | chr5 | 1.6E+08 | 1.6E+08 | + | AluSx3 | chr5 |
| AluSz | chr5 | 1.6E+08 | 1.6E+08 | − | AluSx | chr5 |
| AluSx3 | chr5 | 1.6E+08 | 1.6E+08 | − | AluSx | chr5 |
| AluSx | chr5 | 1.6E+08 | 1.6E+08 | + | AluJb | chr5 |
| AluSz | chr5 | 1.68E+08 | 1.68E+08 | + | AluSx | chr5 |
| AluSx3 | chr5 | 1.72E+08 | 1.72E+08 | + | AluSz | chr5 |
| AluSx | chr5 | 1.72E+08 | 1.72E+08 | + | AluSz | chr5 |
| AluSx3 | chr5 | 1.76E+08 | 1.76E+08 | − | AluY | chr5 |
| AluSz6 | chr5 | 1.76E+08 | 1.76E+08 | − | AluY | chr5 |
| AluSq2 | chr5 | 1.76E+08 | 1.76E+08 | − | AluY | chr5 |
| AluY | chr5 | 1.76E+08 | 1.76E+08 | − | AluSp | chr5 |
| AluSc | chr5 | 1.76E+08 | 1.76E+08 | + | AluSx1 | chr5 |
| AluJb | chr5 | 1.76E+08 | 1.76E+08 | + | AluSx1 | chr5 |
| AluSx1 | chr5 | 1.76E+08 | 1.76E+08 | − | AluSx | chr5 |
| AluSg | chr5 | 1.77E+08 | 1.77E+08 | − | AluJb | chr5 |
| AluSx1 | chr5 | 1.77E+08 | 1.77E+08 | − | AluSx1 | chr5 |
| AluSx1 | chr5 | 1.77E+08 | 1.77E+08 | − | AluSp | chr5 |
| AluSz | chr5 | 1.78E+08 | 1.78E+08 | − | AluY | chr5 |
| AluSz | chr5 | 1.78E+08 | 1.78E+08 | − | AluJb | chr5 |
| AluY | chr5 | 1.78E+08 | 1.78E+08 | − | AluJb | chr5 |
| AluJo | chr5 | 1.78E+08 | 1.78E+08 | − | AluY | chr5 |
| AluSx1 | chr5 | 1.79E+08 | 1.79E+08 | + | AluSx | chr5 |
| AluSx1 | chr5 | 1.79E+08 | 1.79E+08 | + | AluSz6 | chr5 |
| AluY | chr5 | 1.79E+08 | 1.79E+08 | − | AluJb | chr5 |
| AluSc5 | chr5 | 1.8E+08 | 1.8E+08 | + | AluSx1 | chr5 |
| AluSz6 | chr5 | 1.81E+08 | 1.81E+08 | + | AluSz | chr5 |
| AluSg4 | chr5 | 1.81E+08 | 1.81E+08 | + | AluSz | chr5 |
| AluSz | chr5 | 1.81E+08 | 1.81E+08 | − | AluSp | chr5 |
| AluY | chr15 | 23355219 | 23355524 | + | AluSc | chr15 |
| AluSc | chr15 | 23358459 | 23358749 | − | AluSx1 | chr15 |
| AluJr | chr15 | 23818333 | 23818635 | + | AluSq2 | chr15 |
| AluSx | chr15 | 25674875 | 25675172 | + | AluSx | chr15 |
| AluSc | chr15 | 28839707 | 28839983 | + | AluSx | chr15 |
| AluSx1 | chr15 | 28852126 | 28852432 | + | AluSx1 | chr15 |
| AluSp | chr15 | 34523272 | 34523579 | + | AluY | chr15 |
| AluSg | chr15 | 35213357 | 35213654 | + | AluSx1 | chr15 |
| AluJb | chr15 | 38570401 | 38570683 | + | AluSg | chr15 |
| AluSc8 | chr15 | 40686704 | 40687003 | − | AluSg | chr15 |
| AluSx1 | chr15 | 40687004 | 40687284 | − | AluSg | chr15 |
| AluSg | chr15 | 40687858 | 40688150 | + | AluJo | chr15 |
| AluSz | chr15 | 40919084 | 40919392 | − | AluY | chr15 |
| AluSx3 | chr15 | 40982910 | 40983214 | + | AluSx | chr15 |
| AluSx | chr15 | 40983722 | 40984006 | − | AluSx1 | chr15 |
| AluSx3 | chr15 | 40982910 | 40983214 | + | AluSg7 | chr15 |
| AluSx1 | chr15 | 40985041 | 40985363 | + | AluSg7 | chr15 |
| AluSx1 | chr15 | 41589841 | 41590147 | − | AluSx1 | chr15 |
| AluSx1 | chr15 | 41590812 | 41591106 | + | AluSz6 | chr15 |
| AluSz6 | chr15 | 41593187 | 41593416 | − | AluSc | chr15 |
| AluSc | chr15 | 41594412 | 41594719 | + | AluSz6 | chr15 |
| AluSx1 | chr15 | 41810599 | 41810891 | + | AluSx1 | chr15 |
| AluSg | chr15 | 41872427 | 41872726 | + | AluSz | chr15 |
| AluSz | chr15 | 41872825 | 41873128 | − | AluSz6 | chr15 |
| AluSx | chr15 | 42647396 | 42647672 | − | AluSz | chr15 |
| AluSz6 | chr15 | 42833420 | 42833704 | + | AluSz | chr15 |
| AluSx | chr15 | 42832111 | 42832408 | + | AluSz | chr15 |
| AluSp | chr15 | 43949077 | 43949374 | + | AluSx | chr15 |
| AluJb | chr15 | 44034050 | 44034334 | − | AluJb | chr15 |
| AluSz | chr15 | 44031678 | 44031969 | − | AluJb | chr15 |
| AluY | chr15 | 44035077 | 44035372 | + | AluSx3 | chr15 |
| AluJb | chr15 | 44034486 | 44034783 | + | AluSx3 | chr15 |
| AluSx | chr15 | 44311089 | 44311390 | − | AluSq | chr15 |
| AluY | chr15 | 44350739 | 44351011 | + | AluSc8 | chr15 |
| AluSc | chr15 | 44385126 | 44385427 | − | AluSp | chr15 |
| AluSc | chr15 | 44390443 | 44390737 | + | AluSq | chr15 |
| AluSq | chr15 | 44392238 | 44392554 | − | AluSc | chr15 |
| AluSc | chr15 | 44447351 | 44447650 | + | AluSz | chr15 |
| AluSg | chr15 | 44454203 | 44454461 | − | AluSp | chr15 |
| AluSg | chr15 | 44454203 | 44454461 | − | AluY | chr15 |
| AluSx1 | chr15 | 44472701 | 44472995 | + | AluJr | chr15 |
| AluJr | chr15 | 44473243 | 44473531 | − | AluJr4 | chr15 |
| AluSx1 | chr15 | 44472701 | 44472995 | + | AluSx | chr15 |
| AluJr4 | chr15 | 44473819 | 44474118 | + | AluY | chr15 |
| AluSx1 | chr15 | 44472701 | 44472995 | + | AluY | chr15 |
| AluY | chr15 | 44475006 | 44475286 | − | AluSz | chr15 |
| AluSz | chr15 | 44697806 | 44698119 | − | AluSx1 | chr15 |
| AluSx | chr15 | 44727492 | 44727787 | + | AluSz | chr15 |
| AluSx1 | chr15 | 44797650 | 44797963 | + | AluJb | chr15 |
| AluSx1 | chr15 | 44796266 | 44796573 | + | AluJb | chr15 |
| AluJo | chr15 | 44797348 | 44797643 | + | AluJb | chr15 |
| AluSx1 | chr15 | 44796266 | 44796573 | + | AluY | chr15 |
| AluSx1 | chr15 | 44797650 | 44797963 | + | AluY | chr15 |
| AluJo | chr15 | 44797348 | 44797643 | + | AluY | chr15 |
| AluJo | chr15 | 44797348 | 44797643 | + | AluJb | chr15 |
| AluSx1 | chr15 | 44797650 | 44797963 | + | AluJb | chr15 |
| AluY | chr15 | 44798974 | 44799263 | − | AluSz | chr15 |
| AluSx3 | chr15 | 44801388 | 44801704 | − | AluSz | chr15 |
| AluJb | chr15 | 44800274 | 44800592 | − | AluSz | chr15 |
| AluSz6 | chr15 | 44836282 | 44836595 | − | AluSp | chr15 |
| AluSz6 | chr15 | 44836282 | 44836595 | − | AluSc | chr15 |
| AluSx | chr15 | 44858905 | 44859200 | − | AluSx3 | chr15 |
| AluSz6 | chr15 | 44867692 | 44867988 | − | AluSp | chr15 |
| AluSz6 | chr15 | 44867692 | 44867988 | − | AluY | chr15 |
| AluY | chr15 | 45025156 | 45025458 | − | AluSz | chr15 |
| AluJr | chr15 | 45774986 | 45775281 | − | AluSx4 | chr15 |
| AluSc8 | chr15 | 49485913 | 49486206 | − | AluSx4 | chr15 |
| AluSc | chr15 | 50651499 | 50651802 | − | AluSz | chr15 |
| AluSx | chr15 | 50656798 | 50657103 | − | AluSx1 | chr15 |
| AluSx1 | chr15 | 50658605 | 50658883 | − | AluSx1 | chr15 |
| AluSx1 | chr15 | 50658605 | 50658883 | − | AluSq2 | chr15 |
| AluSx1 | chr15 | 50850610 | 50850919 | + | AluSx3 | chr15 |
| AluSz | chr15 | 52202490 | 52202795 | + | AluSx1 | chr15 |
| AluSx1 | chr15 | 52205172 | 52205468 | − | AluSz | chr15 |
| AluSz | chr15 | 52206580 | 52206868 | + | AluSq2 | chr15 |
| AluJb | chr15 | 57233283 | 57233571 | + | AluSq | chr15 |
| AluSx1 | chr15 | 57252629 | 57252927 | − | AluSq | chr15 |
| AluSg | chr15 | 59290092 | 59290401 | − | AluSx1 | chr15 |
| AluJb | chr15 | 59292351 | 59292593 | − | AluSx1 | chr15 |
| AluSz | chr15 | 59656826 | 59657131 | + | AluSq2 | chr15 |
| AluSg7 | chr15 | 59668443 | 59668748 | − | AluSq | chr15 |
| AluSx1 | chr15 | 59668749 | 59669039 | − | AluSq | chr15 |
| AluSz | chr15 | 62153149 | 62153462 | − | AluSz6 | chr15 |
| AluSz | chr15 | 64682379 | 64682682 | + | AluSc8 | chr15 |
| AluJr4 | chr15 | 64827277 | 64827535 | − | AluSp | chr15 |
| AluSc | chr15 | 64859404 | 64859697 | + | AluSc8 | chr15 |
| AluSq2 | chr15 | 65303379 | 65303680 | + | AluSp | chr15 |
| AluSx3 | chr15 | 65441535 | 65441829 | + | AluSg4 | chr15 |
| AluSc | chr15 | 65834287 | 65834594 | + | AluJb | chr15 |
| AluSz6 | chr15 | 65960549 | 65960846 | − | AluSx1 | chr15 |
| AluJr4 | chr15 | 67667526 | 67667785 | + | AluSz | chr15 |
| AluSz | chr15 | 67668121 | 67668424 | − | AluSc | chr15 |
| AluJr | chr15 | 68031337 | 68031640 | + | AluSx | chr15 |
| AluSq | chr15 | 68044381 | 68044673 | − | AluSg4 | chr15 |
| AluY | chr15 | 68351339 | 68351647 | − | AluSx3 | chr15 |
| AluSg7 | chr15 | 68363637 | 68363852 | − | AluY | chr15 |
| AluSx3 | chr15 | 68518376 | 68518674 | + | AluSp | chr15 |
| AluJr | chr15 | 69698926 | 69699227 | + | AluJb | chr15 |
| AluY | chr15 | 72482437 | 72482729 | + | AluSq2 | chr15 |
| AluSq | chr15 | 72790904 | 72791204 | + | AluSz | chr15 |
| AluSq | chr15 | 72790904 | 72791204 | + | AluSc | chr15 |
| AluSz | chr15 | 72825475 | 72825764 | − | AluJr | chr15 |
| AluSz6 | chr15 | 74319004 | 74319289 | − | AluJr | chr15 |
| AluYf4 | chr15 | 74841408 | 74841715 | − | AluSc8 | chr15 |
| AluSz | chr15 | 75138355 | 75138636 | + | AluSx1 | chr15 |
| AluY | chr15 | 75138823 | 75139121 | + | AluSx1 | chr15 |
| AluJb | chr15 | 75138637 | 75138815 | + | AluSx1 | chr15 |
| AluSx3 | chr15 | 75187310 | 75187614 | − | AluJo | chr15 |
| AluSx | chr15 | 75186450 | 75186724 | − | AluJo | chr15 |
| AluSq2 | chr15 | 75840595 | 75840892 | − | AluJr | chr15 |
| AluSx1 | chr15 | 75884749 | 75885055 | + | AluY | chr15 |
| AluY | chr15 | 75885625 | 75885923 | − | AluJb | chr15 |
| AluSp | chr15 | 77536030 | 77536339 | + | AluSz | chr15 |
| AluSz | chr15 | 77536927 | 77537213 | − | AluSx | chr15 |
| AluSx | chr15 | 80357880 | 80358186 | − | AluSx1 | chr15 |
| AluSq2 | chr15 | 80380627 | 80380920 | + | AluSz6 | chr15 |
| AluSc | chr15 | 85185291 | 85185596 | + | AluJb | chr15 |
| AluSc | chr15 | 85250413 | 85250713 | − | AluSz | chr15 |
| AluSx1 | chr15 | 85930286 | 85930581 | + | AluJo | chr15 |
| AluSp | chr15 | 90386213 | 90386515 | − | AluSz | chr15 |
| AluSx | chr15 | 90619685 | 90619990 | + | AluY | chr15 |
| AluJr | chr15 | 90618471 | 90618771 | + | AluY | chr15 |
| AluY | chr15 | 90620472 | 90620775 | − | AluSp | chr15 |
| AluSz | chr15 | 90729157 | 90729465 | − | AluSq | chr15 |
| AluSz | chr15 | 90817103 | 90817402 | − | AluSx1 | chr15 |
| AluSx | chr15 | 90817403 | 90817687 | − | AluSx1 | chr15 |
| AluSx1 | chr15 | 90816801 | 90817102 | − | AluSx1 | chr15 |
| AluSx | chr15 | 90817403 | 90817687 | − | AluSx | chr15 |
| AluSx1 | chr15 | 90816801 | 90817102 | − | AluSx | chr15 |
| AluSx1 | chr15 | 90817689 | 90817931 | + | AluY | chr15 |
| AluSq2 | chr10 | 946004 | 946306 | + | AluSg | chr10 |
| AluSq2 | chr10 | 946004 | 946306 | + | AluSq2 | chr10 |
| AluSp | chr10 | 962354 | 962655 | + | AluJr | chr10 |
| AluJr | chr10 | 962656 | 962935 | − | AluJb | chr10 |
| AluSq | chr10 | 1035055 | 1035354 | − | AluY | chr10 |
| AluSq | chr10 | 1045050 | 1045361 | − | AluSq2 | chr10 |
| AluSz6 | chr10 | 5764136 | 5764437 | + | AluY | chr10 |
| AluJb | chr10 | 5821658 | 5821952 | − | AluJb | chr10 |
| AluJb | chr10 | 5845162 | 5845401 | + | AluSp | chr10 |
| AluJo | chr10 | 5912368 | 5912647 | − | AluSx1 | chr10 |
| AluSz6 | chr10 | 5942779 | 5943087 | + | AluSq2 | chr10 |
| AluSz6 | chr10 | 5942779 | 5943087 | + | AluJo | chr10 |
| AluSz | chr10 | 5969753 | 5970059 | + | AluJr4 | chr10 |
| AluSx1 | chr10 | 6195535 | 6195830 | + | AluSz | chr10 |
| AluY | chr10 | 11585259 | 11585570 | + | AluY | chr10 |
| AluSz | chr10 | 11676391 | 11676694 | − | AluJb | chr10 |
| AluSp | chr10 | 12013250 | 12013550 | − | AluSz | chr10 |
| AluSx3 | chr10 | 12259804 | 12260108 | + | AluSx3 | chr10 |
| AluSc8 | chr10 | 14624364 | 14624675 | − | AluSx | chr10 |
| AluSc | chr10 | 14903732 | 14904025 | + | AluY | chr10 |
| AluSx1 | chr10 | 14979281 | 14979582 | − | AluSx1 | chr10 |
| AluSx | chr10 | 15118503 | 15118801 | − | AluSx1 | chr10 |
| AluSq2 | chr10 | 15143608 | 15143904 | + | AluJb | chr10 |
| AluSx1 | chr10 | 15408126 | 15408418 | − | AluY | chr10 |
| AluSp | chr10 | 17448791 | 17449089 | − | AluSx1 | chr10 |
| AluSp | chr10 | 17448791 | 17449089 | − | AluSp | chr10 |
| AluSp | chr10 | 17451627 | 17451898 | + | AluSq2 | chr10 |
| AluSx1 | chr10 | 17450382 | 17450666 | + | AluSq2 | chr10 |
| AluSc8 | chr10 | 18958640 | 18958943 | + | AluSz | chr10 |
| AluSq2 | chr10 | 21831216 | 21831504 | + | AluSp | chr10 |
| AluSx | chr10 | 21829807 | 21830104 | + | AluSp | chr10 |
| AluJb | chr10 | 21948917 | 21949196 | − | AluSz | chr10 |
| AluSx1 | chr10 | 21948442 | 21948737 | − | AluSz | chr10 |
| AluSq2 | chr10 | 22014366 | 22014666 | + | AluY | chr10 |
| AluSz | chr10 | 27006971 | 27007258 | − | AluSq2 | chr10 |
| AluJb | chr10 | 27061719 | 27061982 | + | AluSz | chr10 |
| AluSq | chr10 | 27430043 | 27430324 | + | AluY | chr10 |
| AluY | chr10 | 27431639 | 27431945 | − | AluSp | chr10 |
| AluSp | chr10 | 28574119 | 28574413 | − | AluY | chr10 |
| AluSp | chr10 | 28811915 | 28812209 | − | AluSq | chr10 |
| AluSq | chr10 | 28836182 | 28836478 | + | AluSq | chr10 |
| AluSq | chr10 | 28836182 | 28836478 | + | AluJb | chr10 |
| AluJb | chr10 | 28837812 | 28838125 | − | AluSx3 | chr10 |
| AluSq | chr10 | 28836839 | 28837152 | − | AluSx3 | chr10 |
| AluSx3 | chr10 | 28838812 | 28839109 | + | AluSx | chr10 |
| AluSx1 | chr10 | 29180551 | 29180832 | − | AluSx1 | chr10 |
| AluSx1 | chr10 | 29182490 | 29182755 | + | AluJb | chr10 |
| AluSx1 | chr10 | 29182490 | 29182755 | + | AluSq2 | chr10 |
| AluSc | chr10 | 29816350 | 29816653 | − | AluSx | chr10 |
| AluSc5 | chr10 | 29815223 | 29815531 | − | AluSx | chr10 |
| AluSx | chr10 | 29931046 | 29931325 | + | AluSz | chr10 |
| AluSx3 | chr10 | 31270525 | 31270821 | + | AluY | chr10 |
| AluSp | chr10 | 32155549 | 32155846 | − | AluSx1 | chr10 |
| AluSx1 | chr10 | 32158183 | 32158488 | + | AluSp | chr10 |
| AluSg | chr10 | 32205554 | 32205842 | − | AluSc8 | chr10 |
| AluSz6 | chr10 | 34910195 | 34910423 | − | AluSc8 | chr10 |
| AluJr | chr10 | 34932566 | 34932871 | + | AluJr | chr10 |
| AluJr | chr10 | 34932566 | 34932871 | + | AluSz | chr10 |
| AluSz | chr10 | 34974525 | 34974821 | − | AluSq2 | chr10 |
| AluSz | chr10 | 34974525 | 34974821 | − | AluSz | chr10 |
| AluSz | chr10 | 34975695 | 34976004 | + | AluSz | chr10 |
| AluSz | chr10 | 34978863 | 34979169 | − | AluSx1 | chr10 |
| AluSx1 | chr10 | 35608665 | 35608898 | + | AluSx | chr10 |
| AluSx | chr10 | 35611858 | 35612155 | − | AluSx | chr10 |
| AluSx | chr10 | 35612665 | 35612960 | + | AluSx | chr10 |
| AluSx | chr10 | 35614110 | 35614395 | − | AluJr | chr10 |
| AluSc | chr10 | 38377360 | 38377663 | − | AluSx1 | chr10 |
| AluSq | chr10 | 43935503 | 43935797 | − | AluSx | chr10 |
| AluSx1 | chr10 | 51726305 | 51726614 | + | AluJb | chr10 |
| AluSc | chr10 | 51733561 | 51733859 | − | AluSx | chr10 |
| AluSx1 | chr10 | 63707017 | 63707328 | − | AluSq2 | chr10 |
| AluSz | chr10 | 64991272 | 64991583 | + | AluSq2 | chr10 |
| AluSx | chr10 | 65207517 | 65207728 | + | AluSz | chr10 |
| AluJo | chr10 | 65217964 | 65218258 | + | AluJb | chr10 |
| AluJb | chr10 | 65251795 | 65252010 | − | AluSp | chr10 |
| AluSz | chr10 | 69697439 | 69697736 | − | AluSc | chr10 |
| AluSg | chr10 | 69817110 | 69817395 | + | AluSx | chr10 |
| AluSx1 | chr10 | 70116319 | 70116604 | − | AluSx1 | chr10 |
| AluSp | chr10 | 70189021 | 70189319 | + | AluSx1 | chr10 |
| AluY | chr10 | 70189320 | 70189498 | + | AluSx1 | chr10 |
| AluSx | chr10 | 70241381 | 70241660 | − | AluSc8 | chr10 |
| AluSc8 | chr10 | 70240248 | 70240551 | − | AluSc8 | chr10 |
| AluSx3 | chr10 | 70279752 | 70280013 | − | AluSz | chr10 |
| AluYa5 | chr10 | 70281340 | 70281653 | − | AluSz | chr10 |
| AluSx3 | chr10 | 70279752 | 70280013 | − | AluSc8 | chr10 |
| AluYa5 | chr10 | 70281340 | 70281653 | − | AluSc8 | chr10 |
| AluSc8 | chr10 | 70282291 | 70282595 | + | AluSz | chr10 |
| AluSz | chr10 | 70281654 | 70281946 | + | AluSz | chr10 |
| AluSc8 | chr10 | 70282291 | 70282595 | + | AluSx | chr10 |
| AluSz | chr10 | 70281654 | 70281946 | + | AluSx | chr10 |
| AluYa5 | chr10 | 70281340 | 70281653 | − | AluY | chr10 |
| AluSx | chr10 | 70283235 | 70283531 | − | AluY | chr10 |
| AluSz | chr10 | 70282868 | 70283149 | − | AluY | chr10 |
| AluY | chr10 | 70283796 | 70284088 | + | AluSz | chr10 |
| AluSc8 | chr10 | 70282291 | 70282595 | + | AluSz | chr10 |
| AluSz | chr10 | 70281654 | 70281946 | + | AluSz | chr10 |
| AluSc8 | chr10 | 70416501 | 70416798 | + | AluY | chr10 |
| AluSc8 | chr10 | 70485915 | 70486206 | − | AluSx1 | chr10 |
| AluSx | chr10 | 70677363 | 70677658 | − | AluSx1 | chr10 |
| AluSx1 | chr10 | 72246239 | 72246542 | + | AluY | chr10 |
| AluJb | chr10 | 72622738 | 72623043 | − | AluJb | chr10 |
| AluSp | chr10 | 73955372 | 73955605 | + | AluSz | chr10 |
| AluSp | chr10 | 73979806 | 73980111 | + | AluSx1 | chr10 |
| AluSp | chr10 | 73979806 | 73980111 | + | AluSg | chr10 |
| AluSx1 | chr10 | 73981301 | 73981483 | − | AluSx | chr10 |
| AluSp | chr10 | 73979806 | 73980111 | + | AluSx1 | chr10 |
| AluSx | chr10 | 73982253 | 73982563 | + | AluSx1 | chr10 |
| AluSx1 | chr10 | 73985777 | 73986076 | − | AluJb | chr10 |
| AluSg | chr10 | 74061962 | 74062269 | − | AluSq | chr10 |
| AluSq | chr10 | 74062309 | 74062618 | + | AluSx | chr10 |
| AluJb | chr10 | 74308940 | 74309248 | − | AluSq2 | chr10 |
| AluSx1 | chr10 | 74500846 | 74501157 | − | AluY | chr10 |
| AluSz | chr10 | 74499456 | 74499732 | − | AluY | chr10 |
| AluSx1 | chr10 | 74997180 | 74997479 | − | AluY | chr10 |
| AluSx | chr10 | 75372318 | 75372624 | + | AluSx | chr10 |
| AluSz6 | chr10 | 75388081 | 75388392 | − | AluSx3 | chr10 |
| AluSq | chr10 | 75537315 | 75537610 | − | AluJb | chr10 |
| AluSq | chr10 | 75537315 | 75537610 | − | AluSp | chr10 |
| AluSp | chr10 | 75546702 | 75547000 | − | AluSz | chr10 |
| AluSz | chr10 | 75775029 | 75775310 | + | AluSx1 | chr10 |
| AluSz | chr10 | 76290402 | 76290693 | + | AluJr4 | chr10 |
| AluSq2 | chr10 | 76354650 | 76354955 | + | AluY | chr10 |
| AluSx | chr10 | 79666376 | 79666683 | − | AluJb | chr10 |
| AluSq | chr10 | 82193606 | 82193900 | + | AluJr | chr10 |
| AluSz | chr10 | 88238570 | 88238885 | + | AluSq | chr10 |
| AluSx1 | chr10 | 88275258 | 88275548 | + | AluSx1 | chr10 |
| AluSx1 | chr10 | 88275622 | 88275918 | − | AluY | chr10 |
| AluSx1 | chr10 | 88857514 | 88857819 | − | AluSp | chr10 |
| AluSz6 | chr10 | 88920465 | 88920764 | + | AluSx | chr10 |
| AluSz6 | chr10 | 93569858 | 93570163 | + | AluY | chr10 |
| AluSx1 | chr10 | 96322862 | 96323147 | − | AluSx | chr10 |
| AluSx | chr10 | 96324812 | 96325103 | + | AluJb | chr10 |
| AluSx | chr10 | 96324812 | 96325103 | + | AluSp | chr10 |
| AluSp | chr10 | 96327042 | 96327352 | − | AluJb | chr10 |
| AluJb | chr10 | 96325658 | 96325936 | − | AluJb | chr10 |
| AluSx | chr10 | 96324812 | 96325103 | + | AluSc | chr10 |
| AluJb | chr10 | 96327381 | 96327661 | + | AluSc | chr10 |
| AluSz | chr10 | 98601120 | 98601417 | − | AluSc | chr10 |
| AluSc | chr10 | 98601871 | 98602153 | + | AluSz | chr10 |
| AluSz | chr10 | 98602376 | 98602687 | − | AluSz | chr10 |
| AluSq | chr10 | 98608133 | 98608445 | + | AluSc8 | chr10 |
| AluSz | chr10 | 98605682 | 98605978 | + | AluSc8 | chr10 |
| AluJb | chr10 | 98606731 | 98607017 | + | AluSc8 | chr10 |
| AluSz | chr10 | 99255535 | 99255833 | − | AluJb | chr10 |
| AluJb | chr10 | 99975991 | 99976271 | + | AluJr | chr10 |
| AluSq2 | chr10 | 1.02E+08 | 1.02E+08 | − | AluSq2 | chr10 |
| AluSx1 | chr10 | 1.02E+08 | 1.02E+08 | + | AluJr | chr10 |
| AluSq2 | chr10 | 1.02E+08 | 1.02E+08 | − | AluJo | chr10 |
| AluJo | chr10 | 1.03E+08 | 1.03E+08 | + | AluY | chr10 |
| AluJb | chr10 | 1.04E+08 | 1.04E+08 | + | AluSx1 | chr10 |
| AluSp | chr10 | 1.04E+08 | 1.04E+08 | + | AluSx1 | chr10 |
| AluSz6 | chr10 | 1.04E+08 | 1.04E+08 | + | AluJb | chr10 |
| AluJb | chr10 | 1.04E+08 | 1.04E+08 | + | AluSx1 | chr10 |
| AluSx | chr10 | 1.04E+08 | 1.04E+08 | − | AluSq | chr10 |
| AluSc8 | chr10 | 1.05E+08 | 1.05E+08 | + | AluSg | chr10 |
| AluJb | chr10 | 1.06E+08 | 1.06E+08 | − | AluSq2 | chr10 |
| AluY | chr10 | 1.06E+08 | 1.06E+08 | + | AluSx1 | chr10 |
| AluSx | chr10 | 1.12E+08 | 1.12E+08 | − | AluSx1 | chr10 |
| AluSx1 | chr10 | 1.12E+08 | 1.12E+08 | + | AluJr | chr10 |
| AluSp | chr10 | 1.14E+08 | 1.14E+08 | + | AluJb | chr10 |
| AluSx | chr10 | 1.15E+08 | 1.15E+08 | + | AluJr | chr10 |
| AluY | chr10 | 1.15E+08 | 1.15E+08 | + | AluSx1 | chr10 |
| AluSx3 | chr10 | 1.15E+08 | 1.15E+08 | + | AluSx1 | chr10 |
| AluY | chr10 | 1.16E+08 | 1.16E+08 | − | AluSx | chr10 |
| AluY | chr10 | 1.16E+08 | 1.16E+08 | + | AluY | chr10 |
| AluY | chr10 | 1.16E+08 | 1.16E+08 | − | AluSx1 | chr10 |
| AluSq | chr10 | 1.16E+08 | 1.16E+08 | + | AluSx4 | chr10 |
| AluSx4 | chr10 | 1.17E+08 | 1.17E+08 | + | AluSx | chr10 |
| AluSz6 | chr10 | 1.19E+08 | 1.19E+08 | − | AluSx1 | chr10 |
| AluJb | chr10 | 1.19E+08 | 1.19E+08 | − | AluSc8 | chr10 |
| AluSc8 | chr10 | 1.21E+08 | 1.21E+08 | + | AluSx1 | chr10 |
| AluY | chr10 | 1.21E+08 | 1.21E+08 | + | AluSg4 | chr10 |
| AluSq2 | chr10 | 1.24E+08 | 1.24E+08 | − | AluSc8 | chr10 |
| AluY | chr10 | 1.24E+08 | 1.24E+08 | + | AluY | chr10 |
| AluY | chr10 | 1.24E+08 | 1.24E+08 | + | AluJb | chr10 |
| AluSp | chr10 | 1.24E+08 | 1.24E+08 | − | AluJb | chr10 |
| AluSq | chr10 | 1.24E+08 | 1.24E+08 | − | AluSx1 | chr10 |
| AluSx1 | chr10 | 1.24E+08 | 1.24E+08 | + | AluSz | chr10 |
| AluYf4 | chr10 | 1.24E+08 | 1.24E+08 | + | AluSp | chr10 |
| AluSz | chr10 | 1.24E+08 | 1.24E+08 | + | AluSp | chr10 |
| AluSc8 | chr10 | 1.26E+08 | 1.26E+08 | − | AluSq2 | chr10 |
| AluY | chr10 | 1.26E+08 | 1.26E+08 | + | AluY | chr10 |
| AluSx | chr10 | 1.26E+08 | 1.26E+08 | + | AluY | chr10 |
| AluJo | chr10 | 1.27E+08 | 1.27E+08 | − | AluSg4 | chr10 |
| AluSg4 | chr10 | 1.27E+08 | 1.27E+08 | + | AluSg7 | chr10 |
| AluSx | chr10 | 1.27E+08 | 1.27E+08 | − | AluY | chr10 |
| AluSx | chr10 | 1.32E+08 | 1.32E+08 | + | AluSc8 | chr10 |
| AluSx | chr10 | 1.32E+08 | 1.32E+08 | + | AluJb | chr10 |
| AluJb | chr10 | 1.32E+08 | 1.32E+08 | − | AluSx | chr10 |
| AluSp | chr10 | 1.32E+08 | 1.32E+08 | − | AluSz | chr10 |
| AluJb | chrX | 2824178 | 2824481 | − | AluSz | chrX |
| AluJo | chrX | 16781129 | 16781410 | + | AluJb | chrX |
| AluSx1 | chrX | 23719909 | 23720188 | + | AluSg7 | chrX |
| AluJr | chrX | 24093856 | 24094120 | − | AluSz | chrX |
| AluSz | chrX | 24094369 | 24094678 | + | AluSx | chrX |
| AluSz | chrX | 24094369 | 24094678 | + | AluSx1 | chrX |
| AluSx1 | chrX | 27192057 | 27192351 | + | AluSp | chrX |
| AluSg | chrX | 45697160 | 45697462 | + | AluSg7 | chrX |
| AluSx1 | chrX | 45704020 | 45704323 | − | AluSx | chrX |
| AluSc8 | chrX | 46462992 | 46463303 | + | AluJb | chrX |
| AluSx | chrX | 47013822 | 47014139 | − | AluY | chrX |
| AluY | chrX | 47014850 | 47015151 | + | AluSp | chrX |
| AluSz6 | chrX | 48575771 | 48576065 | + | AluSc5 | chrX |
| AluY | chrX | 48892922 | 48893229 | − | AluJb | chrX |
| AluY | chrX | 48892922 | 48893229 | − | AluSq | chrX |
| AluJr | chrX | 51551523 | 51551815 | + | AluY | chrX |
| AluSx | chrX | 54589077 | 54589242 | + | AluSg4 | chrX |
| AluSx | chrX | 54589077 | 54589242 | + | AluSx1 | chrX |
| AluJb | chrX | 69361901 | 69362195 | + | AluY | chrX |
| AluSz | chrX | 70513149 | 70513443 | + | AluSc8 | chrX |
| AluSq2 | chrX | 70784725 | 70785016 | + | AluSz6 | chrX |
| AluSq2 | chrX | 73168806 | 73169104 | − | AluSx3 | chrX |
| AluSx3 | chrX | 73170949 | 73171247 | − | AluSx3 | chrX |
| AluJb | chrX | 73208466 | 73208770 | − | AluSq | chrX |
| AluSq | chrX | 73210078 | 73210377 | + | AluSx | chrX |
| AluSx3 | chrX | 73229740 | 73230036 | + | AluSp | chrX |
| AluSz | chrX | 73228573 | 73228879 | + | AluSp | chrX |
| AluSz | chrX | 73434080 | 73434367 | + | AluSx | chrX |
| AluSz | chrX | 73443442 | 73443740 | − | AluJb | chrX |
| AluSx1 | chrX | 73445287 | 73445597 | − | AluSp | chrX |
| AluSg | chrX | 73496210 | 73496486 | − | AluJb | chrX |
| AluSg | chrX | 77026577 | 77026868 | + | AluSc | chrX |
| AluSx | chrX | 77027495 | 77027806 | + | AluSc | chrX |
| AluSg | chrX | 77026577 | 77026868 | + | AluJr | chrX |
| AluSc | chrX | 77027969 | 77028267 | − | AluJb | chrX |
| AluJr | chrX | 77028330 | 77028543 | − | AluJb | chrX |
| AluJb | chrX | 77030883 | 77031188 | + | AluSx | chrX |
| AluSx | chrX | 77105691 | 77105981 | + | AluSq2 | chrX |
| AluSx1 | chrX | 77105086 | 77105403 | + | AluSq2 | chrX |
| AluJo | chrX | 77104269 | 77104530 | + | AluSq2 | chrX |
| AluJb | chrX | 84344123 | 84344408 | − | AluSq2 | chrX |
| AluY | chrX | 1.01E+08 | 1.01E+08 | − | AluSg | chrX |
| AluSx1 | chrX | 1.01E+08 | 1.01E+08 | − | AluSg | chrX |
| AluJb | chrX | 1.01E+08 | 1.01E+08 | − | AluSg | chrX |
| AluSx | chrX | 1.01E+08 | 1.01E+08 | + | AluSx | chrX |
| AluJr | chrX | 1.03E+08 | 1.03E+08 | + | AluSz | chrX |
| AluJb | chrX | 1.09E+08 | 1.09E+08 | − | AluSx1 | chrX |
| AluY | chrX | 1.11E+08 | 1.11E+08 | + | AluSq2 | chrX |
| AluY | chrX | 1.11E+08 | 1.11E+08 | − | AluJo | chrX |
| AluY | chrX | 1.15E+08 | 1.15E+08 | − | AluSz6 | chrX |
| AluY | chrX | 1.15E+08 | 1.15E+08 | − | AluSq2 | chrX |
| AluJr | chrX | 1.19E+08 | 1.19E+08 | − | AluSx | chrX |
| AluSg4 | chrX | 1.19E+08 | 1.19E+08 | + | AluSx1 | chrX |
| AluSp | chrX | 1.23E+08 | 1.23E+08 | − | AluSc8 | chrX |
| AluSq2 | chrX | 1.23E+08 | 1.23E+08 | − | AluSx | chrX |
| AluSx | chrX | 1.23E+08 | 1.23E+08 | − | AluSx | chrX |
| AluSx | chrX | 1.23E+08 | 1.23E+08 | + | AluSp | chrX |
| AluSp | chrX | 1.23E+08 | 1.23E+08 | − | AluY | chrX |
| AluSq2 | chrX | 1.23E+08 | 1.23E+08 | − | AluY | chrX |
| AluJb | chrX | 1.23E+08 | 1.23E+08 | + | AluSz6 | chrX |
| AluJb | chrX | 1.23E+08 | 1.23E+08 | + | AluSz | chrX |
| AluSp | chrX | 1.23E+08 | 1.23E+08 | − | AluSz | chrX |
| AluSz | chrX | 1.23E+08 | 1.23E+08 | − | AluSz | chrX |
| AluSp | chrX | 1.23E+08 | 1.23E+08 | − | AluSz | chrX |
| AluSz | chrX | 1.23E+08 | 1.23E+08 | + | AluSz | chrX |
| AluSz | chrX | 1.23E+08 | 1.23E+08 | + | AluSz | chrX |
| AluSz | chrX | 1.23E+08 | 1.23E+08 | − | AluJr4 | chrX |
| AluJr4 | chrX | 1.23E+08 | 1.23E+08 | + | AluSz6 | chrX |
| AluSz6 | chrX | 1.23E+08 | 1.23E+08 | − | AluSp | chrX |
| AluSx3 | chrX | 1.23E+08 | 1.23E+08 | − | AluSc | chrX |
| AluSx3 | chrX | 1.23E+08 | 1.23E+08 | − | AluSg | chrX |
| AluSz | chrX | 1.23E+08 | 1.23E+08 | − | AluSx | chrX |
| AluJo | chrX | 1.23E+08 | 1.23E+08 | − | AluJb | chrX |
| AluY | chrX | 1.29E+08 | 1.29E+08 | − | AluSg | chrX |
| AluSq | chrX | 1.29E+08 | 1.29E+08 | + | AluSz6 | chrX |
| AluJb | chrX | 1.29E+08 | 1.29E+08 | − | AluSx3 | chrX |
| AluSz | chrX | 1.32E+08 | 1.32E+08 | + | AluY | chrX |
| AluSp | chrX | 1.34E+08 | 1.34E+08 | + | AluSz | chrX |
| AluJb | chrX | 1.34E+08 | 1.34E+08 | + | AluSz | chrX |
| AluSz | chrX | 1.34E+08 | 1.34E+08 | − | AluSp | chrX |
| AluJo | chrX | 1.34E+08 | 1.34E+08 | − | AluSx | chrX |
| AluJo | chrX | 1.34E+08 | 1.34E+08 | − | AluSg | chrX |
| AluSx | chrX | 1.34E+08 | 1.34E+08 | + | AluY | chrX |
| AluSg | chrX | 1.34E+08 | 1.34E+08 | + | AluY | chrX |
| AluSx | chrX | 1.34E+08 | 1.34E+08 | + | AluSx | chrX |
| AluSg | chrX | 1.34E+08 | 1.34E+08 | + | AluSx | chrX |
| AluSp | chrX | 1.36E+08 | 1.36E+08 | + | AluY | chrX |
| AluSg | chrX | 1.36E+08 | 1.36E+08 | − | AluSx1 | chrX |
| AluJb | chrX | 1.36E+08 | 1.36E+08 | − | AluSx1 | chrX |
| AluSq2 | chrX | 1.53E+08 | 1.53E+08 | + | AluJb | chrX |
| AluSq | chrX | 1.54E+08 | 1.54E+08 | + | AluSx1 | chrX |
| AluSx1 | chrX | 1.54E+08 | 1.54E+08 | − | AluSz6 | chrX |
| AluSx1 | chrX | 1.54E+08 | 1.54E+08 | − | AluSg | chrX |
| AluJr4 | chr6 | 3021209 | 3021491 | − | AluJr | chr6 |
| AluSg7 | chr6 | 15286484 | 15286779 | − | AluY | chr6 |
| AluY | chr6 | 16612081 | 16612389 | − | AluSc | chr6 |
| AluJr | chr6 | 20849009 | 20849315 | − | AluY | chr6 |
| AluY | chr6 | 27078814 | 27079119 | + | AluSg | chr6 |
| AluSz | chr6 | 27090974 | 27091268 | + | AluY | chr6 |
| AluSp | chr6 | 28880751 | 28881047 | + | AluSg | chr6 |
| AluSg | chr6 | 28883706 | 28883991 | − | AluSp | chr6 |
| AluSg | chr6 | 28883706 | 28883991 | − | AluSg | chr6 |
| AluY | chr6 | 30167405 | 30167712 | − | AluSc8 | chr6 |
| AluSc8 | chr6 | 30169341 | 30169645 | + | AluJb | chr6 |
| AluY | chr6 | 30597333 | 30597620 | − | AluJb | chr6 |
| AluSz | chr6 | 30597016 | 30597315 | − | AluJb | chr6 |
| AluY | chr6 | 30597333 | 30597620 | − | AluSx1 | chr6 |
| AluSz | chr6 | 30597016 | 30597315 | − | AluSx1 | chr6 |
| AluSq2 | chr6 | 30700810 | 30701112 | − | AluJb | chr6 |
| AluSx1 | chr6 | 31166203 | 31166512 | − | AluJb | chr6 |
| AluSx3 | chr6 | 31640225 | 31640517 | − | AluSz | chr6 |
| AluSx1 | chr6 | 31715501 | 31715794 | + | AluSq2 | chr6 |
| AluSx1 | chr6 | 31715501 | 31715794 | + | AluSg | chr6 |
| AluSq2 | chr6 | 31716122 | 31716418 | − | AluSz | chr6 |
| AluSg | chr6 | 31716692 | 31716990 | − | AluSz | chr6 |
| AluSz | chr6 | 31717037 | 31717332 | + | AluY | chr6 |
| AluSx1 | chr6 | 31715501 | 31715794 | + | AluY | chr6 |
| AluSx1 | chr6 | 31823686 | 31823981 | − | AluSx1 | chr6 |
| AluSx1 | chr6 | 31824037 | 31824336 | + | AluSg | chr6 |
| AluSg | chr6 | 31824462 | 31824755 | − | AluSx1 | chr6 |
| AluSx1 | chr6 | 31823686 | 31823981 | − | AluSx1 | chr6 |
| AluSg4 | chr6 | 32132708 | 32132988 | − | AluSx1 | chr6 |
| AluSx1 | chr6 | 33274783 | 33275092 | + | AluSx1 | chr6 |
| AluSp | chr6 | 33274474 | 33274772 | + | AluSx1 | chr6 |
| AluSx1 | chr6 | 33275169 | 33275478 | − | AluSp | chr6 |
| AluSp | chr6 | 33274474 | 33274772 | + | AluSp | chr6 |
| AluSp | chr6 | 33276890 | 33277173 | + | AluSp | chr6 |
| AluSx1 | chr6 | 33274783 | 33275092 | + | AluSp | chr6 |
| AluSg | chr6 | 33611688 | 33611989 | + | AluSg | chr6 |
| AluY | chr6 | 34388506 | 34388813 | + | AluSz | chr6 |
| AluSz | chr6 | 34390087 | 34390389 | − | AluSz | chr6 |
| AluJr | chr6 | 34723018 | 34723279 | + | AluSx1 | chr6 |
| AluSx | chr6 | 34913746 | 34914057 | − | AluSz | chr6 |
| AluY | chr6 | 35321729 | 35322030 | + | AluSx1 | chr6 |
| AluJb | chr6 | 35804248 | 35804531 | + | AluJb | chr6 |
| AluJb | chr6 | 35804248 | 35804531 | + | AluSq2 | chr6 |
| AluSz6 | chr6 | 35821730 | 35822025 | − | AluY | chr6 |
| AluSq2 | chr6 | 35850914 | 35851187 | + | AluSx1 | chr6 |
| AluSc | chr6 | 36042388 | 36042690 | − | AluY | chr6 |
| AluSc | chr6 | 36468257 | 36468556 | + | AluJr4 | chr6 |
| AluSg7 | chr6 | 36479125 | 36479432 | − | AluSx | chr6 |
| AluSx | chr6 | 36479554 | 36479864 | + | AluSg | chr6 |
| AluSx | chr6 | 36479554 | 36479864 | + | AluSc | chr6 |
| AluSc | chr6 | 36481095 | 36481392 | − | AluSx | chr6 |
| AluY | chr6 | 36510847 | 36511141 | − | AluJb | chr6 |
| AluSx1 | chr6 | 36509597 | 36509911 | − | AluSx1 | chr6 |
| AluY | chr6 | 36510847 | 36511141 | − | AluSx1 | chr6 |
| AluSc8 | chr6 | 37796423 | 37796725 | − | AluSx | chr6 |
| AluSg4 | chr6 | 37918156 | 37918450 | − | AluSc8 | chr6 |
| AluSx | chr6 | 41961146 | 41961410 | − | AluSx3 | chr6 |
| AluSz | chr6 | 42046584 | 42046889 | + | AluSq2 | chr6 |
| AluSg | chr6 | 42068533 | 42068832 | + | AluSc | chr6 |
| AluJb | chr6 | 42172513 | 42172811 | + | AluSp | chr6 |
| AluSp | chr6 | 42173033 | 42173310 | − | AluSc | chr6 |
| AluSc | chr6 | 42173678 | 42173986 | + | AluSq2 | chr6 |
| AluJb | chr6 | 42172513 | 42172811 | + | AluSq2 | chr6 |
| AluSc | chr6 | 42173678 | 42173986 | + | AluSz | chr6 |
| AluSx1 | chr6 | 42182845 | 42183113 | + | AluSg | chr6 |
| AluJb | chr6 | 42183165 | 42183456 | − | AluSq2 | chr6 |
| AluSg | chr6 | 42183752 | 42184059 | − | AluSq2 | chr6 |
| AluSx1 | chr6 | 42405643 | 42405958 | − | AluSx | chr6 |
| AluSp | chr6 | 42578587 | 42578883 | − | AluSc | chr6 |
| AluSz6 | chr6 | 42614587 | 42614859 | − | AluSx3 | chr6 |
| AluSg | chr6 | 42753512 | 42753769 | + | AluSq | chr6 |
| AluSq2 | chr6 | 42753016 | 42753317 | + | AluSq | chr6 |
| AluSq | chr6 | 42755958 | 42756259 | − | AluSz6 | chr6 |
| AluSq2 | chr6 | 42760371 | 42760679 | + | AluSx | chr6 |
| AluY | chr6 | 42848922 | 42849227 | + | AluSz | chr6 |
| AluSz | chr6 | 42849476 | 42849798 | − | AluSz | chr6 |
| AluSz6 | chr6 | 42997339 | 42997657 | − | AluSg | chr6 |
| AluSg | chr6 | 42998326 | 42998618 | + | AluSz | chr6 |
| AluSg | chr6 | 43101031 | 43101328 | + | AluSz | chr6 |
| AluSx1 | chr6 | 43526752 | 43527042 | + | AluJb | chr6 |
| AluSg | chr6 | 43555484 | 43555783 | + | AluSx | chr6 |
| AluSg | chr6 | 43555484 | 43555783 | + | AluYk4 | chr6 |
| AluSc | chr6 | 43583405 | 43583708 | − | AluSg4 | chr6 |
| AluSg4 | chr6 | 43584049 | 43584348 | + | AluSc | chr6 |
| AluSg4 | chr6 | 43584049 | 43584348 | + | AluSz | chr6 |
| AluSz | chr6 | 43586378 | 43586662 | − | AluJb | chr6 |
| AluSc | chr6 | 43585903 | 43586188 | − | AluJb | chr6 |
| AluJb | chr6 | 44059127 | 44059439 | − | AluSx | chr6 |
| AluSx | chr6 | 44875132 | 44875426 | + | AluY | chr6 |
| AluSx1 | chr6 | 52243613 | 52243921 | + | AluY | chr6 |
| AluSg | chr6 | 52243309 | 52243596 | + | AluY | chr6 |
| AluSz | chr6 | 52421067 | 52421379 | − | AluSz | chr6 |
| AluSx | chr6 | 52547977 | 52548256 | + | AluSp | chr6 |
| AluSg | chr6 | 52856555 | 52856847 | − | AluSz | chr6 |
| AluSx1 | chr6 | 53424098 | 53424412 | + | AluSg | chr6 |
| AluSx | chr6 | 56628188 | 56628475 | + | AluSx | chr6 |
| AluSx1 | chr6 | 56652655 | 56652953 | + | AluSz | chr6 |
| AluSz | chr6 | 56713551 | 56713848 | + | AluSc | chr6 |
| AluJr | chr6 | 56930284 | 56930501 | − | AluSq2 | chr6 |
| AluSg | chr6 | 58277282 | 58277586 | + | AluSx1 | chr6 |
| AluSx3 | chr6 | 58277597 | 58277906 | + | AluSx1 | chr6 |
| AluSg | chr6 | 58277282 | 58277586 | + | AluSq2 | chr6 |
| AluSx3 | chr6 | 58277597 | 58277906 | + | AluSq2 | chr6 |
| AluSc | chr6 | 58284293 | 58284593 | + | AluSq2 | chr6 |
| AluSx3 | chr6 | 58284594 | 58284887 | + | AluSq2 | chr6 |
| AluSz6 | chr6 | 64061382 | 64061662 | − | AluJo | chr6 |
| AluY | chr6 | 64397886 | 64398101 | + | AluSx | chr6 |
| AluSx | chr6 | 71128013 | 71128319 | − | AluSg | chr6 |
| AluSx1 | chr6 | 71139758 | 71140061 | + | AluY | chr6 |
| AluSq2 | chr6 | 71150108 | 71150378 | + | AluSg | chr6 |
| AluJb | chr6 | 71383817 | 71384115 | − | AluSx | chr6 |
| AluSz | chr6 | 76334829 | 76335130 | − | AluSx1 | chr6 |
| AluSz6 | chr6 | 76354192 | 76354500 | − | AluSz6 | chr6 |
| AluSx1 | chr6 | 76511376 | 76511670 | + | AluJb | chr6 |
| AluSp | chr6 | 87939138 | 87939443 | − | AluY | chr6 |
| AluSg4 | chr6 | 90543852 | 90544142 | + | AluSx | chr6 |
| AluSg | chr6 | 96516911 | 96517226 | + | AluSc8 | chr6 |
| AluSq2 | chr6 | 99865560 | 99865865 | + | AluSz | chr6 |
| AluSz | chr6 | 99869769 | 99870041 | − | AluSz6 | chr6 |
| AluSx1 | chr6 | 1.07E+08 | 1.07E+08 | + | AluSp | chr6 |
| AluSc | chr6 | 1.08E+08 | 1.08E+08 | − | AluSx3 | chr6 |
| AluSx | chr6 | 1.11E+08 | 1.11E+08 | − | AluSx | chr6 |
| AluY | chr6 | 1.12E+08 | 1.12E+08 | + | AluSz | chr6 |
| AluSq2 | chr6 | 1.12E+08 | 1.12E+08 | + | AluSz | chr6 |
| AluY | chr6 | 1.31E+08 | 1.31E+08 | − | AluSx1 | chr6 |
| AluSz | chr6 | 1.31E+08 | 1.31E+08 | + | AluY | chr6 |
| AluSx | chr6 | 1.33E+08 | 1.33E+08 | − | AluJb | chr6 |
| AluY | chr6 | 1.38E+08 | 1.38E+08 | + | AluSx1 | chr6 |
| AluSx1 | chr6 | 1.39E+08 | 1.39E+08 | + | AluSp | chr6 |
| AluSz | chr6 | 1.39E+08 | 1.39E+08 | + | AluSq2 | chr6 |
| AluSg | chr6 | 1.39E+08 | 1.39E+08 | + | AluSx | chr6 |
| AluSx | chr6 | 1.39E+08 | 1.39E+08 | + | AluSz | chr6 |
| AluJr | chr6 | 1.39E+08 | 1.39E+08 | + | AluSx1 | chr6 |
| AluSp | chr6 | 1.39E+08 | 1.39E+08 | + | AluSp | chr6 |
| AluSg7 | chr6 | 1.39E+08 | 1.39E+08 | + | AluSz6 | chr6 |
| AluSq2 | chr6 | 1.39E+08 | 1.39E+08 | + | AluSx1 | chr6 |
| AluSp | chr6 | 1.39E+08 | 1.39E+08 | + | AluSp | chr6 |
| AluSx4 | chr6 | 1.39E+08 | 1.39E+08 | + | AluSx | chr6 |
| AluY | chr6 | 1.41E+08 | 1.41E+08 | − | AluSp | chr6 |
| AluY | chr6 | 1.44E+08 | 1.44E+08 | + | AluJo | chr6 |
| AluSx | chr6 | 1.45E+08 | 1.45E+08 | + | AluSz | chr6 |
| AluSg | chr6 | 1.5E+08 | 1.5E+08 | + | AluSx1 | chr6 |
| AluSx3 | chr6 | 1.5E+08 | 1.5E+08 | − | AluSz | chr6 |
| AluJb | chr6 | 1.5E+08 | 1.5E+08 | + | AluSx1 | chr6 |
| AluJr4 | chr6 | 1.5E+08 | 1.5E+08 | + | AluSx1 | chr6 |
| AluSc | chr6 | 1.5E+08 | 1.5E+08 | + | AluSx1 | chr6 |
| AluSp | chr6 | 1.5E+08 | 1.5E+08 | − | AluSz6 | chr6 |
| AluSx1 | chr6 | 1.5E+08 | 1.5E+08 | − | AluSz6 | chr6 |
| AluSx1 | chr6 | 1.5E+08 | 1.5E+08 | − | AluSz | chr6 |
| AluSq | chr6 | 1.5E+08 | 1.5E+08 | + | AluSx1 | chr6 |
| AluSq | chr6 | 1.5E+08 | 1.5E+08 | + | AluSq2 | chr6 |
| AluSq | chr6 | 1.5E+08 | 1.5E+08 | + | AluSp | chr6 |
| AluJb | chr6 | 1.5E+08 | 1.5E+08 | − | AluY | chr6 |
| AluSx | chr6 | 1.51E+08 | 1.51E+08 | − | AluSq2 | chr6 |
| AluJo | chr6 | 1.52E+08 | 1.52E+08 | + | AluSx | chr6 |
| AluSx | chr6 | 1.52E+08 | 1.52E+08 | − | AluSc5 | chr6 |
| AluSc | chr6 | 1.59E+08 | 1.59E+08 | − | AluJb | chr6 |
| AluSz | chr6 | 1.6E+08 | 1.6E+08 | − | AluSg | chr6 |
| AluSx1 | chr6 | 1.67E+08 | 1.67E+08 | − | AluSc | chr6 |
| AluSx | chr6 | 1.69E+08 | 1.69E+08 | + | AluJb | chr6 |
| AluSc | chr9 | 146894 | 147200 | + | AluY | chr9 |
| AluSz6 | chr9 | 147779 | 148027 | + | AluY | chr9 |
| AluYf4 | chr9 | 2797389 | 2797689 | + | AluSx1 | chr9 |
| AluSq2 | chr9 | 4738174 | 4738464 | − | AluSc8 | chr9 |
| AluSg | chr9 | 6420061 | 6420354 | − | AluY | chr9 |
| AluJo | chr9 | 6428542 | 6428847 | − | AluSx1 | chr9 |
| AluSz | chr9 | 12999322 | 12999622 | − | AluSx1 | chr9 |
| AluSx1 | chr9 | 12999655 | 12999951 | + | AluSz6 | chr9 |
| AluSz | chr9 | 12999322 | 12999622 | − | AluSx1 | chr9 |
| AluSz6 | chr9 | 13000437 | 13000740 | − | AluSx1 | chr9 |
| AluJr | chr9 | 15167028 | 15167345 | − | AluSp | chr9 |
| AluY | chr9 | 19099150 | 19099463 | − | AluSg7 | chr9 |
| AluSx1 | chr9 | 21973876 | 21974190 | − | AluSq2 | chr9 |
| AluSq2 | chr9 | 21976435 | 21976749 | + | AluSx | chr9 |
| AluSp | chr9 | 33027138 | 33027455 | − | AluSq | chr9 |
| AluSc8 | chr9 | 33862922 | 33863216 | + | AluSz | chr9 |
| AluSc8 | chr9 | 33871715 | 33872009 | − | AluJb | chr9 |
| AluSx | chr9 | 35763597 | 35763908 | + | AluY | chr9 |
| AluSx | chr9 | 36481000 | 36481306 | − | AluSq | chr9 |
| AluSp | chr9 | 37459190 | 37459471 | + | AluY | chr9 |
| AluJo | chr9 | 37503295 | 37503600 | + | AluSx3 | chr9 |
| AluJo | chr9 | 37503295 | 37503600 | + | AluSp | chr9 |
| AluSq2 | chr9 | 67281126 | 67281421 | − | AluJb | chr9 |
| AluJb | chr9 | 67283460 | 67283759 | + | AluJb | chr9 |
| AluJb | chr9 | 67285600 | 67285909 | − | AluSx1 | chr9 |
| AluSq2 | chr9 | 67697055 | 67697353 | + | AluSz | chr9 |
| AluSz | chr9 | 67698272 | 67698570 | − | AluSz | chr9 |
| AluSq | chr9 | 68414652 | 68414959 | + | AluJo | chr9 |
| AluJo | chr9 | 68416268 | 68416574 | − | AluSg4 | chr9 |
| AluSp | chr9 | 69841094 | 69841385 | − | AluY | chr9 |
| AluY | chr9 | 69841533 | 69841824 | + | AluY | chr9 |
| AluSz6 | chr9 | 70207859 | 70208107 | + | AluY | chr9 |
| AluSx1 | chr9 | 70637214 | 70637519 | − | AluY | chr9 |
| AluY | chr9 | 71743441 | 71743740 | − | AluSq2 | chr9 |
| AluSz | chr9 | 71772387 | 71772674 | − | AluSc8 | chr9 |
| AluSc8 | chr9 | 71774513 | 71774803 | + | AluSq2 | chr9 |
| AluSp | chr9 | 74522425 | 74522730 | − | AluSq2 | chr9 |
| AluSp | chr9 | 74558447 | 74558756 | − | AluSz6 | chr9 |
| AluSz6 | chr9 | 77384065 | 77384364 | − | AluSc8 | chr9 |
| AluSp | chr9 | 80488741 | 80489048 | + | AluJb | chr9 |
| AluSz | chr9 | 90174909 | 90175210 | − | AluY | chr9 |
| AluSp | chr9 | 91013491 | 91013781 | − | AluSx | chr9 |
| AluJo | chr9 | 91026437 | 91026729 | − | AluY | chr9 |
| AluJr | chr9 | 94052416 | 94052724 | + | AluSz | chr9 |
| AluSx1 | chr9 | 97055690 | 97056002 | − | AluSg | chr9 |
| AluYc | chr9 | 97246317 | 97246598 | + | AluSx | chr9 |
| AluSg7 | chr9 | 97248908 | 97249214 | + | AluSx | chr9 |
| AluJo | chr9 | 97250506 | 97250798 | − | AluJb | chr9 |
| AluSx | chr9 | 97249362 | 97249663 | − | AluJb | chr9 |
| AluSz | chr9 | 97300917 | 97301203 | + | AluSx | chr9 |
| AluY | chr9 | 97580995 | 97581304 | + | AluJb | chr9 |
| AluSx | chr9 | 97617354 | 97617659 | − | AluSp | chr9 |
| AluSc8 | chr9 | 97773494 | 97773782 | − | AluJo | chr9 |
| AluJo | chr9 | 98520904 | 98521209 | − | AluSc8 | chr9 |
| AluY | chr9 | 98575076 | 98575368 | + | AluSp | chr9 |
| AluJb | chr9 | 99099494 | 99099799 | − | AluSq2 | chr9 |
| AluSq2 | chr9 | 99099982 | 99100287 | + | AluSc | chr9 |
| AluSx | chr9 | 99114581 | 99114901 | − | AluSz | chr9 |
| AluSc | chr9 | 99164284 | 99164566 | + | AluSq | chr9 |
| AluJb | chr9 | 99224926 | 99225213 | + | AluJb | chr9 |
| AluSx | chr9 | 99224452 | 99224746 | + | AluJb | chr9 |
| AluSx | chr9 | 99224161 | 99224445 | + | AluJb | chr9 |
| AluSx | chr9 | 99224161 | 99224445 | + | AluSx1 | chr9 |
| AluJb | chr9 | 99224926 | 99225213 | + | AluSx1 | chr9 |
| AluSx | chr9 | 99224452 | 99224746 | + | AluSx1 | chr9 |
| AluJr | chr9 | 99243743 | 99244042 | + | AluSx3 | chr9 |
| AluJb | chr9 | 99354365 | 99354659 | − | AluSg | chr9 |
| AluSc | chr9 | 99362014 | 99362311 | − | AluSc | chr9 |
| AluSc | chr9 | 99362014 | 99362311 | − | AluSx1 | chr9 |
| AluSx | chr9 | 99663553 | 99663853 | − | AluSx | chr9 |
| AluSx | chr9 | 99663553 | 99663853 | − | AluSq | chr9 |
| AluSz | chr9 | 99708355 | 99708639 | − | AluY | chr9 |
| AluSz | chr9 | 99708355 | 99708639 | − | AluSp | chr9 |
| AluSp | chr9 | 99709156 | 99709408 | + | AluSq2 | chr9 |
| AluY | chr9 | 99708729 | 99709035 | + | AluSq2 | chr9 |
| AluSc | chr9 | 1E+08 | 1E+08 | − | AluSz6 | chr9 |
| AluJr | chr9 | 1.03E+08 | 1.03E+08 | − | AluSq2 | chr9 |
| AluSx | chr9 | 1.03E+08 | 1.03E+08 | − | AluSx1 | chr9 |
| AluSx1 | chr9 | 1.08E+08 | 1.08E+08 | + | AluSx1 | chr9 |
| AluSx | chr9 | 1.1E+08 | 1.1E+08 | + | AluSc8 | chr9 |
| AluSx1 | chr9 | 1.15E+08 | 1.15E+08 | + | AluJb | chr9 |
| AluSz | chr9 | 1.15E+08 | 1.15E+08 | + | AluJb | chr9 |
| AluJb | chr9 | 1.15E+08 | 1.15E+08 | − | AluYk4 | chr9 |
| AluSx | chr9 | 1.15E+08 | 1.15E+08 | + | AluJb | chr9 |
| AluSx | chr9 | 1.24E+08 | 1.24E+08 | + | AluSx | chr9 |
| AluJb | chr9 | 1.24E+08 | 1.24E+08 | + | AluSx | chr9 |
| AluSx1 | chr9 | 1.24E+08 | 1.24E+08 | − | AluSx | chr9 |
| AluSx1 | chr9 | 1.26E+08 | 1.26E+08 | + | AluSp | chr9 |
| AluSp | chr9 | 1.26E+08 | 1.26E+08 | − | AluSz6 | chr9 |
| AluSq2 | chr9 | 1.26E+08 | 1.26E+08 | − | AluJo | chr9 |
| AluJo | chr9 | 1.26E+08 | 1.26E+08 | + | AluSz | chr9 |
| AluJr | chr9 | 1.26E+08 | 1.26E+08 | − | AluJr | chr9 |
| AluJb | chr9 | 1.27E+08 | 1.27E+08 | − | AluJb | chr9 |
| AluJb | chr9 | 1.27E+08 | 1.27E+08 | − | AluJr4 | chr9 |
| AluSc | chr9 | 1.27E+08 | 1.27E+08 | + | AluSz6 | chr9 |
| AluJr | chr9 | 1.27E+08 | 1.27E+08 | − | AluSx4 | chr9 |
| AluSz | chr9 | 1.27E+08 | 1.27E+08 | + | AluSx1 | chr9 |
| AluSx | chr9 | 1.29E+08 | 1.29E+08 | + | AluSx | chr9 |
| AluSg | chr9 | 1.29E+08 | 1.29E+08 | − | AluY | chr9 |
| AluY | chr9 | 1.29E+08 | 1.29E+08 | + | AluSx1 | chr9 |
| AluSc | chr9 | 1.3E+08 | 1.3E+08 | − | AluSx | chr9 |
| AluSc8 | chr9 | 1.3E+08 | 1.3E+08 | − | AluSx1 | chr9 |
| AluSx1 | chr9 | 1.31E+08 | 1.31E+08 | − | AluSx | chr9 |
| AluSx | chr9 | 1.31E+08 | 1.31E+08 | + | AluSx3 | chr9 |
| AluSx1 | chr9 | 1.31E+08 | 1.31E+08 | − | AluSq | chr9 |
| AluSp | chr9 | 1.31E+08 | 1.31E+08 | + | AluSz6 | chr9 |
| AluSz6 | chr9 | 1.31E+08 | 1.31E+08 | − | AluSz | chr9 |
| AluSx | chr9 | 1.31E+08 | 1.31E+08 | − | AluSz | chr9 |
| AluSz | chr9 | 1.31E+08 | 1.31E+08 | + | AluJb | chr9 |
| AluSz | chr9 | 1.31E+08 | 1.31E+08 | + | AluY | chr9 |
| AluSq2 | chr9 | 1.31E+08 | 1.31E+08 | − | AluSq2 | chr9 |
| AluSx | chr9 | 1.31E+08 | 1.31E+08 | − | AluSg | chr9 |
| AluSq | chr9 | 1.31E+08 | 1.31E+08 | − | AluSx1 | chr9 |
| AluSx | chr9 | 1.31E+08 | 1.31E+08 | − | AluSx1 | chr9 |
| AluSx | chr9 | 1.31E+08 | 1.31E+08 | − | AluSx1 | chr9 |
| AluSx | chr9 | 1.31E+08 | 1.31E+08 | − | AluSg7 | chr9 |
| AluSq | chr9 | 1.31E+08 | 1.31E+08 | − | AluSg7 | chr9 |
| AluSx | chr9 | 1.31E+08 | 1.31E+08 | − | AluSg7 | chr9 |
| AluSg7 | chr9 | 1.31E+08 | 1.31E+08 | + | AluSq2 | chr9 |
| AluSx1 | chr9 | 1.31E+08 | 1.31E+08 | + | AluSq2 | chr9 |
| AluSg | chr9 | 1.31E+08 | 1.31E+08 | + | AluSq2 | chr9 |
| AluSg | chr9 | 1.31E+08 | 1.31E+08 | + | AluSz | chr9 |
| AluSx1 | chr9 | 1.31E+08 | 1.31E+08 | + | AluSz | chr9 |
| AluSg7 | chr9 | 1.31E+08 | 1.31E+08 | + | AluSz | chr9 |
| AluSx1 | chr9 | 1.31E+08 | 1.31E+08 | + | AluSp | chr9 |
| AluSg7 | chr9 | 1.31E+08 | 1.31E+08 | + | AluSp | chr9 |
| AluSg | chr9 | 1.31E+08 | 1.31E+08 | + | AluSp | chr9 |
| AluSg | chr9 | 1.31E+08 | 1.31E+08 | + | AluSx1 | chr9 |
| AluSz | chr9 | 1.31E+08 | 1.31E+08 | − | AluSx1 | chr9 |
| AluSx3 | chr9 | 1.31E+08 | 1.31E+08 | − | AluSx1 | chr9 |
| AluJo | chr9 | 1.31E+08 | 1.31E+08 | − | AluSx1 | chr9 |
| AluSx | chr9 | 1.31E+08 | 1.31E+08 | − | AluSx1 | chr9 |
| AluSx3 | chr9 | 1.31E+08 | 1.31E+08 | − | AluSx | chr9 |
| AluSz | chr9 | 1.31E+08 | 1.31E+08 | − | AluSx | chr9 |
| AluSx | chr9 | 1.31E+08 | 1.31E+08 | − | AluSx | chr9 |
| AluJo | chr9 | 1.31E+08 | 1.31E+08 | − | AluSx | chr9 |
| AluSz | chr9 | 1.31E+08 | 1.31E+08 | − | AluSx | chr9 |
| AluJo | chr9 | 1.31E+08 | 1.31E+08 | − | AluSx | chr9 |
| AluSz | chr9 | 1.31E+08 | 1.31E+08 | − | AluY | chr9 |
| AluJo | chr9 | 1.31E+08 | 1.31E+08 | − | AluY | chr9 |
| AluY | chr9 | 1.31E+08 | 1.31E+08 | − | AluSx | chr9 |
| AluSz | chr9 | 1.31E+08 | 1.31E+08 | − | AluJb | chr9 |
| AluY | chr9 | 1.31E+08 | 1.31E+08 | − | AluJb | chr9 |
| AluY | chr9 | 1.31E+08 | 1.31E+08 | + | AluSx1 | chr9 |
| AluY | chr9 | 1.31E+08 | 1.31E+08 | − | AluSx | chr9 |
| AluSq | chr9 | 1.31E+08 | 1.31E+08 | − | AluSx | chr9 |
| AluSg | chr9 | 1.31E+08 | 1.31E+08 | − | AluSx | chr9 |
| AluSg | chr9 | 1.31E+08 | 1.31E+08 | − | AluSx1 | chr9 |
| AluSx | chr9 | 1.31E+08 | 1.31E+08 | + | AluSz | chr9 |
| AluSx | chr9 | 1.31E+08 | 1.31E+08 | + | AluSz | chr9 |
| AluSx1 | chr9 | 1.31E+08 | 1.31E+08 | + | AluSz | chr9 |
| AluSx1 | chr9 | 1.31E+08 | 1.31E+08 | + | AluSz | chr9 |
| AluSz | chr9 | 1.31E+08 | 1.31E+08 | − | AluSg | chr9 |
| AluSz | chr9 | 1.31E+08 | 1.31E+08 | − | AluSg | chr9 |
| AluSp | chr9 | 1.31E+08 | 1.31E+08 | + | AluSx3 | chr9 |
| AluSg | chr9 | 1.31E+08 | 1.31E+08 | + | AluSx3 | chr9 |
| AluSx3 | chr9 | 1.31E+08 | 1.31E+08 | − | AluSq2 | chr9 |
| AluSz | chr9 | 1.31E+08 | 1.31E+08 | − | AluSq2 | chr9 |
| AluSz | chr9 | 1.31E+08 | 1.31E+08 | − | AluSq2 | chr9 |
| AluSx3 | chr9 | 1.31E+08 | 1.31E+08 | − | AluSc | chr9 |
| AluSz | chr9 | 1.31E+08 | 1.31E+08 | − | AluSc | chr9 |
| AluSz | chr9 | 1.31E+08 | 1.31E+08 | − | AluSc | chr9 |
| AluSz | chr9 | 1.31E+08 | 1.31E+08 | − | AluSq2 | chr9 |
| AluSz | chr9 | 1.31E+08 | 1.31E+08 | − | AluSq2 | chr9 |
| AluSx3 | chr9 | 1.31E+08 | 1.31E+08 | − | AluSq2 | chr9 |
| AluY | chr9 | 1.31E+08 | 1.31E+08 | − | AluSg | chr9 |
| AluY | chr9 | 1.31E+08 | 1.31E+08 | − | AluJo | chr9 |
| AluY | chr9 | 1.31E+08 | 1.31E+08 | − | AluSx1 | chr9 |
| AluSg | chr9 | 1.31E+08 | 1.31E+08 | + | AluSx1 | chr9 |
| AluSx1 | chr9 | 1.31E+08 | 1.31E+08 | + | AluSx1 | chr9 |
| AluJo | chr9 | 1.31E+08 | 1.31E+08 | + | AluSx1 | chr9 |
| AluSg | chr9 | 1.31E+08 | 1.31E+08 | + | AluY | chr9 |
| AluSx1 | chr9 | 1.31E+08 | 1.31E+08 | + | AluY | chr9 |
| AluJo | chr9 | 1.31E+08 | 1.31E+08 | + | AluY | chr9 |
| AluSx1 | chr9 | 1.31E+08 | 1.31E+08 | + | AluSp | chr9 |
| AluJo | chr9 | 1.31E+08 | 1.31E+08 | + | AluSp | chr9 |
| AluSx1 | chr9 | 1.31E+08 | 1.31E+08 | + | AluSq | chr9 |
| AluJo | chr9 | 1.31E+08 | 1.31E+08 | + | AluSq | chr9 |
| AluSz | chr9 | 1.31E+08 | 1.31E+08 | + | AluY | chr9 |
| AluSz | chr9 | 1.31E+08 | 1.31E+08 | + | AluY | chr9 |
| AluSz6 | chr9 | 1.32E+08 | 1.32E+08 | − | AluY | chr9 |
| AluY | chr9 | 1.32E+08 | 1.32E+08 | − | AluSz6 | chr9 |
| AluSz6 | chr9 | 1.32E+08 | 1.32E+08 | + | AluSq2 | chr9 |
| AluSz | chr9 | 1.32E+08 | 1.32E+08 | + | AluJb | chr9 |
| AluSx1 | chr9 | 1.32E+08 | 1.32E+08 | − | AluSp | chr9 |
| AluY | chr9 | 1.32E+08 | 1.32E+08 | − | AluSx | chr9 |
| AluSx | chr9 | 1.32E+08 | 1.32E+08 | + | AluJb | chr9 |
| AluJb | chr9 | 1.32E+08 | 1.32E+08 | − | AluSz | chr9 |
| AluJb | chr9 | 1.32E+08 | 1.32E+08 | − | AluSx | chr9 |
| AluJb | chr9 | 1.32E+08 | 1.32E+08 | − | AluY | chr9 |
| AluY | chr9 | 1.32E+08 | 1.32E+08 | + | AluSg | chr9 |
| AluSz | chr9 | 1.32E+08 | 1.32E+08 | + | AluSg | chr9 |
| AluSx | chr9 | 1.32E+08 | 1.32E+08 | + | AluSg | chr9 |
| AluSg | chr9 | 1.32E+08 | 1.32E+08 | − | AluJb | chr9 |
| AluSz | chr9 | 1.32E+08 | 1.32E+08 | + | AluSx1 | chr9 |
| AluSq | chr9 | 1.32E+08 | 1.32E+08 | + | AluSz | chr9 |
| AluY | chr9 | 1.32E+08 | 1.32E+08 | − | AluSz | chr9 |
| AluSx1 | chr9 | 1.33E+08 | 1.33E+08 | + | AluSp | chr9 |
| AluY | chr9 | 1.33E+08 | 1.33E+08 | + | AluSp | chr9 |
| AluSz6 | chr9 | 1.33E+08 | 1.33E+08 | − | AluSz | chr9 |
| AluSz6 | chr9 | 1.33E+08 | 1.33E+08 | − | AluY | chr9 |
| AluSp | chr9 | 1.33E+08 | 1.33E+08 | + | AluSz6 | chr9 |
| AluSx3 | chr9 | 1.34E+08 | 1.34E+08 | + | AluSc | chr9 |
| AluSx3 | chr9 | 1.34E+08 | 1.34E+08 | + | AluSc5 | chr9 |
| AluSc5 | chr9 | 1.34E+08 | 1.34E+08 | − | AluSx1 | chr9 |
| AluSc | chr9 | 1.34E+08 | 1.34E+08 | − | AluSx1 | chr9 |
| AluSz | chr9 | 1.34E+08 | 1.34E+08 | − | AluSp | chr9 |
| AluSz | chr9 | 1.34E+08 | 1.34E+08 | − | AluSz | chr9 |
| AluJb | chr9 | 1.34E+08 | 1.34E+08 | + | AluSc8 | chr9 |
| AluSx1 | chr9 | 1.34E+08 | 1.34E+08 | + | AluJb | chr9 |
| AluY | chr9 | 1.34E+08 | 1.34E+08 | + | AluSz6 | chr9 |
| AluSx1 | chr9 | 1.34E+08 | 1.34E+08 | + | AluSz6 | chr9 |
| AluSc8 | chr9 | 1.34E+08 | 1.34E+08 | + | AluSx | chr9 |
| AluSx | chr9 | 1.34E+08 | 1.34E+08 | − | AluSc8 | chr9 |
| AluJb | chr9 | 1.34E+08 | 1.34E+08 | − | AluSz | chr9 |
| AluSz | chr9 | 1.35E+08 | 1.35E+08 | + | AluY | chr9 |
| AluSz | chr9 | 1.35E+08 | 1.35E+08 | − | AluSz | chr9 |
| AluSx1 | chr9 | 1.36E+08 | 1.36E+08 | − | AluJb | chr9 |
| AluSq2 | chr9 | 1.36E+08 | 1.36E+08 | − | AluJb | chr9 |
| AluJb | chr9 | 1.39E+08 | 1.39E+08 | + | AluY | chr9 |
| AluSx1 | chr9 | 1.4E+08 | 1.4E+08 | + | AluSg7 | chr9 |
| AluSz | chr9 | 1.41E+08 | 1.41E+08 | + | AluSx1 | chr9 |
| AluSx1 | chr9 | 1.41E+08 | 1.41E+08 | − | AluJo | chr9 |
| AluJo | chr9 | 1.41E+08 | 1.41E+08 | + | AluSx3 | chr9 |
| AluY | chr9 | 1.41E+08 | 1.41E+08 | + | AluSg7 | chr9 |
| AluY | chr2 | 9350691 | 9350970 | − | AluSp | chr2 |
| AluY | chr2 | 9537881 | 9538186 | − | AluJr | chr2 |
| AluY | chr2 | 9535725 | 9536035 | − | AluJr | chr2 |
| AluJb | chr2 | 9720114 | 9720394 | − | AluSx1 | chr2 |
| AluY | chr2 | 10339626 | 10339935 | + | AluSq2 | chr2 |
| AluSp | chr2 | 10819204 | 10819503 | + | AluSz6 | chr2 |
| AluSx1 | chr2 | 11388552 | 11388864 | + | AluSc8 | chr2 |
| AluSq2 | chr2 | 11415101 | 11415402 | + | AluSx1 | chr2 |
| AluSc | chr2 | 24227244 | 24227543 | + | AluSq2 | chr2 |
| AluSx | chr2 | 25046560 | 25046863 | − | AluSx1 | chr2 |
| AluSz | chr2 | 26034632 | 26034921 | − | AluSq2 | chr2 |
| AluSp | chr2 | 26260226 | 26260522 | + | AluSq2 | chr2 |
| AluSq2 | chr2 | 26260758 | 26261048 | − | AluY | chr2 |
| AluSp | chr2 | 26260226 | 26260522 | + | AluSx | chr2 |
| AluY | chr2 | 26261510 | 26261812 | + | AluSx | chr2 |
| AluSp | chr2 | 26260226 | 26260522 | + | AluSx1 | chr2 |
| AluY | chr2 | 26261510 | 26261812 | + | AluSx1 | chr2 |
| AluSx1 | chr2 | 26262219 | 26262517 | − | AluSq2 | chr2 |
| AluSx | chr2 | 26261885 | 26262189 | − | AluSq2 | chr2 |
| AluSq2 | chr2 | 26299531 | 26299829 | + | AluJo | chr2 |
| AluSx | chr2 | 27339113 | 27339421 | + | AluSx | chr2 |
| AluSz | chr2 | 29007500 | 29007811 | + | AluSc | chr2 |
| AluSz | chr2 | 29012460 | 29012768 | − | AluSz6 | chr2 |
| AluSz | chr2 | 29012460 | 29012768 | − | AluSp | chr2 |
| AluY | chr2 | 29162694 | 29162998 | + | AluSp | chr2 |
| AluY | chr2 | 32163434 | 32163741 | + | AluJb | chr2 |
| AluSg | chr2 | 32178157 | 32178454 | + | AluSz | chr2 |
| AluSx1 | chr2 | 32523935 | 32524242 | − | AluSp | chr2 |
| AluSx1 | chr2 | 38561171 | 38561471 | − | AluSp | chr2 |
| AluSc8 | chr2 | 39007515 | 39007778 | − | AluY | chr2 |
| AluSz | chr2 | 39280012 | 39280306 | + | AluY | chr2 |
| AluSq | chr2 | 39707393 | 39707703 | + | AluSp | chr2 |
| AluJb | chr2 | 39715712 | 39716005 | − | AluSz | chr2 |
| AluY | chr2 | 39716687 | 39716980 | − | AluSx | chr2 |
| AluSz6 | chr2 | 39716019 | 39716323 | − | AluSx | chr2 |
| AluJb | chr2 | 39715712 | 39716005 | − | AluSx | chr2 |
| AluSx | chr2 | 39717419 | 39717726 | + | AluSz | chr2 |
| AluSx | chr2 | 39717419 | 39717726 | + | AluY | chr2 |
| AluSz | chr2 | 39717020 | 39717317 | + | AluY | chr2 |
| AluSp | chr2 | 42485552 | 42485839 | − | AluSx1 | chr2 |
| AluSx3 | chr2 | 42508252 | 42508552 | + | AluJb | chr2 |
| AluSz | chr2 | 43809356 | 43809650 | + | AluSz | chr2 |
| AluSz6 | chr2 | 44008224 | 44008523 | + | AluY | chr2 |
| AluSg | chr2 | 44024655 | 44024956 | − | AluJb | chr2 |
| AluSz | chr2 | 44213269 | 44213569 | − | AluSx1 | chr2 |
| AluSx | chr2 | 44581718 | 44582015 | − | AluSz6 | chr2 |
| AluSz6 | chr2 | 44582405 | 44582695 | + | AluJb | chr2 |
| AluJb | chr2 | 47555375 | 47555673 | + | AluJo | chr2 |
| AluSx | chr2 | 47570912 | 47571212 | + | AluSq2 | chr2 |
| AluSx1 | chr2 | 47568894 | 47569203 | + | AluSq2 | chr2 |
| AluY | chr2 | 54098396 | 54098689 | − | AluSq | chr2 |
| AluSx | chr2 | 54237919 | 54238105 | − | AluSq2 | chr2 |
| AluY | chr2 | 55756276 | 55756570 | + | AluY | chr2 |
| AluSx1 | chr2 | 55846791 | 55847093 | + | AluSx1 | chr2 |
| AluY | chr2 | 61247112 | 61247382 | + | AluSx | chr2 |
| AluJb | chr2 | 61250958 | 61251227 | − | AluY | chr2 |
| AluY | chr2 | 61251663 | 61251968 | + | AluSg4 | chr2 |
| AluJb | chr2 | 61250958 | 61251227 | − | AluJr | chr2 |
| AluSg4 | chr2 | 61252194 | 61252450 | − | AluJr | chr2 |
| AluSp | chr2 | 61276628 | 61276921 | + | AluY | chr2 |
| AluSp | chr2 | 61276628 | 61276921 | + | AluSq2 | chr2 |
| AluSx | chr2 | 61277244 | 61277529 | + | AluSq2 | chr2 |
| AluY | chr2 | 61277752 | 61278054 | − | AluSx3 | chr2 |
| AluSq2 | chr2 | 61279777 | 61280069 | − | AluSx3 | chr2 |
| AluSq2 | chr2 | 61279777 | 61280069 | − | AluSq2 | chr2 |
| AluY | chr2 | 61277752 | 61278054 | − | AluSq2 | chr2 |
| AluSq2 | chr2 | 61280413 | 61280694 | + | AluY | chr2 |
| AluSx3 | chr2 | 61280096 | 61280375 | + | AluY | chr2 |
| AluSg | chr2 | 61315668 | 61315953 | − | AluSx1 | chr2 |
| AluSp | chr2 | 61421809 | 61422105 | + | AluSc | chr2 |
| AluSq2 | chr2 | 61667654 | 61667952 | − | AluY | chr2 |
| AluY | chr2 | 61700312 | 61700610 | + | AluSp | chr2 |
| AluSp | chr2 | 61700016 | 61700311 | + | AluSp | chr2 |
| AluSp | chr2 | 61702170 | 61702470 | − | AluSx | chr2 |
| AluSx1 | chr2 | 61729603 | 61729893 | + | AluSx1 | chr2 |
| AluSx1 | chr2 | 61729603 | 61729893 | + | AluSx | chr2 |
| AluSq2 | chr2 | 61736151 | 61736439 | − | AluSz6 | chr2 |
| AluSp | chr2 | 61736491 | 61736785 | + | AluSp | chr2 |
| AluSz6 | chr2 | 61737133 | 61737430 | + | AluSp | chr2 |
| AluSg | chr2 | 61746096 | 61746383 | − | AluY | chr2 |
| AluSq | chr2 | 61744060 | 61744335 | − | AluY | chr2 |
| AluSx1 | chr2 | 62097772 | 62098087 | + | AluSq2 | chr2 |
| AluSg | chr2 | 63088260 | 63088562 | − | AluSz | chr2 |
| AluSc | chr2 | 64166902 | 64167189 | − | AluSz | chr2 |
| AluSx1 | chr2 | 64236982 | 64237275 | − | AluSc | chr2 |
| AluSx1 | chr2 | 64243745 | 64244042 | − | AluSx4 | chr2 |
| AluSg | chr2 | 65350686 | 65350971 | + | AluSz | chr2 |
| AluSp | chr2 | 65354585 | 65354872 | + | AluY | chr2 |
| AluSx1 | chr2 | 65463207 | 65463484 | + | AluSz6 | chr2 |
| AluY | chr2 | 65589398 | 65589703 | − | AluSq | chr2 |
| AluY | chr2 | 65589398 | 65589703 | − | AluSp | chr2 |
| AluSx | chr2 | 69587178 | 69587483 | + | AluJo | chr2 |
| AluSx | chr2 | 69587178 | 69587483 | + | AluSx | chr2 |
| AluSq2 | chr2 | 69688728 | 69689023 | − | AluSg | chr2 |
| AluSg | chr2 | 69689531 | 69689824 | + | AluSx3 | chr2 |
| AluJb | chr2 | 69975840 | 69976144 | − | AluSx | chr2 |
| AluJb | chr2 | 69982470 | 69982749 | + | AluSx | chr2 |
| AluJb | chr2 | 69982470 | 69982749 | + | AluY | chr2 |
| AluJb | chr2 | 70036619 | 70036909 | − | AluSx | chr2 |
| AluSg | chr2 | 70461709 | 70461990 | − | AluSp | chr2 |
| AluSg | chr2 | 70461709 | 70461990 | − | AluSx | chr2 |
| AluSg | chr2 | 70461709 | 70461990 | − | AluSz | chr2 |
| AluY | chr2 | 71561772 | 71562071 | + | AluJo | chr2 |
| AluSz | chr2 | 73204826 | 73205131 | + | AluSx1 | chr2 |
| AluSx | chr2 | 75931808 | 75932092 | − | AluY | chr2 |
| AluSq2 | chr2 | 78515922 | 78516220 | − | AluSz | chr2 |
| AluY | chr2 | 78515626 | 78515918 | − | AluSz | chr2 |
| AluSg4 | chr2 | 78530669 | 78530972 | − | AluJb | chr2 |
| AluSx1 | chr2 | 78543336 | 78543620 | + | AluY | chr2 |
| AluSx | chr2 | 85193614 | 85193927 | + | AluSg7 | chr2 |
| AluSx1 | chr2 | 85193035 | 85193294 | + | AluSg7 | chr2 |
| AluSx | chr2 | 86250872 | 86251170 | − | AluSq2 | chr2 |
| AluJo | chr2 | 86249720 | 86250002 | − | AluSq2 | chr2 |
| AluSx | chr2 | 86381996 | 86382285 | − | AluSz | chr2 |
| AluSz6 | chr2 | 86986751 | 86986933 | + | AluSp | chr2 |
| AluY | chr2 | 87192424 | 87192737 | − | AluSx1 | chr2 |
| AluSx1 | chr2 | 88102490 | 88102789 | − | AluY | chr2 |
| AluSx1 | chr2 | 88102490 | 88102789 | − | AluSc | chr2 |
| AluSq | chr2 | 88347606 | 88347882 | − | AluSz | chr2 |
| AluJr | chr2 | 91811305 | 91811616 | + | AluSq2 | chr2 |
| AluY | chr2 | 91832852 | 91833148 | − | AluSx | chr2 |
| AluSq | chr2 | 91832254 | 91832497 | − | AluSx | chr2 |
| AluY | chr2 | 91832852 | 91833148 | − | AluSx | chr2 |
| AluSx | chr2 | 91834423 | 91834710 | + | AluSc | chr2 |
| AluSx | chr2 | 91834855 | 91835121 | + | AluSc | chr2 |
| AluSx | chr2 | 91834855 | 91835121 | + | AluSc | chr2 |
| AluJo | chr2 | 91837719 | 91837989 | − | AluSp | chr2 |
| AluSc | chr2 | 91837998 | 91838303 | − | AluSp | chr2 |
| AluSc | chr2 | 91837998 | 91838303 | − | AluJb | chr2 |
| AluJo | chr2 | 91837719 | 91837989 | − | AluJb | chr2 |
| AluSp | chr2 | 91840608 | 91840937 | + | AluSc5 | chr2 |
| AluJb | chr2 | 91840938 | 91841281 | + | AluSc5 | chr2 |
| AluY | chr2 | 96935380 | 96935681 | − | AluSz | chr2 |
| AluJb | chr2 | 97294107 | 97294415 | + | AluSp | chr2 |
| AluSx1 | chr2 | 1E+08 | 1E+08 | + | AluSq2 | chr2 |
| AluSz | chr2 | 1E+08 | 1E+08 | + | AluSq2 | chr2 |
| AluSz | chr2 | 1.02E+08 | 1.02E+08 | + | AluSq | chr2 |
| AluSq | chr2 | 1.02E+08 | 1.02E+08 | − | AluSq | chr2 |
| AluSg7 | chr2 | 1.02E+08 | 1.02E+08 | + | AluSx1 | chr2 |
| AluSx1 | chr2 | 1.06E+08 | 1.06E+08 | + | AluY | chr2 |
| AluSx | chr2 | 1.09E+08 | 1.09E+08 | + | AluSg7 | chr2 |
| AluY | chr2 | 1.09E+08 | 1.09E+08 | − | AluSx1 | chr2 |
| AluSx1 | chr2 | 1.09E+08 | 1.09E+08 | + | AluY | chr2 |
| AluY | chr2 | 1.09E+08 | 1.09E+08 | − | AluSx1 | chr2 |
| AluSx | chr2 | 1.09E+08 | 1.09E+08 | + | AluJr | chr2 |
| AluSx | chr2 | 1.09E+08 | 1.09E+08 | + | AluSc | chr2 |
| AluSc | chr2 | 1.09E+08 | 1.09E+08 | − | AluJb | chr2 |
| AluJr | chr2 | 1.09E+08 | 1.09E+08 | − | AluJb | chr2 |
| AluSc | chr2 | 1.1E+08 | 1.1E+08 | − | AluSp | chr2 |
| AluSp | chr2 | 1.13E+08 | 1.13E+08 | − | AluSx | chr2 |
| AluSx1 | chr2 | 1.13E+08 | 1.13E+08 | − | AluY | chr2 |
| AluSp | chr2 | 1.14E+08 | 1.14E+08 | + | AluSc | chr2 |
| AluY | chr2 | 1.14E+08 | 1.14E+08 | + | AluSz6 | chr2 |
| AluSx3 | chr2 | 1.14E+08 | 1.14E+08 | − | AluSz | chr2 |
| AluSz | chr2 | 1.14E+08 | 1.14E+08 | + | AluY | chr2 |
| AluSq | chr2 | 1.15E+08 | 1.15E+08 | − | AluSg | chr2 |
| AluSg7 | chr2 | 1.15E+08 | 1.15E+08 | + | AluY | chr2 |
| AluY | chr2 | 1.21E+08 | 1.21E+08 | + | AluJb | chr2 |
| AluSz | chr2 | 1.21E+08 | 1.21E+08 | + | AluSc | chr2 |
| AluSq | chr2 | 1.22E+08 | 1.22E+08 | − | AluY | chr2 |
| AluJb | chr2 | 1.28E+08 | 1.28E+08 | + | AluSq2 | chr2 |
| AluSq2 | chr2 | 1.29E+08 | 1.29E+08 | + | AluSq2 | chr2 |
| AluSz | chr2 | 1.29E+08 | 1.29E+08 | + | AluSz | chr2 |
| AluSz | chr2 | 1.29E+08 | 1.29E+08 | − | AluY | chr2 |
| AluSx | chr2 | 1.29E+08 | 1.29E+08 | − | AluJb | chr2 |
| AluJr | chr2 | 1.29E+08 | 1.29E+08 | − | AluJo | chr2 |
| AluSc | chr2 | 1.29E+08 | 1.29E+08 | + | AluSx1 | chr2 |
| AluSg | chr2 | 1.29E+08 | 1.29E+08 | + | AluSx1 | chr2 |
| AluSx | chr2 | 1.31E+08 | 1.31E+08 | − | AluSz | chr2 |
| AluSq2 | chr2 | 1.31E+08 | 1.31E+08 | − | AluSc5 | chr2 |
| AluSx1 | chr2 | 1.32E+08 | 1.32E+08 | + | AluSx3 | chr2 |
| AluSq2 | chr2 | 1.35E+08 | 1.35E+08 | + | AluSc8 | chr2 |
| AluJr | chr2 | 1.35E+08 | 1.35E+08 | + | AluSz | chr2 |
| AluSx | chr2 | 1.35E+08 | 1.35E+08 | − | AluSx | chr2 |
| AluSz | chr2 | 1.36E+08 | 1.36E+08 | + | AluSg | chr2 |
| AluJb | chr2 | 1.36E+08 | 1.36E+08 | + | AluSz | chr2 |
| AluSx | chr2 | 1.36E+08 | 1.36E+08 | − | AluSc | chr2 |
| AluSx | chr2 | 1.53E+08 | 1.53E+08 | − | AluSx3 | chr2 |
| AluSq2 | chr2 | 1.53E+08 | 1.53E+08 | − | AluJb | chr2 |
| AluSx | chr2 | 1.57E+08 | 1.57E+08 | − | AluSx | chr2 |
| AluSx | chr2 | 1.57E+08 | 1.57E+08 | − | AluSx1 | chr2 |
| AluSx1 | chr2 | 1.59E+08 | 1.59E+08 | + | AluSx3 | chr2 |
| AluSx4 | chr2 | 1.6E+08 | 1.6E+08 | − | AluSq | chr2 |
| AluSx3 | chr2 | 1.6E+08 | 1.6E+08 | + | AluSx1 | chr2 |
| AluJb | chr2 | 1.71E+08 | 1.71E+08 | + | AluSz6 | chr2 |
| AluJr | chr2 | 1.71E+08 | 1.71E+08 | − | AluY | chr2 |
| AluJb | chr2 | 1.72E+08 | 1.72E+08 | − | AluSx3 | chr2 |
| AluSx | chr2 | 1.72E+08 | 1.72E+08 | − | AluSx1 | chr2 |
| AluY | chr2 | 1.72E+08 | 1.72E+08 | − | AluSx1 | chr2 |
| AluY | chr2 | 1.72E+08 | 1.72E+08 | + | AluSz6 | chr2 |
| AluSp | chr2 | 1.73E+08 | 1.73E+08 | + | AluSx3 | chr2 |
| AluSx1 | chr2 | 1.73E+08 | 1.73E+08 | − | AluSp | chr2 |
| AluJb | chr2 | 1.73E+08 | 1.73E+08 | + | AluSc8 | chr2 |
| AluSx1 | chr2 | 1.73E+08 | 1.73E+08 | + | AluSc8 | chr2 |
| AluSp | chr2 | 1.75E+08 | 1.75E+08 | − | AluJb | chr2 |
| AluSc | chr2 | 1.78E+08 | 1.78E+08 | + | AluY | chr2 |
| AluSc | chr2 | 1.78E+08 | 1.78E+08 | + | AluSx | chr2 |
| AluSp | chr2 | 1.78E+08 | 1.78E+08 | − | AluJb | chr2 |
| AluJb | chr2 | 1.78E+08 | 1.78E+08 | − | AluY | chr2 |
| AluSc8 | chr2 | 2.01E+08 | 2.01E+08 | − | AluJb | chr2 |
| AluSz6 | chr2 | 2.02E+08 | 2.02E+08 | − | AluSc8 | chr2 |
| AluSx1 | chr2 | 2.02E+08 | 2.02E+08 | − | AluJo | chr2 |
| AluSx1 | chr2 | 2.03E+08 | 2.03E+08 | − | AluSz | chr2 |
| AluYc | chr2 | 2.03E+08 | 2.03E+08 | + | AluSx | chr2 |
| AluY | chr2 | 2.03E+08 | 2.03E+08 | + | AluSx | chr2 |
| AluSx | chr2 | 2.03E+08 | 2.03E+08 | − | AluSp | chr2 |
| AluSp | chr2 | 2.03E+08 | 2.03E+08 | + | AluSg | chr2 |
| AluSg | chr2 | 2.03E+08 | 2.03E+08 | − | AluSq2 | chr2 |
| AluSq2 | chr2 | 2.03E+08 | 2.03E+08 | + | AluSg | chr2 |
| AluSq2 | chr2 | 2.03E+08 | 2.03E+08 | + | AluSx | chr2 |
| AluSq2 | chr2 | 2.03E+08 | 2.03E+08 | + | AluSp | chr2 |
| AluSq | chr2 | 2.03E+08 | 2.03E+08 | + | AluSp | chr2 |
| AluSg | chr2 | 2.04E+08 | 2.04E+08 | + | AluY | chr2 |
| AluSz | chr2 | 2.04E+08 | 2.04E+08 | − | AluJb | chr2 |
| AluSp | chr2 | 2.04E+08 | 2.04E+08 | + | AluSg | chr2 |
| AluSp | chr2 | 2.04E+08 | 2.04E+08 | + | AluSg | chr2 |
| AluJb | chr2 | 2.04E+08 | 2.04E+08 | + | AluSx | chr2 |
| AluSz | chr2 | 2.04E+08 | 2.04E+08 | − | AluSq2 | chr2 |
| AluSx | chr2 | 2.04E+08 | 2.04E+08 | − | AluSq2 | chr2 |
| AluSg | chr2 | 2.04E+08 | 2.04E+08 | − | AluSq2 | chr2 |
| AluSx | chr2 | 2.04E+08 | 2.04E+08 | − | AluSx1 | chr2 |
| AluSz | chr2 | 2.04E+08 | 2.04E+08 | − | AluSx1 | chr2 |
| AluSg | chr2 | 2.04E+08 | 2.04E+08 | − | AluSx1 | chr2 |
| AluSx1 | chr2 | 2.04E+08 | 2.04E+08 | + | AluSc8 | chr2 |
| AluSq2 | chr2 | 2.04E+08 | 2.04E+08 | + | AluSc8 | chr2 |
| AluJb | chr2 | 2.04E+08 | 2.04E+08 | + | AluSc8 | chr2 |
| AluSg | chr2 | 2.04E+08 | 2.04E+08 | − | AluJb | chr2 |
| AluSx | chr2 | 2.04E+08 | 2.04E+08 | − | AluJb | chr2 |
| AluSz | chr2 | 2.04E+08 | 2.04E+08 | − | AluJb | chr2 |
| AluSc8 | chr2 | 2.04E+08 | 2.04E+08 | − | AluJb | chr2 |
| AluSx1 | chr2 | 2.04E+08 | 2.04E+08 | + | AluSc | chr2 |
| AluSq2 | chr2 | 2.04E+08 | 2.04E+08 | + | AluSc | chr2 |
| AluJb | chr2 | 2.04E+08 | 2.04E+08 | + | AluSc | chr2 |
| AluJb | chr2 | 2.04E+08 | 2.04E+08 | + | AluSc | chr2 |
| AluSc8 | chr2 | 2.04E+08 | 2.04E+08 | − | AluY | chr2 |
| AluSc | chr2 | 2.04E+08 | 2.04E+08 | − | AluY | chr2 |
| AluSx1 | chr2 | 2.04E+08 | 2.04E+08 | + | AluSz | chr2 |
| AluSg7 | chr2 | 2.07E+08 | 2.07E+08 | − | AluJb | chr2 |
| AluSg7 | chr2 | 2.07E+08 | 2.07E+08 | − | AluSx | chr2 |
| AluSx | chr2 | 2.07E+08 | 2.07E+08 | + | AluSx | chr2 |
| AluJb | chr2 | 2.07E+08 | 2.07E+08 | + | AluSx | chr2 |
| AluSg | chr2 | 2.07E+08 | 2.07E+08 | + | AluSx | chr2 |
| AluSx | chr2 | 2.07E+08 | 2.07E+08 | + | AluSz | chr2 |
| AluJb | chr2 | 2.07E+08 | 2.07E+08 | + | AluSz | chr2 |
| AluSx | chr2 | 2.07E+08 | 2.07E+08 | − | AluSx | chr2 |
| AluSg | chr2 | 2.09E+08 | 2.09E+08 | + | AluSq2 | chr2 |
| AluSq2 | chr2 | 2.16E+08 | 2.16E+08 | − | AluSx | chr2 |
| AluSx | chr2 | 2.16E+08 | 2.16E+08 | + | AluJb | chr2 |
| AluY | chr2 | 2.16E+08 | 2.16E+08 | + | AluSz | chr2 |
| AluSx1 | chr2 | 2.19E+08 | 2.19E+08 | − | AluSg | chr2 |
| AluY | chr2 | 2.19E+08 | 2.19E+08 | − | AluSx1 | chr2 |
| AluSx1 | chr2 | 2.19E+08 | 2.19E+08 | + | AluSq | chr2 |
| AluSp | chr2 | 2.19E+08 | 2.19E+08 | + | AluSq | chr2 |
| AluSx1 | chr2 | 2.19E+08 | 2.19E+08 | + | AluSx1 | chr2 |
| AluSp | chr2 | 2.19E+08 | 2.19E+08 | + | AluSx1 | chr2 |
| AluSz | chr2 | 2.19E+08 | 2.19E+08 | + | AluYf5 | chr2 |
| AluJo | chr2 | 2.19E+08 | 2.19E+08 | + | AluJb | chr2 |
| AluSq10 | chr2 | 2.19E+08 | 2.19E+08 | + | AluSz | chr2 |
| AluJb | chr2 | 2.2E+08 | 2.2E+08 | + | AluSp | chr2 |
| AluSp | chr2 | 2.2E+08 | 2.2E+08 | − | AluJr | chr2 |
| AluJr | chr2 | 2.2E+08 | 2.2E+08 | + | AluSp | chr2 |
| AluJr | chr2 | 2.2E+08 | 2.2E+08 | + | AluSz | chr2 |
| AluJb | chr2 | 2.23E+08 | 2.23E+08 | − | AluY | chr2 |
| AluJb | chr2 | 2.24E+08 | 2.24E+08 | − | AluSc8 | chr2 |
| AluSx | chr2 | 2.24E+08 | 2.24E+08 | + | AluJb | chr2 |
| AluJo | chr2 | 2.25E+08 | 2.25E+08 | − | AluSq | chr2 |
| AluSx1 | chr2 | 2.25E+08 | 2.25E+08 | + | AluSx | chr2 |
| AluSx | chr2 | 2.25E+08 | 2.25E+08 | − | AluY | chr2 |
| AluSz | chr2 | 2.25E+08 | 2.25E+08 | + | AluSx1 | chr2 |
| AluSx1 | chr2 | 2.25E+08 | 2.25E+08 | − | AluSq2 | chr2 |
| AluSz | chr2 | 2.25E+08 | 2.25E+08 | + | AluY | chr2 |
| AluSq2 | chr2 | 2.25E+08 | 2.25E+08 | − | AluJr | chr2 |
| AluSq2 | chr2 | 2.25E+08 | 2.25E+08 | − | AluSz | chr2 |
| AluSz | chr2 | 2.25E+08 | 2.25E+08 | + | AluSx | chr2 |
| AluSq2 | chr2 | 2.25E+08 | 2.25E+08 | − | AluSp | chr2 |
| AluSz | chr2 | 2.25E+08 | 2.25E+08 | + | AluSc | chr2 |
| AluSz | chr2 | 2.25E+08 | 2.25E+08 | − | AluSz | chr2 |
| AluYf4 | chr2 | 2.25E+08 | 2.25E+08 | − | AluSz | chr2 |
| AluSq2 | chr2 | 2.25E+08 | 2.25E+08 | + | AluSz6 | chr2 |
| AluJo | chr2 | 2.25E+08 | 2.25E+08 | + | AluSz | chr2 |
| AluSz6 | chr2 | 2.25E+08 | 2.25E+08 | + | AluSz | chr2 |
| AluSx | chr2 | 2.31E+08 | 2.31E+08 | − | AluSx | chr2 |
| AluJb | chr2 | 2.32E+08 | 2.32E+08 | − | AluSx | chr2 |
| AluSx | chr2 | 2.32E+08 | 2.32E+08 | − | AluSz | chr2 |
| AluSz | chr2 | 2.32E+08 | 2.32E+08 | + | AluSc | chr2 |
| AluJo | chr2 | 2.32E+08 | 2.32E+08 | − | AluSq2 | chr2 |
| AluSx3 | chr2 | 2.32E+08 | 2.32E+08 | − | AluSz | chr2 |
| AluSx1 | chr2 | 2.34E+08 | 2.34E+08 | + | AluY | chr2 |
| AluSx1 | chr2 | 2.34E+08 | 2.34E+08 | + | AluSq | chr2 |
| AluSz | chr2 | 2.34E+08 | 2.34E+08 | + | AluJr | chr2 |
| AluSx1 | chr2 | 2.38E+08 | 2.38E+08 | − | AluSz | chr2 |
| AluSx | chr2 | 2.39E+08 | 2.39E+08 | − | AluSx1 | chr2 |
| AluSx | chr2 | 2.39E+08 | 2.39E+08 | − | AluSz | chr2 |
| AluJb | chr2 | 2.39E+08 | 2.39E+08 | + | AluY | chr2 |
| AluSq | chr2 | 2.41E+08 | 2.41E+08 | + | AluJb | chr2 |
| AluSc | chr2 | 2.42E+08 | 2.42E+08 | − | AluSq | chr2 |
| AluJb | chr2 | 2.42E+08 | 2.42E+08 | − | AluSq | chr2 |
| AluY | chr2 | 2.42E+08 | 2.42E+08 | + | AluSc8 | chr2 |
| AluSx1 | chr2 | 2.42E+08 | 2.42E+08 | + | AluY | chr2 |
| AluSx | chr2 | 2.42E+08 | 2.42E+08 | − | AluY | chr2 |
| AluSq2 | chr2 | 2.43E+08 | 2.43E+08 | − | AluSq2 | chr2 |
| AluY | chr1 | 1338227 | 1338535 | + | AluY | chr1 |
| AluSg | chr1 | 1496465 | 1496761 | − | AluSg7 | chr1 |
| AluSg | chr1 | 1660829 | 1661117 | − | AluJo | chr1 |
| AluSz6 | chr1 | 1819072 | 1819351 | − | AluSq | chr1 |
| AluSz | chr1 | 6431823 | 6432125 | + | AluSz | chr1 |
| AluSz | chr1 | 6432608 | 6432909 | − | AluSq2 | chr1 |
| AluJb | chr1 | 6442591 | 6442885 | + | AluSq2 | chr1 |
| AluSc | chr1 | 6442192 | 6442488 | + | AluSq2 | chr1 |
| AluSq | chr1 | 6583359 | 6583663 | + | AluY | chr1 |
| AluY | chr1 | 8034408 | 8034701 | + | AluSq | chr1 |
| AluSx | chr1 | 8665520 | 8665830 | + | AluSx1 | chr1 |
| AluSx | chr1 | 9161111 | 9161414 | − | AluSx | chr1 |
| AluSx | chr1 | 9162187 | 9162494 | + | AluSq2 | chr1 |
| AluSx1 | chr1 | 9326884 | 9327178 | − | AluJr | chr1 |
| AluJr | chr1 | 9328031 | 9328325 | + | AluSx | chr1 |
| AluSx | chr1 | 9987371 | 9987657 | − | AluSp | chr1 |
| AluSx3 | chr1 | 9987737 | 9988029 | − | AluSp | chr1 |
| AluSx3 | chr1 | 9987737 | 9988029 | − | AluSq2 | chr1 |
| AluSx | chr1 | 9987371 | 9987657 | − | AluSq2 | chr1 |
| AluSc | chr1 | 10042886 | 10043177 | + | AluSx1 | chr1 |
| AluSx3 | chr1 | 10043431 | 10043727 | + | AluSx1 | chr1 |
| AluSx3 | chr1 | 10043241 | 10043430 | + | AluSx1 | chr1 |
| AluSx | chr1 | 10515891 | 10516199 | + | AluSx1 | chr1 |
| AluSx | chr1 | 10515891 | 10516199 | + | AluSx3 | chr1 |
| AluJr4 | chr1 | 10517205 | 10517422 | − | AluSx | chr1 |
| AluSc | chr1 | 11080684 | 11080980 | + | AluSq2 | chr1 |
| AluSq | chr1 | 11087999 | 11088284 | + | AluY | chr1 |
| AluSq | chr1 | 11087999 | 11088284 | + | AluSp | chr1 |
| AluSx | chr1 | 11117833 | 11118135 | − | AluSp | chr1 |
| AluSx | chr1 | 15955809 | 15956109 | + | AluSq2 | chr1 |
| AluSq2 | chr1 | 16150335 | 16150642 | − | AluSc5 | chr1 |
| AluSz | chr1 | 19617369 | 19617682 | + | AluSz6 | chr1 |
| AluY | chr1 | 19625648 | 19625972 | + | AluSg7 | chr1 |
| AluSz | chr1 | 20471289 | 20471601 | + | AluSc | chr1 |
| AluSz6 | chr1 | 20975976 | 20976279 | + | AluY | chr1 |
| AluSz | chr1 | 21492937 | 21493256 | + | AluJb | chr1 |
| AluSz | chr1 | 21492937 | 21493256 | + | AluJb | chr1 |
| AluY | chr1 | 21499038 | 21499319 | − | AluSc8 | chr1 |
| AluSq | chr1 | 22387619 | 22387923 | + | AluSx1 | chr1 |
| AluSq | chr1 | 22387619 | 22387923 | + | AluSz | chr1 |
| AluSx | chr1 | 23060913 | 23061214 | − | AluJr | chr1 |
| AluSp | chr1 | 24299239 | 24299535 | − | AluSx | chr1 |
| AluSx3 | chr1 | 24300483 | 24300753 | − | AluSx | chr1 |
| AluJr | chr1 | 26764934 | 26765227 | + | AluSx1 | chr1 |
| AluJr | chr1 | 26764934 | 26765227 | + | AluSg | chr1 |
| AluSq2 | chr1 | 27202704 | 27203008 | − | AluSg7 | chr1 |
| AluSg7 | chr1 | 27204245 | 27204534 | + | AluSc5 | chr1 |
| AluSx1 | chr1 | 27281042 | 27281341 | + | AluSx1 | chr1 |
| AluSg | chr1 | 28825313 | 28825598 | + | AluSp | chr1 |
| AluSg | chr1 | 28825313 | 28825598 | + | AluJb | chr1 |
| AluSx | chr1 | 28839716 | 28840006 | − | AluSx4 | chr1 |
| AluSc | chr1 | 28866585 | 28866878 | − | AluSz | chr1 |
| AluSx3 | chr1 | 28866285 | 28866582 | − | AluSz | chr1 |
| AluSz | chr1 | 28866926 | 28867215 | + | AluSx | chr1 |
| AluSz | chr1 | 28866926 | 28867215 | + | AluJb | chr1 |
| AluJb | chr1 | 28869950 | 28870248 | − | AluSx | chr1 |
| AluSx | chr1 | 28871926 | 28872220 | + | AluSz | chr1 |
| AluY | chr1 | 28889077 | 28889390 | − | AluSx1 | chr1 |
| AluY | chr1 | 28889077 | 28889390 | − | AluSx | chr1 |
| AluSq | chr1 | 28896762 | 28897059 | − | AluY | chr1 |
| AluY | chr1 | 28899165 | 28899466 | + | AluSg | chr1 |
| AluSg | chr1 | 28952499 | 28952789 | − | AluSq | chr1 |
| AluSx3 | chr1 | 28963897 | 28964185 | + | AluSx | chr1 |
| AluSx3 | chr1 | 28963897 | 28964185 | + | AluYc | chr1 |
| AluYc | chr1 | 28965300 | 28965585 | − | AluSq | chr1 |
| AluSx | chr1 | 28965004 | 28965297 | − | AluSq | chr1 |
| AluSx | chr1 | 28965004 | 28965297 | − | AluJb | chr1 |
| AluYc | chr1 | 28965300 | 28965585 | − | AluJb | chr1 |
| AluSz | chr1 | 29500002 | 29500261 | − | AluSx1 | chr1 |
| AluSg | chr1 | 31419204 | 31419499 | + | AluJb | chr1 |
| AluSx1 | chr1 | 31522671 | 31522965 | − | AluSz6 | chr1 |
| AluSz6 | chr1 | 31523051 | 31523332 | + | AluJb | chr1 |
| AluJb | chr1 | 31526324 | 31526622 | − | AluSx | chr1 |
| AluSq2 | chr1 | 32707712 | 32708016 | + | AluY | chr1 |
| AluSq2 | chr1 | 33124880 | 33125186 | + | AluSq2 | chr1 |
| AluSx3 | chr1 | 33139037 | 33139336 | − | AluY | chr1 |
| AluSx3 | chr1 | 33139037 | 33139336 | − | AluY | chr1 |
| AluY | chr1 | 33141089 | 33141390 | + | AluSx1 | chr1 |
| AluSg4 | chr1 | 33148092 | 33148383 | + | AluSx1 | chr1 |
| AluSx | chr1 | 35565469 | 35565791 | − | AluSz6 | chr1 |
| AluSz6 | chr1 | 35565941 | 35566237 | + | AluJb | chr1 |
| AluSz6 | chr1 | 35565941 | 35566237 | + | AluSx1 | chr1 |
| AluSx3 | chr1 | 36763046 | 36763343 | − | AluSc | chr1 |
| AluSx1 | chr1 | 39461431 | 39461713 | + | AluSx | chr1 |
| AluSx1 | chr1 | 39461431 | 39461713 | + | AluSc8 | chr1 |
| AluSg | chr1 | 40508866 | 40509166 | − | AluSz | chr1 |
| AluSz | chr1 | 40509267 | 40509572 | + | AluSz | chr1 |
| AluSx1 | chr1 | 41163753 | 41164050 | + | AluSx | chr1 |
| AluSx | chr1 | 41164122 | 41164358 | − | AluSx3 | chr1 |
| AluJb | chr1 | 44459836 | 44460098 | + | AluY | chr1 |
| AluSp | chr1 | 45985255 | 45985564 | − | AluSg | chr1 |
| AluSx4 | chr1 | 46075591 | 46075887 | + | AluSx | chr1 |
| AluJo | chr1 | 46160831 | 46161130 | − | AluSp | chr1 |
| AluY | chr1 | 46817332 | 46817635 | − | AluSx | chr1 |
| AluSq | chr1 | 51419125 | 51419438 | + | AluY | chr1 |
| AluSz | chr1 | 52875001 | 52875288 | + | AluSx1 | chr1 |
| AluSz6 | chr1 | 62181055 | 62181347 | + | AluY | chr1 |
| AluJb | chr1 | 70649228 | 70649535 | − | AluSz | chr1 |
| AluJb | chr1 | 92986516 | 92986819 | − | AluSz | chr1 |
| AluSg | chr1 | 93800889 | 93801190 | + | AluYc | chr1 |
| AluJb | chr1 | 1.01E+08 | 1.01E+08 | − | AluSc | chr1 |
| AluSz6 | chr1 | 1.1E+08 | 1.1E+08 | − | AluSc | chr1 |
| AluJr | chr1 | 1.1E+08 | 1.1E+08 | + | AluJb | chr1 |
| AluSz | chr1 | 1.1E+08 | 1.1E+08 | − | AluSx1 | chr1 |
| AluSz | chr1 | 1.11E+08 | 1.11E+08 | − | AluSx1 | chr1 |
| AluJo | chr1 | 1.11E+08 | 1.11E+08 | − | AluSx1 | chr1 |
| AluSz | chr1 | 1.11E+08 | 1.11E+08 | − | AluSc8 | chr1 |
| AluJb | chr1 | 1.13E+08 | 1.13E+08 | − | AluSx | chr1 |
| AluSz6 | chr1 | 1.13E+08 | 1.13E+08 | + | AluSx | chr1 |
| AluSq2 | chr1 | 1.13E+08 | 1.13E+08 | + | AluSx1 | chr1 |
| AluSx | chr1 | 1.14E+08 | 1.14E+08 | − | AluSc8 | chr1 |
| AluSq2 | chr1 | 1.14E+08 | 1.14E+08 | − | AluY | chr1 |
| AluSx3 | chr1 | 1.46E+08 | 1.46E+08 | − | AluJr | chr1 |
| AluSc8 | chr1 | 1.46E+08 | 1.46E+08 | − | AluSq | chr1 |
| AluY | chr1 | 1.48E+08 | 1.48E+08 | + | AluSx | chr1 |
| AluSq2 | chr1 | 1.5E+08 | 1.5E+08 | + | AluY | chr1 |
| AluSz | chr1 | 1.5E+08 | 1.5E+08 | + | AluSq2 | chr1 |
| AluSz | chr1 | 1.5E+08 | 1.5E+08 | + | AluY | chr1 |
| AluSq2 | chr1 | 1.5E+08 | 1.5E+08 | − | AluSx | chr1 |
| AluY | chr1 | 1.51E+08 | 1.51E+08 | − | AluJb | chr1 |
| AluSq | chr1 | 1.51E+08 | 1.51E+08 | − | AluSg | chr1 |
| AluSx1 | chr1 | 1.51E+08 | 1.51E+08 | − | AluSg | chr1 |
| AluSq | chr1 | 1.51E+08 | 1.51E+08 | − | AluSz | chr1 |
| AluSg | chr1 | 1.51E+08 | 1.51E+08 | + | AluSq2 | chr1 |
| AluSz | chr1 | 1.51E+08 | 1.51E+08 | + | AluSq2 | chr1 |
| AluSp | chr1 | 1.51E+08 | 1.51E+08 | − | AluSz | chr1 |
| AluSc | chr1 | 1.51E+08 | 1.51E+08 | + | AluSp | chr1 |
| AluSp | chr1 | 1.51E+08 | 1.51E+08 | − | AluSz6 | chr1 |
| AluSp | chr1 | 1.51E+08 | 1.51E+08 | − | AluY | chr1 |
| AluY | chr1 | 1.51E+08 | 1.51E+08 | + | AluSg | chr1 |
| AluSx | chr1 | 1.51E+08 | 1.51E+08 | + | AluSg | chr1 |
| AluSx | chr1 | 1.51E+08 | 1.51E+08 | + | AluSp | chr1 |
| AluSz6 | chr1 | 1.51E+08 | 1.51E+08 | − | AluSz | chr1 |
| AluSz6 | chr1 | 1.51E+08 | 1.51E+08 | − | AluSq2 | chr1 |
| AluSz | chr1 | 1.51E+08 | 1.51E+08 | + | AluSq2 | chr1 |
| AluSq2 | chr1 | 1.51E+08 | 1.51E+08 | + | AluSq2 | chr1 |
| AluSq2 | chr1 | 1.51E+08 | 1.51E+08 | − | AluSq2 | chr1 |
| AluSx1 | chr1 | 1.51E+08 | 1.51E+08 | + | AluSq | chr1 |
| AluSx1 | chr1 | 1.51E+08 | 1.51E+08 | + | AluSp | chr1 |
| AluSq2 | chr1 | 1.51E+08 | 1.51E+08 | + | AluSg | chr1 |
| AluSz6 | chr1 | 1.55E+08 | 1.55E+08 | + | AluSx | chr1 |
| AluSx | chr1 | 1.55E+08 | 1.55E+08 | − | AluY | chr1 |
| AluSp | chr1 | 1.55E+08 | 1.55E+08 | − | AluSx | chr1 |
| AluSx | chr1 | 1.55E+08 | 1.55E+08 | + | AluSz6 | chr1 |
| AluJb | chr1 | 1.55E+08 | 1.55E+08 | + | AluSz | chr1 |
| AluSx | chr1 | 1.56E+08 | 1.56E+08 | − | AluSp | chr1 |
| AluSq2 | chr1 | 1.56E+08 | 1.56E+08 | + | AluSx3 | chr1 |
| AluSx | chr1 | 1.57E+08 | 1.57E+08 | + | AluSx | chr1 |
| AluSp | chr1 | 1.61E+08 | 1.61E+08 | + | AluY | chr1 |
| AluSp | chr1 | 1.61E+08 | 1.61E+08 | + | AluSg | chr1 |
| AluSg | chr1 | 1.61E+08 | 1.61E+08 | − | AluSq2 | chr1 |
| AluY | chr1 | 1.61E+08 | 1.61E+08 | − | AluSq2 | chr1 |
| AluSg | chr1 | 1.61E+08 | 1.61E+08 | − | AluSz | chr1 |
| AluY | chr1 | 1.61E+08 | 1.61E+08 | − | AluSz | chr1 |
| AluSq2 | chr1 | 1.61E+08 | 1.61E+08 | + | AluSx1 | chr1 |
| AluSz | chr1 | 1.61E+08 | 1.61E+08 | + | AluSx1 | chr1 |
| AluSg | chr1 | 1.61E+08 | 1.61E+08 | + | AluSp | chr1 |
| AluSx3 | chr1 | 1.61E+08 | 1.61E+08 | + | AluJb | chr1 |
| AluY | chr1 | 1.61E+08 | 1.61E+08 | + | AluJb | chr1 |
| AluSz6 | chr1 | 1.76E+08 | 1.76E+08 | − | AluSx4 | chr1 |
| AluJb | chr1 | 1.76E+08 | 1.76E+08 | + | AluJb | chr1 |
| AluSx1 | chr1 | 1.79E+08 | 1.79E+08 | − | AluJb | chr1 |
| AluSg | chr1 | 2.02E+08 | 2.02E+08 | + | AluSx | chr1 |
| AluSg | chr1 | 2.02E+08 | 2.02E+08 | + | AluSq2 | chr1 |
| AluY | chr1 | 2.03E+08 | 2.03E+08 | − | AluSz | chr1 |
| AluSq2 | chr1 | 2.03E+08 | 2.03E+08 | − | AluSx | chr1 |
| AluYc | chr1 | 2.03E+08 | 2.03E+08 | − | AluJb | chr1 |
| AluSx1 | chr1 | 2.04E+08 | 2.04E+08 | + | AluY | chr1 |
| AluSx1 | chr1 | 2.04E+08 | 2.04E+08 | + | AluSz | chr1 |
| AluSx | chr1 | 2.05E+08 | 2.05E+08 | + | AluSx | chr1 |
| AluJb | chr1 | 2.05E+08 | 2.05E+08 | + | AluSx | chr1 |
| AluJr4 | chr1 | 2.12E+08 | 2.12E+08 | + | AluY | chr1 |
| AluSp | chr1 | 2.12E+08 | 2.12E+08 | + | AluSc | chr1 |
| AluSx | chr1 | 2.13E+08 | 2.13E+08 | − | AluSx1 | chr1 |
| AluSz | chr1 | 2.18E+08 | 2.18E+08 | − | AluSz6 | chr1 |
| AluJb | chr1 | 2.22E+08 | 2.22E+08 | − | AluSg | chr1 |
| AluJo | chr1 | 2.26E+08 | 2.26E+08 | − | AluSx1 | chr1 |
| AluSq4 | chr1 | 2.26E+08 | 2.26E+08 | − | AluSp | chr1 |
| AluSx1 | chr1 | 2.3E+08 | 2.3E+08 | − | AluJb | chr1 |
| AluSx1 | chr1 | 2.31E+08 | 2.31E+08 | − | AluSx1 | chr1 |
| AluY | chr1 | 2.35E+08 | 2.35E+08 | − | AluSg | chr1 |
| AluSx | chr1 | 2.35E+08 | 2.35E+08 | − | AluSg | chr1 |
| AluSc8 | chr1 | 2.35E+08 | 2.35E+08 | + | AluSx3 | chr1 |
| AluSg7 | chr1 | 2.35E+08 | 2.35E+08 | − | AluJb | chr1 |
| AluSx | chr1 | 2.36E+08 | 2.36E+08 | + | AluSg4 | chr1 |
| AluSz | chr1 | 2.4E+08 | 2.4E+08 | − | AluSx1 | chr1 |
| AluSx1 | chr1 | 2.44E+08 | 2.44E+08 | − | AluSg | chr1 |
| AluSz | chr1 | 2.47E+08 | 2.47E+08 | + | AluJb | chr1 |
| AluSz | chr1 | 2.47E+08 | 2.47E+08 | − | AluSp | chr1 |
| AluSz6 | chr1 | 2.48E+08 | 2.48E+08 | − | AluSq | chr1 |
| AluSx1 | chr19 | 1032777 | 1033077 | − | AluJo | chr19 |
| AluSx1 | chr19 | 1586671 | 1586965 | − | AluSq2 | chr19 |
| AluY | chr19 | 1586969 | 1587263 | − | AluSq2 | chr19 |
| AluJr4 | chr19 | 1587404 | 1587707 | − | AluSq2 | chr19 |
| AluSq4 | chr19 | 1777713 | 1778017 | + | AluJo | chr19 |
| AluSx3 | chr19 | 1824783 | 1825035 | − | AluSx1 | chr19 |
| AluSc | chr19 | 2133827 | 2134124 | + | AluSx | chr19 |
| AluJb | chr19 | 2134139 | 2134443 | + | AluSx | chr19 |
| AluSx | chr19 | 2134607 | 2134899 | − | AluSx | chr19 |
| AluSg | chr19 | 2419197 | 2419499 | − | AluSq | chr19 |
| AluSq | chr19 | 2420921 | 2421218 | + | AluY | chr19 |
| AluSc5 | chr19 | 3540258 | 3540552 | − | AluSx | chr19 |
| AluSc5 | chr19 | 3540258 | 3540552 | − | AluSg4 | chr19 |
| AluJr | chr19 | 3758257 | 3758556 | − | AluSg | chr19 |
| AluSx | chr19 | 5020876 | 5021177 | + | AluSc | chr19 |
| AluJb | chr19 | 7084367 | 7084663 | − | AluSx1 | chr19 |
| AluY | chr19 | 7086364 | 7086670 | − | AluSx1 | chr19 |
| AluJb | chr19 | 7085194 | 7085474 | − | AluSx1 | chr19 |
| AluSp | chr19 | 9635176 | 9635484 | + | AluY | chr19 |
| AluJo | chr19 | 9672281 | 9672596 | + | AluSx3 | chr19 |
| AluSx3 | chr19 | 9672838 | 9673138 | − | AluSx | chr19 |
| AluSz | chr19 | 9935767 | 9936062 | + | AluSx3 | chr19 |
| AluSx | chr19 | 10681249 | 10681426 | + | AluJr | chr19 |
| AluJb | chr19 | 10768578 | 10768875 | − | AluSz | chr19 |
| AluSg7 | chr19 | 10917916 | 10918215 | − | AluSx1 | chr19 |
| AluSx1 | chr19 | 10918216 | 10918499 | − | AluSx1 | chr19 |
| AluSx | chr19 | 11040938 | 11041249 | − | AluY | chr19 |
| AluY | chr19 | 11041824 | 11042126 | + | AluSq | chr19 |
| AluY | chr19 | 11041824 | 11042126 | + | AluSg4 | chr19 |
| AluSg4 | chr19 | 11043190 | 11043489 | − | AluJb | chr19 |
| AluSq | chr19 | 11042299 | 11042604 | − | AluJb | chr19 |
| AluSx | chr19 | 11040938 | 11041249 | − | AluJb | chr19 |
| AluSx1 | chr19 | 11206227 | 11206434 | − | AluJb | chr19 |
| AluSp | chr19 | 11934497 | 11934795 | + | AluSg | chr19 |
| AluSq2 | chr19 | 11933381 | 11933676 | + | AluSg | chr19 |
| AluSx3 | chr19 | 11944935 | 11945240 | − | AluSq2 | chr19 |
| AluSx3 | chr19 | 11944935 | 11945240 | − | AluY | chr19 |
| AluSx3 | chr19 | 12024198 | 12024503 | − | AluSq2 | chr19 |
| AluY | chr19 | 12045714 | 12046009 | + | AluSg | chr19 |
| AluY | chr19 | 12188646 | 12188943 | − | AluSz | chr19 |
| AluY | chr19 | 12188646 | 12188943 | − | AluSq2 | chr19 |
| AluY | chr19 | 12188646 | 12188943 | − | AluY | chr19 |
| AluSz | chr19 | 12190194 | 12190472 | + | AluSz | chr19 |
| AluSq2 | chr19 | 12190479 | 12190781 | + | AluSz | chr19 |
| AluY | chr19 | 12190882 | 12191186 | + | AluSz | chr19 |
| AluY | chr19 | 12424517 | 12424817 | − | AluSp | chr19 |
| AluSz | chr19 | 12634223 | 12634524 | − | AluSz6 | chr19 |
| AluY | chr19 | 12742780 | 12743076 | − | AluSx1 | chr19 |
| AluSq | chr19 | 12842655 | 12842968 | + | AluSx | chr19 |
| AluY | chr19 | 13846585 | 13846879 | + | AluSx1 | chr19 |
| AluSx1 | chr19 | 13847061 | 13847373 | − | AluSx1 | chr19 |
| AluSz | chr19 | 13883818 | 13884121 | − | AluJr | chr19 |
| AluJr | chr19 | 13911674 | 13911986 | + | AluY | chr19 |
| AluY | chr19 | 13912788 | 13913089 | − | AluSx | chr19 |
| AluSz | chr19 | 14657117 | 14657346 | + | AluSg | chr19 |
| AluSz | chr19 | 16661260 | 16661541 | + | AluSz | chr19 |
| AluSz | chr19 | 16661260 | 16661541 | + | AluSg | chr19 |
| AluSz6 | chr19 | 16743347 | 16743642 | − | AluSx1 | chr19 |
| AluSz6 | chr19 | 16743347 | 16743642 | − | AluSz6 | chr19 |
| AluY | chr19 | 16749932 | 16750232 | − | AluSx1 | chr19 |
| AluY | chr19 | 16749932 | 16750232 | − | AluSx1 | chr19 |
| AluSz | chr19 | 16793782 | 16794080 | − | AluJo | chr19 |
| AluSc5 | chr19 | 17348221 | 17348518 | − | AluJo | chr19 |
| AluSp | chr19 | 17531991 | 17532292 | − | AluSx1 | chr19 |
| AluSz6 | chr19 | 17925110 | 17925419 | − | AluJb | chr19 |
| AluSz6 | chr19 | 17925110 | 17925419 | − | AluSx | chr19 |
| AluSx1 | chr19 | 18475142 | 18475430 | + | AluSx1 | chr19 |
| AluSx1 | chr19 | 18477274 | 18477580 | − | AluJo | chr19 |
| AluSx1 | chr19 | 18477274 | 18477580 | − | AluSz6 | chr19 |
| AluSx1 | chr19 | 18670817 | 18671117 | − | AluSq2 | chr19 |
| AluSg | chr19 | 18671522 | 18671814 | − | AluSq2 | chr19 |
| AluY | chr19 | 19432804 | 19433106 | − | AluJb | chr19 |
| AluY | chr19 | 19512022 | 19512320 | − | AluSc | chr19 |
| AluSc | chr19 | 19513216 | 19513522 | + | AluSx | chr19 |
| AluSg | chr19 | 19695433 | 19695720 | − | AluSx | chr19 |
| AluSc5 | chr19 | 19791456 | 19791747 | + | AluSx3 | chr19 |
| AluJb | chr19 | 19792278 | 19792562 | + | AluSx3 | chr19 |
| AluJb | chr19 | 19792278 | 19792562 | + | AluSz | chr19 |
| AluSc5 | chr19 | 19791456 | 19791747 | + | AluSz | chr19 |
| AluSc5 | chr19 | 19791456 | 19791747 | + | AluSc8 | chr19 |
| AluJb | chr19 | 19792278 | 19792562 | + | AluSc8 | chr19 |
| AluSp | chr19 | 19898169 | 19898483 | + | AluSq2 | chr19 |
| AluSq2 | chr19 | 19899197 | 19899498 | − | AluSq | chr19 |
| AluSp | chr19 | 19898169 | 19898483 | + | AluSx | chr19 |
| AluSq | chr19 | 19899949 | 19900249 | + | AluSx | chr19 |
| AluSx | chr19 | 19900469 | 19900766 | − | AluY | chr19 |
| AluSx3 | chr19 | 19911235 | 19911523 | − | AluSx | chr19 |
| AluSq2 | chr19 | 20309320 | 20309617 | − | AluSp | chr19 |
| AluSg | chr19 | 20335937 | 20336246 | + | AluY | chr19 |
| AluSp | chr19 | 20842793 | 20843074 | + | AluSg | chr19 |
| AluSc8 | chr19 | 21354516 | 21354822 | − | AluSz | chr19 |
| AluSq2 | chr19 | 21469709 | 21470014 | − | AluSg | chr19 |
| AluY | chr19 | 21473395 | 21473692 | + | AluSc | chr19 |
| AluSg | chr19 | 21472798 | 21473091 | + | AluSc | chr19 |
| AluSg | chr19 | 21474387 | 21474690 | + | AluSc | chr19 |
| AluSx1 | chr19 | 21589874 | 21590068 | + | AluY | chr19 |
| AluJo | chr19 | 21924485 | 21924782 | − | AluSx1 | chr19 |
| AluSx3 | chr19 | 24237727 | 24238034 | + | AluSg | chr19 |
| AluSz | chr19 | 33528976 | 33529186 | + | AluY | chr19 |
| AluSc | chr19 | 34677863 | 34678168 | + | AluSx | chr19 |
| AluJr | chr19 | 36253331 | 36253652 | + | AluSx | chr19 |
| AluSg | chr19 | 36253655 | 36253948 | + | AluSx | chr19 |
| AluSg | chr19 | 36253655 | 36253948 | + | AluY | chr19 |
| AluJr | chr19 | 36253331 | 36253652 | + | AluY | chr19 |
| AluSx | chr19 | 36808056 | 36808359 | − | AluSz6 | chr19 |
| AluSx | chr19 | 36808056 | 36808359 | − | AluJb | chr19 |
| AluJb | chr19 | 36810144 | 36810442 | + | AluY | chr19 |
| AluY | chr19 | 36810716 | 36811010 | − | AluY | chr19 |
| AluSx1 | chr19 | 37817881 | 37818182 | − | AluSp | chr19 |
| AluSz | chr19 | 37817567 | 37817863 | − | AluSp | chr19 |
| AluSp | chr19 | 38220597 | 38220908 | + | AluSz | chr19 |
| AluSp | chr19 | 38220597 | 38220908 | + | AluSz | chr19 |
| AluSz | chr19 | 38222629 | 38222917 | − | AluSc | chr19 |
| AluSx | chr19 | 38592068 | 38592350 | − | AluSq | chr19 |
| AluSx1 | chr19 | 39159632 | 39159925 | − | AluSc5 | chr19 |
| AluY | chr19 | 39319248 | 39319560 | − | AluSx1 | chr19 |
| AluSp | chr19 | 39981381 | 39981686 | + | AluSp | chr19 |
| AluSp | chr19 | 39981381 | 39981686 | + | AluSx | chr19 |
| AluJr | chr19 | 43022299 | 43022589 | + | AluY | chr19 |
| AluSp | chr19 | 44801625 | 44801914 | − | AluJb | chr19 |
| AluSx | chr19 | 45173665 | 45173933 | + | AluSg | chr19 |
| AluSx1 | chr19 | 45298217 | 45298513 | − | AluJr | chr19 |
| AluSg | chr19 | 45949976 | 45950277 | + | AluSq | chr19 |
| AluSx | chr19 | 46360093 | 46360384 | + | AluSp | chr19 |
| AluSz6 | chr19 | 47246875 | 47247189 | + | AluJo | chr19 |
| AluSz6 | chr19 | 47283601 | 47283914 | − | AluSz6 | chr19 |
| AluSz6 | chr19 | 47283946 | 47284255 | + | AluSz | chr19 |
| AluSx | chr19 | 47374599 | 47374899 | − | AluSx1 | chr19 |
| AluSq | chr19 | 47796892 | 47797184 | + | AluSx1 | chr19 |
| AluSz | chr19 | 48119622 | 48119917 | + | AluSz6 | chr19 |
| AluY | chr19 | 48962217 | 48962518 | + | AluJo | chr19 |
| AluJo | chr19 | 48962664 | 48962951 | − | AluSz | chr19 |
| AluSz | chr19 | 48963004 | 48963291 | + | AluSp | chr19 |
| AluY | chr19 | 48962217 | 48962518 | + | AluSp | chr19 |
| AluSx | chr19 | 48962001 | 48962216 | + | AluSp | chr19 |
| AluSg | chr19 | 49126011 | 49126217 | − | AluSc8 | chr19 |
| AluJb | chr19 | 49212265 | 49212448 | + | AluSp | chr19 |
| AluJb | chr19 | 49647689 | 49647981 | + | AluSx | chr19 |
| AluSg | chr19 | 51305759 | 51306042 | + | AluJo | chr19 |
| AluSq2 | chr19 | 52566605 | 52566900 | − | AluYb8 | chr19 |
| AluYb8 | chr19 | 52567316 | 52567635 | + | AluSx1 | chr19 |
| AluY | chr19 | 52655998 | 52656290 | − | AluSc | chr19 |
| AluSx1 | chr19 | 52696834 | 52697108 | − | AluJo | chr19 |
| AluSq2 | chr19 | 53070211 | 53070504 | − | AluSx | chr19 |
| AluSq2 | chr19 | 53147986 | 53148284 | + | AluSx | chr19 |
| AluSc8 | chr19 | 53146674 | 53146973 | + | AluSx | chr19 |
| AluSp | chr19 | 53153506 | 53153809 | − | AluSg | chr19 |
| AluSg | chr19 | 53205603 | 53205900 | + | AluSx | chr19 |
| AluSp | chr19 | 53204772 | 53205081 | + | AluSx | chr19 |
| AluSq2 | chr19 | 53205271 | 53205587 | + | AluSx | chr19 |
| AluSp | chr19 | 53204772 | 53205081 | + | AluY | chr19 |
| AluSp | chr19 | 53429708 | 53430016 | − | AluSp | chr19 |
| AluSc8 | chr19 | 53430020 | 53430314 | − | AluSp | chr19 |
| AluSg | chr19 | 53442897 | 53443207 | − | AluSp | chr19 |
| AluSg | chr19 | 53442897 | 53443207 | − | AluJb | chr19 |
| AluSp | chr19 | 53443238 | 53443538 | + | AluJb | chr19 |
| AluJb | chr19 | 53443584 | 53443856 | + | AluJb | chr19 |
| AluSx3 | chr19 | 53717339 | 53717635 | − | AluSz6 | chr19 |
| AluSc | chr19 | 53718425 | 53718729 | − | AluSz6 | chr19 |
| AluSg | chr19 | 54954218 | 54954508 | + | AluSz6 | chr19 |
| AluSz6 | chr19 | 54954733 | 54955026 | − | AluY | chr19 |
| AluSz6 | chr19 | 54954733 | 54955026 | − | AluSx4 | chr19 |
| AluSz6 | chr19 | 57727439 | 57727735 | + | AluSg | chr19 |
| AluSg | chr19 | 58355587 | 58355847 | + | AluSz | chr19 |
| AluSz | chr19 | 58373137 | 58373440 | − | AluSc | chr19 |
| AluSz | chr19 | 58372236 | 58372510 | − | AluSc | chr19 |
| AluSg7 | chr19 | 58376794 | 58377073 | + | AluJb | chr19 |
| AluSc | chr19 | 58375131 | 58375425 | + | AluJb | chr19 |
| AluJb | chr19 | 58377331 | 58377623 | − | AluY | chr19 |
| AluSx1 | chr19 | 58375497 | 58375798 | − | AluY | chr19 |
| AluSx1 | chr19 | 58375497 | 58375798 | − | AluSz | chr19 |
| AluJb | chr19 | 58377331 | 58377623 | − | AluSz | chr19 |
| AluSz | chr19 | 58378129 | 58378424 | + | AluSx | chr19 |
| AluY | chr19 | 58377770 | 58378063 | + | AluSx | chr19 |
| AluJb | chr19 | 58377331 | 58377623 | − | AluSp | chr19 |
| AluSx | chr19 | 58378732 | 58378945 | − | AluSp | chr19 |
| AluSp | chr19 | 58747589 | 58747898 | + | AluSx3 | chr19 |
| AluY | chr19 | 58765326 | 58765600 | + | AluY | chr19 |
| AluJb | chr19 | 58774546 | 58774820 | + | AluY | chr19 |
| AluJb | chr19 | 58776964 | 58777240 | − | AluSz | chr19 |
| AluY | chr19 | 58776687 | 58776960 | − | AluSz | chr19 |
| AluSg | chr19 | 58781671 | 58781965 | − | AluSx1 | chr19 |
| AluSz6 | chr19 | 58808317 | 58808624 | + | AluSz | chr19 |
| AluSz6 | chr19 | 58808317 | 58808624 | + | AluJb | chr19 |
| AluSp | chr19 | 59077532 | 59077829 | + | AluJb | chr19 |

| Starts | End2 | Repeat2_Sign | Repeat1_Strand | Repeat2_Strand |
|---|---|---|---|---|
| 20789015 | 20789312 | + | antiSense | antiSense |
| 20792348 | 20792644 | − | antiSense | antiSense |
| 21696962 | 21697264 | + | antiSense | antiSense |
| 21890218 | 21890518 | + | antiSense | antiSense |
| 21892623 | 21892929 | − | antiSense | antiSense |
| 21939830 | 21940134 | − | antiSense | antiSense |
| 23301936 | 23302106 | + | Sense | Sense |
| 23302107 | 23302410 | + | Sense | Sense |
| 23384706 | 23385002 | − | antiSense | antiSense |
| 23385156 | 23385426 | + | antiSense | antiSense |
| 23501803 | 23502105 | − | antiSense | antiSense |
| 31433177 | 31433407 | + | antiSense | antiSense |
| 31486080 | 31486368 | + | antiSense | antiSense |
| 34904824 | 34905134 | − | antiSense | antiSense |
| 34913678 | 34913971 | + | antiSense | antiSense |
| 34928705 | 34928987 | + | antiSense | antiSense |
| 35042830 | 35043144 | − | antiSense | antiSense |
| 35046008 | 35046283 | + | antiSense | antiSense |
| 35091505 | 35091795 | + | antiSense | antiSense |
| 35094131 | 35094420 | − | antiSense | antiSense |
| 35096371 | 35096673 | + | antiSense | antiSense |
| 35423146 | 35423450 | + | antiSense | antiSense |
| 35423146 | 35423450 | + | antiSense | antiSense |
| 35423548 | 35423848 | + | antiSense | antiSense |
| 35423548 | 35423848 | + | antiSense | antiSense |
| 35428084 | 35428387 | + | antiSense | antiSense |
| 35495869 | 35496172 | + | Sense | Sense |
| 35496229 | 35496535 | − | Sense | Sense |
| 36013972 | 36014254 | + | antiSense | antiSense |
| 45435393 | 45435680 | + | Sense | Sense |
| 45586193 | 45586474 | − | antiSense | antiSense |
| 50039905 | 50040210 | + | antiSense | antiSense |
| 50041758 | 50042059 | − | antiSense | antiSense |
| 50301210 | 50301501 | − | antiSense | antiSense |
| 50681545 | 50681852 | − | antiSense | antiSense |
| 50746884 | 50747177 | − | antiSense | antiSense |
| 50764689 | 50764978 | + | antiSense | antiSense |
| 50764689 | 50764978 | + | antiSense | antiSense |
| 50793784 | 50794076 | + | Sense | Sense |
| 50794178 | 50794468 | − | Sense | Sense |
| 51122484 | 51122780 | + | antiSense | antiSense |
| 51269791 | 51270089 | + | antiSense | antiSense |
| 51270603 | 51270913 | − | antiSense | antiSense |
| 55063980 | 55064290 | − | Sense | Sense |
| 55064303 | 55064604 | − | Sense | Sense |
| 55074542 | 55074754 | + | Sense | Sense |
| 55523226 | 55523526 | − | Sense | Sense |
| 56022345 | 56022641 | + | antiSense | antiSense |
| 57942508 | 57942808 | − | antiSense | antiSense |
| 58781557 | 58781852 | + | Sense | Sense |
| 58892924 | 58893220 | − | antiSense | antiSense |
| 60567016 | 60567312 | − | Sense | Sense |
| 61184456 | 61184725 | + | antiSense | antiSense |
| 61214953 | 61215248 | − | Sense | Sense |
| 61214953 | 61215248 | − | Sense | Sense |
| 63947188 | 63947463 | − | antiSense | antiSense |
| 63993606 | 63993900 | + | antiSense | antiSense |
| 63993606 | 63993900 | + | antiSense | antiSense |
| 64986200 | 64986517 | + | Sense | Sense |
| 69417283 | 69417577 | − | antiSense | antiSense |
| 71391304 | 71391609 | − | Sense | Sense |
| 71391700 | 71392004 | − | Sense | Sense |
| 71937906 | 71938184 | + | Sense | Sense |
| 71947198 | 71947510 | + | Sense | Sense |
| 73660368 | 73660664 | − | Sense | Sense |
| 73919817 | 73920138 | − | antiSense | antiSense |
| 75202830 | 75203127 | + | Sense | Sense |
| 75524390 | 75524694 | + | antiSense | antiSense |
| 78025382 | 78025675 | + | antiSense | antiSense |
| 92526616 | 92526902 | − | antiSense | antiSense |
| 92527079 | 92527375 | − | antiSense | antiSense |
| 92587224 | 92587529 | + | antiSense | antiSense |
| 92587224 | 92587529 | + | antiSense | antiSense |
| 92634309 | 92634610 | + | Sense | Sense |
| 93736759 | 93737057 | + | antiSense | antiSense |
| 1.01E+08 | 1.01E+08 | + | Sense | Sense |
| 1.01E+08 | 1.01E+08 | − | Sense | Sense |
| 1.03E+08 | 1.03E+08 | + | Sense | Sense |
| 1.03E+08 | 1.03E+08 | + | Sense | Sense |
| 1.03E+08 | 1.03E+08 | + | Sense | Sense |
| 1.03E+08 | 1.03E+08 | + | Sense | Sense |
| 1.03E+08 | 1.03E+08 | + | Sense | Sense |
| 1.03E+08 | 1.03E+08 | − | Sense | Sense |
| 1.03E+08 | 1.03E+08 | + | Sense | Sense |
| 1.04E+08 | 1.04E+08 | − | Sense | Sense |
| 1.04E+08 | 1.04E+08 | + | Sense | Sense |
| 1.04E+08 | 1.04E+08 | + | Sense | Sense |
| 1.04E+08 | 1.04E+08 | − | Sense | Sense |
| 1.04E+08 | 1.04E+08 | + | Sense | Sense |
| 1.04E+08 | 1.04E+08 | + | Sense | Sense |
| 1.04E+08 | 1.04E+08 | − | Sense | Sense |
| 1.04E+08 | 1.04E+08 | − | Sense | Sense |
| 1.04E+08 | 1.04E+08 | + | Sense | Sense |
| 1.04E+08 | 1.04E+08 | + | Sense | Sense |
| 1.04E+08 | 1.04E+08 | + | Sense | Sense |
| 1.04E+08 | 1.04E+08 | − | antiSense | antiSense |
| 1.04E+08 | 1.04E+08 | − | antiSense | antiSense |
| 1.04E+08 | 1.04E+08 | + | antiSense | antiSense |
| 1.04E+08 | 1.04E+08 | − | Sense | Sense |
| 1.05E+08 | 1.05E+08 | + | antiSense | antiSense |
| 1.06E+08 | 1.06E+08 | − | antiSense | antiSense |
| 1.07E+08 | 1.07E+08 | − | Sense | Sense |
| 177304 | 177606 | + | antiSense | antiSense |
| 8199712 | 8200011 | − | antiSense | antiSense |
| 8225158 | 8225456 | − | antiSense | antiSense |
| 8228383 | 8228667 | + | antiSense | antiSense |
| 8228781 | 8229074 | − | antiSense | antiSense |
| 8632435 | 8632726 | + | antiSense | antiSense |
| 8634849 | 8635159 | − | antiSense | antiSense |
| 8699693 | 8699998 | − | antiSense | antiSense |
| 8711092 | 8711347 | − | antiSense | antiSense |
| 11701258 | 11701561 | + | antiSense | antiSense |
| 11701258 | 11701561 | + | antiSense | antiSense |
| 23046914 | 23047206 | + | antiSense | antiSense |
| 25055805 | 25056100 | + | Sense | Sense |
| 28500922 | 28501234 | − | Sense | Sense |
| 33355332 | 33355634 | + | antiSense | antiSense |
| 38074395 | 38074699 | + | Sense | Sense |
| 38401580 | 38401890 | + | antiSense | antiSense |
| 38829696 | 38830003 | − | Sense | Sense |
| 41401222 | 41401525 | − | Sense | Sense |
| 42152964 | 42153256 | + | Sense | Sense |
| 42172011 | 42172318 | + | Sense | Sense |
| 42383804 | 42384106 | − | antiSense | antiSense |
| 42384132 | 42384411 | − | antiSense | antiSense |
| 42939361 | 42939660 | − | Sense | Sense |
| 42999180 | 42999486 | + | Sense | Sense |
| 48693219 | 48693509 | + | antiSense | antiSense |
| 48798172 | 48798475 | + | antiSense | antiSense |
| 48858608 | 48858885 | + | antiSense | antiSense |
| 48860187 | 48860490 | + | antiSense | antiSense |
| 54904893 | 54905174 | + | antiSense | antiSense |
| 54970821 | 54971122 | + | antiSense | antiSense |
| 54973647 | 54973940 | − | antiSense | antiSense |
| 56739076 | 56739369 | + | Sense | Sense |
| 56739076 | 56739369 | + | Sense | Sense |
| 56803630 | 56803930 | + | Sense | Sense |
| 56804699 | 56805000 | + | Sense | Sense |
| 56829765 | 56830032 | − | Sense | Sense |
| 56979211 | 56979520 | + | antiSense | antiSense |
| 56979963 | 56980265 | + | antiSense | antiSense |
| 56981920 | 56982157 | − | antiSense | antiSense |
| 57131883 | 57132171 | − | Sense | Sense |
| 64115519 | 64115815 | + | Sense | Sense |
| 66596969 | 66597252 | + | Sense | Sense |
| 66599583 | 66599879 | − | Sense | Sense |
| 66600053 | 66600351 | + | Sense | Sense |
| 66600354 | 66600654 | + | Sense | Sense |
| 66642881 | 66643183 | + | Sense | Sense |
| 67606847 | 67607144 | + | Sense | Sense |
| 74229874 | 74230154 | + | Sense | Sense |
| 74655695 | 74656006 | − | antiSense | antiSense |
| 74785610 | 74785902 | + | antiSense | antiSense |
| 80888658 | 80888970 | + | antiSense | antiSense |
| 81078857 | 81079157 | + | antiSense | antiSense |
| 82589333 | 82589629 | − | antiSense | antiSense |
| 82612893 | 82613202 | + | antiSense | antiSense |
| 82615652 | 82615957 | − | antiSense | antiSense |
| 82616490 | 82616794 | + | antiSense | antiSense |
| 95661730 | 95662040 | − | Sense | Sense |
| 95688722 | 95689025 | − | Sense | Sense |
| 95708009 | 95708309 | + | Sense | Sense |
| 95754616 | 95754787 | − | Sense | Sense |
| 95865257 | 95865562 | + | Sense | Sense |
| 95873790 | 95874078 | − | Sense | Sense |
| 95874175 | 95874469 | − | Sense | Sense |
| 95915316 | 95915616 | + | Sense | Sense |
| 96051401 | 96051691 | + | Sense | Sense |
| 98678073 | 98678374 | + | Sense | Sense |
| 98678925 | 98679224 | − | Sense | Sense |
| 98681864 | 98682166 | + | Sense | Sense |
| 98681864 | 98682166 | + | Sense | Sense |
| 98706870 | 98707167 | + | Sense | Sense |
| 1.02E+08 | 1.02E+08 | + | antiSense | antiSense |
| 1.03E+08 | 1.03E+08 | − | Sense | Sense |
| 1.03E+08 | 1.03E+08 | − | antiSense | antiSense |
| 1.03E+08 | 1.03E+08 | − | antiSense | antiSense |
| 1.03E+08 | 1.03E+08 | + | antiSense | antiSense |
| 1.03E+08 | 1.03E+08 | + | antiSense | antiSense |
| 1.04E+08 | 1.04E+08 | + | Sense | Sense |
| 1.21E+08 | 1.21E+08 | + | antiSense | antiSense |
| 1.21E+08 | 1.21E+08 | − | Sense | Sense |
| 1.24E+08 | 1.24E+08 | + | antiSense | antiSense |
| 1.24E+08 | 1.24E+08 | − | antiSense | antiSense |
| 1.26E+08 | 1.26E+08 | + | antiSense | antiSense |
| 1.26E+08 | 1.26E+08 | + | antiSense | antiSense |
| 1.26E+08 | 1.26E+08 | − | antiSense | antiSense |
| 1.26E+08 | 1.26E+08 | − | Sense | Sense |
| 1.26E+08 | 1.26E+08 | + | Sense | Sense |
| 1.26E+08 | 1.26E+08 | − | Sense | Sense |
| 1.26E+08 | 1.26E+08 | − | Sense | Sense |
| 1.28E+08 | 1.28E+08 | + | antiSense | antiSense |
| 1.28E+08 | 1.28E+08 | + | antiSense | antiSense |
| 1.28E+08 | 1.28E+08 | + | antiSense | antiSense |
| 1.29E+08 | 1.29E+08 | − | Sense | Sense |
| 1.29E+08 | 1.29E+08 | + | Sense | Sense |
| 1.29E+08 | 1.29E+08 | + | Sense | Sense |
| 1.29E+08 | 1.29E+08 | + | Sense | Sense |
| 1.29E+08 | 1.29E+08 | + | Sense | Sense |
| 1.29E+08 | 1.29E+08 | + | Sense | Sense |
| 1.29E+08 | 1.29E+08 | − | Sense | Sense |
| 1.29E+08 | 1.29E+08 | + | Sense | Sense |
| 1.29E+08 | 1.29E+08 | − | Sense | Sense |
| 1.31E+08 | 1.31E+08 | − | antiSense | antiSense |
| 1.31E+08 | 1.31E+08 | + | antiSense | antiSense |
| 1.31E+08 | 1.31E+08 | − | antiSense | antiSense |
| 1.34E+08 | 1.34E+08 | − | antiSense | antiSense |
| 1.35E+08 | 1.35E+08 | + | antiSense | antiSense |
| 1.42E+08 | 1.42E+08 | + | antiSense | antiSense |
| 1.42E+08 | 1.42E+08 | + | antiSense | antiSense |
| 1.42E+08 | 1.42E+08 | + | antiSense | antiSense |
| 1.42E+08 | 1.42E+08 | − | antiSense | antiSense |
| 1.42E+08 | 1.42E+08 | + | antiSense | antiSense |
| 1.42E+08 | 1.42E+08 | + | antiSense | antiSense |
| 1.42E+08 | 1.42E+08 | + | antiSense | antiSense |
| 1.44E+08 | 1.44E+08 | + | antiSense | antiSense |
| 1.45E+08 | 1.45E+08 | + | Sense | Sense |
| 1.45E+08 | 1.45E+08 | + | Sense | Sense |
| 1.45E+08 | 1.45E+08 | − | Sense | Sense |
| 1.46E+08 | 1.46E+08 | − | antiSense | antiSense |
| 1.46E+08 | 1.46E+08 | + | antiSense | antiSense |
| 1.46E+08 | 1.46E+08 | − | Sense | Sense |
| 1274008 | 1274313 | + | antiSense | antiSense |
| 1275134 | 1275431 | − | antiSense | antiSense |
| 1607237 | 1607547 | + | antiSense | antiSense |
| 1609778 | 1610097 | + | antiSense | antiSense |
| 2209729 | 2210029 | + | Sense | Sense |
| 2501259 | 2501567 | + | Sense | Sense |
| 2503266 | 2503539 | − | Sense | Sense |
| 4267884 | 4268176 | − | antiSense | antiSense |
| 4432704 | 4432988 | + | Sense | Sense |
| 4831979 | 4832251 | + | antiSense | antiSense |
| 4832253 | 4832454 | + | antiSense | antiSense |
| 4833045 | 4833344 | − | antiSense | antiSense |
| 4833045 | 4833344 | − | antiSense | antiSense |
| 4833776 | 4834008 | + | antiSense | antiSense |
| 4835057 | 4835351 | − | antiSense | antiSense |
| 4838318 | 4838622 | + | antiSense | antiSense |
| 4930410 | 4930724 | − | Sense | Sense |
| 4930992 | 4931272 | + | Sense | Sense |
| 6221649 | 6221939 | + | antiSense | antiSense |
| 8174987 | 8175282 | + | Sense | Sense |
| 8175301 | 8175606 | + | Sense | Sense |
| 8176111 | 8176418 | − | Sense | Sense |
| 8176111 | 8176418 | − | Sense | Sense |
| 12913494 | 12913785 | − | antiSense | antiSense |
| 15886908 | 15887200 | − | antiSense | Sense |
| 16250156 | 16250464 | − | antiSense | antiSense |
| 17737418 | 17737703 | − | antiSense | antiSense |
| 17871699 | 17871998 | + | antiSense | antiSense |
| 17872372 | 17872669 | − | antiSense | antiSense |
| 18177246 | 18177550 | − | antiSense | antiSense |
| 19157173 | 19157474 | + | Sense | Sense |
| 20511113 | 20511425 | − | antiSense | antiSense |
| 20804546 | 20804834 | − | Sense | Sense |
| 20804845 | 20805148 | − | Sense | Sense |
| 20944201 | 20944495 | − | antiSense | antiSense |
| 20944629 | 20944920 | + | antiSense | antiSense |
| 26480018 | 26480315 | + | Sense | Sense |
| 26936785 | 26937086 | + | antiSense | antiSense |
| 27119048 | 27119349 | + | antiSense | antiSense |
| 27121235 | 27121537 | − | antiSense | antiSense |
| 27121830 | 27122125 | + | antiSense | antiSense |
| 27131358 | 27131651 | − | antiSense | antiSense |
| 27274398 | 27274627 | + | antiSense | antiSense |
| 27498631 | 27498937 | + | antiSense | antiSense |
| 27500411 | 27500703 | − | antiSense | antiSense |
| 27500799 | 27501089 | − | antiSense | antiSense |
| 27501552 | 27501842 | + | antiSense | antiSense |
| 27501552 | 27501842 | + | antiSense | antiSense |
| 27502371 | 27502681 | − | antiSense | antiSense |
| 27502796 | 27503083 | + | antiSense | antiSense |
| 27502796 | 27503083 | + | antiSense | antiSense |
| 27502796 | 27503083 | + | antiSense | antiSense |
| 27599256 | 27599557 | − | antiSense | antiSense |
| 27599256 | 27599557 | − | antiSense | antiSense |
| 27606833 | 27607132 | − | antiSense | antiSense |
| 27607723 | 27608026 | + | antiSense | antiSense |
| 27608791 | 27609078 | − | antiSense | antiSense |
| 27609297 | 27609600 | + | antiSense | antiSense |
| 27609297 | 27609600 | + | antiSense | antiSense |
| 27732515 | 27732807 | + | Sense | Sense |
| 27752100 | 27752390 | − | Sense | Sense |
| 28080533 | 28080834 | + | antiSense | antiSense |
| 28777599 | 28777895 | + | Sense | Sense |
| 29063946 | 29064240 | − | Sense | Sense |
| 29063946 | 29064240 | − | Sense | Sense |
| 29064241 | 29064535 | − | Sense | Sense |
| 29064241 | 29064535 | − | Sense | Sense |
| 29078929 | 29079193 | − | Sense | Sense |
| 29081226 | 29081529 | + | Sense | Sense |
| 29081226 | 29081529 | + | Sense | Sense |
| 29082885 | 29083177 | − | Sense | Sense |
| 29097974 | 29098271 | + | Sense | Sense |
| 29110361 | 29110651 | + | Sense | Sense |
| 29111777 | 29112065 | − | Sense | Sense |
| 29114736 | 29115026 | + | Sense | Sense |
| 29115065 | 29115371 | + | Sense | Sense |
| 29144174 | 29144464 | + | antiSense | antiSense |
| 29144174 | 29144464 | + | antiSense | antiSense |
| 29145461 | 29145771 | − | antiSense | antiSense |
| 29193551 | 29193853 | + | Sense | Sense |
| 29459278 | 29459570 | + | Sense | Sense |
| 30189585 | 30189892 | − | antiSense | antiSense |
| 30287621 | 30287896 | − | Sense | Sense |
| 30287951 | 30288245 | + | Sense | Sense |
| 30544852 | 30545181 | − | Sense | Sense |
| 30656869 | 30657161 | − | antiSense | antiSense |
| 30799892 | 30800196 | + | Sense | Sense |
| 33382412 | 33382728 | + | antiSense | antiSense |
| 33405320 | 33405620 | − | antiSense | antiSense |
| 33898360 | 33898651 | + | Sense | Sense |
| 33937645 | 33937943 | + | Sense | Sense |
| 34873651 | 34873946 | + | antiSense | antiSense |
| 34875832 | 34876107 | − | antiSense | antiSense |
| 34924969 | 34925254 | + | Sense | Sense |
| 35313691 | 35313988 | + | Sense | Sense |
| 35313995 | 35314306 | + | Sense | Sense |
| 35682866 | 35683166 | + | antiSense | antiSense |
| 35683488 | 35683797 | + | antiSense | antiSense |
| 35993966 | 35994278 | − | antiSense | antiSense |
| 36395025 | 36395305 | + | antiSense | antiSense |
| 36395025 | 36395305 | + | antiSense | antiSense |
| 36880327 | 36880535 | + | Sense | Sense |
| 37413229 | 37413484 | − | antiSense | antiSense |
| 37551396 | 37551694 | + | antiSense | antiSense |
| 37599250 | 37599552 | − | antiSense | antiSense |
| 38439491 | 38439798 | − | Sense | Sense |
| 38632836 | 38633133 | + | antiSense | antiSense |
| 39931076 | 39931374 | + | antiSense | antiSense |
| 39935586 | 39935879 | − | antiSense | antiSense |
| 39990366 | 39990644 | + | antiSense | antiSense |
| 40008761 | 40009054 | + | antiSense | antiSense |
| 40031320 | 40031618 | + | antiSense | antiSense |
| 40160435 | 40160727 | − | antiSense | antiSense |
| 40160435 | 40160727 | − | antiSense | antiSense |
| 40160435 | 40160727 | − | antiSense | antiSense |
| 40470977 | 40471277 | + | antiSense | antiSense |
| 40624070 | 40624369 | − | Sense | Sense |
| 40624070 | 40624369 | − | Sense | Sense |
| 40624070 | 40624369 | − | Sense | Sense |
| 41285643 | 41285951 | − | Sense | Sense |
| 41379874 | 41380176 | + | Sense | Sense |
| 42256736 | 42257037 | + | antiSense | antiSense |
| 42519117 | 42519399 | + | antiSense | antiSense |
| 42522861 | 42523173 | − | antiSense | antiSense |
| 42534238 | 42534530 | − | antiSense | antiSense |
| 43127076 | 43127390 | − | antiSense | antiSense |
| 43219919 | 43220222 | − | Sense | Sense |
| 44182701 | 44183001 | + | antiSense | antiSense |
| 44183011 | 44183231 | + | antiSense | antiSense |
| 44190091 | 44190386 | − | antiSense | antiSense |
| 44190091 | 44190386 | − | antiSense | antiSense |
| 44192423 | 44192725 | − | antiSense | antiSense |
| 44192423 | 44192725 | − | antiSense | antiSense |
| 45173668 | 45173976 | − | antiSense | antiSense |
| 46193645 | 46193876 | − | Sense | Sense |
| 47016817 | 47017117 | − | antiSense | antiSense |
| 47422922 | 47423221 | + | antiSense | antiSense |
| 48719125 | 48719354 | + | Sense | Sense |
| 48808020 | 48808319 | + | Sense | Sense |
| 49104511 | 49104811 | + | antiSense | antiSense |
| 49188955 | 49189257 | − | antiSense | antiSense |
| 53036978 | 53037277 | − | antiSense | antiSense |
| 53037442 | 53037741 | + | antiSense | antiSense |
| 54909304 | 54909606 | − | antiSense | antiSense |
| 55180172 | 55180428 | + | Sense | Sense |
| 55381780 | 55382084 | − | Sense | Sense |
| 55535461 | 55535760 | − | Sense | Sense |
| 55541523 | 55541822 | + | Sense | Sense |
| 55547636 | 55547938 | − | Sense | Sense |
| 55725000 | 55725302 | − | Sense | Sense |
| 55915368 | 55915678 | + | antiSense | antiSense |
| 56088198 | 56088496 | − | Sense | Sense |
| 56090557 | 56090838 | + | Sense | Sense |
| 56463186 | 56463485 | + | antiSense | antiSense |
| 56463508 | 56463805 | + | antiSense | antiSense |
| 56477005 | 56477300 | − | antiSense | antiSense |
| 57281473 | 57281780 | + | Sense | Sense |
| 57281781 | 57282096 | + | Sense | Sense |
| 57352205 | 57352502 | − | Sense | Sense |
| 57709050 | 57709346 | + | Sense | Sense |
| 57770446 | 57770721 | + | Sense | Sense |
| 57884912 | 57885216 | + | Sense | Sense |
| 57921753 | 57922058 | + | Sense | Sense |
| 57921753 | 57922058 | + | Sense | Sense |
| 58038937 | 58039237 | − | antiSense | antiSense |
| 58039244 | 58039545 | − | antiSense | antiSense |
| 58585324 | 58585521 | − | antiSense | antiSense |
| 59923746 | 59924047 | − | antiSense | antiSense |
| 59923746 | 59924047 | − | antiSense | antiSense |
| 60046953 | 60047260 | − | antiSense | antiSense |
| 60084002 | 60084299 | + | antiSense | antiSense |
| 60084322 | 60084621 | + | antiSense | antiSense |
| 60119643 | 60119950 | + | antiSense | antiSense |
| 60553663 | 60553941 | + | antiSense | antiSense |
| 60553663 | 60553941 | + | antiSense | antiSense |
| 60554161 | 60554463 | + | antiSense | antiSense |
| 60554161 | 60554463 | + | antiSense | antiSense |
| 62464920 | 62465233 | + | Sense | Sense |
| 62479839 | 62480131 | − | antiSense | antiSense |
| 62634175 | 62634483 | − | antiSense | antiSense |
| 64386060 | 64386357 | + | Sense | Sense |
| 65354402 | 65354713 | + | antiSense | antiSense |
| 65406929 | 65407214 | + | Sense | Sense |
| 65406929 | 65407214 | + | Sense | Sense |
| 65434430 | 65434730 | − | Sense | Sense |
| 65875249 | 65875554 | − | Sense | Sense |
| 66118355 | 66118591 | − | Sense | Sense |
| 66439230 | 66439552 | − | Sense | Sense |
| 70364933 | 70365210 | − | antiSense | antiSense |
| 70381166 | 70381482 | + | antiSense | antiSense |
| 70492086 | 70492379 | + | antiSense | antiSense |
| 73097733 | 73098029 | − | Sense | Sense |
| 73099426 | 73099722 | − | Sense | Sense |
| 73100509 | 73100844 | − | Sense | Sense |
| 73138373 | 73138652 | + | antiSense | antiSense |
| 73533891 | 73534191 | − | Sense | Sense |
| 73940829 | 73941126 | − | antiSense | antiSense |
| 73976510 | 73976803 | − | antiSense | Sense |
| 74390962 | 74391256 | − | antiSense | antiSense |
| 74555796 | 74556066 | + | Sense | Sense |
| 74751895 | 74752203 | − | Sense | Sense |
| 74752205 | 74752504 | − | Sense | Sense |
| 74762217 | 74762526 | + | Sense | Sense |
| 74781881 | 74782174 | + | Sense | Sense |
| 74782227 | 74782522 | − | Sense | Sense |
| 74957592 | 74957885 | + | antiSense | antiSense |
| 74959420 | 74959719 | + | antiSense | antiSense |
| 75460146 | 75460459 | − | Sense | Sense |
| 76178173 | 76178476 | + | antiSense | antiSense |
| 76191266 | 76191575 | − | Sense | Sense |
| 76191587 | 76191876 | − | Sense | Sense |
| 76192478 | 76192771 | + | Sense | Sense |
| 76192478 | 76192771 | + | Sense | Sense |
| 76194066 | 76194320 | + | Sense | Sense |
| 76194066 | 76194320 | + | Sense | Sense |
| 76196252 | 76196516 | − | Sense | Sense |
| 76402116 | 76402416 | + | Sense | Sense |
| 78014196 | 78014503 | + | Sense | Sense |
| 78015922 | 78016218 | − | Sense | Sense |
| 79221749 | 79222056 | − | antiSense | antiSense |
| 79271160 | 79271457 | + | Sense | Sense |
| 79528019 | 79528282 | − | antiSense | antiSense |
| 79599940 | 79600179 | + | antiSense | antiSense |
| 79645925 | 79646213 | + | antiSense | antiSense |
| 79647638 | 79647939 | − | antiSense | antiSense |
| 79648221 | 79648530 | − | antiSense | antiSense |
| 79840017 | 79840331 | + | antiSense | antiSense |
| 79841437 | 79841740 | + | antiSense | antiSense |
| 79841746 | 79842055 | + | antiSense | antiSense |
| 79844839 | 79845124 | − | antiSense | antiSense |
| 79853564 | 79853831 | + | Sense | Sense |
| 80488387 | 80488691 | − | Sense | Sense |
| 80504671 | 80504982 | + | Sense | Sense |
| 80585859 | 80586168 | − | antiSense | antiSense |
| 684295 | 684534 | + | antiSense | antiSense |
| 704606 | 704905 | − | antiSense | antiSense |
| 3601559 | 3601857 | + | Sense | Sense |
| 3601559 | 3601857 | + | Sense | Sense |
| 3608049 | 3608334 | + | Sense | Sense |
| 3608841 | 3609126 | − | Sense | Sense |
| 9368664 | 9368957 | + | Sense | Sense |
| 9370037 | 9370328 | + | Sense | Sense |
| 12967631 | 12967939 | + | Sense | Sense |
| 19170630 | 19170927 | + | antiSense | antiSense |
| 19170630 | 19170927 | + | antiSense | antiSense |
| 19787620 | 19787910 | − | Sense | Sense |
| 21691253 | 21691545 | + | Sense | Sense |
| 23635149 | 23635444 | + | antiSense | antiSense |
| 29650003 | 29650299 | − | Sense | Sense |
| 29650668 | 29650965 | − | Sense | Sense |
| 29651121 | 29651395 | − | Sense | Sense |
| 43676185 | 43676483 | + | antiSense | antiSense |
| 61063954 | 61064236 | + | antiSense | antiSense |
| 61065789 | 61066082 | + | antiSense | antiSense |
| 374161 | 374460 | + | Sense | Sense |
| 375190 | 375493 | − | Sense | Sense |
| 375736 | 376034 | − | Sense | Sense |
| 459213 | 459522 | − | antiSense | antiSense |
| 479939 | 480245 | − | antiSense | antiSense |
| 489392 | 489690 | − | antiSense | antiSense |
| 766320 | 766597 | − | antiSense | antiSense |
| 771314 | 771597 | + | antiSense | antiSense |
| 772487 | 772730 | − | antiSense | antiSense |
| 772731 | 773030 | − | antiSense | antiSense |
| 773176 | 773350 | + | antiSense | antiSense |
| 1863347 | 1863645 | − | Sense | Sense |
| 1886123 | 1886418 | − | Sense | Sense |
| 1887927 | 1888233 | − | Sense | Sense |
| 2393668 | 2393893 | + | antiSense | antiSense |
| 2573611 | 2573910 | + | Sense | Sense |
| 3086872 | 3087151 | − | Sense | Sense |
| 3945841 | 3946068 | − | antiSense | antiSense |
| 3946069 | 3946378 | − | antiSense | antiSense |
| 3946913 | 3947218 | + | antiSense | antiSense |
| 6791474 | 6791768 | + | Sense | Sense |
| 6791474 | 6791768 | + | Sense | Sense |
| 17807303 | 17807588 | + | antiSense | antiSense |
| 39524385 | 39524673 | − | antiSense | antiSense |
| 39524677 | 39524971 | − | antiSense | antiSense |
| 39525143 | 39525443 | + | antiSense | antiSense |
| 39525143 | 39525443 | + | antiSense | antiSense |
| 39550533 | 39550833 | + | antiSense | antiSense |
| 39550913 | 39551192 | − | antiSense | antiSense |
| 39551899 | 39552201 | − | antiSense | antiSense |
| 39552527 | 39552829 | − | antiSense | antiSense |
| 39606237 | 39606530 | − | antiSense | antiSense |
| 39713378 | 39713669 | + | Sense | Sense |
| 39718845 | 39719148 | − | Sense | Sense |
| 39726085 | 39726404 | + | Sense | Sense |
| 39726085 | 39726404 | + | Sense | Sense |
| 39760017 | 39760325 | + | Sense | Sense |
| 39760017 | 39760325 | + | Sense | Sense |
| 39833841 | 39834125 | − | antiSense | antiSense |
| 39833841 | 39834125 | − | antiSense | antiSense |
| 39833841 | 39834125 | − | antiSense | antiSense |
| 39833841 | 39834125 | − | antiSense | antiSense |
| 39897647 | 39897953 | − | antiSense | antiSense |
| 39898105 | 39898401 | + | antiSense | antiSense |
| 39976449 | 39976748 | + | antiSense | antiSense |
| 48840053 | 48840357 | + | Sense | Sense |
| 56268605 | 56268913 | − | Sense | Sense |
| 56280154 | 56280450 | + | Sense | Sense |
| 56280154 | 56280450 | + | Sense | Sense |
| 56760781 | 56761081 | − | Sense | Sense |
| 71582279 | 71582592 | + | Sense | Sense |
| 71584447 | 71584731 | − | Sense | Sense |
| 71770196 | 71770493 | + | Sense | Sense |
| 73923392 | 73923688 | − | antiSense | antiSense |
| 74106521 | 74106827 | − | antiSense | antiSense |
| 76683800 | 76684101 | + | Sense | Sense |
| 76693968 | 76694260 | + | Sense | Sense |
| 76858671 | 76858940 | − | antiSense | antiSense |
| 77866224 | 77866498 | − | antiSense | antiSense |
| 84381775 | 84382069 | + | antiSense | antiSense |
| 87845359 | 87845650 | − | antiSense | antiSense |
| 87852907 | 87853210 | + | antiSense | antiSense |
| 87853219 | 87853517 | + | antiSense | antiSense |
| 87905474 | 87905774 | − | Sense | Sense |
| 87918633 | 87918926 | + | Sense | Sense |
| 89182834 | 89183172 | − | antiSense | antiSense |
| 89182834 | 89183172 | − | antiSense | antiSense |
| 99462544 | 99462846 | + | antiSense | antiSense |
| 99731378 | 99731688 | + | Sense | Sense |
| 1.1E+08 | 1.1E+08 | − | Sense | Sense |
| 1.29E+08 | 1.29E+08 | − | Sense | Sense |
| 1.29E+08 | 1.29E+08 | − | Sense | Sense |
| 1.29E+08 | 1.29E+08 | + | antiSense | antiSense |
| 1.29E+08 | 1.29E+08 | − | Sense | Sense |
| 1.29E+08 | 1.29E+08 | − | Sense | Sense |
| 1.29E+08 | 1.29E+08 | − | Sense | Sense |
| 1.4E+08 | 1.4E+08 | + | Sense | Sense |
| 1.4E+08 | 1.4E+08 | + | Sense | Sense |
| 1.4E+08 | 1.4E+08 | − | antiSense | antiSense |
| 1.4E+08 | 1.4E+08 | − | Sense | Sense |
| 1.41E+08 | 1.41E+08 | − | antiSense | antiSense |
| 1.41E+08 | 1.41E+08 | + | antiSense | antiSense |
| 1.52E+08 | 1.52E+08 | − | antiSense | antiSense |
| 1.52E+08 | 1.52E+08 | − | Sense | Sense |
| 1.52E+08 | 1.52E+08 | + | Sense | antiSense |
| 1.52E+08 | 1.52E+08 | + | antiSense | antiSense |
| 1.52E+08 | 1.52E+08 | − | antiSense | antiSense |
| 1.53E+08 | 1.53E+08 | + | antiSense | antiSense |
| 1.54E+08 | 1.54E+08 | + | antiSense | antiSense |
| 1.54E+08 | 1.54E+08 | + | antiSense | antiSense |
| 1.6E+08 | 1.6E+08 | − | Sense | Sense |
| 1.66E+08 | 1.66E+08 | + | Sense | Sense |
| 1.7E+08 | 1.7E+08 | − | antiSense | antiSense |
| 1.86E+08 | 1.86E+08 | + | Sense | Sense |
| 1.86E+08 | 1.86E+08 | + | Sense | Sense |
| 1.86E+08 | 1.86E+08 | − | Sense | Sense |
| 265338 | 265652 | + | antiSense | antiSense |
| 266377 | 266678 | − | antiSense | antiSense |
| 267019 | 267315 | − | antiSense | antiSense |
| 267424 | 267720 | + | antiSense | antiSense |
| 267424 | 267720 | + | antiSense | antiSense |
| 1343561 | 1343859 | + | antiSense | antiSense |
| 1409829 | 1410119 | + | Sense | Sense |
| 1410344 | 1410639 | + | Sense | Sense |
| 1523803 | 1524098 | + | antiSense | antiSense |
| 1567842 | 1568138 | − | antiSense | antiSense |
| 2912444 | 2912747 | + | Sense | Sense |
| 2912444 | 2912747 | + | Sense | Sense |
| 3084282 | 3084582 | − | antiSense | antiSense |
| 3084715 | 3085009 | − | antiSense | antiSense |
| 3514023 | 3514311 | − | Sense | Sense |
| 4332861 | 4333168 | − | antiSense | antiSense |
| 4399288 | 4399569 | + | antiSense | antiSense |
| 4510597 | 4510899 | + | antiSense | antiSense |
| 4512198 | 4512498 | + | antiSense | antiSense |
| 4512861 | 4513155 | − | antiSense | antiSense |
| 4518019 | 4518324 | + | antiSense | antiSense |
| 4518336 | 4518636 | + | antiSense | antiSense |
| 4531068 | 4531379 | + | Sense | Sense |
| 4533611 | 4533912 | − | Sense | Sense |
| 4678550 | 4678845 | − | Sense | Sense |
| 4678550 | 4678845 | − | Sense | Sense |
| 4678550 | 4678845 | − | Sense | Sense |
| 5136451 | 5136671 | − | Sense | Sense |
| 8792520 | 8792832 | + | Sense | Sense |
| 8910316 | 8910614 | + | Sense | Sense |
| 11863637 | 11863942 | − | antiSense | antiSense |
| 11929042 | 11929327 | − | antiSense | antiSense |
| 11931215 | 11931504 | − | antiSense | antiSense |
| 11958412 | 11958689 | + | Sense | Sense |
| 11992299 | 11992606 | − | antiSense | antiSense |
| 11993910 | 11994200 | + | antiSense | antiSense |
| 11996447 | 11996743 | − | antiSense | antiSense |
| 12005409 | 12005707 | − | antiSense | antiSense |
| 12005922 | 12006230 | + | antiSense | antiSense |
| 12128599 | 12128887 | + | Sense | Sense |
| 15793121 | 15793335 | − | Sense | Sense |
| 15793964 | 15794251 | − | Sense | Sense |
| 15794296 | 15794607 | − | Sense | Sense |
| 15794811 | 15795109 | + | Sense | Sense |
| 15794811 | 15795109 | + | Sense | Sense |
| 19066063 | 19066369 | − | Sense | Sense |
| 19131404 | 19131703 | − | Sense | Sense |
| 19474257 | 19474531 | − | Sense | Sense |
| 19715403 | 19715705 | − | antiSense | antiSense |
| 21967139 | 21967444 | − | Sense | Sense |
| 21967583 | 21967873 | + | Sense | Sense |
| 22408124 | 22408395 | + | antiSense | antiSense |
| 22506539 | 22506839 | − | Sense | Sense |
| 22520447 | 22520712 | − | Sense | Sense |
| 23537432 | 23537744 | − | antiSense | antiSense |
| 27489209 | 27489521 | + | antiSense | antiSense |
| 28211196 | 28211489 | + | antiSense | antiSense |
| 28968682 | 28968980 | + | Sense | Sense |
| 29418724 | 29419034 | − | antiSense | antiSense |
| 29595578 | 29595885 | − | antiSense | antiSense |
| 29707531 | 29707817 | + | Sense | Sense |
| 29707822 | 29708125 | + | Sense | Sense |
| 29709026 | 29709334 | + | Sense | Sense |
| 30591676 | 30591975 | − | antiSense | antiSense |
| 30592230 | 30592498 | + | antiSense | antiSense |
| 30592499 | 30592803 | + | antiSense | antiSense |
| 30592499 | 30592803 | + | antiSense | antiSense |
| 30593127 | 30593421 | + | antiSense | antiSense |
| 30782657 | 30782969 | + | Sense | Sense |
| 30895166 | 30895459 | + | Sense | antiSense |
| 30945404 | 30945695 | + | Sense | Sense |
| 31074488 | 31074751 | − | antiSense | antiSense |
| 31133920 | 31134212 | + | Sense | Sense |
| 31134225 | 31134514 | + | Sense | Sense |
| 47447758 | 47448031 | − | antiSense | antiSense |
| 52539607 | 52539902 | + | antiSense | antiSense |
| 53136762 | 53137056 | − | Sense | Sense |
| 53198579 | 53198866 | − | Sense | Sense |
| 53728422 | 53728723 | − | antiSense | antiSense |
| 56478814 | 56479115 | + | antiSense | antiSense |
| 57204704 | 57204996 | + | antiSense | antiSense |
| 58653328 | 58653627 | − | antiSense | antiSense |
| 58655176 | 58655475 | − | antiSense | antiSense |
| 66719877 | 66720177 | + | antiSense | antiSense |
| 66720184 | 66720470 | + | antiSense | antiSense |
| 66858214 | 66858506 | + | antiSense | antiSense |
| 67170778 | 67171075 | + | Sense | Sense |
| 67608248 | 67608544 | − | Sense | Sense |
| 67717991 | 67718305 | + | antiSense | antiSense |
| 67717991 | 67718305 | + | antiSense | antiSense |
| 67885104 | 67885391 | + | Sense | Sense |
| 68780049 | 68780347 | + | Sense | Sense |
| 68800642 | 68800929 | − | Sense | Sense |
| 69160868 | 69161156 | + | antiSense | antiSense |
| 69195574 | 69195865 | − | Sense | Sense |
| 69377732 | 69378034 | − | Sense | Sense |
| 69411235 | 69411497 | − | antiSense | antiSense |
| 69414283 | 69414574 | + | antiSense | antiSense |
| 69464121 | 69464422 | − | Sense | Sense |
| 69464577 | 69464875 | − | Sense | Sense |
| 69465394 | 69465691 | + | Sense | Sense |
| 69465394 | 69465691 | + | Sense | Sense |
| 69487449 | 69487751 | − | Sense | Sense |
| 69487449 | 69487751 | − | Sense | Sense |
| 69487449 | 69487751 | − | Sense | Sense |
| 69488790 | 69489095 | − | Sense | Sense |
| 69488790 | 69489095 | − | Sense | Sense |
| 69488790 | 69489095 | − | Sense | Sense |
| 69489299 | 69489615 | − | Sense | Sense |
| 69489299 | 69489615 | − | Sense | Sense |
| 69668927 | 69669213 | − | Sense | Sense |
| 69698649 | 69698937 | + | Sense | Sense |
| 69711774 | 69712081 | + | Sense | Sense |
| 69712136 | 69712415 | − | Sense | Sense |
| 69723463 | 69723747 | − | Sense | Sense |
| 69723463 | 69723747 | − | Sense | Sense |
| 70298219 | 70298519 | − | antiSense | antiSense |
| 70298219 | 70298519 | − | antiSense | antiSense |
| 70411113 | 70411407 | + | antiSense | antiSense |
| 70412069 | 70412326 | + | antiSense | antiSense |
| 70463539 | 70463806 | − | antiSense | antiSense |
| 70610751 | 70611050 | + | Sense | Sense |
| 71959903 | 71960191 | + | Sense | Sense |
| 71960254 | 71960547 | − | Sense | Sense |
| 71961122 | 71961405 | + | Sense | Sense |
| 71961122 | 71961405 | + | Sense | Sense |
| 74363548 | 74363843 | + | antiSense | antiSense |
| 74363868 | 74364158 | − | antiSense | antiSense |
| 74363868 | 74364158 | − | antiSense | antiSense |
| 74674556 | 74674849 | + | antiSense | antiSense |
| 74675541 | 74675852 | + | antiSense | antiSense |
| 75485166 | 75485446 | + | antiSense | antiSense |
| 75508182 | 75508472 | − | antiSense | antiSense |
| 75510168 | 75510483 | + | antiSense | antiSense |
| 75626839 | 75627147 | + | antiSense | antiSense |
| 75629923 | 75630221 | − | antiSense | antiSense |
| 78157343 | 78157649 | − | Sense | Sense |
| 81089021 | 81089308 | + | antiSense | antiSense |
| 81089322 | 81089622 | − | antiSense | antiSense |
| 84107838 | 84108095 | − | antiSense | antiSense |
| 85017621 | 85017926 | − | antiSense | antiSense |
| 87390931 | 87391233 | − | antiSense | antiSense |
| 87771195 | 87771496 | − | antiSense | antiSense |
| 87777064 | 87777373 | + | antiSense | antiSense |
| 89472262 | 89472537 | + | antiSense | antiSense |
| 89559542 | 89559829 | + | Sense | Sense |
| 89582726 | 89582986 | + | Sense | Sense |
| 89582726 | 89582986 | + | Sense | Sense |
| 89582987 | 89583298 | + | Sense | Sense |
| 89582987 | 89583298 | + | Sense | Sense |
| 89595007 | 89595212 | − | Sense | Sense |
| 89595222 | 89595519 | − | Sense | Sense |
| 89719747 | 89720059 | + | antiSense | antiSense |
| 89719747 | 89720059 | + | antiSense | antiSense |
| 89755227 | 89755524 | + | Sense | Sense |
| 89879847 | 89880140 | − | antiSense | antiSense |
| 89879847 | 89880140 | − | antiSense | antiSense |
| 89932096 | 89932395 | + | Sense | Sense |
| 90054616 | 90054897 | − | Sense | Sense |
| 9488178 | 9488470 | − | Sense | Sense |
| 9843644 | 9843954 | + | Sense | Sense |
| 10041707 | 10042023 | − | Sense | Sense |
| 10193472 | 10193766 | + | Sense | Sense |
| 10194166 | 10194473 | − | Sense | Sense |
| 10323384 | 10323664 | + | Sense | Sense |
| 11736524 | 11736821 | − | antiSense | antiSense |
| 12444839 | 12445154 | − | Sense | Sense |
| 12578218 | 12578516 | + | Sense | Sense |
| 15919035 | 15919341 | + | Sense | Sense |
| 17752163 | 17752488 | − | antiSense | antiSense |
| 20016437 | 20016735 | + | Sense | Sense |
| 23291145 | 23291446 | − | Sense | Sense |
| 23897518 | 23897805 | − | Sense | Sense |
| 27519108 | 27519404 | − | antiSense | antiSense |
| 28285941 | 28286237 | + | Sense | Sense |
| 30662768 | 30663072 | − | Sense | Sense |
| 31680824 | 31681104 | − | Sense | Sense |
| 31736235 | 31736535 | − | antiSense | antiSense |
| 31771553 | 31771866 | + | antiSense | antiSense |
| 31951845 | 31952157 | + | antiSense | antiSense |
| 32284741 | 32285042 | + | Sense | Sense |
| 32796459 | 32796761 | + | Sense | Sense |
| 34274144 | 34274446 | − | Sense | Sense |
| 40432887 | 40433196 | + | antiSense | antiSense |
| 40507400 | 40507689 | + | Sense | Sense |
| 44480523 | 44480829 | + | antiSense | antiSense |
| 44538234 | 44538533 | + | antiSense | antiSense |
| 44801880 | 44802178 | − | antiSense | antiSense |
| 44802179 | 44802483 | − | antiSense | antiSense |
| 47178162 | 47178450 | + | antiSense | antiSense |
| 47488898 | 47489208 | + | antiSense | antiSense |
| 47510856 | 47511148 | − | antiSense | antiSense |
| 47948328 | 47948623 | − | antiSense | antiSense |
| 48290588 | 48290892 | − | Sense | Sense |
| 48291336 | 48291631 | + | Sense | Sense |
| 48291967 | 48292261 | − | Sense | Sense |
| 48292855 | 48293064 | + | Sense | Sense |
| 48292855 | 48293064 | + | Sense | Sense |
| 48293251 | 48293543 | + | Sense | Sense |
| 48293251 | 48293543 | + | Sense | Sense |
| 48293673 | 48293979 | − | Sense | Sense |
| 48293673 | 48293979 | − | Sense | Sense |
| 48293673 | 48293979 | − | Sense | Sense |
| 48294108 | 48294405 | − | Sense | Sense |
| 48294108 | 48294405 | − | Sense | Sense |
| 48294108 | 48294405 | − | Sense | Sense |
| 48294983 | 48295290 | − | Sense | Sense |
| 48294983 | 48295290 | − | Sense | Sense |
| 48748051 | 48748341 | − | antiSense | antiSense |
| 48751273 | 48751550 | + | antiSense | antiSense |
| 48751649 | 48751799 | − | antiSense | antiSense |
| 48751837 | 48752123 | − | antiSense | antiSense |
| 49030455 | 49030752 | + | Sense | Sense |
| 49047558 | 49047852 | + | Sense | Sense |
| 49099806 | 49100090 | − | antiSense | antiSense |
| 49123681 | 49123979 | − | antiSense | antiSense |
| 49123681 | 49123979 | − | antiSense | antiSense |
| 49398227 | 49398534 | + | antiSense | antiSense |
| 49461277 | 49461583 | − | antiSense | antiSense |
| 50059756 | 50060061 | − | Sense | Sense |
| 50071075 | 50071372 | − | Sense | Sense |
| 50073200 | 50073491 | − | Sense | Sense |
| 50372389 | 50372678 | − | antiSense | antiSense |
| 50372389 | 50372678 | − | antiSense | antiSense |
| 56699718 | 56700009 | + | antiSense | antiSense |
| 57577934 | 57578217 | + | antiSense | antiSense |
| 57829837 | 57830154 | + | Sense | Sense |
| 58011404 | 58011668 | + | Sense | Sense |
| 73103158 | 73103367 | − | Sense | Sense |
| 75463010 | 75463313 | − | antiSense | antiSense |
| 75463358 | 75463617 | + | antiSense | antiSense |
| 75465732 | 75466018 | + | antiSense | antiSense |
| 75466396 | 75466689 | − | antiSense | antiSense |
| 75466396 | 75466689 | − | antiSense | antiSense |
| 75467966 | 75468250 | − | antiSense | antiSense |
| 75474428 | 75474742 | − | antiSense | antiSense |
| 75783575 | 75783884 | − | antiSense | antiSense |
| 75783970 | 75784275 | − | antiSense | antiSense |
| 78965120 | 78965412 | + | antiSense | antiSense |
| 88138362 | 88138653 | + | Sense | Sense |
| 93848587 | 93848893 | − | Sense | Sense |
| 1.01E+08 | 1.1E+08 | − | Sense | Sense |
| 1.13E+08 | 1.13E+08 | + | Sense | Sense |
| 1.14E+08 | 1.14E+08 | + | Sense | Sense |
| 1.16E+08 | 1.16E+08 | − | Sense | Sense |
| 1.25E+08 | 1.25E+08 | − | antiSense | antiSense |
| 1.25E+08 | 1.25E+08 | + | antiSense | antiSense |
| 1.29E+08 | 1.29E+08 | − | antiSense | antiSense |
| 1.29E+08 | 1.29E+08 | − | antiSense | antiSense |
| 1.29E+08 | 1.29E+08 | − | antiSense | antiSense |
| 1.29E+08 | 1.29E+08 | − | antiSense | antiSense |
| 1.29E+08 | 1.29E+08 | − | antiSense | antiSense |
| 1.29E+08 | 1.29E+08 | − | antiSense | antiSense |
| 1.3E+08 | 1.3E+08 | + | antiSense | antiSense |
| 1.3E+08 | 1.3E+08 | − | antiSense | antiSense |
| 1.31E+08 | 1.31E+08 | + | Sense | Sense |
| 1.31E+08 | 1.31E+08 | + | antiSense | antiSense |
| 1.36E+08 | 1.36E+08 | + | antiSense | antiSense |
| 1.36E+08 | 1.36E+08 | − | antiSense | antiSense |
| 1.36E+08 | 1.36E+08 | + | antiSense | antiSense |
| 1.36E+08 | 1.36E+08 | + | antiSense | antiSense |
| 1.37E+08 | 1.37E+08 | − | Sense | Sense |
| 1.38E+08 | 1.38E+08 | + | Sense | Sense |
| 1.38E+08 | 1.38E+08 | + | antiSense | antiSense |
| 1.39E+08 | 1.39E+08 | − | antiSense | antiSense |
| 1.39E+08 | 1.39E+08 | + | antiSense | antiSense |
| 1.42E+08 | 1.42E+08 | + | antiSense | antiSense |
| 1.42E+08 | 1.42E+08 | − | antiSense | antiSense |
| 1.42E+08 | 1.42E+08 | + | antiSense | antiSense |
| 1.42E+08 | 1.42E+08 | + | antiSense | antiSense |
| 1.42E+08 | 1.42E+08 | − | antiSense | antiSense |
| 1.42E+08 | 1.42E+08 | + | antiSense | antiSense |
| 1.42E+08 | 1.42E+08 | + | antiSense | antiSense |
| 1.42E+08 | 1.42E+08 | + | antiSense | antiSense |
| 1.42E+08 | 1.42E+08 | + | antiSense | antiSense |
| 1.42E+08 | 1.42E+08 | + | antiSense | antiSense |
| 1.42E+08 | 1.42E+08 | + | antiSense | antiSense |
| 1.42E+08 | 1.42E+08 | − | Sense | Sense |
| 1.49E+08 | 1.49E+08 | − | antiSense | antiSense |
| 1.56E+08 | 1.56E+08 | + | antiSense | antiSense |
| 1.56E+08 | 1.56E+08 | + | antiSense | antiSense |
| 1.56E+08 | 1.56E+08 | + | antiSense | antiSense |
| 1.56E+08 | 1.56E+08 | + | antiSense | antiSense |
| 1.56E+08 | 1.56E+08 | + | antiSense | antiSense |
| 1.56E+08 | 1.56E+08 | − | antiSense | antiSense |
| 1.56E+08 | 1.56E+08 | − | antiSense | antiSense |
| 1.56E+08 | 1.56E+08 | + | antiSense | antiSense |
| 1.56E+08 | 1.56E+08 | + | Sense | Sense |
| 1.56E+08 | 1.56E+08 | − | Sense | Sense |
| 1.69E+08 | 1.69E+08 | − | antiSense | antiSense |
| 1.7E+08 | 1.7E+08 | + | Sense | Sense |
| 1.7E+08 | 1.7E+08 | + | Sense | Sense |
| 1.7E+08 | 1.7E+08 | − | antiSense | antiSense |
| 1.7E+08 | 1.7E+08 | − | Sense | Sense |
| 1.72E+08 | 1.72E+08 | + | Sense | Sense |
| 1.72E+08 | 1.72E+08 | + | Sense | Sense |
| 1.72E+08 | 1.72E+08 | − | antiSense | antiSense |
| 1.73E+08 | 1.73E+08 | − | Sense | Sense |
| 1.77E+08 | 1.77E+08 | + | antiSense | antiSense |
| 1.77E+08 | 1.77E+08 | − | antiSense | antiSense |
| 1.77E+08 | 1.77E+08 | − | antiSense | antiSense |
| 1.77E+08 | 1.77E+08 | − | antiSense | antiSense |
| 1.77E+08 | 1.77E+08 | + | antiSense | antiSense |
| 1.77E+08 | 1.77E+08 | + | antiSense | antiSense |
| 1.77E+08 | 1.77E+08 | − | antiSense | antiSense |
| 1.77E+08 | 1.77E+08 | − | antiSense | antiSense |
| 1.77E+08 | 1.77E+08 | + | antiSense | antiSense |
| 1.77E+08 | 1.77E+08 | − | antiSense | antiSense |
| 1.79E+08 | 1.79E+08 | + | Sense | Sense |
| 1.79E+08 | 1.79E+08 | + | Sense | Sense |
| 1.81E+08 | 1.81E+08 | − | Sense | Sense |
| 1.81E+08 | 1.81E+08 | − | Sense | Sense |
| 1.83E+08 | 1.83E+08 | + | antiSense | antiSense |
| 1.83E+08 | 1.83E+08 | + | Sense | Sense |
| 1.83E+08 | 1.83E+08 | + | Sense | Sense |
| 1.83E+08 | 1.83E+08 | − | Sense | Sense |
| 1.83E+08 | 1.83E+08 | + | Sense | Sense |
| 1.84E+08 | 1.84E+08 | − | Sense | Sense |
| 1.84E+08 | 1.84E+08 | + | Sense | Sense |
| 1.84E+08 | 1.84E+08 | + | Sense | Sense |
| 1.84E+08 | 1.84E+08 | + | Sense | Sense |
| 1.84E+08 | 1.84E+08 | + | Sense | Sense |
| 1.85E+08 | 1.85E+08 | + | antiSense | antiSense |
| 1.85E+08 | 1.85E+08 | + | antiSense | antiSense |
| 1.85E+08 | 1.85E+08 | + | antiSense | antiSense |
| 1.85E+08 | 1.85E+08 | − | antiSense | antiSense |
| 1.85E+08 | 1.85E+08 | − | antiSense | antiSense |
| 1.85E+08 | 1.85E+08 | − | antiSense | antiSense |
| 1.85E+08 | 1.85E+08 | + | antiSense | antiSense |
| 1.85E+08 | 1.85E+08 | − | antiSense | antiSense |
| 1.85E+08 | 1.85E+08 | + | antiSense | antiSense |
| 1.86E+08 | 1.86E+08 | − | antiSense | antiSense |
| 1.86E+08 | 1.86E+08 | − | antiSense | antiSense |
| 1.86E+08 | 1.86E+08 | + | antiSense | antiSense |
| 1.86E+08 | 1.86E+08 | + | antiSense | antiSense |
| 1.86E+08 | 1.86E+08 | − | antiSense | antiSense |
| 1.91E+08 | 1.91E+08 | + | Sense | Sense |
| 1.94E+08 | 1.94E+08 | − | Sense | Sense |
| 1.94E+08 | 1.94E+08 | + | Sense | Sense |
| 1.94E+08 | 1.94E+08 | − | Sense | Sense |
| 1.94E+08 | 1.94E+08 | − | antiSense | antiSense |
| 1.94E+08 | 1.94E+08 | − | antiSense | antiSense |
| 1.95E+08 | 1.95E+08 | − | Sense | Sense |
| 1.96E+08 | 1.96E+08 | + | antiSense | antiSense |
| 1.96E+08 | 1.96E+08 | − | antiSense | antiSense |
| 1.96E+08 | 1.96E+08 | + | antiSense | antiSense |
| 1.96E+08 | 1.96E+08 | + | antiSense | antiSense |
| 1.96E+08 | 1.96E+08 | − | antiSense | antiSense |
| 1.96E+08 | 1.96E+08 | + | antiSense | antiSense |
| 1.96E+08 | 1.96E+08 | + | antiSense | antiSense |
| 1.96E+08 | 1.96E+08 | + | antiSense | antiSense |
| 1.96E+08 | 1.96E+08 | + | antiSense | antiSense |
| 1.96E+08 | 1.96E+08 | − | Sense | Sense |
| 1.96E+08 | 1.96E+08 | − | Sense | Sense |
| 1.96E+08 | 1.96E+08 | − | Sense | Sense |
| 1.96E+08 | 1.96E+08 | + | Sense | Sense |
| 1.96E+08 | 1.96E+08 | − | Sense | Sense |
| 1.97E+08 | 1.97E+08 | + | Sense | Sense |
| 1.97E+08 | 1.97E+08 | − | Sense | Sense |
| 1.97E+08 | 1.97E+08 | + | Sense | Sense |
| 1.97E+08 | 1.97E+08 | + | Sense | Sense |
| 1.97E+08 | 1.97E+08 | − | Sense | Sense |
| 1.97E+08 | 1.97E+08 | − | Sense | Sense |
| 1.97E+08 | 1.97E+08 | − | Sense | Sense |
| 1.97E+08 | 1.97E+08 | + | antiSense | antiSense |
| 1.98E+08 | 1.98E+08 | − | Sense | Sense |
| 1.98E+08 | 1.98E+08 | + | Sense | Sense |
| 758002 | 758293 | − | antiSense | antiSense |
| 761002 | 761299 | + | antiSense | antiSense |
| 2231758 | 2232066 | − | antiSense | antiSense |
| 2409830 | 2410129 | + | Sense | Sense |
| 5369034 | 5369338 | + | antiSense | antiSense |
| 5539078 | 5539391 | + | antiSense | antiSense |
| 5539078 | 5539391 | + | antiSense | antiSense |
| 6418900 | 6419204 | − | Sense | Sense |
| 6418900 | 6419204 | − | Sense | Sense |
| 6427978 | 6428270 | − | Sense | Sense |
| 11087141 | 11087419 | + | Sense | Sense |
| 20219786 | 20220093 | + | antiSense | antiSense |
| 20220189 | 20220470 | + | antiSense | antiSense |
| 23341309 | 23341601 | + | Sense | Sense |
| 23342404 | 23342680 | − | Sense | Sense |
| 23395270 | 23395569 | + | antiSense | antiSense |
| 23395895 | 23396207 | + | antiSense | antiSense |
| 23453510 | 23453783 | + | antiSense | antiSense |
| 23491612 | 23491922 | − | antiSense | antiSense |
| 23491612 | 23491922 | − | antiSense | antiSense |
| 23553687 | 23553943 | − | antiSense | antiSense |
| 23558289 | 23558606 | − | antiSense | antiSense |
| 23558731 | 23559010 | + | antiSense | antiSense |
| 24867948 | 24868255 | + | antiSense | antiSense |
| 25160462 | 25160761 | + | antiSense | antiSense |
| 25160882 | 25161177 | + | antiSense | antiSense |
| 25160882 | 25161177 | + | antiSense | antiSense |
| 25161266 | 25161555 | + | antiSense | antiSense |
| 25161266 | 25161555 | + | antiSense | antiSense |
| 27855373 | 27855653 | + | Sense | Sense |
| 32533155 | 32533452 | − | antiSense | antiSense |
| 32636129 | 32636421 | + | antiSense | antiSense |
| 32916631 | 32916928 | − | antiSense | antiSense |
| 36286453 | 36286761 | + | Sense | Sense |
| 36287819 | 36288115 | − | Sense | Sense |
| 38764186 | 38764490 | − | antiSense | antiSense |
| 40101892 | 40102178 | − | Sense | Sense |
| 43934347 | 43934646 | + | antiSense | antiSense |
| 44020656 | 44020961 | + | antiSense | antiSense |
| 44043623 | 44043933 | − | antiSense | antiSense |
| 44043623 | 44043933 | − | antiSense | antiSense |
| 44616948 | 44617242 | + | antiSense | antiSense |
| 44728031 | 44728328 | − | Sense | Sense |
| 45048999 | 45049302 | − | Sense | Sense |
| 56106555 | 56106861 | + | antiSense | antiSense |
| 56121163 | 56121465 | + | Sense | Sense |
| 56124493 | 56124772 | − | Sense | Sense |
| 64013222 | 64013520 | + | antiSense | antiSense |
| 64020908 | 64021201 | + | antiSense | antiSense |
| 64278849 | 64279142 | + | Sense | Sense |
| 64284037 | 64284334 | + | Sense | Sense |
| 64460901 | 64461187 | + | antiSense | antiSense |
| 64462703 | 64463005 | − | antiSense | antiSense |
| 64463585 | 64463882 | + | antiSense | antiSense |
| 64463585 | 64463882 | + | antiSense | antiSense |
| 64537352 | 64537652 | − | antiSense | antiSense |
| 64537352 | 64537652 | − | antiSense | antiSense |
| 64538466 | 64538773 | + | antiSense | antiSense |
| 64539207 | 64539509 | − | antiSense | antiSense |
| 64554693 | 64555001 | + | Sense | Sense |
| 65191291 | 65191539 | + | antiSense | Sense |
| 65341289 | 65341506 | + | Sense | Sense |
| 65368494 | 65368779 | + | Sense | Sense |
| 65601785 | 65602073 | + | Sense | Sense |
| 65619158 | 65619381 | − | Sense | Sense |
| 65619158 | 65619381 | − | Sense | Sense |
| 65620049 | 65620356 | + | Sense | Sense |
| 65620049 | 65620356 | + | Sense | Sense |
| 65620523 | 65620823 | − | Sense | Sense |
| 65620523 | 65620823 | − | Sense | Sense |
| 65620523 | 65620823 | − | Sense | Sense |
| 65621310 | 65621625 | − | Sense | Sense |
| 65621310 | 65621625 | − | Sense | Sense |
| 65621310 | 65621625 | − | Sense | Sense |
| 65622829 | 65623131 | − | Sense | Sense |
| 65623784 | 65624086 | + | Sense | Sense |
| 65623784 | 65624086 | + | Sense | Sense |
| 65623784 | 65624086 | + | Sense | Sense |
| 65625320 | 65625624 | − | Sense | Sense |
| 65992171 | 65992483 | − | antiSense | antiSense |
| 65992171 | 65992483 | − | antiSense | antiSense |
| 66001537 | 66001828 | − | antiSense | antiSense |
| 66004609 | 66004901 | + | antiSense | antiSense |
| 66005211 | 66005468 | − | antiSense | antiSense |
| 66005666 | 66005938 | + | antiSense | antiSense |
| 66008275 | 66008560 | − | antiSense | antiSense |
| 66008673 | 66008971 | − | antiSense | antiSense |
| 66009219 | 66009511 | + | antiSense | antiSense |
| 66009557 | 66009849 | + | antiSense | antiSense |
| 66009557 | 66009849 | + | antiSense | antiSense |
| 66010265 | 66010555 | − | antiSense | antiSense |
| 66010265 | 66010555 | − | antiSense | antiSense |
| 66012145 | 66012440 | − | antiSense | antiSense |
| 66012145 | 66012440 | − | antiSense | antiSense |
| 66013972 | 66014262 | + | antiSense | antiSense |
| 66015851 | 66016147 | − | antiSense | antiSense |
| 66016148 | 66016443 | − | antiSense | antiSense |
| 66121527 | 66121828 | + | Sense | Sense |
| 66123240 | 66123548 | + | Sense | Sense |
| 66123720 | 66124023 | − | Sense | Sense |
| 66123720 | 66124023 | − | Sense | Sense |
| 66124159 | 66124467 | + | Sense | Sense |
| 66124159 | 66124467 | + | Sense | Sense |
| 66149151 | 66149466 | + | Sense | Sense |
| 66149620 | 66149921 | − | Sense | Sense |
| 66151801 | 66152100 | − | Sense | Sense |
| 66152101 | 66152412 | − | Sense | Sense |
| 66154508 | 66154808 | + | Sense | Sense |
| 66154508 | 66154808 | + | Sense | Sense |
| 66155085 | 66155385 | − | Sense | Sense |
| 66161395 | 66161705 | + | Sense | Sense |
| 66161395 | 66161705 | + | Sense | Sense |
| 66167955 | 66168258 | − | Sense | Sense |
| 66173263 | 66173575 | − | Sense | Sense |
| 66173576 | 66173807 | + | Sense | Sense |
| 66174001 | 66174286 | − | Sense | Sense |
| 66181749 | 66182057 | − | Sense | Sense |
| 66181749 | 66182057 | − | Sense | Sense |
| 66183077 | 66183350 | + | Sense | Sense |
| 66187948 | 66188249 | − | Sense | Sense |
| 66203927 | 66204236 | − | antiSense | antiSense |
| 66217199 | 66217495 | + | Sense | Sense |
| 66218689 | 66218991 | − | Sense | Sense |
| 66220000 | 66220310 | + | Sense | Sense |
| 66252022 | 66252320 | − | Sense | Sense |
| 66318983 | 66319287 | − | Sense | Sense |
| 66466738 | 66467050 | + | Sense | Sense |
| 66739539 | 66739848 | + | antiSense | antiSense |
| 66740050 | 66740360 | + | antiSense | antiSense |
| 66777801 | 66778109 | − | Sense | Sense |
| 66780903 | 66781207 | + | Sense | Sense |
| 66781898 | 66782187 | − | Sense | Sense |
| 66783924 | 66784224 | − | Sense | Sense |
| 72369513 | 72369821 | − | Sense | Sense |
| 72407513 | 72407804 | + | Sense | Sense |
| 72871499 | 72871807 | − | antiSense | antiSense |
| 72874243 | 72874548 | + | antiSense | antiSense |
| 72897987 | 72898269 | − | antiSense | antiSense |
| 72953292 | 72953589 | − | antiSense | antiSense |
| 72953590 | 72953881 | − | antiSense | antiSense |
| 72954879 | 72955187 | + | antiSense | antiSense |
| 72954879 | 72955187 | + | antiSense | antiSense |
| 72955195 | 72955510 | + | antiSense | antiSense |
| 72955195 | 72955510 | + | antiSense | antiSense |
| 73020744 | 73021055 | − | antiSense | antiSense |
| 73022638 | 73022938 | + | antiSense | antiSense |
| 73023567 | 73023874 | − | antiSense | antiSense |
| 73025865 | 73026170 | + | antiSense | antiSense |
| 73150569 | 73150869 | − | antiSense | antiSense |
| 73873500 | 73873806 | + | Sense | Sense |
| 73875900 | 73876208 | − | Sense | Sense |
| 74107897 | 74108194 | + | Sense | Sense |
| 74107897 | 74108194 | + | Sense | Sense |
| 74277036 | 74277329 | + | antiSense | antiSense |
| 74277793 | 74278104 | − | antiSense | antiSense |
| 74282181 | 74282493 | − | antiSense | antiSense |
| 74284105 | 74284407 | − | antiSense | antiSense |
| 74286246 | 74286554 | − | antiSense | antiSense |
| 74286558 | 74286857 | − | antiSense | antiSense |
| 74296485 | 74296736 | + | antiSense | antiSense |
| 74297935 | 74298239 | + | antiSense | antiSense |
| 74298618 | 74298809 | + | antiSense | antiSense |
| 74298618 | 74298809 | + | antiSense | antiSense |
| 74344658 | 74344931 | − | Sense | Sense |
| 75103378 | 75103682 | + | antiSense | antiSense |
| 75118235 | 75118538 | − | antiSense | antiSense |
| 75119212 | 75119513 | + | antiSense | antiSense |
| 75119722 | 75120029 | + | antiSense | antiSense |
| 75512557 | 75512851 | + | Sense | Sense |
| 75513216 | 75513510 | − | Sense | Sense |
| 75513529 | 75513835 | − | Sense | Sense |
| 75675964 | 75676266 | − | antiSense | antiSense |
| 76239686 | 76239967 | + | Sense | Sense |
| 76242825 | 76243105 | − | Sense | Sense |
| 76242825 | 76243105 | − | Sense | antiSense |
| 76638168 | 76638481 | − | Sense | Sense |
| 76638168 | 76638481 | − | Sense | Sense |
| 76639029 | 76639337 | + | Sense | Sense |
| 76639029 | 76639337 | + | Sense | Sense |
| 76640343 | 76640644 | + | Sense | Sense |
| 76640343 | 76640644 | + | Sense | Sense |
| 76695622 | 76695922 | − | Sense | Sense |
| 77186311 | 77186583 | − | Sense | Sense |
| 77186311 | 77186583 | − | Sense | Sense |
| 77238916 | 77239221 | + | Sense | Sense |
| 77254173 | 77254474 | + | Sense | Sense |
| 77254560 | 77254785 | − | Sense | Sense |
| 77263713 | 77264056 | − | Sense | Sense |
| 77269923 | 77270229 | + | Sense | Sense |
| 77308673 | 77308981 | + | antiSense | antiSense |
| 77309343 | 77309606 | − | antiSense | antiSense |
| 77310097 | 77310405 | + | antiSense | antiSense |
| 77319371 | 77319671 | − | antiSense | antiSense |
| 77320661 | 77320962 | + | antiSense | antiSense |
| 77369057 | 77369368 | − | Sense | Sense |
| 77370293 | 77370603 | − | Sense | Sense |
| 77385813 | 77386112 | + | Sense | Sense |
| 83598116 | 83598421 | − | antiSense | antiSense |
| 83609638 | 83609940 | + | antiSense | antiSense |
| 87527825 | 87528133 | + | Sense | Sense |
| 97883629 | 97883934 | − | Sense | Sense |
| 97968104 | 97968404 | + | antiSense | antiSense |
| 98940808 | 98941118 | + | Sense | Sense |
| 98958281 | 98958582 | + | Sense | Sense |
| 99074664 | 99074963 | − | Sense | Sense |
| 99075296 | 99075593 | + | Sense | Sense |
| 99075296 | 99075593 | + | Sense | Sense |
| 99164875 | 99165169 | + | Sense | Sense |
| 99164875 | 99165169 | + | Sense | Sense |
| 99165885 | 99166171 | − | Sense | Sense |
| 99719392 | 99719699 | + | Sense | Sense |
| 99721150 | 99721442 | − | Sense | Sense |
| 99748688 | 99748999 | − | Sense | Sense |
| 99749002 | 99749306 | − | Sense | Sense |
| 99941426 | 99941712 | + | Sense | Sense |
| 1E+08 | 1E+08 | − | antiSense | antiSense |
| 1E+08 | 1E+08 | − | Sense | Sense |
| 1E+08 | 1E+08 | − | antiSense | antiSense |
| 1.01E+08 | 1.01E+08 | + | antiSense | antiSense |
| 1.01E+08 | 1.01E+08 | − | Sense | Sense |
| 1.02E+08 | 1.02E+08 | + | antiSense | antiSense |
| 1.05E+08 | 1.05E+08 | − | Sense | Sense |
| 1.05E+08 | 1.05E+08 | − | antiSense | antiSense |
| 1.05E+08 | 1.05E+08 | + | antiSense | antiSense |
| 1.05E+08 | 1.05E+08 | + | antiSense | antiSense |
| 1.05E+08 | 1.05E+08 | − | Sense | Sense |
| 1.05E+08 | 1.05E+08 | − | antiSense | antiSense |
| 1.05E+08 | 1.05E+08 | − | antiSense | antiSense |
| 1.05E+08 | 1.05E+08 | + | antiSense | antiSense |
| 1.05E+08 | 1.05E+08 | − | antiSense | antiSense |
| 1.05E+08 | 1.05E+08 | − | antiSense | antiSense |
| 1.05E+08 | 1.05E+08 | − | antiSense | antiSense |
| 1.05E+08 | 1.05E+08 | + | antiSense | antiSense |
| 1.07E+08 | 1.07E+08 | + | Sense | Sense |
| 1.16E+08 | 1.16E+08 | + | Sense | Sense |
| 1.16E+08 | 1.16E+08 | − | Sense | Sense |
| 1.21E+08 | 1.21E+08 | − | antiSense | antiSense |
| 1.27E+08 | 1.27E+08 | + | Sense | Sense |
| 1.28E+08 | 1.28E+08 | + | Sense | Sense |
| 1.28E+08 | 1.28E+08 | + | Sense | Sense |
| 1.29E+08 | 1.29E+08 | − | antiSense | antiSense |
| 1.3E+08 | 1.3E+08 | + | antiSense | antiSense |
| 1.3E+08 | 1.3E+08 | − | antiSense | antiSense |
| 1.39E+08 | 1.39E+08 | − | Sense | Sense |
| 1.4E+08 | 1.4E+08 | − | antiSense | antiSense |
| 1.4E+08 | 1.4E+08 | + | antiSense | antiSense |
| 1.41E+08 | 1.41E+08 | + | Sense | Sense |
| 1.46E+08 | 1.46E+08 | − | Sense | Sense |
| 1.49E+08 | 1.49E+08 | − | Sense | Sense |
| 1.51E+08 | 1.51E+08 | + | Sense | Sense |
| 1.51E+08 | 1.51E+08 | − | Sense | Sense |
| 1.52E+08 | 1.52E+08 | − | antiSense | antiSense |
| 1.52E+08 | 1.52E+08 | − | antiSense | antiSense |
| 1.52E+08 | 1.52E+08 | + | antiSense | antiSense |
| 1.52E+08 | 1.52E+08 | + | antiSense | antiSense |
| 1.52E+08 | 1.52E+08 | − | antiSense | antiSense |
| 1.52E+08 | 1.52E+08 | + | antiSense | antiSense |
| 1.52E+08 | 1.52E+08 | − | antiSense | antiSense |
| 1.52E+08 | 1.52E+08 | + | antiSense | antiSense |
| 1.52E+08 | 1.52E+08 | − | antiSense | antiSense |
| 1.58E+08 | 1.58E+08 | − | antiSense | antiSense |
| 1.59E+08 | 1.59E+08 | − | antiSense | antiSense |
| 1.59E+08 | 1.59E+08 | − | Sense | Sense |
| 18110303 | 18110614 | + | antiSense | antiSense |
| 18120213 | 18120443 | − | antiSense | antiSense |
| 18568782 | 18569081 | + | Sense | Sense |
| 18580697 | 18580997 | + | Sense | Sense |
| 20927249 | 20927554 | − | Sense | Sense |
| 21309344 | 21309617 | + | antiSense | antiSense |
| 21928974 | 21929259 | + | Sense | Sense |
| 21971548 | 21971855 | − | Sense | Sense |
| 24150310 | 24150607 | − | Sense | Sense |
| 24150883 | 24151184 | − | Sense | Sense |
| 24378307 | 24378604 | + | antiSense | antiSense |
| 24378307 | 24378604 | + | antiSense | antiSense |
| 24378307 | 24378604 | + | Sense | antiSense |
| 25519158 | 25519457 | − | Sense | Sense |
| 25867337 | 25867575 | + | Sense | Sense |
| 28295314 | 28295610 | − | antiSense | antiSense |
| 28295909 | 28296217 | + | antiSense | antiSense |
| 28297588 | 28297875 | + | antiSense | antiSense |
| 29194291 | 29194595 | − | antiSense | antiSense |
| 29288879 | 29289182 | + | Sense | Sense |
| 29289967 | 29290241 | − | Sense | Sense |
| 29291386 | 29291668 | + | Sense | Sense |
| 29496958 | 29497263 | + | Sense | Sense |
| 29901855 | 29902146 | − | antiSense | antiSense |
| 29902380 | 29902676 | + | antiSense | antiSense |
| 29969477 | 29969771 | + | antiSense | antiSense |
| 30286226 | 30286521 | + | Sense | Sense |
| 30287433 | 30287715 | − | Sense | Sense |
| 30883904 | 30884204 | + | antiSense | antiSense |
| 30885209 | 30885526 | − | antiSense | antiSense |
| 31051983 | 31052261 | − | antiSense | antiSense |
| 31053610 | 31053893 | + | antiSense | antiSense |
| 31055356 | 31055638 | − | antiSense | antiSense |
| 31360062 | 31360373 | + | antiSense | antiSense |
| 31360062 | 31360373 | + | antiSense | antiSense |
| 31360062 | 31360373 | + | antiSense | antiSense |
| 31362479 | 31362799 | + | antiSense | antiSense |
| 32140120 | 32140418 | − | antiSense | antiSense |
| 33918978 | 33919295 | − | antiSense | antiSense |
| 34150325 | 34150622 | − | antiSense | antiSense |
| 35805292 | 35805586 | + | Sense | Sense |
| 36159327 | 36159622 | + | antiSense | antiSense |
| 36420857 | 36421154 | − | antiSense | antiSense |
| 37424292 | 37424601 | + | Sense | Sense |
| 38275226 | 38275514 | + | Sense | Sense |
| 38341491 | 38341803 | − | Sense | Sense |
| 38343510 | 38343811 | − | Sense | Sense |
| 38703704 | 38703994 | − | antiSense | antiSense |
| 38751016 | 38751322 | − | antiSense | antiSense |
| 38751016 | 38751322 | − | antiSense | antiSense |
| 38751633 | 38751896 | − | antiSense | antiSense |
| 38751633 | 38751896 | − | antiSense | antiSense |
| 38752262 | 38752564 | + | antiSense | antiSense |
| 38752262 | 38752564 | + | antiSense | antiSense |
| 38753459 | 38753743 | + | antiSense | antiSense |
| 38753459 | 38753743 | + | antiSense | antiSense |
| 39059180 | 39059406 | + | Sense | Sense |
| 39060853 | 39061148 | − | Sense | Sense |
| 40911416 | 40911633 | + | antiSense | antiSense |
| 41326130 | 41326432 | − | Sense | Sense |
| 41326130 | 41326432 | − | Sense | Sense |
| 41327440 | 41327732 | + | Sense | Sense |
| 41353070 | 41353358 | + | Sense | Sense |
| 41353368 | 41353652 | + | Sense | Sense |
| 41353835 | 41354124 | − | Sense | Sense |
| 41353835 | 41354124 | − | Sense | Sense |
| 41359358 | 41359653 | + | Sense | Sense |
| 41539579 | 41539882 | − | Sense | Sense |
| 41821080 | 41821389 | + | Sense | Sense |
| 42210288 | 42210583 | + | Sense | Sense |
| 42247479 | 42247798 | − | Sense | Sense |
| 42247479 | 42247798 | − | Sense | Sense |
| 42249758 | 42250061 | + | Sense | Sense |
| 42250155 | 42250465 | − | Sense | Sense |
| 42250155 | 42250465 | − | Sense | Sense |
| 42285723 | 42286017 | + | Sense | Sense |
| 42960434 | 42960736 | − | antiSense | antiSense |
| 42967485 | 42967727 | − | antiSense | antiSense |
| 42974861 | 42975163 | + | antiSense | antiSense |
| 42984965 | 42985265 | + | antiSense | antiSense |
| 43224688 | 43224990 | − | antiSense | antiSense |
| 43257783 | 43258071 | − | Sense | Sense |
| 43321205 | 43321503 | + | antiSense | antiSense |
| 44345206 | 44345531 | − | Sense | Sense |
| 45166282 | 45166585 | − | Sense | Sense |
| 45172050 | 45172352 | + | Sense | Sense |
| 45186613 | 45186910 | − | Sense | Sense |
| 45187303 | 45187604 | + | Sense | Sense |
| 45188693 | 45189001 | − | Sense | Sense |
| 45188693 | 45189001 | − | Sense | Sense |
| 45248673 | 45248981 | − | Sense | Sense |
| 45250116 | 45250412 | − | Sense | Sense |
| 45529305 | 45529613 | + | antiSense | antiSense |
| 45537433 | 45537727 | + | antiSense | antiSense |
| 45738396 | 45738690 | + | Sense | Sense |
| 45738870 | 45739163 | + | Sense | Sense |
| 46091371 | 46091671 | − | Sense | Sense |
| 46550569 | 46550874 | − | Sense | Sense |
| 50272821 | 50273074 | − | Sense | Sense |
| 50849606 | 50849899 | + | Sense | Sense |
| 50849900 | 50850192 | + | Sense | Sense |
| 19710320 | 19710600 | + | Sense | Sense |
| 19710320 | 19710600 | + | Sense | Sense |
| 19711170 | 19711480 | − | Sense | Sense |
| 20194717 | 20195018 | + | Sense | Sense |
| 20195025 | 20195330 | − | Sense | Sense |
| 20195985 | 20196287 | + | Sense | Sense |
| 20195985 | 20196287 | + | Sense | Sense |
| 20285495 | 20285805 | + | antiSense | antiSense |
| 28227045 | 28227336 | − | Sense | Sense |
| 43659219 | 43659491 | + | Sense | Sense |
| 45126843 | 45127147 | + | antiSense | antiSense |
| 45605010 | 45605299 | + | Sense | Sense |
| 45900733 | 45901024 | + | antiSense | antiSense |
| 45901029 | 45901331 | + | antiSense | antiSense |
| 45945727 | 45945994 | + | Sense | Sense |
| 50240566 | 50240876 | + | antiSense | antiSense |
| 50576799 | 50577096 | − | Sense | Sense |
| 50597795 | 50598088 | − | antiSense | antiSense |
| 52852024 | 52852324 | − | antiSense | antiSense |
| 53041933 | 53042244 | − | Sense | Sense |
| 53265187 | 53265416 | − | Sense | Sense |
| 73354184 | 73354491 | + | antiSense | antiSense |
| 73385459 | 73385757 | + | Sense | Sense |
| 73642561 | 73642864 | + | Sense | Sense |
| 80111251 | 80111545 | − | Sense | Sense |
| 95237770 | 95238072 | + | antiSense | antiSense |
| 96123708 | 96124005 | − | antiSense | antiSense |
| 96147717 | 96148025 | + | antiSense | antiSense |
| 98807760 | 98808072 | − | Sense | Sense |
| 98819654 | 98819963 | − | Sense | Sense |
| 98910236 | 98910520 | − | Sense | Sense |
| 99116750 | 99117049 | − | antiSense | antiSense |
| 1E+08 | 1E+08 | − | Sense | Sense |
| 1.11E+08 | 1.11E+08 | + | Sense | Sense |
| 1.13E+08 | 1.13E+08 | + | Sense | Sense |
| 470365 | 470659 | − | antiSense | antiSense |
| 976008 | 976305 | + | Sense | Sense |
| 1119677 | 1119954 | − | Sense | Sense |
| 2961889 | 2962191 | + | Sense | Sense |
| 3029394 | 3029692 | + | Sense | Sense |
| 4436892 | 4437131 | − | Sense | Sense |
| 4463892 | 4464196 | − | Sense | Sense |
| 6430723 | 6431013 | + | Sense | Sense |
| 6996055 | 6996377 | + | Sense | Sense |
| 6997951 | 6998240 | − | Sense | Sense |
| 7250582 | 7250895 | + | antiSense | antiSense |
| 7250582 | 7250895 | + | antiSense | antiSense |
| 8950204 | 8950536 | + | Sense | Sense |
| 9811997 | 9812300 | − | Sense | Sense |
| 12350932 | 12351150 | − | antiSense | antiSense |
| 12352618 | 12352928 | + | antiSense | antiSense |
| 12373524 | 12373833 | − | antiSense | antiSense |
| 12527285 | 12527585 | + | Sense | Sense |
| 13050541 | 13050843 | − | Sense | Sense |
| 13051185 | 13051492 | + | Sense | Sense |
| 13056204 | 13056520 | − | Sense | Sense |
| 13056204 | 13056520 | − | Sense | Sense |
| 13057819 | 13058114 | − | Sense | Sense |
| 13057819 | 13058114 | − | Sense | Sense |
| 14324350 | 14324647 | − | Sense | Sense |
| 14912206 | 14912501 | + | antiSense | antiSense |
| 15818269 | 15818564 | − | antiSense | antiSense |
| 19399277 | 19399564 | + | Sense | Sense |
| 26616318 | 26616645 | + | antiSense | antiSense |
| 27940151 | 27940446 | + | Sense | Sense |
| 27946192 | 27946504 | + | Sense | Sense |
| 31432042 | 31432344 | − | antiSense | antiSense |
| 31733758 | 31734039 | + | antiSense | antiSense |
| 31885280 | 31885544 | − | Sense | Sense |
| 31901478 | 31901772 | − | antiSense | antiSense |
| 32142049 | 32142349 | + | Sense | Sense |
| 32148331 | 32148628 | + | Sense | Sense |
| 32148331 | 32148628 | + | Sense | Sense |
| 32150691 | 32150894 | + | Sense | Sense |
| 32286716 | 32287013 | − | Sense | Sense |
| 32559206 | 32559513 | + | Sense | Sense |
| 32638371 | 32638677 | − | Sense | Sense |
| 32897366 | 32897664 | + | antiSense | antiSense |
| 32897366 | 32897664 | + | antiSense | antiSense |
| 32897366 | 32897664 | + | antiSense | antiSense |
| 39822491 | 39822774 | + | antiSense | antiSense |
| 42627691 | 42628009 | + | antiSense | antiSense |
| 42754261 | 42754594 | − | Sense | Sense |
| 46669744 | 46670043 | − | Sense | Sense |
| 49051736 | 49052016 | + | antiSense | antiSense |
| 49722367 | 49722669 | + | Sense | Sense |
| 49749178 | 49749480 | − | Sense | Sense |
| 49773049 | 49773362 | + | Sense | Sense |
| 49782177 | 49782458 | − | Sense | Sense |
| 49863812 | 49864114 | − | Sense | Sense |
| 49886597 | 49886911 | + | Sense | Sense |
| 49903355 | 49903623 | + | Sense | Sense |
| 50546571 | 50546854 | + | antiSense | antiSense |
| 50553379 | 50553673 | − | antiSense | antiSense |
| 50553806 | 50554117 | + | antiSense | antiSense |
| 50555278 | 50555583 | + | antiSense | antiSense |
| 50556393 | 50556686 | − | antiSense | antiSense |
| 50556393 | 50556686 | − | antiSense | antiSense |
| 50596792 | 50597095 | − | antiSense | antiSense |
| 50851719 | 50852017 | − | Sense | Sense |
| 50853328 | 50853629 | + | Sense | Sense |
| 50910783 | 50911001 | − | Sense | Sense |
| 50929152 | 50929453 | − | Sense | Sense |
| 50938398 | 50938698 | − | Sense | Sense |
| 50938398 | 50938698 | − | Sense | Sense |
| 51325211 | 51325520 | + | Sense | Sense |
| 51607645 | 51607919 | + | antiSense | antiSense |
| 53570519 | 53570814 | − | antiSense | antiSense |
| 53690932 | 53691234 | + | Sense | Sense |
| 53841147 | 53841388 | + | Sense | Sense |
| 53842734 | 53843033 | − | Sense | Sense |
| 53843155 | 53843462 | − | Sense | Sense |
| 53863545 | 53863829 | − | Sense | Sense |
| 53863888 | 53864186 | + | Sense | Sense |
| 53864197 | 53864456 | − | Sense | Sense |
| 54065995 | 54066264 | − | antiSense | antiSense |
| 54067326 | 54067619 | + | antiSense | antiSense |
| 54721781 | 54722073 | + | Sense | Sense |
| 54726816 | 54727131 | − | Sense | Sense |
| 56082227 | 56082520 | + | Sense | Sense |
| 56220692 | 56220992 | + | antiSense | antiSense |
| 56220692 | 56220992 | + | Sense | Sense |
| 56228045 | 56228347 | − | Sense | Sense |
| 56691786 | 56692081 | − | antiSense | antiSense |
| 56707384 | 56707689 | − | antiSense | antiSense |
| 57072619 | 57072906 | + | antiSense | antiSense |
| 57072914 | 57073215 | + | antiSense | antiSense |
| 57074162 | 57074465 | − | antiSense | antiSense |
| 57075650 | 57075929 | − | antiSense | antiSense |
| 57075650 | 57075929 | − | antiSense | antiSense |
| 57650593 | 57650900 | − | antiSense | antiSense |
| 57792905 | 57793209 | + | antiSense | antiSense |
| 57807631 | 57807931 | − | antiSense | antiSense |
| 57821139 | 57821432 | − | antiSense | antiSense |
| 57822532 | 57822828 | − | antiSense | antiSense |
| 58178700 | 58179008 | − | Sense | Sense |
| 62791781 | 62792071 | − | Sense | Sense |
| 62907445 | 62907759 | + | Sense | Sense |
| 64267438 | 64267603 | − | Sense | Sense |
| 64857638 | 64857941 | − | Sense | Sense |
| 64887129 | 64887418 | + | Sense | Sense |
| 64887129 | 64887418 | + | Sense | Sense |
| 65024028 | 65024330 | + | Sense | Sense |
| 65032079 | 65032376 | + | Sense | Sense |
| 65032079 | 65032376 | + | Sense | Sense |
| 65055452 | 65055760 | − | Sense | Sense |
| 65055452 | 65055760 | − | Sense | Sense |
| 65055918 | 65056226 | + | Sense | Sense |
| 65193605 | 65193908 | + | Sense | Sense |
| 66239033 | 66239348 | − | Sense | Sense |
| 66255623 | 66255923 | − | Sense | Sense |
| 66515061 | 66515335 | − | antiSense | antiSense |
| 66516915 | 66517204 | − | antiSense | antiSense |
| 66544204 | 66544492 | − | antiSense | antiSense |
| 69031096 | 69031408 | + | Sense | Sense |
| 69032443 | 69032740 | − | Sense | Sense |
| 69159994 | 69160284 | + | Sense | Sense |
| 69160372 | 69160664 | − | Sense | Sense |
| 69161098 | 69161317 | + | Sense | Sense |
| 69161098 | 69161317 | + | Sense | Sense |
| 69215221 | 69215528 | + | Sense | Sense |
| 69215639 | 69215930 | − | Sense | Sense |
| 70815355 | 70815660 | − | Sense | Sense |
| 72054802 | 72055104 | + | antiSense | antiSense |
| 72055259 | 72055559 | − | antiSense | antiSense |
| 74524586 | 74524896 | − | antiSense | antiSense |
| 76951116 | 76951411 | + | antiSense | antiSense |
| 80194514 | 80194811 | − | antiSense | antiSense |
| 80194844 | 80195155 | + | antiSense | antiSense |
| 81220890 | 81221188 | + | antiSense | antiSense |
| 83163398 | 83163688 | − | Sense | Sense |
| 83258713 | 83259001 | + | Sense | Sense |
| 89830210 | 89830523 | + | antiSense | antiSense |
| 92497618 | 92497919 | + | antiSense | antiSense |
| 93777326 | 93777612 | + | Sense | Sense |
| 93782967 | 93783201 | + | Sense | Sense |
| 93786031 | 93786332 | − | Sense | Sense |
| 93786031 | 93786332 | − | Sense | Sense |
| 93786031 | 93786332 | − | Sense | Sense |
| 94725134 | 94725447 | − | antiSense | antiSense |
| 96607927 | 96608238 | + | antiSense | antiSense |
| 98886064 | 98886368 | + | antiSense | antiSense |
| 98918070 | 98918372 | + | Sense | Sense |
| 1.03E+08 | 1.03E+08 | + | Sense | Sense |
| 1.04E+08 | 1.04E+08 | + | antiSense | antiSense |
| 1.05E+08 | 1.05E+08 | − | Sense | Sense |
| 1.05E+08 | 1.05E+08 | − | Sense | Sense |
| 1.09E+08 | 1.09E+08 | + | antiSense | antiSense |
| 1.09E+08 | 1.09E+08 | + | antiSense | antiSense |
| 1.09E+08 | 1.09E+08 | + | antiSense | antiSense |
| 1.11E+08 | 1.11E+08 | − | Sense | Sense |
| 1.11E+08 | 1.11E+08 | + | Sense | Sense |
| 1.11E+08 | 1.11E+08 | − | antiSense | antiSense |
| 1.11E+08 | 1.11E+08 | − | antiSense | antiSense |
| 1.11E+08 | 1.11E+08 | − | antiSense | antiSense |
| 1.11E+08 | 1.11E+08 | + | antiSense | antiSense |
| 1.11E+08 | 1.11E+08 | + | antiSense | antiSense |
| 1.11E+08 | 1.11E+08 | + | antiSense | antiSense |
| 1.11E+08 | 1.11E+08 | + | antiSense | antiSense |
| 1.11E+08 | 1.11E+08 | − | antiSense | antiSense |
| 1.11E+08 | 1.11E+08 | − | antiSense | antiSense |
| 1.11E+08 | 1.11E+08 | − | antiSense | antiSense |
| 1.11E+08 | 1.11E+08 | + | antiSense | antiSense |
| 1.11E+08 | 1.11E+08 | + | antiSense | antiSense |
| 1.11E+08 | 1.11E+08 | + | antiSense | antiSense |
| 1.11E+08 | 1.11E+08 | + | antiSense | antiSense |
| 1.11E+08 | 1.11E+08 | + | antiSense | antiSense |
| 1.11E+08 | 1.11E+08 | − | antiSense | antiSense |
| 1.11E+08 | 1.11E+08 | + | antiSense | antiSense |
| 1.11E+08 | 1.11E+08 | + | antiSense | antiSense |
| 1.11E+08 | 1.11E+08 | + | antiSense | antiSense |
| 1.11E+08 | 1.11E+08 | + | antiSense | antiSense |
| 1.12E+08 | 1.12E+08 | − | antiSense | antiSense |
| 1.12E+08 | 1.12E+08 | − | antiSense | antiSense |
| 1.12E+08 | 1.12E+08 | + | antiSense | antiSense |
| 1.12E+08 | 1.12E+08 | − | antiSense | antiSense |
| 1.12E+08 | 1.12E+08 | − | antiSense | antiSense |
| 1.12E+08 | 1.12E+08 | + | antiSense | antiSense |
| 1.12E+08 | 1.12E+08 | + | antiSense | antiSense |
| 1.12E+08 | 1.12E+08 | + | antiSense | antiSense |
| 1.12E+08 | 1.12E+08 | + | antiSense | antiSense |
| 1.12E+08 | 1.12E+08 | − | antiSense | antiSense |
| 1.12E+08 | 1.12E+08 | − | antiSense | antiSense |
| 1.12E+08 | 1.12E+08 | + | antiSense | antiSense |
| 1.12E+08 | 1.12E+08 | − | antiSense | antiSense |
| 1.12E+08 | 1.12E+08 | − | antiSense | antiSense |
| 1.12E+08 | 1.12E+08 | + | antiSense | antiSense |
| 1.12E+08 | 1.12E+08 | − | antiSense | antiSense |
| 1.12E+08 | 1.12E+08 | − | antiSense | antiSense |
| 1.12E+08 | 1.12E+08 | − | Sense | Sense |
| 1.12E+08 | 1.12E+08 | + | Sense | Sense |
| 1.12E+08 | 1.12E+08 | + | Sense | Sense |
| 1.12E+08 | 1.12E+08 | + | antiSense | antiSense |
| 1.13E+08 | 1.13E+08 | − | antiSense | antiSense |
| 1.13E+08 | 1.13E+08 | + | antiSense | antiSense |
| 1.13E+08 | 1.13E+08 | − | Sense | Sense |
| 1.13E+08 | 1.13E+08 | + | Sense | Sense |
| 1.14E+08 | 1.14E+08 | − | antiSense | antiSense |
| 1.14E+08 | 1.14E+08 | + | Sense | Sense |
| 1.14E+08 | 1.14E+08 | + | Sense | Sense |
| 1.14E+08 | 1.14E+08 | − | Sense | Sense |
| 1.14E+08 | 1.14E+08 | − | Sense | Sense |
| 1.17E+08 | 1.17E+08 | + | antiSense | antiSense |
| 1.17E+08 | 1.17E+08 | + | antiSense | antiSense |
| 1.17E+08 | 1.17E+08 | − | antiSense | antiSense |
| 1.17E+08 | 1.17E+08 | + | antiSense | antiSense |
| 1.17E+08 | 1.17E+08 | − | antiSense | antiSense |
| 1.17E+08 | 1.17E+08 | + | Sense | Sense |
| 1.17E+08 | 1.17E+08 | + | antiSense | antiSense |
| 1.19E+08 | 1.19E+08 | − | Sense | Sense |
| 1.21E+08 | 1.21E+08 | + | antiSense | antiSense |
| 1.21E+08 | 1.21E+08 | + | antiSense | antiSense |
| 1.21E+08 | 1.21E+08 | + | antiSense | antiSense |
| 1.21E+08 | 1.21E+08 | − | antiSense | antiSense |
| 1.21E+08 | 1.21E+08 | − | antiSense | antiSense |
| 1.21E+08 | 1.21E+08 | + | antiSense | antiSense |
| 1.21E+08 | 1.21E+08 | + | antiSense | antiSense |
| 1.21E+08 | 1.21E+08 | + | Sense | Sense |
| 1.21E+08 | 1.21E+08 | − | Sense | Sense |
| 1.21E+08 | 1.21E+08 | − | Sense | Sense |
| 1.21E+08 | 1.21E+08 | + | antiSense | antiSense |
| 1.21E+08 | 1.21E+08 | − | antiSense | antiSense |
| 1.21E+08 | 1.21E+08 | + | antiSense | antiSense |
| 1.21E+08 | 1.21E+08 | + | antiSense | antiSense |
| 1.21E+08 | 1.21E+08 | + | Sense | Sense |
| 1.21E+08 | 1.21E+08 | + | Sense | Sense |
| 1.21E+08 | 1.21E+08 | − | Sense | antiSense |
| 1.21E+08 | 1.21E+08 | + | antiSense | antiSense |
| 1.21E+08 | 1.21E+08 | + | antiSense | antiSense |
| 1.21E+08 | 1.21E+08 | − | antiSense | antiSense |
| 1.21E+08 | 1.21E+08 | − | antiSense | antiSense |
| 1.22E+08 | 1.22E+08 | − | antiSense | antiSense |
| 1.22E+08 | 1.22E+08 | − | antiSense | antiSense |
| 1.22E+08 | 1.22E+08 | − | antiSense | antiSense |
| 1.22E+08 | 1.22E+08 | − | Sense | Sense |
| 1.22E+08 | 1.22E+08 | + | Sense | Sense |
| 1.22E+08 | 1.22E+08 | − | Sense | Sense |
| 1.22E+08 | 1.22E+08 | − | Sense | Sense |
| 1.22E+08 | 1.22E+08 | − | Sense | Sense |
| 1.22E+08 | 1.22E+08 | − | Sense | Sense |
| 1.22E+08 | 1.22E+08 | + | Sense | Sense |
| 1.22E+08 | 1.22E+08 | + | antiSense | antiSense |
| 1.22E+08 | 1.22E+08 | − | Sense | Sense |
| 1.22E+08 | 1.22E+08 | − | antiSense | antiSense |
| 1.22E+08 | 1.22E+08 | − | Sense | Sense |
| 1.22E+08 | 1.22E+08 | + | Sense | Sense |
| 1.23E+08 | 1.23E+08 | − | Sense | Sense |
| 1.23E+08 | 1.23E+08 | − | Sense | Sense |
| 1.23E+08 | 1.23E+08 | − | Sense | Sense |
| 1.23E+08 | 1.23E+08 | − | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | − | Sense | Sense |
| 1.23E+08 | 1.23E+08 | − | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | − | Sense | Sense |
| 1.23E+08 | 1.23E+08 | − | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | − | Sense | Sense |
| 1.23E+08 | 1.23E+08 | − | Sense | Sense |
| 1.23E+08 | 1.23E+08 | − | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | − | antiSense | antiSense |
| 1.23E+08 | 1.23E+08 | + | antiSense | antiSense |
| 1.23E+08 | 1.23E+08 | − | Sense | Sense |
| 1.23E+08 | 1.23E+08 | + | Sense | Sense |
| 1.24E+08 | 1.24E+08 | + | antiSense | antiSense |
| 1.24E+08 | 1.24E+08 | + | antiSense | antiSense |
| 1.24E+08 | 1.24E+08 | − | antiSense | antiSense |
| 1.32E+08 | 1.32E+08 | + | Sense | Sense |
| 1.32E+08 | 1.32E+08 | − | Sense | Sense |
| 1.33E+08 | 1.33E+08 | − | antiSense | antiSense |
| 1.33E+08 | 1.33E+08 | − | antiSense | antiSense |
| 1.33E+08 | 1.33E+08 | − | antiSense | antiSense |
| 1.33E+08 | 1.33E+08 | + | antiSense | antiSense |
| 1.33E+08 | 1.33E+08 | + | antiSense | antiSense |
| 1.33E+08 | 1.33E+08 | + | antiSense | antiSense |
| 1.33E+08 | 1.33E+08 | + | antiSense | antiSense |
| 1.33E+08 | 1.33E+08 | − | antiSense | antiSense |
| 1.33E+08 | 1.33E+08 | − | antiSense | antiSense |
| 1.33E+08 | 1.33E+08 | − | antiSense | antiSense |
| 1.34E+08 | 1.34E+08 | + | Sense | Sense |
| 1.34E+08 | 1.34E+08 | − | Sense | Sense |
| 1.34E+08 | 1.34E+08 | + | antiSense | antiSense |
| 9945824 | 9946121 | − | antiSense | antiSense |
| 9945824 | 9946121 | − | antiSense | antiSense |
| 11089237 | 11089543 | + | antiSense | antiSense |
| 11089714 | 11090007 | − | antiSense | antiSense |
| 11091813 | 11092107 | + | antiSense | antiSense |
| 11092171 | 11092483 | − | antiSense | antiSense |
| 11092484 | 11092737 | − | antiSense | antiSense |
| 11094648 | 11094933 | + | antiSense | antiSense |
| 11094648 | 11094933 | + | antiSense | antiSense |
| 11096512 | 11096813 | − | antiSense | antiSense |
| 11096848 | 11097154 | + | antiSense | antiSense |
| 11097269 | 11097543 | + | antiSense | antiSense |
| 11179613 | 11179913 | − | Sense | Sense |
| 11179613 | 11179913 | − | Sense | Sense |
| 20366427 | 20366740 | + | Sense | Sense |
| 33099826 | 33100116 | + | antiSense | antiSense |
| 34637012 | 34637301 | + | Sense | Sense |
| 34729837 | 34730133 | − | Sense | Sense |
| 36246625 | 36246932 | + | antiSense | antiSense |
| 36249148 | 36249453 | − | antiSense | antiSense |
| 37526727 | 37527024 | − | antiSense | antiSense |
| 38254734 | 38255060 | − | antiSense | antiSense |
| 38391365 | 38391594 | + | Sense | Sense |
| 40674704 | 40674989 | + | antiSense | antiSense |
| 44332069 | 44332361 | + | Sense | Sense |
| 44332069 | 44332361 | + | Sense | Sense |
| 44399572 | 44399873 | + | Sense | Sense |
| 44399572 | 44399873 | + | Sense | Sense |
| 46214901 | 46215203 | + | antiSense | antiSense |
| 46215269 | 46215576 | + | antiSense | antiSense |
| 47638640 | 47638938 | − | antiSense | antiSense |
| 47698987 | 47699213 | + | antiSense | antiSense |
| 298876 | 299137 | + | antiSense | antiSense |
| 298876 | 299137 | + | antiSense | antiSense |
| 299312 | 299605 | + | antiSense | antiSense |
| 299312 | 299605 | + | antiSense | antiSense |
| 299606 | 299902 | + | antiSense | antiSense |
| 299606 | 299902 | + | antiSense | antiSense |
| 3807911 | 3808200 | − | Sense | Sense |
| 3808397 | 3808708 | − | Sense | Sense |
| 3810557 | 3810856 | + | Sense | Sense |
| 3810557 | 3810856 | + | Sense | Sense |
| 3810557 | 3810856 | + | Sense | Sense |
| 3810874 | 3811173 | − | Sense | Sense |
| 3812604 | 3812903 | + | Sense | Sense |
| 3812604 | 3812903 | + | Sense | Sense |
| 3812904 | 3813214 | + | Sense | Sense |
| 3812904 | 3813214 | + | Sense | Sense |
| 3813262 | 3813557 | − | Sense | Sense |
| 3813262 | 3813557 | − | Sense | Sense |
| 3813262 | 3813557 | − | Sense | Sense |
| 3813564 | 3813859 | − | Sense | Sense |
| 3813564 | 3813859 | − | Sense | Sense |
| 3813564 | 3813859 | − | Sense | Sense |
| 3849816 | 3850119 | − | Sense | Sense |
| 3849816 | 3850119 | − | Sense | Sense |
| 3850247 | 3850549 | − | Sense | Sense |
| 3850247 | 3850549 | − | Sense | Sense |
| 3851534 | 3851827 | − | Sense | Sense |
| 3878840 | 3879142 | − | Sense | Sense |
| 3878840 | 3879142 | − | Sense | Sense |
| 3886790 | 3887087 | + | Sense | Sense |
| 3901212 | 3901508 | + | Sense | Sense |
| 3902357 | 3902655 | − | Sense | Sense |
| 3909123 | 3909424 | + | Sense | Sense |
| 5092479 | 5092789 | − | antiSense | antiSense |
| 5092479 | 5092789 | − | antiSense | antiSense |
| 5561524 | 5561823 | − | antiSense | antiSense |
| 5569976 | 5570264 | + | antiSense | antiSense |
| 5570905 | 5571199 | + | antiSense | antiSense |
| 5981026 | 5981336 | + | Sense | Sense |
| 6054409 | 6054719 | + | antiSense | antiSense |
| 10413654 | 10413951 | − | antiSense | antiSense |
| 10460372 | 10460669 | + | Sense | Sense |
| 17548223 | 17548522 | + | antiSense | antiSense |
| 17957740 | 17958047 | + | Sense | Sense |
| 17967552 | 17967838 | + | Sense | Sense |
| 17967552 | 17967838 | + | Sense | Sense |
| 21296611 | 21296913 | + | Sense | Sense |
| 21296611 | 21296913 | + | Sense | Sense |
| 25367234 | 25367539 | + | antiSense | antiSense |
| 25391628 | 25391921 | + | Sense | Sense |
| 25559149 | 25559448 | + | antiSense | antiSense |
| 25559149 | 25559448 | + | antiSense | antiSense |
| 30189148 | 30189457 | − | antiSense | antiSense |
| 30190072 | 30190361 | + | antiSense | antiSense |
| 30191362 | 30191655 | − | antiSense | antiSense |
| 30191362 | 30191655 | − | antiSense | antiSense |
| 32085354 | 32085652 | − | Sense | Sense |
| 32093308 | 32093607 | + | Sense | Sense |
| 32098005 | 32098316 | − | Sense | Sense |
| 32100557 | 32100857 | − | Sense | Sense |
| 32235077 | 32235389 | − | Sense | Sense |
| 32862475 | 32862757 | + | antiSense | antiSense |
| 32862933 | 32863237 | − | antiSense | antiSense |
| 32865292 | 32865585 | + | antiSense | antiSense |
| 32887708 | 32888012 | + | antiSense | antiSense |
| 32959080 | 32959389 | − | Sense | Sense |
| 32960782 | 32961078 | − | Sense | Sense |
| 32961086 | 32961363 | − | Sense | Sense |
| 33055478 | 33055686 | − | Sense | Sense |
| 33055840 | 33056138 | − | Sense | Sense |
| 33056314 | 33056624 | + | Sense | Sense |
| 33056314 | 33056624 | + | Sense | Sense |
| 33057102 | 33057395 | − | Sense | Sense |
| 33057102 | 33057395 | − | Sense | Sense |
| 33075462 | 33075761 | + | Sense | Sense |
| 33084808 | 33085099 | − | Sense | Sense |
| 33395671 | 33395973 | + | antiSense | antiSense |
| 33617768 | 33618066 | + | antiSense | antiSense |
| 33869255 | 33869548 | − | antiSense | antiSense |
| 33990100 | 33990418 | + | antiSense | antiSense |
| 34173679 | 34173986 | + | antiSense | antiSense |
| 34176544 | 34176853 | + | antiSense | antiSense |
| 34198387 | 34198680 | + | antiSense | antiSense |
| 34198387 | 34198680 | + | antiSense | antiSense |
| 34198387 | 34198680 | + | antiSense | antiSense |
| 34199323 | 34199618 | − | antiSense | antiSense |
| 34199782 | 34200079 | + | antiSense | antiSense |
| 34200082 | 34200351 | − | antiSense | antiSense |
| 34200082 | 34200351 | − | antiSense | antiSense |
| 34200623 | 34200918 | − | antiSense | antiSense |
| 34200623 | 34200918 | − | antiSense | antiSense |
| 34201089 | 34201400 | − | antiSense | antiSense |
| 34201089 | 34201400 | − | antiSense | antiSense |
| 34201458 | 34201771 | + | antiSense | antiSense |
| 34201458 | 34201771 | + | antiSense | antiSense |
| 34201458 | 34201771 | + | antiSense | antiSense |
| 34201458 | 34201771 | + | antiSense | antiSense |
| 34202306 | 34202607 | + | antiSense | antiSense |
| 34202306 | 34202607 | + | antiSense | antiSense |
| 34202306 | 34202607 | + | antiSense | antiSense |
| 34202306 | 34202607 | + | antiSense | antiSense |
| 34203054 | 34203334 | + | antiSense | antiSense |
| 34217933 | 34218247 | + | antiSense | antiSense |
| 34222660 | 34222964 | − | antiSense | antiSense |
| 34224440 | 34224679 | + | antiSense | antiSense |
| 34265176 | 34265475 | + | antiSense | antiSense |
| 34272819 | 34273107 | − | antiSense | antiSense |
| 34311365 | 34311659 | − | antiSense | antiSense |
| 34311705 | 34311968 | + | antiSense | antiSense |
| 34311972 | 34312267 | + | antiSense | antiSense |
| 34314050 | 34314352 | − | antiSense | antiSense |
| 34314050 | 34314352 | − | antiSense | antiSense |
| 34325503 | 34325793 | + | antiSense | antiSense |
| 34711889 | 34712220 | + | Sense | Sense |
| 35527035 | 35527342 | + | Sense | Sense |
| 35528654 | 35528948 | − | Sense | Sense |
| 35535200 | 35535494 | − | Sense | Sense |
| 35535495 | 35535827 | − | Sense | Sense |
| 35537790 | 35538110 | + | Sense | Sense |
| 35537790 | 35538110 | + | Sense | Sense |
| 35543132 | 35543432 | + | Sense | Sense |
| 35543132 | 35543432 | + | Sense | Sense |
| 37641177 | 37641467 | − | Sense | Sense |
| 40100592 | 40100901 | + | antiSense | antiSense |
| 42205988 | 42206288 | − | Sense | Sense |
| 42206289 | 42206584 | − | Sense | Sense |
| 42206843 | 42207139 | + | Sense | Sense |
| 42206843 | 42207139 | + | Sense | Sense |
| 42207652 | 42207955 | − | Sense | Sense |
| 42207652 | 42207955 | − | Sense | Sense |
| 42209086 | 42209388 | + | Sense | Sense |
| 42209086 | 42209388 | + | Sense | Sense |
| 42209086 | 42209388 | + | Sense | Sense |
| 42209504 | 42209814 | − | Sense | Sense |
| 42209504 | 42209814 | − | Sense | Sense |
| 42216018 | 42216306 | + | Sense | Sense |
| 42934528 | 42934829 | − | antiSense | antiSense |
| 42977141 | 42977453 | − | antiSense | antiSense |
| 42993107 | 42993407 | − | Sense | Sense |
| 42997606 | 42997918 | + | Sense | Sense |
| 42998655 | 42998966 | − | Sense | Sense |
| 43000001 | 43000142 | − | Sense | Sense |
| 43051675 | 43051979 | + | Sense | Sense |
| 43053628 | 43053918 | − | Sense | Sense |
| 43055162 | 43055462 | + | Sense | Sense |
| 43674797 | 43675101 | + | Sense | Sense |
| 43707646 | 43707871 | + | Sense | Sense |
| 44467873 | 44468186 | + | Sense | Sense |
| 44514278 | 44514584 | + | Sense | antiSense |
| 44514594 | 44514868 | + | antiSense | antiSense |
| 44525071 | 44525374 | − | Sense | Sense |
| 45870553 | 45870860 | − | antiSense | antiSense |
| 45873519 | 45873799 | + | antiSense | antiSense |
| 45952981 | 45953283 | + | antiSense | antiSense |
| 46143915 | 46144199 | − | Sense | Sense |
| 46144214 | 46144523 | − | Sense | Sense |
| 46145383 | 46145682 | + | Sense | Sense |
| 46145383 | 46145682 | + | Sense | Sense |
| 46145686 | 46145959 | + | Sense | Sense |
| 46145686 | 46145959 | + | Sense | Sense |
| 46146183 | 46146494 | − | Sense | Sense |
| 46146183 | 46146494 | − | Sense | Sense |
| 46146809 | 46147126 | + | Sense | Sense |
| 46146809 | 46147126 | + | Sense | Sense |
| 46146809 | 46147126 | + | Sense | Sense |
| 46147152 | 46147468 | − | Sense | Sense |
| 46147152 | 46147468 | − | Sense | Sense |
| 46147152 | 46147468 | − | Sense | Sense |
| 46147603 | 46147897 | + | Sense | Sense |
| 46147603 | 46147897 | + | Sense | Sense |
| 46147898 | 46148187 | + | Sense | Sense |
| 46147898 | 46148187 | + | Sense | Sense |
| 46159800 | 46160079 | + | Sense | Sense |
| 47578119 | 47578413 | + | Sense | Sense |
| 47578119 | 47578413 | + | Sense | Sense |
| 47726230 | 47726522 | − | antiSense | antiSense |
| 47779957 | 47780260 | − | antiSense | antiSense |
| 47779957 | 47780260 | − | antiSense | antiSense |
| 47780488 | 47780779 | + | antiSense | antiSense |
| 47780840 | 47781126 | − | antiSense | antiSense |
| 47780840 | 47781126 | − | antiSense | antiSense |
| 47780840 | 47781126 | − | antiSense | antiSense |
| 47800470 | 47800750 | − | antiSense | antiSense |
| 48326027 | 48326321 | + | antiSense | antiSense |
| 48326945 | 48327254 | − | antiSense | antiSense |
| 48433583 | 48433881 | + | Sense | Sense |
| 48556950 | 48557248 | − | Sense | Sense |
| 48559078 | 48559373 | + | Sense | Sense |
| 48560597 | 48560899 | − | Sense | Sense |
| 48561136 | 48561441 | + | Sense | Sense |
| 48790144 | 48790442 | − | antiSense | Sense |
| 48790551 | 48790864 | + | Sense | Sense |
| 49355702 | 49355981 | + | Sense | Sense |
| 49358615 | 49358880 | − | Sense | Sense |
| 49358889 | 49359202 | − | Sense | Sense |
| 49360164 | 49360446 | + | Sense | Sense |
| 49360164 | 49360446 | + | Sense | Sense |
| 49360640 | 49360944 | − | Sense | Sense |
| 49555235 | 49555536 | + | antiSense | antiSense |
| 49559892 | 49560194 | − | antiSense | antiSense |
| 50354961 | 50355274 | + | antiSense | antiSense |
| 51713140 | 51713453 | + | Sense | Sense |
| 52088333 | 52088641 | − | antiSense | antiSense |
| 52114284 | 52114588 | − | antiSense | antiSense |
| 52119947 | 52120235 | + | antiSense | antiSense |
| 52190329 | 52190622 | + | antiSense | antiSense |
| 56301854 | 56302153 | − | Sense | Sense |
| 60742091 | 60742398 | − | Sense | Sense |
| 60742399 | 60742624 | + | Sense | Sense |
| 60865459 | 60865775 | − | Sense | Sense |
| 60865782 | 60866070 | − | Sense | Sense |
| 61382240 | 61382528 | − | Sense | Sense |
| 61486838 | 61487149 | + | antiSense | antiSense |
| 61487321 | 61487633 | + | antiSense | antiSense |
| 62228506 | 62228815 | − | antiSense | antiSense |
| 62341214 | 62341511 | − | Sense | Sense |
| 62344942 | 62345242 | + | Sense | Sense |
| 62344942 | 62345242 | + | Sense | Sense |
| 62345496 | 62345798 | + | Sense | Sense |
| 62345496 | 62345798 | + | Sense | Sense |
| 62346002 | 62346286 | − | Sense | Sense |
| 62346002 | 62346286 | − | Sense | Sense |
| 62498637 | 62498935 | + | Sense | Sense |
| 62498637 | 62498935 | + | Sense | Sense |
| 62502394 | 62502695 | − | Sense | Sense |
| 62503727 | 62504030 | − | Sense | Sense |
| 62530931 | 62531231 | − | Sense | Sense |
| 62531654 | 62531956 | + | Sense | Sense |
| 62552663 | 62552960 | + | Sense | Sense |
| 62552663 | 62552960 | + | Sense | Sense |
| 62553139 | 62553431 | − | Sense | Sense |
| 62553543 | 62553835 | + | Sense | Sense |
| 62553543 | 62553835 | + | Sense | Sense |
| 62553543 | 62553835 | + | Sense | Sense |
| 62618240 | 62618542 | + | Sense | Sense |
| 62652139 | 62652431 | − | Sense | Sense |
| 508485 | 508761 | + | Sense | Sense |
| 508762 | 509062 | + | Sense | Sense |
| 511949 | 512230 | − | Sense | Sense |
| 588858 | 589153 | + | Sense | Sense |
| 701403 | 701708 | − | Sense | Sense |
| 746134 | 746427 | + | antiSense | antiSense |
| 927535 | 927840 | + | Sense | Sense |
| 3007857 | 3008154 | − | antiSense | antiSense |
| 3034799 | 3035055 | + | antiSense | antiSense |
| 3812481 | 3812792 | + | antiSense | antiSense |
| 3814528 | 3814814 | − | antiSense | antiSense |
| 3814919 | 3815223 | + | antiSense | antiSense |
| 3814919 | 3815223 | + | antiSense | antiSense |
| 4135944 | 4136240 | − | Sense | Sense |
| 8708743 | 8708937 | + | Sense | Sense |
| 9315471 | 9315757 | + | antiSense | antiSense |
| 9315471 | 9315757 | + | antiSense | antiSense |
| 9315761 | 9316052 | + | antiSense | antiSense |
| 9315761 | 9316052 | + | antiSense | antiSense |
| 9318643 | 9318931 | − | antiSense | antiSense |
| 9318643 | 9318931 | − | antiSense | antiSense |
| 9318643 | 9318931 | − | antiSense | antiSense |
| 9321385 | 9321678 | + | antiSense | antiSense |
| 9436583 | 9436884 | + | Sense | Sense |
| 9831541 | 9831841 | + | Sense | Sense |
| 11923224 | 11923523 | + | Sense | Sense |
| 12892151 | 12892454 | + | Sense | Sense |
| 13021407 | 13021703 | + | antiSense | antiSense |
| 13040290 | 13040572 | + | Sense | Sense |
| 14298822 | 14299111 | − | antiSense | antiSense |
| 14608301 | 14608567 | − | antiSense | antiSense |
| 17152967 | 17153267 | − | antiSense | antiSense |
| 17152967 | 17153267 | − | antiSense | antiSense |
| 19784155 | 19784457 | − | Sense | Sense |
| 20387636 | 20387925 | − | Sense | Sense |
| 27232634 | 27232932 | + | antiSense | antiSense |
| 32878378 | 32878671 | − | Sense | Sense |
| 33098274 | 33098569 | − | Sense | Sense |
| 43823528 | 43823835 | − | Sense | Sense |
| 43846400 | 43846694 | + | Sense | Sense |
| 43881880 | 43882185 | − | Sense | Sense |
| 46491208 | 46491527 | − | antiSense | antiSense |
| 46492849 | 46493122 | + | antiSense | antiSense |
| 46597055 | 46597364 | − | antiSense | antiSense |
| 46607677 | 46607960 | + | antiSense | antiSense |
| 46641298 | 46641594 | + | Sense | Sense |
| 47247821 | 47248123 | − | Sense | Sense |
| 47502652 | 47502981 | − | antiSense | antiSense |
| 47503474 | 47503804 | + | antiSense | antiSense |
| 47532453 | 47532754 | + | antiSense | antiSense |
| 47535017 | 47535306 | − | antiSense | antiSense |
| 47555997 | 47556306 | + | antiSense | antiSense |
| 47555997 | 47556306 | + | antiSense | antiSense |
| 47557309 | 47557613 | − | antiSense | antiSense |
| 47558029 | 47558334 | + | antiSense | antiSense |
| 47558029 | 47558334 | + | antiSense | antiSense |
| 47569571 | 47569859 | + | antiSense | antiSense |
| 47863234 | 47863548 | − | antiSense | antiSense |
| 47864260 | 47864560 | + | antiSense | antiSense |
| 57536497 | 57536806 | + | Sense | Sense |
| 57536497 | 57536806 | + | Sense | Sense |
| 57537516 | 57537830 | + | Sense | Sense |
| 57537516 | 57537830 | + | Sense | Sense |
| 57537845 | 57538153 | + | Sense | Sense |
| 57539398 | 57539703 | − | Sense | Sense |
| 57539398 | 57539703 | − | Sense | Sense |
| 57539398 | 57539703 | − | Sense | Sense |
| 57541127 | 57541419 | + | Sense | Sense |
| 57550476 | 57550778 | + | Sense | Sense |
| 57550476 | 57550778 | + | Sense | Sense |
| 57550863 | 57551144 | + | Sense | Sense |
| 57551384 | 57551693 | − | Sense | Sense |
| 57551384 | 57551693 | − | Sense | Sense |
| 57551975 | 57552277 | − | Sense | Sense |
| 57551975 | 57552277 | − | Sense | Sense |
| 57553043 | 57553318 | − | Sense | Sense |
| 57553043 | 57553318 | − | Sense | Sense |
| 57553724 | 57554026 | + | Sense | Sense |
| 57553724 | 57554026 | + | Sense | Sense |
| 57553724 | 57554026 | + | Sense | Sense |
| 57578145 | 57578450 | + | Sense | Sense |
| 59360257 | 59360570 | − | antiSense | antiSense |
| 61177584 | 61177855 | − | antiSense | antiSense |
| 61180822 | 61181123 | + | antiSense | antiSense |
| 62409056 | 62409350 | − | antiSense | antiSense |
| 62409387 | 62409698 | + | antiSense | antiSense |
| 62518080 | 62518379 | + | antiSense | antiSense |
| 62518591 | 62518793 | + | antiSense | antiSense |
| 62518591 | 62518793 | + | antiSense | antiSense |
| 62587561 | 62587852 | + | antiSense | antiSense |
| 62588500 | 62588792 | + | antiSense | antiSense |
| 62605952 | 62606257 | + | antiSense | antiSense |
| 62655274 | 62655568 | − | Sense | Sense |
| 63587595 | 63587891 | + | Sense | Sense |
| 63627744 | 63628049 | + | Sense | Sense |
| 63943685 | 63943980 | − | antiSense | antiSense |
| 63950515 | 63950756 | − | antiSense | antiSense |
| 64075705 | 64076010 | + | Sense | Sense |
| 64076011 | 64076304 | + | Sense | Sense |
| 64077124 | 64077426 | + | Sense | Sense |
| 64077710 | 64078005 | − | Sense | Sense |
| 64077710 | 64078005 | − | Sense | Sense |
| 64077710 | 64078005 | − | Sense | Sense |
| 64078009 | 64078319 | − | Sense | Sense |
| 64078009 | 64078319 | − | Sense | Sense |
| 64078009 | 64078319 | − | Sense | Sense |
| 65031018 | 65031312 | + | Sense | Sense |
| 65090795 | 65090976 | − | Sense | Sense |
| 65090980 | 65091292 | − | Sense | Sense |
| 65092384 | 65092677 | − | Sense | Sense |
| 65140441 | 65140721 | + | antiSense | antiSense |
| 65541884 | 65542179 | − | antiSense | antiSense |
| 66124418 | 66124687 | − | Sense | Sense |
| 66124688 | 66124996 | + | Sense | Sense |
| 66417759 | 66418044 | + | Sense | Sense |
| 66418821 | 66419118 | − | Sense | Sense |
| 66419600 | 66419862 | − | Sense | Sense |
| 66514411 | 66514706 | − | Sense | Sense |
| 66519928 | 66520223 | − | Sense | Sense |
| 66561297 | 66561612 | + | Sense | Sense |
| 66562322 | 66562614 | + | Sense | Sense |
| 66562762 | 66563072 | − | Sense | Sense |
| 66562762 | 66563072 | − | Sense | Sense |
| 66898874 | 66899171 | − | Sense | Sense |
| 66915153 | 66915447 | − | Sense | Sense |
| 67062607 | 67062896 | + | Sense | Sense |
| 67854666 | 67854949 | + | antiSense | antiSense |
| 67861727 | 67862017 | + | antiSense | antiSense |
| 68245422 | 68245715 | − | Sense | Sense |
| 70907763 | 70908065 | + | antiSense | antiSense |
| 71658272 | 71658541 | + | Sense | Sense |
| 71999250 | 71999557 | + | antiSense | antiSense |
| 71999250 | 71999557 | + | antiSense | antiSense |
| 72000002 | 72000232 | − | antiSense | antiSense |
| 72001711 | 72002016 | + | antiSense | antiSense |
| 72002358 | 72002650 | − | antiSense | antiSense |
| 72002358 | 72002650 | − | antiSense | antiSense |
| 72002806 | 72003109 | + | antiSense | antiSense |
| 72002806 | 72003109 | + | antiSense | antiSense |
| 74169596 | 74169896 | + | antiSense | antiSense |
| 75751619 | 75751898 | − | Sense | Sense |
| 76076438 | 76076710 | − | antiSense | antiSense |
| 76960030 | 76960329 | + | antiSense | antiSense |
| 76960030 | 76960329 | + | antiSense | antiSense |
| 78174450 | 78174780 | + | antiSense | antiSense |
| 82788106 | 82788395 | − | Sense | Sense |
| 82790670 | 82790964 | + | Sense | Sense |
| 82790670 | 82790964 | + | Sense | Sense |
| 82793831 | 82794133 | − | Sense | Sense |
| 82901038 | 82901337 | − | antiSense | antiSense |
| 1.02E+08 | 1.02E+08 | + | Sense | Sense |
| 1.02E+08 | 1.02E+08 | + | Sense | Sense |
| 1.02E+08 | 1.02E+08 | − | Sense | Sense |
| 1.02E+08 | 1.02E+08 | + | Sense | Sense |
| 1.02E+08 | 1.02E+08 | + | Sense | Sense |
| 1.02E+08 | 1.02E+08 | + | Sense | Sense |
| 1.02E+08 | 1.02E+08 | + | Sense | Sense |
| 1.02E+08 | 1.02E+08 | + | Sense | Sense |
| 1.03E+08 | 1.03E+08 | − | antiSense | antiSense |
| 1.17E+08 | 1.17E+08 | + | antiSense | antiSense |
| 1.19E+08 | 1.19E+08 | − | antiSense | antiSense |
| 1.19E+08 | 1.19E+08 | − | Sense | Sense |
| 1.19E+08 | 1.19E+08 | − | Sense | Sense |
| 1.19E+08 | 1.19E+08 | − | Sense | Sense |
| 1.19E+08 | 1.19E+08 | − | Sense | Sense |
| 1.19E+08 | 1.19E+08 | − | Sense | Sense |
| 1.19E+08 | 1.19E+08 | + | Sense | Sense |
| 1.19E+08 | 1.19E+08 | + | Sense | Sense |
| 1.19E+08 | 1.19E+08 | + | Sense | Sense |
| 1.19E+08 | 1.19E+08 | − | Sense | Sense |
| 1.19E+08 | 1.19E+08 | − | Sense | Sense |
| 1.19E+08 | 1.19E+08 | + | Sense | Sense |
| 1.19E+08 | 1.19E+08 | + | Sense | Sense |
| 1.23E+08 | 1.23E+08 | − | Sense | Sense |
| 1.26E+08 | 1.26E+08 | − | Sense | Sense |
| 1.3E+08 | 1.3E+08 | − | antiSense | antiSense |
| 1.3E+08 | 1.3E+08 | − | Sense | Sense |
| 1.3E+08 | 1.3E+08 | − | Sense | Sense |
| 14176591 | 14176851 | − | Sense | Sense |
| 16926529 | 16926833 | + | antiSense | antiSense |
| 20743780 | 20744060 | − | Sense | Sense |
| 20745835 | 20746146 | − | Sense | Sense |
| 21463023 | 21463334 | + | Sense | Sense |
| 21463023 | 21463334 | + | Sense | Sense |
| 32165407 | 32165707 | + | antiSense | antiSense |
| 32166640 | 32166928 | + | antiSense | antiSense |
| 32167271 | 32167577 | − | antiSense | antiSense |
| 32167271 | 32167577 | − | antiSense | antiSense |
| 33451479 | 33451777 | − | Sense | Sense |
| 33994299 | 33994582 | − | antiSense | antiSense |
| 34668965 | 34669259 | − | Sense | Sense |
| 34908277 | 34908572 | − | antiSense | antiSense |
| 37038087 | 37038391 | − | Sense | Sense |
| 37288884 | 37289181 | + | antiSense | antiSense |
| 37290298 | 37290602 | + | antiSense | antiSense |
| 43129047 | 43129337 | − | Sense | Sense |
| 43129757 | 43130057 | − | Sense | Sense |
| 43130086 | 43130391 | + | Sense | Sense |
| 43130086 | 43130391 | + | Sense | Sense |
| 43311146 | 43311445 | + | antiSense | antiSense |
| 43381494 | 43381761 | − | antiSense | antiSense |
| 43521213 | 43521518 | − | Sense | Sense |
| 60190718 | 60191016 | + | antiSense | antiSense |
| 60674387 | 60674681 | − | Sense | Sense |
| 64843495 | 64843790 | − | antiSense | antiSense |
| 64964289 | 64964585 | + | antiSense | antiSense |
| 65243554 | 65243854 | + | Sense | Sense |
| 65244591 | 65244891 | − | Sense | Sense |
| 68663059 | 68663350 | + | antiSense | antiSense |
| 69370860 | 69371029 | − | Sense | Sense |
| 69371669 | 69371981 | − | Sense | Sense |
| 69371669 | 69371981 | − | Sense | Sense |
| 70817119 | 70817418 | − | Sense | Sense |
| 77430358 | 77430666 | − | antiSense | antiSense |
| 78717768 | 78718071 | + | antiSense | antiSense |
| 78799074 | 78799377 | + | antiSense | antiSense |
| 79449990 | 79450301 | − | antiSense | antiSense |
| 79742799 | 79743109 | + | Sense | Sense |
| 79777640 | 79777939 | − | antiSense | antiSense |
| 79778674 | 79778986 | − | antiSense | antiSense |
| 1.03E+08 | 1.03E+08 | + | Sense | Sense |
| 1.04E+08 | 1.04E+08 | + | Sense | Sense |
| 1.04E+08 | 1.04E+08 | + | Sense | Sense |
| 1.12E+08 | 1.12E+08 | − | Sense | Sense |
| 1.12E+08 | 1.12E+08 | + | Sense | Sense |
| 1.12E+08 | 1.12E+08 | + | Sense | Sense |
| 1.26E+08 | 1.26E+08 | − | Sense | Sense |
| 1.26E+08 | 1.26E+08 | + | Sense | Sense |
| 1.3E+08 | 1.3E+08 | + | antiSense | antiSense |
| 1.31E+08 | 1.31E+08 | − | Sense | Sense |
| 1.31E+08 | 1.31E+08 | + | Sense | Sense |
| 1.31E+08 | 1.31E+08 | + | Sense | Sense |
| 1.32E+08 | 1.32E+08 | − | antiSense | antiSense |
| 1.32E+08 | 1.32E+08 | − | antiSense | antiSense |
| 1.32E+08 | 1.32E+08 | − | antiSense | antiSense |
| 1.32E+08 | 1.32E+08 | + | antiSense | antiSense |
| 1.32E+08 | 1.32E+08 | − | Sense | Sense |
| 1.34E+08 | 1.34E+08 | + | antiSense | antiSense |
| 1.34E+08 | 1.34E+08 | + | Sense | Sense |
| 1.34E+08 | 1.34E+08 | + | Sense | Sense |
| 1.34E+08 | 1.34E+08 | − | antiSense | antiSense |
| 1.34E+08 | 1.34E+08 | − | antiSense | antiSense |
| 1.34E+08 | 1.34E+08 | + | Sense | Sense |
| 1.34E+08 | 1.34E+08 | + | Sense | Sense |
| 1.34E+08 | 1.34E+08 | + | Sense | Sense |
| 1.34E+08 | 1.34E+08 | + | Sense | Sense |
| 1.34E+08 | 1.34E+08 | + | Sense | Sense |
| 1.34E+08 | 1.34E+08 | + | Sense | Sense |
| 1.34E+08 | 1.34E+08 | + | Sense | Sense |
| 1.34E+08 | 1.34E+08 | + | Sense | Sense |
| 1.34E+08 | 1.34E+08 | + | Sense | Sense |
| 1.37E+08 | 1.37E+08 | + | antiSense | antiSense |
| 1.37E+08 | 1.37E+08 | − | antiSense | antiSense |
| 1.38E+08 | 1.38E+08 | − | antiSense | antiSense |
| 1.38E+08 | 1.38E+08 | + | Sense | Sense |
| 1.39E+08 | 1.39E+08 | − | Sense | Sense |
| 1.39E+08 | 1.39E+08 | + | Sense | Sense |
| 1.39E+08 | 1.39E+08 | − | Sense | Sense |
| 1.39E+08 | 1.39E+08 | − | antiSense | antiSense |
| 1.39E+08 | 1.39E+08 | + | antiSense | antiSense |
| 1.39E+08 | 1.39E+08 | + | Sense | Sense |
| 1.4E+08 | 1.4E+08 | + | Sense | Sense |
| 1.4E+08 | 1.4E+08 | − | Sense | Sense |
| 1.41E+08 | 1.41E+08 | − | antiSense | antiSense |
| 1.42E+08 | 1.42E+08 | + | Sense | Sense |
| 1.42E+08 | 1.42E+08 | − | Sense | Sense |
| 1.42E+08 | 1.42E+08 | + | Sense | Sense |
| 1.42E+08 | 1.42E+08 | − | Sense | Sense |
| 1.46E+08 | 1.46E+08 | + | antiSense | antiSense |
| 1.46E+08 | 1.46E+08 | + | Sense | Sense |
| 1.49E+08 | 1.49E+08 | + | antiSense | antiSense |
| 1.49E+08 | 1.49E+08 | + | antiSense | antiSense |
| 1.51E+08 | 1.51E+08 | + | Sense | Sense |
| 1.54E+08 | 1.54E+08 | − | Sense | Sense |
| 1.57E+08 | 1.57E+08 | + | antiSense | antiSense |
| 1.6E+08 | 1.6E+08 | − | antiSense | antiSense |
| 1.6E+08 | 1.6E+08 | − | antiSense | antiSense |
| 1.6E+08 | 1.6E+08 | + | antiSense | antiSense |
| 1.6E+08 | 1.6E+08 | + | antiSense | antiSense |
| 1.6E+08 | 1.6E+08 | − | antiSense | antiSense |
| 1.6E+08 | 1.6E+08 | + | antiSense | antiSense |
| 1.6E+08 | 1.6E+08 | + | antiSense | antiSense |
| 1.6E+08 | 1.6E+08 | − | antiSense | antiSense |
| 1.68E+08 | 1.68E+08 | − | Sense | Sense |
| 1.72E+08 | 1.72E+08 | − | Sense | Sense |
| 1.72E+08 | 1.72E+08 | − | Sense | Sense |
| 1.76E+08 | 1.76E+08 | + | Sense | Sense |
| 1.76E+08 | 1.76E+08 | + | Sense | Sense |
| 1.76E+08 | 1.76E+08 | + | antiSense | antiSense |
| 1.76E+08 | 1.76E+08 | + | Sense | Sense |
| 1.76E+08 | 1.76E+08 | − | Sense | Sense |
| 1.76E+08 | 1.76E+08 | − | Sense | Sense |
| 1.76E+08 | 1.76E+08 | + | Sense | Sense |
| 1.77E+08 | 1.77E+08 | + | Sense | Sense |
| 1.77E+08 | 1.77E+08 | + | Sense | Sense |
| 1.77E+08 | 1.77E+08 | + | antiSense | antiSense |
| 1.78E+08 | 1.78E+08 | + | antiSense | antiSense |
| 1.78E+08 | 1.78E+08 | + | antiSense | antiSense |
| 1.78E+08 | 1.78E+08 | + | antiSense | antiSense |
| 1.78E+08 | 1.78E+08 | + | antiSense | antiSense |
| 1.79E+08 | 1.79E+08 | − | Sense | Sense |
| 1.79E+08 | 1.79E+08 | − | Sense | Sense |
| 1.79E+08 | 1.79E+08 | + | antiSense | antiSense |
| 1.8E+08 | 1.8E+08 | − | Sense | Sense |
| 1.81E+08 | 1.81E+08 | − | Sense | Sense |
| 1.81E+08 | 1.81E+08 | − | Sense | Sense |
| 1.81E+08 | 1.81E+08 | + | Sense | Sense |
| 23358459 | 23358749 | − | antiSense | antiSense |
| 23360537 | 23360842 | + | antiSense | antiSense |
| 23818749 | 23819051 | − | Sense | Sense |
| 25676486 | 25676789 | − | antiSense | antiSense |
| 28840516 | 28840817 | − | Sense | Sense |
| 28852602 | 28852903 | − | Sense | Sense |
| 34523779 | 34524090 | − | antiSense | antiSense |
| 35214011 | 35214312 | − | antiSense | antiSense |
| 38571030 | 38571326 | − | Sense | Sense |
| 40687858 | 40688150 | + | Sense | Sense |
| 40687858 | 40688150 | + | Sense | Sense |
| 40689306 | 40689582 | − | Sense | Sense |
| 40919432 | 40919728 | + | Sense | Sense |
| 40983722 | 40984006 | − | antiSense | antiSense |
| 40985041 | 40985363 | + | antiSense | antiSense |
| 40985458 | 40985750 | − | antiSense | antiSense |
| 40985458 | 40985750 | − | antiSense | antiSense |
| 41590812 | 41591106 | + | Sense | Sense |
| 41593187 | 41593416 | − | Sense | Sense |
| 41594412 | 41594719 | + | Sense | Sense |
| 41597437 | 41597694 | − | Sense | Sense |
| 41812133 | 41812431 | − | antiSense | antiSense |
| 41872825 | 41873128 | − | Sense | Sense |
| 41873129 | 41873410 | + | Sense | Sense |
| 42647947 | 42648238 | + | Sense | Sense |
| 42835397 | 42835698 | − | antiSense | antiSense |
| 42835397 | 42835698 | − | antiSense | antiSense |
| 43949933 | 43950227 | − | antiSense | antiSense |
| 44034486 | 44034783 | + | antiSense | antiSense |
| 44034486 | 44034783 | + | antiSense | antiSense |
| 44036005 | 44036315 | − | antiSense | antiSense |
| 44036005 | 44036315 | − | antiSense | antiSense |
| 44311652 | 44311965 | + | antiSense | antiSense |
| 44351710 | 44352011 | − | antiSense | antiSense |
| 44388123 | 44388427 | + | antiSense | antiSense |
| 44392238 | 44392554 | − | antiSense | antiSense |
| 44393067 | 44393375 | + | antiSense | antiSense |
| 44448350 | 44448641 | − | antiSense | antiSense |
| 44454784 | 44455087 | + | antiSense | antiSense |
| 44455091 | 44455391 | + | antiSense | antiSense |
| 44473243 | 44473531 | − | antiSense | antiSense |
| 44473819 | 44474118 | + | antiSense | antiSense |
| 44474213 | 44474508 | − | antiSense | antiSense |
| 44475006 | 44475286 | − | antiSense | antiSense |
| 44475006 | 44475286 | − | antiSense | antiSense |
| 44477708 | 44478004 | + | antiSense | antiSense |
| 44698318 | 44698612 | + | Sense | Sense |
| 44727807 | 44728104 | − | Sense | Sense |
| 44798611 | 44798912 | − | Sense | Sense |
| 44798611 | 44798912 | − | Sense | Sense |
| 44798611 | 44798912 | − | Sense | Sense |
| 44798974 | 44799263 | − | Sense | Sense |
| 44798974 | 44799263 | − | Sense | Sense |
| 44798974 | 44799263 | − | Sense | Sense |
| 44800274 | 44800592 | − | Sense | Sense |
| 44800274 | 44800592 | − | Sense | Sense |
| 44801863 | 44802127 | + | Sense | Sense |
| 44801863 | 44802127 | + | Sense | Sense |
| 44801863 | 44802127 | + | Sense | Sense |
| 44836621 | 44836922 | + | Sense | Sense |
| 44836939 | 44837214 | + | Sense | Sense |
| 44859268 | 44859565 | + | Sense | Sense |
| 44868059 | 44868365 | + | antiSense | antiSense |
| 44868565 | 44868865 | + | antiSense | antiSense |
| 45025607 | 45025904 | + | Sense | Sense |
| 45775789 | 45776086 | + | antiSense | antiSense |
| 49486243 | 49486533 | + | Sense | Sense |
| 50653174 | 50653474 | + | Sense | Sense |
| 50659418 | 50659698 | + | Sense | Sense |
| 50659418 | 50659698 | + | Sense | Sense |
| 50660503 | 50660786 | + | Sense | Sense |
| 50851256 | 50851547 | − | antiSense | antiSense |
| 52205172 | 52205468 | − | Sense | Sense |
| 52206580 | 52206868 | + | Sense | Sense |
| 52206931 | 52207226 | − | Sense | Sense |
| 57233572 | 57233871 | − | Sense | Sense |
| 57253056 | 57253343 | + | Sense | Sense |
| 59292629 | 59292923 | + | Sense | Sense |
| 59292629 | 59292923 | + | Sense | Sense |
| 59657222 | 59657516 | − | antiSense | antiSense |
| 59669391 | 59669703 | + | Sense | Sense |
| 59669391 | 59669703 | + | Sense | Sense |
| 62154017 | 62154327 | + | antiSense | antiSense |
| 64683355 | 64683653 | − | Sense | Sense |
| 64829416 | 64829729 | + | Sense | Sense |
| 64859851 | 64860158 | − | Sense | Sense |
| 65306661 | 65306963 | − | antiSense | antiSense |
| 65442399 | 65442696 | − | antiSense | antiSense |
| 65834613 | 65834899 | − | Sense | Sense |
| 65960877 | 65961174 | + | antiSense | antiSense |
| 67668121 | 67668424 | − | Sense | Sense |
| 67669175 | 67669483 | + | Sense | Sense |
| 68031807 | 68032118 | − | Sense | Sense |
| 68044736 | 68045045 | + | Sense | Sense |
| 68351768 | 68352072 | + | Sense | Sense |
| 68364455 | 68364766 | + | Sense | Sense |
| 68518807 | 68519102 | − | antiSense | antiSense |
| 69699488 | 69699769 | − | Sense | Sense |
| 72483547 | 72483842 | − | antiSense | antiSense |
| 72791310 | 72791607 | − | Sense | Sense |
| 72792434 | 72792722 | − | Sense | Sense |
| 72825987 | 72826260 | + | Sense | Sense |
| 74319611 | 74319922 | + | Sense | Sense |
| 74841757 | 74841933 | + | Sense | Sense |
| 75139428 | 75139734 | − | antiSense | antiSense |
| 75139428 | 75139734 | − | antiSense | antiSense |
| 75139428 | 75139734 | − | antiSense | antiSense |
| 75187653 | 75187943 | + | Sense | Sense |
| 75187653 | 75187943 | + | Sense | Sense |
| 75842069 | 75842314 | + | antiSense | antiSense |
| 75885625 | 75885923 | − | antiSense | antiSense |
| 75885994 | 75886291 | + | antiSense | antiSense |
| 77536927 | 77537213 | − | antiSense | antiSense |
| 77537766 | 77538069 | + | antiSense | antiSense |
| 80359015 | 80359258 | + | Sense | Sense |
| 80381602 | 80381897 | − | Sense | Sense |
| 85185719 | 85185995 | − | antiSense | antiSense |
| 85251158 | 85251465 | + | antiSense | antiSense |
| 85930582 | 85930855 | − | Sense | Sense |
| 90386588 | 90386888 | + | antiSense | antiSense |
| 90620472 | 90620775 | − | antiSense | antiSense |
| 90620472 | 90620775 | − | antiSense | antiSense |
| 90621158 | 90621473 | + | antiSense | antiSense |
| 90729570 | 90729882 | + | Sense | Sense |
| 90817689 | 90817931 | + | Sense | Sense |
| 90817689 | 90817931 | + | Sense | Sense |
| 90817689 | 90817931 | + | Sense | Sense |
| 90819391 | 90819686 | + | Sense | Sense |
| 90819391 | 90819686 | + | Sense | Sense |
| 90819824 | 90820120 | − | Sense | Sense |
| 946554 | 946857 | − | antiSense | antiSense |
| 946860 | 947153 | − | antiSense | antiSense |
| 962656 | 962935 | − | antiSense | antiSense |
| 964291 | 964596 | + | antiSense | antiSense |
| 1036381 | 1036687 | + | Sense | Sense |
| 1045617 | 1045928 | + | Sense | Sense |
| 5765007 | 5765317 | − | Sense | Sense |
| 5821953 | 5822199 | + | antiSense | antiSense |
| 5846796 | 5847093 | − | antiSense | antiSense |
| 5914160 | 5914460 | + | antiSense | antiSense |
| 5943519 | 5943826 | − | Sense | Sense |
| 5946019 | 5946295 | − | Sense | Sense |
| 5970109 | 5970402 | − | Sense | Sense |
| 6196212 | 6196508 | − | Sense | Sense |
| 11585803 | 11586109 | − | antiSense | antiSense |
| 11676729 | 11677026 | + | antiSense | antiSense |
| 12013781 | 12014092 | + | antiSense | antiSense |
| 12260234 | 12260537 | − | Sense | Sense |
| 14624804 | 14625115 | + | antiSense | antiSense |
| 14904030 | 14904326 | − | antiSense | antiSense |
| 14979609 | 14979916 | + | antiSense | antiSense |
| 15119600 | 15119895 | + | antiSense | antiSense |
| 15144194 | 15144484 | − | Sense | Sense |
| 15408916 | 15409233 | + | antiSense | antiSense |
| 17450382 | 17450666 | + | Sense | Sense |
| 17451627 | 17451898 | + | Sense | Sense |
| 17453209 | 17453481 | − | Sense | Sense |
| 17453209 | 17453481 | − | Sense | Sense |
| 18960111 | 18960397 | − | Sense | Sense |
| 21832416 | 21832707 | − | Sense | Sense |
| 21832416 | 21832707 | − | Sense | Sense |
| 21949264 | 21949565 | + | Sense | Sense |
| 21949264 | 21949565 | + | Sense | Sense |
| 22014682 | 22014979 | − | Sense | Sense |
| 27007408 | 27007708 | + | Sense | Sense |
| 27062142 | 27062428 | − | antiSense | antiSense |
| 27431639 | 27431945 | − | antiSense | antiSense |
| 27432017 | 27432319 | + | antiSense | antiSense |
| 28574995 | 28575304 | + | antiSense | antiSense |
| 28814007 | 28814318 | + | antiSense | antiSense |
| 28836839 | 28837152 | − | Sense | Sense |
| 28837812 | 28838125 | − | Sense | Sense |
| 28838812 | 28839109 | + | Sense | Sense |
| 28838812 | 28839109 | + | Sense | Sense |
| 28840749 | 28841044 | − | Sense | Sense |
| 29182490 | 29182755 | + | antiSense | antiSense |
| 29183521 | 29183781 | − | antiSense | antiSense |
| 29184382 | 29184614 | − | antiSense | antiSense |
| 29817850 | 29818132 | + | antiSense | antiSense |
| 29817850 | 29818132 | + | antiSense | antiSense |
| 29931622 | 29931914 | − | antiSense | antiSense |
| 31271019 | 31271329 | − | antiSense | antiSense |
| 32158183 | 32158488 | + | antiSense | antiSense |
| 32159151 | 32159456 | − | antiSense | antiSense |
| 32206055 | 32206355 | + | antiSense | antiSense |
| 34912627 | 34912923 | + | antiSense | antiSense |
| 34933782 | 34934080 | − | antiSense | antiSense |
| 34934152 | 34934433 | − | antiSense | antiSense |
| 34975382 | 34975690 | + | antiSense | antiSense |
| 34975695 | 34976004 | + | antiSense | antiSense |
| 34978863 | 34979169 | − | antiSense | antiSense |
| 34981521 | 34981809 | + | antiSense | antiSense |
| 35611858 | 35612155 | − | antiSense | antiSense |
| 35612665 | 35612960 | + | antiSense | antiSense |
| 35614110 | 35614395 | − | antiSense | antiSense |
| 35615545 | 35615814 | + | antiSense | antiSense |
| 38378064 | 38378367 | + | antiSense | antiSense |
| 43935823 | 43936134 | + | Sense | Sense |
| 51726703 | 51726981 | − | Sense | Sense |
| 51736672 | 51736943 | + | Sense | Sense |
| 63709114 | 63709394 | + | Sense | Sense |
| 64991604 | 64991897 | − | antiSense | antiSense |
| 65207905 | 65208216 | − | antiSense | antiSense |
| 65218474 | 65218781 | − | antiSense | antiSense |
| 65253793 | 65254085 | + | antiSense | antiSense |
| 69697918 | 69698224 | + | antiSense | antiSense |
| 69817466 | 69817757 | − | antiSense | antiSense |
| 70116635 | 70116947 | + | antiSense | antiSense |
| 70189640 | 70189940 | − | antiSense | antiSense |
| 70189640 | 70189940 | − | antiSense | antiSense |
| 70241689 | 70241990 | + | antiSense | antiSense |
| 70241689 | 70241990 | + | antiSense | antiSense |
| 70281654 | 70281946 | + | antiSense | antiSense |
| 70281654 | 70281946 | + | antiSense | antiSense |
| 70282291 | 70282595 | + | antiSense | antiSense |
| 70282291 | 70282595 | + | antiSense | antiSense |
| 70282868 | 70283149 | − | antiSense | antiSense |
| 70282868 | 70283149 | − | antiSense | antiSense |
| 70283235 | 70283531 | − | antiSense | antiSense |
| 70283235 | 70283531 | − | antiSense | antiSense |
| 70283796 | 70284088 | + | antiSense | antiSense |
| 70283796 | 70284088 | + | antiSense | antiSense |
| 70283796 | 70284088 | + | antiSense | antiSense |
| 70284886 | 70285191 | − | antiSense | antiSense |
| 70284886 | 70285191 | − | antiSense | antiSense |
| 70284886 | 70285191 | − | antiSense | antiSense |
| 70416934 | 70417232 | − | Sense | Sense |
| 70488430 | 70488724 | + | Sense | Sense |
| 70680420 | 70680736 | + | Sense | Sense |
| 72246887 | 72247187 | − | Sense | Sense |
| 72625864 | 72626166 | + | Sense | Sense |
| 73956038 | 73956339 | − | antiSense | antiSense |
| 73981301 | 73981483 | − | Sense | Sense |
| 73981485 | 73981785 | − | Sense | Sense |
| 73982253 | 73982563 | + | Sense | Sense |
| 73982841 | 73983123 | − | Sense | Sense |
| 73982841 | 73983123 | − | Sense | Sense |
| 73986150 | 73986449 | + | Sense | Sense |
| 74062309 | 74062618 | + | Sense | Sense |
| 74062799 | 74063093 | − | Sense | Sense |
| 74309408 | 74309708 | + | antiSense | antiSense |
| 74501505 | 74501809 | + | Sense | Sense |
| 74501505 | 74501809 | + | Sense | Sense |
| 75000189 | 75000487 | + | antiSense | antiSense |
| 75372891 | 75373195 | − | antiSense | antiSense |
| 75389826 | 75390127 | + | Sense | Sense |
| 75538615 | 75538814 | + | Sense | Sense |
| 75539040 | 75539337 | + | Sense | Sense |
| 75547328 | 75547642 | + | Sense | Sense |
| 75775432 | 75775742 | − | Sense | Sense |
| 76291288 | 76291589 | − | Sense | Sense |
| 76355130 | 76355429 | − | Sense | Sense |
| 79667056 | 79667339 | + | antiSense | antiSense |
| 82195872 | 82196172 | − | Sense | Sense |
| 88239000 | 88239310 | − | antiSense | antiSense |
| 88275622 | 88275918 | − | antiSense | antiSense |
| 88276562 | 88276816 | + | antiSense | antiSense |
| 88857989 | 88858287 | + | Sense | Sense |
| 88923704 | 88924014 | − | Sense | Sense |
| 93570247 | 93570564 | − | Sense | Sense |
| 96324812 | 96325103 | + | Sense | Sense |
| 96325658 | 96325936 | − | Sense | Sense |
| 96327042 | 96327352 | − | Sense | Sense |
| 96327381 | 96327661 | + | Sense | Sense |
| 96327381 | 96327661 | + | Sense | Sense |
| 96327663 | 96327964 | − | Sense | Sense |
| 96327663 | 96327964 | − | Sense | Sense |
| 98601871 | 98602153 | + | Sense | Sense |
| 98602376 | 98602687 | − | Sense | Sense |
| 98605682 | 98605978 | + | Sense | Sense |
| 98608819 | 98609129 | − | Sense | Sense |
| 98608819 | 98609129 | − | Sense | Sense |
| 98608819 | 98609129 | − | Sense | Sense |
| 99256000 | 99256295 | + | antiSense | antiSense |
| 99976348 | 99976635 | − | Sense | Sense |
| 1.02E+08 | 1.02E+08 | + | Sense | Sense |
| 1.02E+08 | 1.02E+08 | − | antiSense | antiSense |
| 1.02E+08 | 1.02E+08 | + | antiSense | antiSense |
| 1.03E+08 | 1.03E+08 | − | Sense | Sense |
| 1.04E+08 | 1.04E+08 | − | antiSense | antiSense |
| 1.04E+08 | 1.04E+08 | − | antiSense | antiSense |
| 1.04E+08 | 1.04E+08 | − | antiSense | antiSense |
| 1.04E+08 | 1.04E+08 | − | Sense | Sense |
| 1.04E+08 | 1.04E+08 | + | antiSense | antiSense |
| 1.05E+08 | 1.05E+08 | − | antiSense | antiSense |
| 1.06E+08 | 1.06E+08 | + | antiSense | antiSense |
| 1.06E+08 | 1.06E+08 | − | Sense | Sense |
| 1.12E+08 | 1.12E+08 | + | Sense | Sense |
| 1.12E+08 | 1.12E+08 | − | Sense | Sense |
| 1.14E+08 | 1.14E+08 | − | Sense | Sense |
| 1.15E+08 | 1.15E+08 | − | Sense | Sense |
| 1.15E+08 | 1.15E+08 | − | Sense | Sense |
| 1.15E+08 | 1.15E+08 | − | Sense | Sense |
| 1.16E+08 | 1.16E+08 | + | Sense | Sense |
| 1.16E+08 | 1.16E+08 | − | Sense | Sense |
| 1.16E+08 | 1.16E+08 | + | Sense | Sense |
| 1.16E+08 | 1.16E+08 | − | antiSense | antiSense |
| 1.17E+08 | 1.17E+08 | − | Sense | Sense |
| 1.19E+08 | 1.19E+08 | + | antiSense | antiSense |
| 1.19E+08 | 1.19E+08 | + | antiSense | antiSense |
| 1.21E+08 | 1.21E+08 | − | antiSense | antiSense |
| 1.21E+08 | 1.21E+08 | − | antiSense | antiSense |
| 1.24E+08 | 1.24E+08 | + | antiSense | antiSense |
| 1.24E+08 | 1.24E+08 | − | antiSense | antiSense |
| 1.24E+08 | 1.24E+08 | − | Sense | Sense |
| 1.24E+08 | 1.24E+08 | + | Sense | Sense |
| 1.24E+08 | 1.24E+08 | + | Sense | Sense |
| 1.24E+08 | 1.24E+08 | − | Sense | Sense |
| 1.24E+08 | 1.24E+08 | − | Sense | Sense |
| 1.24E+08 | 1.24E+08 | − | Sense | Sense |
| 1.26E+08 | 1.26E+08 | + | antiSense | antiSense |
| 1.26E+08 | 1.26E+08 | − | antiSense | antiSense |
| 1.26E+08 | 1.26E+08 | − | antiSense | antiSense |
| 1.27E+08 | 1.27E+08 | + | Sense | Sense |
| 1.27E+08 | 1.27E+08 | − | Sense | Sense |
| 1.27E+08 | 1.27E+08 | + | Sense | Sense |
| 1.32E+08 | 1.32E+08 | − | Sense | Sense |
| 1.32E+08 | 1.32E+08 | − | Sense | Sense |
| 1.32E+08 | 1.32E+08 | + | Sense | Sense |
| 1.32E+08 | 1.32E+08 | + | Sense | Sense |
| 2824873 | 2825184 | + | antiSense | antiSense |
| 16782819 | 16783121 | − | Sense | Sense |
| 23720368 | 23720667 | − | antiSense | antiSense |
| 24094369 | 24094678 | + | Sense | Sense |
| 24095219 | 24095482 | − | Sense | Sense |
| 24096144 | 24096441 | − | Sense | Sense |
| 27192372 | 27192672 | − | antiSense | antiSense |
| 45699439 | 45699738 | − | antiSense | antiSense |
| 45707073 | 45707348 | + | antiSense | antiSense |
| 46463870 | 46464166 | − | antiSense | antiSense |
| 47014850 | 47015151 | + | Sense | Sense |
| 47015196 | 47015507 | − | Sense | Sense |
| 48576169 | 48576477 | − | antiSense | antiSense |
| 48893413 | 48893707 | + | antiSense | antiSense |
| 48893710 | 48894027 | + | antiSense | antiSense |
| 51552199 | 51552513 | − | Sense | Sense |
| 54590831 | 54591138 | − | Sense | Sense |
| 54591157 | 54591467 | − | Sense | Sense |
| 69362767 | 69363071 | − | Sense | Sense |
| 70513481 | 70513779 | − | Sense | Sense |
| 70785086 | 70785378 | − | Sense | Sense |
| 73171970 | 73172269 | + | Sense | Sense |
| 73171970 | 73172269 | + | Sense | Sense |
| 73210078 | 73210377 | + | Sense | Sense |
| 73210682 | 73210979 | − | Sense | Sense |
| 73230166 | 73230470 | − | Sense | Sense |
| 73230166 | 73230470 | − | Sense | Sense |
| 73436603 | 73436895 | − | antiSense | antiSense |
| 73444833 | 73445091 | + | antiSense | antiSense |
| 73448223 | 73448519 | + | antiSense | antiSense |
| 73496843 | 73497132 | + | antiSense | antiSense |
| 77027969 | 77028267 | − | antiSense | antiSense |
| 77027969 | 77028267 | − | antiSense | antiSense |
| 77028330 | 77028543 | − | antiSense | antiSense |
| 77030883 | 77031188 | + | antiSense | antiSense |
| 77030883 | 77031188 | + | antiSense | antiSense |
| 77031198 | 77031517 | − | antiSense | antiSense |
| 77107412 | 77107699 | − | antiSense | antiSense |
| 77107412 | 77107699 | − | antiSense | antiSense |
| 77107412 | 77107699 | − | antiSense | antiSense |
| 84345964 | 84346279 | + | Sense | Sense |
| 1.01E+08 | 1.01E+08 | + | antiSense | antiSense |
| 1.01E+08 | 1.01E+08 | + | antiSense | antiSense |
| 1.01E+08 | 1.01E+08 | + | antiSense | antiSense |
| 1.01E+08 | 1.01E+08 | − | Sense | Sense |
| 1.03E+08 | 1.03E+08 | − | antiSense | antiSense |
| 1.09E+08 | 1.09E+08 | + | Sense | Sense |
| 1.11E+08 | 1.11E+08 | − | antiSense | antiSense |
| 1.11E+08 | 1.11E+08 | + | Sense | Sense |
| 1.15E+08 | 1.15E+08 | + | Sense | Sense |
| 1.15E+08 | 1.15E+08 | + | Sense | Sense |
| 1.19E+08 | 1.19E+08 | + | Sense | Sense |
| 1.19E+08 | 1.19E+08 | − | antiSense | antiSense |
| 1.23E+08 | 1.23E+08 | + | antiSense | antiSense |
| 1.23E+08 | 1.23E+08 | + | Sense | Sense |
| 1.23E+08 | 1.23E+08 | + | Sense | Sense |
| 1.23E+08 | 1.23E+08 | − | Sense | Sense |
| 1.23E+08 | 1.23E+08 | + | Sense | Sense |
| 1.23E+08 | 1.23E+08 | + | Sense | Sense |
| 1.23E+08 | 1.23E+08 | − | Sense | Sense |
| 1.23E+08 | 1.23E+08 | − | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | − | Sense | Sense |
| 1.23E+08 | 1.23E+08 | − | Sense | Sense |
| 1.23E+08 | 1.23E+08 | + | Sense | Sense |
| 1.23E+08 | 1.23E+08 | − | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.23E+08 | 1.23E+08 | + | Sense | Sense |
| 1.29E+08 | 1.29E+08 | + | antiSense | antiSense |
| 1.29E+08 | 1.29E+08 | − | antiSense | antiSense |
| 1.29E+08 | 1.29E+08 | + | antiSense | antiSense |
| 1.32E+08 | 1.32E+08 | − | antiSense | antiSense |
| 1.34E+08 | 1.34E+08 | − | antiSense | antiSense |
| 1.34E+08 | 1.34E+08 | − | antiSense | antiSense |
| 1.34E+08 | 1.34E+08 | + | antiSense | antiSense |
| 1.34E+08 | 1.34E+08 | + | Sense | Sense |
| 1.34E+08 | 1.34E+08 | + | Sense | Sense |
| 1.34E+08 | 1.34E+08 | − | Sense | Sense |
| 1.34E+08 | 1.34E+08 | − | Sense | Sense |
| 1.34E+08 | 1.34E+08 | − | Sense | Sense |
| 1.34E+08 | 1.34E+08 | − | Sense | Sense |
| 1.36E+08 | 1.36E+08 | − | antiSense | antiSense |
| 1.36E+08 | 1.36E+08 | + | Sense | Sense |
| 1.36E+08 | 1.36E+08 | + | Sense | Sense |
| 1.53E+08 | 1.53E+08 | − | antiSense | antiSense |
| 1.54E+08 | 1.54E+08 | − | antiSense | antiSense |
| 1.54E+08 | 1.54E+08 | + | antiSense | antiSense |
| 1.54E+08 | 1.54E+08 | + | antiSense | antiSense |
| 3022369 | 3022659 | + | antiSense | antiSense |
| 15286810 | 15287105 | + | Sense | Sense |
| 16612845 | 16613139 | + | antiSense | antiSense |
| 20849533 | 20849832 | + | Sense | Sense |
| 27079279 | 27079587 | − | antiSense | antiSense |
| 27092600 | 27092892 | − | antiSense | antiSense |
| 28883706 | 28883991 | − | antiSense | antiSense |
| 28884048 | 28884343 | + | antiSense | antiSense |
| 28886373 | 28886664 | + | antiSense | antiSense |
| 30169341 | 30169645 | + | antiSense | antiSense |
| 30170820 | 30171116 | − | antiSense | antiSense |
| 30597897 | 30598125 | + | Sense | Sense |
| 30597897 | 30598125 | + | Sense | Sense |
| 30598136 | 30598431 | + | Sense | Sense |
| 30598136 | 30598431 | + | Sense | Sense |
| 30701546 | 30701849 | + | antiSense | antiSense |
| 31166545 | 31166858 | + | Sense | Sense |
| 31640761 | 31641051 | + | Sense | Sense |
| 31716122 | 31716418 | − | Sense | Sense |
| 31716692 | 31716990 | − | Sense | Sense |
| 31717037 | 31717332 | + | Sense | Sense |
| 31717037 | 31717332 | + | Sense | Sense |
| 31718773 | 31719072 | − | Sense | Sense |
| 31718773 | 31719072 | − | Sense | Sense |
| 31824037 | 31824336 | + | antiSense | antiSense |
| 31824462 | 31824755 | − | antiSense | antiSense |
| 31826110 | 31826416 | + | antiSense | antiSense |
| 31826110 | 31826416 | + | antiSense | antiSense |
| 32133045 | 32133360 | + | Sense | Sense |
| 33275169 | 33275478 | − | antiSense | antiSense |
| 33275169 | 33275478 | − | antiSense | antiSense |
| 33276890 | 33277173 | + | antiSense | antiSense |
| 33277498 | 33277799 | − | antiSense | antiSense |
| 33277498 | 33277799 | − | antiSense | antiSense |
| 33277498 | 33277799 | − | antiSense | antiSense |
| 33613608 | 33613912 | − | Sense | Sense |
| 34390087 | 34390389 | − | antiSense | antiSense |
| 34390577 | 34390841 | + | antiSense | antiSense |
| 34724463 | 34724761 | − | antiSense | antiSense |
| 34914083 | 34914377 | + | Sense | Sense |
| 35322079 | 35322371 | − | Sense | Sense |
| 35805341 | 35805633 | − | antiSense | antiSense |
| 35805646 | 35805943 | − | antiSense | antiSense |
| 35822797 | 35823100 | + | antiSense | antiSense |
| 35853860 | 35854156 | − | antiSense | antiSense |
| 36042722 | 36043026 | + | Sense | Sense |
| 36468738 | 36469029 | − | antiSense | antiSense |
| 36479554 | 36479864 | + | antiSense | antiSense |
| 36480810 | 36481094 | − | antiSense | antiSense |
| 36481095 | 36481392 | − | antiSense | antiSense |
| 36482481 | 36482645 | + | antiSense | antiSense |
| 36512381 | 36512649 | + | antiSense | antiSense |
| 36512785 | 36513095 | + | antiSense | antiSense |
| 36512785 | 36513095 | + | antiSense | antiSense |
| 37797031 | 37797340 | + | Sense | Sense |
| 37919087 | 37919396 | + | Sense | Sense |
| 41961440 | 41961693 | + | antiSense | antiSense |
| 42048099 | 42048414 | − | Sense | Sense |
| 42069960 | 42070262 | − | antiSense | antiSense |
| 42173033 | 42173310 | − | antiSense | antiSense |
| 42173678 | 42173986 | + | antiSense | antiSense |
| 42174107 | 42174359 | − | antiSense | antiSense |
| 42174107 | 42174359 | − | antiSense | antiSense |
| 42176738 | 42177035 | − | antiSense | antiSense |
| 42183752 | 42184059 | − | antiSense | antiSense |
| 42184267 | 42184571 | + | antiSense | antiSense |
| 42184267 | 42184571 | + | antiSense | antiSense |
| 42406066 | 42406377 | + | antiSense | antiSense |
| 42578919 | 42579212 | + | Sense | Sense |
| 42615038 | 42615351 | + | Sense | Sense |
| 42755958 | 42756259 | − | Sense | Sense |
| 42755958 | 42756259 | − | Sense | Sense |
| 42757098 | 42757397 | + | Sense | Sense |
| 42760692 | 42760869 | − | Sense | Sense |
| 42849476 | 42849798 | − | Sense | Sense |
| 42850061 | 42850352 | + | Sense | Sense |
| 42998326 | 42998618 | + | Sense | Sense |
| 43000018 | 43000303 | − | Sense | Sense |
| 43101778 | 43102076 | − | Sense | Sense |
| 43527570 | 43527855 | − | antiSense | antiSense |
| 43556199 | 43556489 | − | Sense | Sense |
| 43556571 | 43556874 | − | Sense | Sense |
| 43584049 | 43584348 | + | Sense | Sense |
| 43585903 | 43586188 | − | Sense | Sense |
| 43586378 | 43586662 | − | Sense | Sense |
| 43586833 | 43587131 | + | antiSense | antiSense |
| 43586833 | 43587131 | + | antiSense | antiSense |
| 44059490 | 44059783 | + | antiSense | antiSense |
| 44875878 | 44876187 | − | antiSense | antiSense |
| 52244414 | 52244724 | − | Sense | Sense |
| 52244414 | 52244724 | − | Sense | Sense |
| 52421444 | 52421742 | + | antiSense | antiSense |
| 52548398 | 52548711 | − | Sense | Sense |
| 52857617 | 52857907 | + | antiSense | antiSense |
| 53424552 | 53424847 | − | antiSense | antiSense |
| 56631324 | 56631628 | − | antiSense | antiSense |
| 56653001 | 56653279 | − | antiSense | antiSense |
| 56713933 | 56714232 | − | antiSense | antiSense |
| 56932201 | 56932509 | + | Sense | Sense |
| 58278229 | 58278527 | − | antiSense | antiSense |
| 58278229 | 58278527 | − | antiSense | antiSense |
| 58279886 | 58280182 | − | antiSense | antiSense |
| 58279886 | 58280182 | − | antiSense | antiSense |
| 58284916 | 58285219 | − | antiSense | antiSense |
| 58284916 | 58285219 | − | antiSense | antiSense |
| 64062287 | 64062541 | + | antiSense | antiSense |
| 64398232 | 64398523 | − | Sense | Sense |
| 71128842 | 71129136 | + | Sense | Sense |
| 71142781 | 71143076 | − | Sense | Sense |
| 71151389 | 71151685 | − | Sense | Sense |
| 71385072 | 71385377 | + | Sense | Sense |
| 76335303 | 76335608 | + | Sense | Sense |
| 76355050 | 76355350 | + | Sense | Sense |
| 76512197 | 76512501 | − | Sense | Sense |
| 87940170 | 87940470 | + | Sense | Sense |
| 90544150 | 90544446 | − | Sense | Sense |
| 96517496 | 96517805 | − | Sense | Sense |
| 99866024 | 99866310 | − | antiSense | antiSense |
| 99870468 | 99870775 | + | antiSense | antiSense |
| 1.07E+08 | 1.07E+08 | − | Sense | Sense |
| 1.08E+08 | 1.08E+08 | + | Sense | Sense |
| 1.11E+08 | 1.11E+08 | + | Sense | Sense |
| 1.12E+08 | 1.12E+08 | − | Sense | Sense |
| 1.12E+08 | 1.12E+08 | − | Sense | Sense |
| 1.31E+08 | 1.31E+08 | + | antiSense | antiSense |
| 1.31E+08 | 1.31E+08 | − | antiSense | antiSense |
| 1.33E+08 | 1.33E+08 | + | antiSense | antiSense |
| 1.38E+08 | 1.38E+08 | − | antiSense | antiSense |
| 1.39E+08 | 1.39E+08 | − | Sense | Sense |
| 1.39E+08 | 1.39E+08 | − | antiSense | antiSense |
| 1.39E+08 | 1.39E+08 | − | antiSense | antiSense |
| 1.39E+08 | 1.39E+08 | − | antiSense | antiSense |
| 1.39E+08 | 1.39E+08 | − | antiSense | antiSense |
| 1.39E+08 | 1.39E+08 | − | antiSense | antiSense |
| 1.39E+08 | 1.39E+08 | − | antiSense | antiSense |
| 1.39E+08 | 1.39E+08 | − | Sense | Sense |
| 1.39E+08 | 1.39E+08 | − | antiSense | antiSense |
| 1.39E+08 | 1.39E+08 | − | antiSense | antiSense |
| 1.41E+08 | 1.41E+08 | + | antiSense | antiSense |
| 1.44E+08 | 1.44E+08 | − | antiSense | antiSense |
| 1.45E+08 | 1.45E+08 | − | Sense | Sense |
| 1.5E+08 | 1.5E+08 | − | Sense | Sense |
| 1.5E+08 | 1.5E+08 | + | antiSense | antiSense |
| 1.5E+08 | 1.5E+08 | − | antiSense | antiSense |
| 1.5E+08 | 1.5E+08 | − | antiSense | antiSense |
| 1.5E+08 | 1.5E+08 | − | antiSense | antiSense |
| 1.5E+08 | 1.5E+08 | + | antiSense | antiSense |
| 1.5E+08 | 1.5E+08 | + | antiSense | antiSense |
| 1.5E+08 | 1.5E+08 | + | antiSense | antiSense |
| 1.5E+08 | 1.5E+08 | − | Sense | Sense |
| 1.5E+08 | 1.5E+08 | − | Sense | Sense |
| 1.5E+08 | 1.5E+08 | − | Sense | Sense |
| 1.5E+08 | 1.5E+08 | + | antiSense | antiSense |
| 1.51E+08 | 1.51E+08 | + | Sense | Sense |
| 1.52E+08 | 1.52E+08 | − | Sense | Sense |
| 1.52E+08 | 1.52E+08 | + | Sense | Sense |
| 1.59E+08 | 1.59E+08 | + | antiSense | antiSense |
| 1.6E+08 | 1.6E+08 | + | Sense | Sense |
| 1.67E+08 | 1.67E+08 | + | antiSense | antiSense |
| 1.69E+08 | 1.69E+08 | − | Sense | Sense |
| 150117 | 150425 | − | antiSense | antiSense |
| 150117 | 150425 | − | antiSense | antiSense |
| 2797729 | 2798012 | − | antiSense | antiSense |
| 4739650 | 4739865 | + | antiSense | antiSense |
| 6420423 | 6420734 | + | Sense | Sense |
| 6428882 | 6429180 | + | Sense | Sense |
| 12999655 | 12999951 | + | antiSense | antiSense |
| 13000437 | 13000740 | − | antiSense | antiSense |
| 13001149 | 13001433 | + | antiSense | antiSense |
| 13001149 | 13001433 | + | antiSense | antiSense |
| 15167469 | 15167756 | + | antiSense | antiSense |
| 19099912 | 19100204 | + | antiSense | antiSense |
| 21976435 | 21976749 | + | antiSense | antiSense |
| 21977344 | 21977664 | − | antiSense | antiSense |
| 33027571 | 33027864 | + | Sense | Sense |
| 33863883 | 33864124 | − | Sense | Sense |
| 33872104 | 33872401 | + | Sense | Sense |
| 35765822 | 35766125 | − | Sense | Sense |
| 36483102 | 36483397 | + | antiSense | antiSense |
| 37459710 | 37460006 | − | antiSense | antiSense |
| 37504383 | 37504695 | − | Sense | Sense |
| 37505479 | 37505757 | − | Sense | Sense |
| 67283460 | 67283759 | + | antiSense | antiSense |
| 67285600 | 67285909 | − | antiSense | antiSense |
| 67286085 | 67286383 | + | antiSense | antiSense |
| 67698272 | 67698570 | − | antiSense | antiSense |
| 67698668 | 67698962 | + | antiSense | antiSense |
| 68416268 | 68416574 | − | Sense | Sense |
| 68416600 | 68416884 | + | Sense | Sense |
| 69841533 | 69841824 | + | antiSense | antiSense |
| 69842778 | 69843082 | − | antiSense | antiSense |
| 70210200 | 70210509 | − | antiSense | antiSense |
| 70638601 | 70638909 | + | antiSense | antiSense |
| 71744664 | 71744960 | + | Sense | Sense |
| 71774513 | 71774803 | + | Sense | Sense |
| 71776160 | 71776442 | − | Sense | Sense |
| 74523036 | 74523329 | + | antiSense | antiSense |
| 74558870 | 74559163 | + | Sense | Sense |
| 77384394 | 77384669 | + | antiSense | antiSense |
| 80489543 | 80489836 | − | antiSense | antiSense |
| 90176160 | 90176455 | + | Sense | Sense |
| 91015479 | 91015778 | + | Sense | Sense |
| 91026807 | 91027108 | + | Sense | Sense |
| 94054556 | 94054858 | − | antiSense | antiSense |
| 97056440 | 97056736 | + | Sense | Sense |
| 97249362 | 97249663 | − | antiSense | antiSense |
| 97249362 | 97249663 | − | antiSense | antiSense |
| 97250877 | 97251184 | + | antiSense | antiSense |
| 97250877 | 97251184 | + | antiSense | antiSense |
| 97301465 | 97301755 | − | antiSense | antiSense |
| 97581857 | 97582154 | − | Sense | Sense |
| 97617689 | 97617998 | + | Sense | Sense |
| 97776469 | 97776776 | + | Sense | Sense |
| 98521253 | 98521544 | + | antiSense | antiSense |
| 98575567 | 98575871 | − | antiSense | antiSense |
| 99099982 | 99100287 | + | antiSense | antiSense |
| 99101392 | 99101690 | − | antiSense | antiSense |
| 99116767 | 99117073 | + | antiSense | antiSense |
| 99164723 | 99165051 | − | Sense | Sense |
| 99225261 | 99225527 | − | Sense | Sense |
| 99225261 | 99225527 | − | Sense | Sense |
| 99225261 | 99225527 | − | Sense | Sense |
| 99225536 | 99225836 | − | Sense | Sense |
| 99225536 | 99225836 | − | Sense | Sense |
| 99225536 | 99225836 | − | Sense | Sense |
| 99244221 | 99244519 | − | Sense | Sense |
| 99354776 | 99355074 | + | antiSense | antiSense |
| 99363499 | 99363787 | + | antiSense | antiSense |
| 99363798 | 99364097 | + | antiSense | antiSense |
| 99663957 | 99664175 | + | antiSense | antiSense |
| 99664434 | 99664722 | + | antiSense | antiSense |
| 99708729 | 99709035 | + | antiSense | antiSense |
| 99709156 | 99709408 | + | antiSense | antiSense |
| 99711037 | 99711339 | − | antiSense | antiSense |
| 99711037 | 99711339 | − | antiSense | antiSense |
| 1E+08 | 1E+08 | + | antiSense | antiSense |
| 1.03E+08 | 1.03E+08 | + | Sense | Sense |
| 1.03E+08 | 1.03E+08 | + | Sense | Sense |
| 1.08E+08 | 1.08E+08 | − | antiSense | antiSense |
| 1.1E+08 | 1.1E+08 | − | Sense | Sense |
| 1.15E+08 | 1.15E+08 | − | Sense | Sense |
| 1.15E+08 | 1.15E+08 | − | antiSense | antiSense |
| 1.15E+08 | 1.15E+08 | + | antiSense | antiSense |
| 1.15E+08 | 1.15E+08 | − | antiSense | antiSense |
| 1.24E+08 | 1.24E+08 | − | antiSense | antiSense |
| 1.24E+08 | 1.24E+08 | − | antiSense | antiSense |
| 1.24E+08 | 1.24E+08 | + | Sense | Sense |
| 1.26E+08 | 1.26E+08 | − | antiSense | antiSense |
| 1.26E+08 | 1.26E+08 | + | antiSense | antiSense |
| 1.26E+08 | 1.26E+08 | + | Sense | Sense |
| 1.26E+08 | 1.26E+08 | − | Sense | Sense |
| 1.26E+08 | 1.26E+08 | + | Sense | Sense |
| 1.27E+08 | 1.27E+08 | + | antiSense | antiSense |
| 1.27E+08 | 1.27E+08 | + | antiSense | antiSense |
| 1.27E+08 | 1.27E+08 | − | antiSense | antiSense |
| 1.27E+08 | 1.27E+08 | + | antiSense | antiSense |
| 1.27E+08 | 1.27E+08 | − | antiSense | antiSense |
| 1.29E+08 | 1.29E+08 | − | Sense | Sense |
| 1.29E+08 | 1.29E+08 | + | Sense | Sense |
| 1.29E+08 | 1.29E+08 | − | Sense | Sense |
| 1.3E+08 | 1.3E+08 | + | Sense | Sense |
| 1.3E+08 | 1.3E+08 | + | Sense | Sense |
| 1.31E+08 | 1.31E+08 | + | Sense | Sense |
| 1.31E+08 | 1.31E+08 | − | antiSense | antiSense |
| 1.31E+08 | 1.31E+08 | + | antiSense | antiSense |
| 1.31E+08 | 1.31E+08 | − | Sense | Sense |
| 1.31E+08 | 1.31E+08 | + | Sense | Sense |
| 1.31E+08 | 1.31E+08 | + | Sense | Sense |
| 1.31E+08 | 1.31E+08 | − | Sense | Sense |
| 1.31E+08 | 1.31E+08 | − | Sense | Sense |
| 1.31E+08 | 1.31E+08 | + | antiSense | antiSense |
| 1.31E+08 | 1.31E+08 | + | Sense | Sense |
| 1.31E+08 | 1.31E+08 | + | Sense | Sense |
| 1.31E+08 | 1.31E+08 | + | Sense | Sense |
| 1.31E+08 | 1.31E+08 | + | Sense | Sense |
| 1.31E+08 | 1.31E+08 | + | Sense | Sense |
| 1.31E+08 | 1.31E+08 | + | Sense | Sense |
| 1.31E+08 | 1.31E+08 | + | Sense | Sense |
| 1.31E+08 | 1.31E+08 | − | Sense | Sense |
| 1.31E+08 | 1.31E+08 | − | Sense | Sense |
| 1.31E+08 | 1.31E+08 | − | Sense | Sense |
| 1.31E+08 | 1.31E+08 | − | Sense | Sense |
| 1.31E+08 | 1.31E+08 | − | Sense | Sense |
| 1.31E+08 | 1.31E+08 | − | Sense | Sense |
| 1.31E+08 | 1.31E+08 | − | Sense | Sense |
| 1.31E+08 | 1.31E+08 | − | Sense | Sense |
| 1.31E+08 | 1.31E+08 | − | Sense | Sense |
| 1.31E+08 | 1.31E+08 | − | Sense | Sense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | Sense | Sense |
| 1.31E+08 | 1.31E+08 | + | Sense | Sense |
| 1.31E+08 | 1.31E+08 | + | Sense | Sense |
| 1.31E+08 | 1.31E+08 | − | Sense | Sense |
| 1.31E+08 | 1.31E+08 | + | Sense | Sense |
| 1.31E+08 | 1.31E+08 | + | Sense | Sense |
| 1.31E+08 | 1.31E+08 | + | antiSense | antiSense |
| 1.31E+08 | 1.31E+08 | + | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | + | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.31E+08 | 1.31E+08 | − | antiSense | antiSense |
| 1.32E+08 | 1.32E+08 | + | antiSense | antiSense |
| 1.32E+08 | 1.32E+08 | + | Sense | Sense |
| 1.32E+08 | 1.32E+08 | − | Sense | Sense |
| 1.32E+08 | 1.32E+08 | − | Sense | Sense |
| 1.32E+08 | 1.32E+08 | + | Sense | Sense |
| 1.32E+08 | 1.32E+08 | + | Sense | Sense |
| 1.32E+08 | 1.32E+08 | − | Sense | Sense |
| 1.32E+08 | 1.32E+08 | + | Sense | Sense |
| 1.32E+08 | 1.32E+08 | + | Sense | Sense |
| 1.32E+08 | 1.32E+08 | + | Sense | Sense |
| 1.32E+08 | 1.32E+08 | − | Sense | Sense |
| 1.32E+08 | 1.32E+08 | − | Sense | Sense |
| 1.32E+08 | 1.32E+08 | − | Sense | Sense |
| 1.32E+08 | 1.32E+08 | + | Sense | Sense |
| 1.32E+08 | 1.32E+08 | − | antiSense | antiSense |
| 1.32E+08 | 1.32E+08 | − | Sense | Sense |
| 1.32E+08 | 1.32E+08 | + | Sense | Sense |
| 1.33E+08 | 1.33E+08 | − | Sense | Sense |
| 1.33E+08 | 1.33E+08 | − | Sense | Sense |
| 1.33E+08 | 1.33E+08 | + | antiSense | antiSense |
| 1.33E+08 | 1.33E+08 | + | antiSense | Sense |
| 1.33E+08 | 1.33E+08 | − | Sense | Sense |
| 1.34E+08 | 1.34E+08 | − | Sense | Sense |
| 1.34E+08 | 1.34E+08 | − | Sense | Sense |
| 1.34E+08 | 1.34E+08 | + | Sense | Sense |
| 1.34E+08 | 1.34E+08 | + | Sense | Sense |
| 1.34E+08 | 1.34E+08 | + | Sense | Sense |
| 1.34E+08 | 1.34E+08 | + | Sense | Sense |
| 1.34E+08 | 1.34E+08 | − | Sense | Sense |
| 1.34E+08 | 1.34E+08 | − | Sense | Sense |
| 1.34E+08 | 1.34E+08 | − | Sense | Sense |
| 1.34E+08 | 1.34E+08 | − | Sense | Sense |
| 1.34E+08 | 1.34E+08 | − | Sense | Sense |
| 1.34E+08 | 1.34E+08 | + | Sense | Sense |
| 1.34E+08 | 1.34E+08 | + | Sense | Sense |
| 1.35E+08 | 1.35E+08 | − | antiSense | antiSense |
| 1.35E+08 | 1.35E+08 | + | antiSense | antiSense |
| 1.36E+08 | 1.36E+08 | + | Sense | Sense |
| 1.36E+08 | 1.36E+08 | + | Sense | Sense |
| 1.39E+08 | 1.39E+08 | − | Sense | Sense |
| 1.4E+08 | 1.4E+08 | − | antiSense | antiSense |
| 1.41E+08 | 1.41E+08 | − | Sense | Sense |
| 1.41E+08 | 1.41E+08 | + | Sense | Sense |
| 1.41E+08 | 1.41E+08 | − | Sense | Sense |
| 1.41E+08 | 1.41E+08 | − | Sense | Sense |
| 9351003 | 9351300 | + | Sense | Sense |
| 9538358 | 9538654 | + | Sense | Sense |
| 9538358 | 9538654 | + | Sense | Sense |
| 9723120 | 9723436 | + | antiSense | antiSense |
| 10339969 | 10340257 | − | Sense | Sense |
| 10819745 | 10820046 | − | antiSense | antiSense |
| 11389027 | 11389321 | − | antiSense | antiSense |
| 11415822 | 11416125 | − | antiSense | antiSense |
| 24229077 | 24229376 | − | Sense | Sense |
| 25047476 | 25047774 | + | Sense | Sense |
| 26035046 | 26035355 | + | antiSense | antiSense |
| 26260758 | 26261048 | − | Sense | Sense |
| 26261510 | 26261812 | + | Sense | Sense |
| 26261885 | 26262189 | − | Sense | Sense |
| 26261885 | 26262189 | − | Sense | Sense |
| 26262219 | 26262517 | − | Sense | Sense |
| 26262219 | 26262517 | − | Sense | Sense |
| 26264493 | 26264799 | + | Sense | Sense |
| 26264493 | 26264799 | + | Sense | Sense |
| 26302529 | 26302827 | − | Sense | Sense |
| 27339443 | 27339736 | − | antiSense | antiSense |
| 29007932 | 29008239 | − | Sense | Sense |
| 29012865 | 29013159 | + | Sense | Sense |
| 29014925 | 29015236 | + | Sense | Sense |
| 29163090 | 29163394 | − | Sense | Sense |
| 32163856 | 32164150 | − | antiSense | antiSense |
| 32178524 | 32178825 | − | antiSense | antiSense |
| 32525055 | 32525353 | + | Sense | Sense |
| 38561562 | 38561863 | + | antiSense | antiSense |
| 39009660 | 39009955 | + | Sense | Sense |
| 39281069 | 39281366 | − | antiSense | antiSense |
| 39707925 | 39708217 | − | Sense | Sense |
| 39717020 | 39717317 | + | Sense | Sense |
| 39717419 | 39717726 | + | Sense | Sense |
| 39717419 | 39717726 | + | Sense | Sense |
| 39717419 | 39717726 | + | Sense | Sense |
| 39718011 | 39718305 | − | Sense | Sense |
| 39718368 | 39718660 | − | Sense | Sense |
| 39718368 | 39718660 | − | Sense | Sense |
| 42486105 | 42486396 | + | Sense | Sense |
| 42509554 | 42509831 | − | Sense | Sense |
| 43809709 | 43810007 | − | antiSense | antiSense |
| 44008736 | 44009039 | − | antiSense | Sense |
| 44024984 | 44025264 | + | Sense | Sense |
| 44215101 | 44215394 | + | antiSense | antiSense |
| 44582405 | 44582695 | + | antiSense | antiSense |
| 44583950 | 44584241 | − | antiSense | antiSense |
| 47557676 | 47557972 | − | antiSense | antiSense |
| 47571586 | 47571873 | − | antiSense | antiSense |
| 47571586 | 47571873 | − | antiSense | antiSense |
| 54098870 | 54099159 | + | antiSense | antiSense |
| 54241013 | 54241317 | + | Sense | Sense |
| 55756781 | 55757047 | − | Sense | Sense |
| 55847962 | 55848250 | − | Sense | Sense |
| 61247778 | 61248089 | − | Sense | Sense |
| 61251663 | 61251968 | + | Sense | Sense |
| 61252194 | 61252450 | − | Sense | Sense |
| 61254176 | 61254492 | + | Sense | Sense |
| 61254176 | 61254492 | + | Sense | Sense |
| 61277752 | 61278054 | − | Sense | Sense |
| 61279777 | 61280069 | − | Sense | Sense |
| 61279777 | 61280069 | − | Sense | Sense |
| 61280096 | 61280375 | + | Sense | Sense |
| 61280096 | 61280375 | + | Sense | Sense |
| 61280413 | 61280694 | + | Sense | Sense |
| 61280413 | 61280694 | + | Sense | Sense |
| 61280953 | 61281251 | − | Sense | Sense |
| 61280953 | 61281251 | − | Sense | Sense |
| 61316364 | 61316661 | + | Sense | Sense |
| 61424916 | 61425193 | − | antiSense | antiSense |
| 61667984 | 61668276 | + | antiSense | antiSense |
| 61702170 | 61702470 | − | Sense | Sense |
| 61702170 | 61702470 | − | Sense | Sense |
| 61704068 | 61704345 | + | Sense | Sense |
| 61730092 | 61730391 | − | antiSense | antiSense |
| 61732694 | 61732987 | − | antiSense | antiSense |
| 61737133 | 61737430 | + | antiSense | antiSense |
| 61738258 | 61738561 | − | antiSense | antiSense |
| 61738258 | 61738561 | − | antiSense | antiSense |
| 61746559 | 61746858 | + | antiSense | antiSense |
| 61746559 | 61746858 | + | antiSense | antiSense |
| 62100692 | 62100999 | − | antiSense | antiSense |
| 63088597 | 63088904 | + | Sense | Sense |
| 64168888 | 64169184 | + | antiSense | antiSense |
| 64238420 | 64238713 | + | antiSense | antiSense |
| 64244195 | 64244500 | + | antiSense | antiSense |
| 65351120 | 65351414 | − | antiSense | antiSense |
| 65354876 | 65355167 | − | antiSense | antiSense |
| 65463706 | 65463958 | − | Sense | Sense |
| 65589749 | 65590022 | + | antiSense | antiSense |
| 65590169 | 65590396 | + | antiSense | antiSense |
| 69587575 | 69587862 | − | antiSense | antiSense |
| 69587869 | 69588166 | − | antiSense | antiSense |
| 69689531 | 69689824 | + | antiSense | antiSense |
| 69690636 | 69690942 | − | antiSense | antiSense |
| 69978376 | 69978660 | + | Sense | Sense |
| 69984067 | 69984360 | − | Sense | Sense |
| 69984400 | 69984704 | − | Sense | Sense |
| 70037209 | 70037520 | + | Sense | Sense |
| 70462266 | 70462556 | + | antiSense | antiSense |
| 70464254 | 70464543 | + | antiSense | antiSense |
| 70464715 | 70465011 | + | antiSense | antiSense |
| 71562183 | 71562471 | − | Sense | Sense |
| 73205400 | 73205711 | − | antiSense | antiSense |
| 75932468 | 75932766 | + | antiSense | antiSense |
| 78516817 | 78517112 | + | antiSense | antiSense |
| 78516817 | 78517112 | + | antiSense | antiSense |
| 78531038 | 78531345 | + | antiSense | antiSense |
| 78543739 | 78544024 | − | antiSense | antiSense |
| 85195733 | 85195993 | − | antiSense | antiSense |
| 85195733 | 85195993 | − | antiSense | antiSense |
| 86251979 | 86252270 | + | antiSense | antiSense |
| 86251979 | 86252270 | + | antiSense | antiSense |
| 86383434 | 86383737 | + | antiSense | antiSense |
| 86987172 | 86987466 | − | Sense | Sense |
| 87193773 | 87194072 | + | Sense | Sense |
| 88105098 | 88105391 | + | antiSense | antiSense |
| 88105402 | 88105710 | + | antiSense | antiSense |
| 88349859 | 88350164 | + | antiSense | antiSense |
| 91811636 | 91811939 | − | antiSense | antiSense |
| 91834423 | 91834710 | + | antiSense | antiSense |
| 91834423 | 91834710 | + | antiSense | antiSense |
| 91834855 | 91835121 | + | antiSense | antiSense |
| 91836299 | 91836593 | − | antiSense | antiSense |
| 91836299 | 91836593 | − | antiSense | antiSense |
| 91837998 | 91838303 | − | antiSense | antiSense |
| 91840608 | 91840937 | + | antiSense | antiSense |
| 91840608 | 91840937 | + | antiSense | antiSense |
| 91840938 | 91841281 | + | antiSense | antiSense |
| 91840938 | 91841281 | + | antiSense | antiSense |
| 91842543 | 91842857 | − | antiSense | antiSense |
| 91842543 | 91842857 | − | antiSense | antiSense |
| 96936265 | 96936569 | + | Sense | Sense |
| 97295061 | 97295343 | − | antiSense | antiSense |
| 1E+08 | 1E+08 | − | antiSense | antiSense |
| 1E+08 | 1E+08 | − | antiSense | antiSense |
| 1.02E+08 | 1.02E+08 | − | antiSense | antiSense |
| 1.02E+08 | 1.02E+08 | + | antiSense | antiSense |
| 1.02E+08 | 1.02E+08 | − | Sense | Sense |
| 1.06E+08 | 1.06E+08 | − | Sense | Sense |
| 1.09E+08 | 1.09E+08 | − | antiSense | antiSense |
| 1.09E+08 | 1.09E+08 | + | Sense | Sense |
| 1.09E+08 | 1.09E+08 | − | Sense | Sense |
| 1.09E+08 | 1.09E+08 | + | Sense | Sense |
| 1.09E+08 | 1.09E+08 | − | Sense | Sense |
| 1.09E+08 | 1.09E+08 | − | Sense | Sense |
| 1.09E+08 | 1.09E+08 | + | Sense | Sense |
| 1.09E+08 | 1.09E+08 | + | Sense | Sense |
| 1.1E+08 | 1.1E+08 | + | antiSense | antiSense |
| 1.13E+08 | 1.13E+08 | + | antiSense | antiSense |
| 1.13E+08 | 1.13E+08 | + | antiSense | antiSense |
| 1.14E+08 | 1.14E+08 | − | Sense | Sense |
| 1.14E+08 | 1.14E+08 | − | Sense | Sense |
| 1.14E+08 | 1.14E+08 | + | antiSense | antiSense |
| 1.14E+08 | 1.14E+08 | − | antiSense | antiSense |
| 1.15E+08 | 1.15E+08 | + | antiSense | antiSense |
| 1.15E+08 | 1.15E+08 | − | Sense | Sense |
| 1.21E+08 | 1.21E+08 | − | Sense | Sense |
| 1.21E+08 | 1.21E+08 | − | antiSense | antiSense |
| 1.22E+08 | 1.22E+08 | + | antiSense | antiSense |
| 1.28E+08 | 1.28E+08 | − | antiSense | antiSense |
| 1.29E+08 | 1.29E+08 | − | antiSense | antiSense |
| 1.29E+08 | 1.29E+08 | − | antiSense | antiSense |
| 1.29E+08 | 1.29E+08 | + | antiSense | antiSense |
| 1.29E+08 | 1.29E+08 | + | antiSense | antiSense |
| 1.29E+08 | 1.29E+08 | + | antiSense | antiSense |
| 1.29E+08 | 1.29E+08 | − | Sense | Sense |
| 1.29E+08 | 1.29E+08 | − | Sense | Sense |
| 1.31E+08 | 1.31E+08 | + | antiSense | antiSense |
| 1.31E+08 | 1.31E+08 | + | Sense | Sense |
| 1.32E+08 | 1.32E+08 | − | Sense | Sense |
| 1.35E+08 | 1.35E+08 | − | Sense | Sense |
| 1.35E+08 | 1.35E+08 | − | Sense | Sense |
| 1.35E+08 | 1.35E+08 | + | Sense | Sense |
| 1.36E+08 | 1.36E+08 | − | Sense | Sense |
| 1.36E+08 | 1.36E+08 | − | Sense | Sense |
| 1.36E+08 | 1.36E+08 | + | Sense | Sense |
| 1.53E+08 | 1.53E+08 | + | Sense | Sense |
| 1.53E+08 | 1.53E+08 | + | Sense | Sense |
| 1.57E+08 | 1.57E+08 | + | antiSense | antiSense |
| 1.57E+08 | 1.57E+08 | + | antiSense | antiSense |
| 1.59E+08 | 1.59E+08 | − | Sense | Sense |
| 1.6E+08 | 1.6E+08 | + | antiSense | antiSense |
| 1.6E+08 | 1.6E+08 | − | antiSense | antiSense |
| 1.71E+08 | 1.71E+08 | − | Sense | Sense |
| 1.71E+08 | 1.71E+08 | + | Sense | Sense |
| 1.72E+08 | 1.72E+08 | + | antiSense | antiSense |
| 1.72E+08 | 1.72E+08 | + | antiSense | antiSense |
| 1.72E+08 | 1.72E+08 | + | antiSense | antiSense |
| 1.72E+08 | 1.72E+08 | − | antiSense | antiSense |
| 1.73E+08 | 1.73E+08 | − | Sense | Sense |
| 1.73E+08 | 1.73E+08 | + | Sense | Sense |
| 1.73E+08 | 1.73E+08 | − | Sense | Sense |
| 1.73E+08 | 1.73E+08 | − | Sense | Sense |
| 1.75E+08 | 1.75E+08 | + | antiSense | antiSense |
| 1.78E+08 | 1.78E+08 | − | antiSense | antiSense |
| 1.78E+08 | 1.78E+08 | − | antiSense | antiSense |
| 1.78E+08 | 1.78E+08 | + | antiSense | antiSense |
| 1.78E+08 | 1.78E+08 | + | antiSense | antiSense |
| 2.01E+08 | 2.01E+08 | + | Sense | Sense |
| 2.02E+08 | 2.02E+08 | + | antiSense | antiSense |
| 2.02E+08 | 2.02E+08 | + | Sense | Sense |
| 2.03E+08 | 2.03E+08 | + | Sense | Sense |
| 2.03E+08 | 2.03E+08 | − | Sense | Sense |
| 2.03E+08 | 2.03E+08 | − | Sense | Sense |
| 2.03E+08 | 2.03E+08 | + | Sense | Sense |
| 2.03E+08 | 2.03E+08 | − | Sense | Sense |
| 2.03E+08 | 2.03E+08 | + | Sense | Sense |
| 2.03E+08 | 2.03E+08 | − | Sense | Sense |
| 2.03E+08 | 2.03E+08 | − | Sense | Sense |
| 2.03E+08 | 2.03E+08 | − | Sense | Sense |
| 2.03E+08 | 2.03E+08 | − | Sense | Sense |
| 2.04E+08 | 2.04E+08 | − | Sense | Sense |
| 2.04E+08 | 2.04E+08 | + | Sense | Sense |
| 2.04E+08 | 2.04E+08 | − | Sense | Sense |
| 2.04E+08 | 2.04E+08 | − | Sense | Sense |
| 2.04E+08 | 2.04E+08 | − | Sense | Sense |
| 2.04E+08 | 2.04E+08 | + | Sense | Sense |
| 2.04E+08 | 2.04E+08 | + | Sense | Sense |
| 2.04E+08 | 2.04E+08 | + | Sense | Sense |
| 2.04E+08 | 2.04E+08 | + | Sense | Sense |
| 2.04E+08 | 2.04E+08 | + | Sense | Sense |
| 2.04E+08 | 2.04E+08 | + | Sense | Sense |
| 2.04E+08 | 2.04E+08 | − | Sense | Sense |
| 2.04E+08 | 2.04E+08 | − | Sense | Sense |
| 2.04E+08 | 2.04E+08 | − | Sense | Sense |
| 2.04E+08 | 2.04E+08 | + | Sense | Sense |
| 2.04E+08 | 2.04E+08 | + | Sense | Sense |
| 2.04E+08 | 2.04E+08 | + | Sense | Sense |
| 2.04E+08 | 2.04E+08 | + | Sense | Sense |
| 2.04E+08 | 2.04E+08 | − | Sense | Sense |
| 2.04E+08 | 2.04E+08 | − | Sense | Sense |
| 2.04E+08 | 2.04E+08 | − | Sense | Sense |
| 2.04E+08 | 2.04E+08 | − | Sense | Sense |
| 2.04E+08 | 2.04E+08 | + | Sense | Sense |
| 2.04E+08 | 2.04E+08 | + | Sense | Sense |
| 2.04E+08 | 2.04E+08 | − | antiSense | antiSense |
| 2.07E+08 | 2.07E+08 | + | antiSense | antiSense |
| 2.07E+08 | 2.07E+08 | + | antiSense | antiSense |
| 2.07E+08 | 2.07E+08 | − | antiSense | antiSense |
| 2.07E+08 | 2.07E+08 | − | antiSense | antiSense |
| 2.07E+08 | 2.07E+08 | − | antiSense | antiSense |
| 2.07E+08 | 2.07E+08 | − | antiSense | antiSense |
| 2.07E+08 | 2.07E+08 | − | antiSense | antiSense |
| 2.07E+08 | 2.07E+08 | + | antiSense | antiSense |
| 2.09E+08 | 2.09E+08 | − | Sense | Sense |
| 2.16E+08 | 2.16E+08 | + | Sense | Sense |
| 2.16E+08 | 2.16E+08 | − | Sense | Sense |
| 2.16E+08 | 2.16E+08 | − | Sense | Sense |
| 2.19E+08 | 2.19E+08 | + | Sense | Sense |
| 2.19E+08 | 2.19E+08 | + | Sense | Sense |
| 2.19E+08 | 2.19E+08 | − | Sense | Sense |
| 2.19E+08 | 2.19E+08 | − | Sense | Sense |
| 2.19E+08 | 2.19E+08 | − | Sense | Sense |
| 2.19E+08 | 2.19E+08 | − | Sense | Sense |
| 2.19E+08 | 2.19E+08 | − | Sense | Sense |
| 2.19E+08 | 2.19E+08 | − | Sense | Sense |
| 2.19E+08 | 2.19E+08 | − | Sense | Sense |
| 2.2E+08 | 2.2E+08 | − | antiSense | antiSense |
| 2.2E+08 | 2.2E+08 | + | antiSense | antiSense |
| 2.2E+08 | 2.2E+08 | − | antiSense | antiSense |
| 2.2E+08 | 2.2E+08 | − | antiSense | antiSense |
| 2.23E+08 | 2.23E+08 | + | Sense | Sense |
| 2.24E+08 | 2.24E+08 | + | antiSense | antiSense |
| 2.24E+08 | 2.24E+08 | − | antiSense | antiSense |
| 2.25E+08 | 2.25E+08 | + | antiSense | antiSense |
| 2.25E+08 | 2.25E+08 | − | antiSense | antiSense |
| 2.25E+08 | 2.25E+08 | + | antiSense | antiSense |
| 2.25E+08 | 2.25E+08 | − | antiSense | antiSense |
| 2.25E+08 | 2.25E+08 | + | antiSense | antiSense |
| 2.25E+08 | 2.25E+08 | − | antiSense | antiSense |
| 2.25E+08 | 2.25E+08 | + | antiSense | antiSense |
| 2.25E+08 | 2.25E+08 | + | antiSense | antiSense |
| 2.25E+08 | 2.25E+08 | − | antiSense | antiSense |
| 2.25E+08 | 2.25E+08 | + | antiSense | antiSense |
| 2.25E+08 | 2.25E+08 | − | antiSense | antiSense |
| 2.25E+08 | 2.25E+08 | + | antiSense | antiSense |
| 2.25E+08 | 2.25E+08 | + | antiSense | antiSense |
| 2.25E+08 | 2.25E+08 | − | antiSense | antiSense |
| 2.25E+08 | 2.25E+08 | − | antiSense | antiSense |
| 2.25E+08 | 2.25E+08 | − | antiSense | antiSense |
| 2.31E+08 | 2.31E+08 | + | antiSense | antiSense |
| 2.32E+08 | 2.32E+08 | + | Sense | Sense |
| 2.32E+08 | 2.32E+08 | + | Sense | Sense |
| 2.32E+08 | 2.32E+08 | − | Sense | Sense |
| 2.32E+08 | 2.32E+08 | + | Sense | Sense |
| 2.32E+08 | 2.32E+08 | + | Sense | Sense |
| 2.34E+08 | 2.34E+08 | − | Sense | Sense |
| 2.34E+08 | 2.34E+08 | − | Sense | Sense |
| 2.34E+08 | 2.34E+08 | − | Sense | Sense |
| 2.38E+08 | 2.38E+08 | + | Sense | Sense |
| 2.39E+08 | 2.39E+08 | + | Sense | Sense |
| 2.39E+08 | 2.39E+08 | + | Sense | Sense |
| 2.39E+08 | 2.39E+08 | − | Sense | Sense |
| 2.41E+08 | 2.41E+08 | − | antiSense | antiSense |
| 2.42E+08 | 2.42E+08 | + | Sense | Sense |
| 2.42E+08 | 2.42E+08 | + | Sense | Sense |
| 2.42E+08 | 2.42E+08 | − | Sense | Sense |
| 2.42E+08 | 2.42E+08 | − | Sense | Sense |
| 2.42E+08 | 2.42E+08 | + | antiSense | antiSense |
| 2.43E+08 | 2.43E+08 | + | Sense | Sense |
| 1338622 | 1338921 | − | antiSense | antiSense |
| 1498632 | 1498938 | + | antiSense | antiSense |
| 1661261 | 1661542 | + | antiSense | antiSense |
| 1819995 | 1820292 | + | antiSense | antiSense |
| 6432608 | 6432909 | − | antiSense | antiSense |
| 6433643 | 6433942 | + | antiSense | antiSense |
| 6442933 | 6443237 | − | antiSense | antiSense |
| 6442933 | 6443237 | − | antiSense | antiSense |
| 6584799 | 6585098 | − | antiSense | antiSense |
| 8034908 | 8035213 | − | Sense | Sense |
| 8667785 | 8668084 | − | antiSense | antiSense |
| 9162187 | 9162494 | + | antiSense | antiSense |
| 9163229 | 9163522 | − | antiSense | antiSense |
| 9328031 | 9328325 | + | Sense | Sense |
| 9331007 | 9331292 | − | Sense | Sense |
| 9988088 | 9988374 | + | antiSense | antiSense |
| 9988088 | 9988374 | + | antiSense | antiSense |
| 9988655 | 9988951 | + | antiSense | antiSense |
| 9988655 | 9988951 | + | antiSense | antiSense |
| 10044109 | 10044404 | − | Sense | Sense |
| 10044109 | 10044404 | − | Sense | Sense |
| 10044109 | 10044404 | − | Sense | Sense |
| 10518592 | 10518893 | − | antiSense | antiSense |
| 10518914 | 10519210 | − | antiSense | antiSense |
| 10520020 | 10520318 | + | antiSense | antiSense |
| 11081198 | 11081497 | − | Sense | Sense |
| 11088759 | 11089038 | − | Sense | Sense |
| 11089692 | 11089977 | − | Sense | Sense |
| 11118336 | 11118640 | + | antiSense | antiSense |
| 15956222 | 15956526 | − | Sense | Sense |
| 16150826 | 16151121 | + | antiSense | antiSense |
| 19618012 | 19618317 | − | antiSense | antiSense |
| 19626107 | 19626401 | − | antiSense | antiSense |
| 20471778 | 20472072 | − | Sense | Sense |
| 20978295 | 20978617 | − | antiSense | antiSense |
| 21493263 | 21493575 | − | antiSense | antiSense |
| 21494923 | 21495218 | − | antiSense | antiSense |
| 21499352 | 21499658 | + | antiSense | antiSense |
| 22388026 | 22388353 | − | Sense | Sense |
| 22388420 | 22388727 | − | Sense | Sense |
| 23061635 | 23061959 | + | Sense | Sense |
| 24301031 | 24301352 | + | antiSense | antiSense |
| 24301031 | 24301352 | + | antiSense | antiSense |
| 26765422 | 26765721 | − | Sense | Sense |
| 26767411 | 26767713 | − | Sense | Sense |
| 27204245 | 27204534 | + | antiSense | antiSense |
| 27206805 | 27207101 | − | antiSense | antiSense |
| 27281356 | 27281648 | − | antiSense | antiSense |
| 28825641 | 28825946 | − | Sense | Sense |
| 28825959 | 28826287 | − | Sense | Sense |
| 28840475 | 28840680 | + | Sense | Sense |
| 28866926 | 28867215 | + | Sense | Sense |
| 28866926 | 28867215 | + | Sense | Sense |
| 28867323 | 28867522 | − | Sense | Sense |
| 28869950 | 28870248 | − | Sense | Sense |
| 28871926 | 28872220 | + | Sense | Sense |
| 28872231 | 28872521 | − | Sense | Sense |
| 28889913 | 28890214 | + | Sense | Sense |
| 28890215 | 28890389 | + | Sense | antiSense |
| 28899165 | 28899466 | + | Sense | Sense |
| 28899536 | 28899840 | − | Sense | Sense |
| 28952810 | 28953123 | + | antiSense | antiSense |
| 28965004 | 28965297 | − | antiSense | antiSense |
| 28965300 | 28965585 | − | antiSense | antiSense |
| 28965676 | 28965953 | + | antiSense | antiSense |
| 28965676 | 28965953 | + | antiSense | antiSense |
| 28968028 | 28968327 | + | antiSense | antiSense |
| 28968028 | 28968327 | + | antiSense | antiSense |
| 29500423 | 29500735 | + | antiSense | antiSense |
| 31420719 | 31420994 | − | antiSense | antiSense |
| 31523051 | 31523332 | + | antiSense | antiSense |
| 31526324 | 31526622 | − | antiSense | antiSense |
| 31526662 | 31526967 | + | antiSense | antiSense |
| 32710664 | 32710946 | − | Sense | Sense |
| 33127990 | 33128274 | − | Sense | Sense |
| 33139418 | 33139703 | + | Sense | Sense |
| 33141089 | 33141390 | + | Sense | Sense |
| 33143844 | 33144127 | − | Sense | Sense |
| 33149222 | 33149533 | − | Sense | Sense |
| 35565941 | 35566237 | + | Sense | Sense |
| 35566406 | 35566703 | − | Sense | Sense |
| 35566708 | 35567001 | − | Sense | Sense |
| 36764760 | 36765054 | + | Sense | Sense |
| 39461769 | 39462047 | − | Sense | Sense |
| 39462189 | 39462499 | − | Sense | Sense |
| 40509267 | 40509572 | + | Sense | Sense |
| 40511217 | 40511524 | − | Sense | Sense |
| 41164122 | 41164358 | − | Sense | Sense |
| 41164555 | 41164864 | + | Sense | Sense |
| 44460101 | 44460412 | − | Sense | Sense |
| 45985599 | 45985897 | + | antiSense | antiSense |
| 46076429 | 46076689 | − | Sense | Sense |
| 46162172 | 46162473 | + | antiSense | antiSense |
| 46817841 | 46818143 | + | Sense | Sense |
| 51419623 | 51419882 | − | antiSense | antiSense |
| 52876179 | 52876473 | − | Sense | Sense |
| 62181465 | 62181776 | − | antiSense | antiSense |
| 70649658 | 70649955 | + | antiSense | antiSense |
| 92986967 | 92987264 | + | antiSense | antiSense |
| 93802494 | 93802783 | − | antiSense | antiSense |
| 1.01E+08 | 1.01E+08 | + | antiSense | antiSense |
| 1.1E+08 | 1.1E+08 | + | antiSense | antiSense |
| 1.1E+08 | 1.1E+08 | − | Sense | Sense |
| 1.1E+08 | 1.1E+08 | + | Sense | Sense |
| 1.11E+08 | 1.11E+08 | + | Sense | Sense |
| 1.11E+08 | 1.11E+08 | + | Sense | Sense |
| 1.11E+08 | 1.11E+08 | + | Sense | Sense |
| 1.13E+08 | 1.13E+08 | + | Sense | Sense |
| 1.13E+08 | 1.13E+08 | − | Sense | Sense |
| 1.13E+08 | 1.13E+08 | − | Sense | Sense |
| 1.14E+08 | 1.14E+08 | + | Sense | Sense |
| 1.14E+08 | 1.14E+08 | + | antiSense | antiSense |
| 1.46E+08 | 1.46E+08 | + | Sense | Sense |
| 1.46E+08 | 1.46E+08 | + | Sense | Sense |
| 1.48E+08 | 1.48E+08 | − | antiSense | antiSense |
| 1.5E+08 | 1.5E+08 | − | antiSense | antiSense |
| 1.5E+08 | 1.5E+08 | − | Sense | Sense |
| 1.5E+08 | 1.5E+08 | − | Sense | Sense |
| 1.5E+08 | 1.5E+08 | + | Sense | Sense |
| 1.51E+08 | 1.51E+08 | + | antiSense | antiSense |
| 1.51E+08 | 1.51E+08 | + | antiSense | antiSense |
| 1.51E+08 | 1.51E+08 | + | antiSense | antiSense |
| 1.51E+08 | 1.51E+08 | + | antiSense | antiSense |
| 1.51E+08 | 1.51E+08 | − | antiSense | antiSense |
| 1.51E+08 | 1.51E+08 | − | antiSense | antiSense |
| 1.51E+08 | 1.51E+08 | + | antiSense | antiSense |
| 1.51E+08 | 1.51E+08 | − | Sense | Sense |
| 1.51E+08 | 1.51E+08 | + | Sense | Sense |
| 1.51E+08 | 1.51E+08 | + | Sense | Sense |
| 1.51E+08 | 1.51E+08 | − | Sense | Sense |
| 1.51E+08 | 1.51E+08 | − | Sense | Sense |
| 1.51E+08 | 1.51E+08 | − | Sense | Sense |
| 1.51E+08 | 1.51E+08 | + | Sense | Sense |
| 1.51E+08 | 1.51E+08 | + | Sense | Sense |
| 1.51E+08 | 1.51E+08 | − | Sense | Sense |
| 1.51E+08 | 1.51E+08 | − | Sense | Sense |
| 1.51E+08 | 1.51E+08 | + | Sense | Sense |
| 1.51E+08 | 1.51E+08 | − | Sense | Sense |
| 1.51E+08 | 1.51E+08 | − | Sense | Sense |
| 1.51E+08 | 1.51E+08 | − | antiSense | antiSense |
| 1.55E+08 | 1.55E+08 | − | antiSense | antiSense |
| 1.55E+08 | 1.55E+08 | + | antiSense | antiSense |
| 1.55E+08 | 1.55E+08 | + | antiSense | antiSense |
| 1.55E+08 | 1.55E+08 | − | antiSense | antiSense |
| 1.55E+08 | 1.55E+08 | − | antiSense | antiSense |
| 1.56E+08 | 1.56E+08 | + | antiSense | antiSense |
| 1.56E+08 | 1.56E+08 | − | Sense | Sense |
| 1.57E+08 | 1.57E+08 | − | antiSense | antiSense |
| 1.61E+08 | 1.61E+08 | − | antiSense | antiSense |
| 1.61E+08 | 1.61E+08 | − | antiSense | antiSense |
| 1.61E+08 | 1.61E+08 | + | antiSense | antiSense |
| 1.61E+08 | 1.61E+08 | + | antiSense | antiSense |
| 1.61E+08 | 1.61E+08 | + | antiSense | antiSense |
| 1.61E+08 | 1.61E+08 | + | antiSense | antiSense |
| 1.61E+08 | 1.61E+08 | − | antiSense | antiSense |
| 1.61E+08 | 1.61E+08 | − | antiSense | antiSense |
| 1.61E+08 | 1.61E+08 | − | antiSense | antiSense |
| 1.61E+08 | 1.61E+08 | − | Sense | Sense |
| 1.61E+08 | 1.61E+08 | − | Sense | Sense |
| 1.76E+08 | 1.76E+08 | + | antiSense | antiSense |
| 1.76E+08 | 1.76E+08 | − | antiSense | antiSense |
| 1.79E+08 | 1.79E+08 | + | Sense | Sense |
| 2.02E+08 | 2.02E+08 | − | Sense | Sense |
| 2.02E+08 | 2.02E+08 | − | Sense | Sense |
| 2.03E+08 | 2.03E+08 | + | antiSense | antiSense |
| 2.03E+08 | 2.03E+08 | + | antiSense | antiSense |
| 2.03E+08 | 2.03E+08 | + | Sense | Sense |
| 2.04E+08 | 2.04E+08 | − | Sense | Sense |
| 2.04E+08 | 2.04E+08 | − | Sense | Sense |
| 2.05E+08 | 2.05E+08 | − | Sense | Sense |
| 2.05E+08 | 2.05E+08 | − | Sense | Sense |
| 2.12E+08 | 2.12E+08 | − | Sense | Sense |
| 2.12E+08 | 2.12E+08 | − | Sense | Sense |
| 2.13E+08 | 2.13E+08 | + | Sense | Sense |
| 2.18E+08 | 2.18E+08 | + | antiSense | antiSense |
| 2.22E+08 | 2.22E+08 | + | Sense | Sense |
| 2.26E+08 | 2.26E+08 | + | Sense | Sense |
| 2.26E+08 | 2.26E+08 | + | antiSense | antiSense |
| 2.3E+08 | 2.3E+08 | + | antiSense | antiSense |
| 2.31E+08 | 2.31E+08 | + | antiSense | antiSense |
| 2.35E+08 | 2.35E+08 | + | antiSense | antiSense |
| 2.35E+08 | 2.35E+08 | + | antiSense | antiSense |
| 2.35E+08 | 2.35E+08 | − | antiSense | antiSense |
| 2.35E+08 | 2.35E+08 | + | antiSense | antiSense |
| 2.36E+08 | 2.36E+08 | − | antiSense | antiSense |
| 2.4E+08 | 2.4E+08 | + | Sense | Sense |
| 2.44E+08 | 2.44E+08 | + | Sense | Sense |
| 2.47E+08 | 2.47E+08 | − | antiSense | antiSense |
| 2.47E+08 | 2.47E+08 | + | antiSense | antiSense |
| 2.48E+08 | 2.48E+08 | + | Sense | Sense |
| 1033235 | 1033525 | + | Sense | Sense |
| 1589057 | 1589370 | + | antiSense | antiSense |
| 1589057 | 1589370 | + | antiSense | antiSense |
| 1589057 | 1589370 | + | antiSense | antiSense |
| 1778106 | 1778381 | − | Sense | Sense |
| 1825036 | 1825324 | + | antiSense | antiSense |
| 2134607 | 2134899 | − | antiSense | antiSense |
| 2134607 | 2134899 | − | antiSense | antiSense |
| 2134940 | 2135229 | + | antiSense | antiSense |
| 2420921 | 2421218 | + | antiSense | antiSense |
| 2421277 | 2421584 | − | antiSense | antiSense |
| 3540597 | 3540887 | + | antiSense | antiSense |
| 3540889 | 3541184 | + | antiSense | antiSense |
| 3758596 | 3758887 | + | antiSense | antiSense |
| 5021648 | 5021944 | − | Sense | Sense |
| 7086755 | 7087046 | + | Sense | Sense |
| 7086755 | 7087046 | + | Sense | Sense |
| 7086755 | 7087046 | + | Sense | Sense |
| 9636179 | 9636462 | − | antiSense | antiSense |
| 9672838 | 9673138 | − | antiSense | antiSense |
| 9676064 | 9676404 | + | antiSense | antiSense |
| 9936063 | 9936356 | − | antiSense | antiSense |
| 10684328 | 10684636 | − | antiSense | antiSense |
| 10771775 | 10772076 | + | Sense | Sense |
| 10918537 | 10918845 | + | Sense | Sense |
| 10918537 | 10918845 | + | Sense | Sense |
| 11041824 | 11042126 | + | Sense | Sense |
| 11042299 | 11042604 | − | Sense | Sense |
| 11043190 | 11043489 | − | Sense | Sense |
| 11043524 | 11043823 | + | Sense | Sense |
| 11043524 | 11043823 | + | Sense | Sense |
| 11043524 | 11043823 | + | Sense | Sense |
| 11206677 | 11206988 | + | Sense | Sense |
| 11935522 | 11935820 | − | Sense | Sense |
| 11935522 | 11935820 | − | Sense | Sense |
| 11945605 | 11945885 | + | Sense | Sense |
| 11945899 | 11946203 | + | Sense | Sense |
| 12024867 | 12025144 | + | Sense | Sense |
| 12046049 | 12046345 | − | Sense | Sense |
| 12190194 | 12190472 | + | Sense | Sense |
| 12190479 | 12190781 | + | Sense | Sense |
| 12190882 | 12191186 | + | Sense | Sense |
| 12191566 | 12191869 | − | Sense | Sense |
| 12191566 | 12191869 | − | Sense | Sense |
| 12191566 | 12191869 | − | Sense | Sense |
| 12424888 | 12425176 | + | antiSense | antiSense |
| 12634991 | 12635279 | + | antiSense | antiSense |
| 12743312 | 12743622 | + | Sense | Sense |
| 12843411 | 12843703 | − | antiSense | antiSense |
| 13847061 | 13847373 | − | Sense | Sense |
| 13847564 | 13847865 | + | Sense | Sense |
| 13884172 | 13884460 | + | Sense | Sense |
| 13912788 | 13913089 | − | Sense | Sense |
| 13913119 | 13913408 | + | Sense | Sense |
| 14660024 | 14660293 | − | Sense | Sense |
| 16662560 | 16662854 | − | antiSense | antiSense |
| 16663149 | 16663443 | − | antiSense | antiSense |
| 16745306 | 16745614 | + | antiSense | antiSense |
| 16745891 | 16746191 | + | antiSense | antiSense |
| 16751462 | 16751729 | + | antiSense | antiSense |
| 16753091 | 16753373 | + | antiSense | antiSense |
| 16794118 | 16794422 | + | Sense | Sense |
| 17348676 | 17348945 | + | antiSense | antiSense |
| 17532469 | 17532769 | + | Sense | Sense |
| 17925450 | 17925737 | + | Sense | Sense |
| 17925741 | 17926039 | + | Sense | Sense |
| 18477274 | 18477580 | − | Sense | Sense |
| 18478666 | 18478961 | + | Sense | Sense |
| 18478966 | 18479269 | + | Sense | Sense |
| 18672083 | 18672381 | + | Sense | Sense |
| 18672083 | 18672381 | + | Sense | Sense |
| 19433126 | 19433435 | + | Sense | Sense |
| 19513216 | 19513522 | + | Sense | Sense |
| 19513571 | 19513881 | − | Sense | Sense |
| 19695934 | 19696144 | + | antiSense | antiSense |
| 19792721 | 19793031 | − | Sense | Sense |
| 19792721 | 19793031 | − | Sense | Sense |
| 19793032 | 19793329 | − | Sense | Sense |
| 19793032 | 19793329 | − | Sense | Sense |
| 19793623 | 19793913 | − | Sense | Sense |
| 19793623 | 19793913 | − | Sense | Sense |
| 19899197 | 19899498 | − | antiSense | antiSense |
| 19899949 | 19900249 | + | antiSense | antiSense |
| 19900469 | 19900766 | − | antiSense | antiSense |
| 19900469 | 19900766 | − | antiSense | antiSense |
| 19903017 | 19903313 | + | antiSense | antiSense |
| 19912018 | 19912316 | + | antiSense | antiSense |
| 20310258 | 20310532 | + | Sense | Sense |
| 20337949 | 20338234 | − | antiSense | antiSense |
| 20843233 | 20843525 | − | antiSense | antiSense |
| 21354950 | 21355259 | + | Sense | Sense |
| 21472798 | 21473091 | + | antiSense | antiSense |
| 21474840 | 21475131 | − | antiSense | antiSense |
| 21474840 | 21475131 | − | antiSense | antiSense |
| 21474840 | 21475131 | − | antiSense | antiSense |
| 21591673 | 21591985 | − | Sense | Sense |
| 21924817 | 21925120 | + | antiSense | antiSense |
| 24238070 | 24238378 | − | Sense | Sense |
| 33531138 | 33531446 | − | antiSense | antiSense |
| 34678186 | 34678494 | − | Sense | Sense |
| 36254111 | 36254406 | − | Sense | Sense |
| 36254111 | 36254406 | − | Sense | Sense |
| 36254407 | 36254706 | − | Sense | Sense |
| 36254407 | 36254706 | − | Sense | Sense |
| 36809180 | 36809478 | + | antiSense | antiSense |
| 36810144 | 36810442 | + | antiSense | antiSense |
| 36810716 | 36811010 | − | antiSense | antiSense |
| 36811381 | 36811694 | + | antiSense | antiSense |
| 37818862 | 37819162 | + | Sense | Sense |
| 37818862 | 37819162 | + | Sense | Sense |
| 38221127 | 38221428 | − | antiSense | antiSense |
| 38222629 | 38222917 | − | antiSense | antiSense |
| 38223581 | 38223883 | + | antiSense | antiSense |
| 38593985 | 38594280 | + | Sense | Sense |
| 39161283 | 39161578 | + | Sense | Sense |
| 39319643 | 39319951 | + | Sense | Sense |
| 39981808 | 39982106 | − | Sense | Sense |
| 39983894 | 39984043 | − | Sense | Sense |
| 43022649 | 43022951 | − | antiSense | antiSense |
| 44801989 | 44802273 | + | antiSense | antiSense |
| 45174398 | 45174691 | − | Sense | Sense |
| 45298514 | 45298793 | + | Sense | Sense |
| 45950521 | 45950831 | − | antiSense | antiSense |
| 46361057 | 46361356 | − | antiSense | antiSense |
| 47247651 | 47247937 | − | antiSense | antiSense |
| 47283946 | 47284255 | + | antiSense | antiSense |
| 47285158 | 47285416 | − | antiSense | antiSense |
| 47376607 | 47376907 | + | Sense | Sense |
| 47798326 | 47798631 | − | Sense | Sense |
| 48120075 | 48120384 | − | Sense | Sense |
| 48962664 | 48962951 | − | Sense | Sense |
| 48963004 | 48963291 | + | Sense | Sense |
| 48963497 | 48963798 | − | Sense | Sense |
| 48963497 | 48963798 | − | Sense | Sense |
| 48963497 | 48963798 | − | Sense | Sense |
| 49126230 | 49126525 | + | Sense | Sense |
| 49212449 | 49212704 | − | antiSense | antiSense |
| 49648176 | 49648458 | − | antiSense | antiSense |
| 51306536 | 51306798 | − | antiSense | antiSense |
| 52567316 | 52567635 | + | antiSense | antiSense |
| 52567907 | 52568174 | − | antiSense | antiSense |
| 52657633 | 52657935 | + | antiSense | antiSense |
| 52697109 | 52697368 | + | Sense | Sense |
| 53071343 | 53071650 | + | Sense | Sense |
| 53149181 | 53149482 | − | antiSense | antiSense |
| 53149181 | 53149482 | − | antiSense | antiSense |
| 53155412 | 53155703 | + | antiSense | antiSense |
| 53206528 | 53206823 | − | antiSense | antiSense |
| 53206528 | 53206823 | − | antiSense | antiSense |
| 53206528 | 53206823 | − | antiSense | antiSense |
| 53206843 | 53207143 | − | antiSense | antiSense |
| 53431254 | 53431547 | + | antiSense | antiSense |
| 53431254 | 53431547 | + | antiSense | antiSense |
| 53443238 | 53443538 | + | antiSense | antiSense |
| 53443584 | 53443856 | + | antiSense | antiSense |
| 53444211 | 53444507 | − | antiSense | antiSense |
| 53444211 | 53444507 | − | antiSense | antiSense |
| 53718924 | 53719213 | + | Sense | Sense |
| 53718924 | 53719213 | + | Sense | Sense |
| 54954733 | 54955026 | − | antiSense | antiSense |
| 54955124 | 54955426 | + | antiSense | antiSense |
| 54955591 | 54955882 | + | antiSense | antiSense |
| 57729153 | 57729413 | − | Sense | Sense |
| 58357767 | 58358043 | − | Sense | Sense |
| 58375131 | 58375425 | + | Sense | Sense |
| 58375131 | 58375425 | + | Sense | Sense |
| 58377331 | 58377623 | − | Sense | Sense |
| 58377331 | 58377623 | − | Sense | Sense |
| 58377770 | 58378063 | + | Sense | Sense |
| 58377770 | 58378063 | + | Sense | Sense |
| 58378129 | 58378424 | + | Sense | Sense |
| 58378129 | 58378424 | + | Sense | Sense |
| 58378732 | 58378945 | − | Sense | Sense |
| 58378732 | 58378945 | − | Sense | Sense |
| 58378966 | 58379275 | + | Sense | Sense |
| 58378966 | 58379275 | + | Sense | Sense |
| 58748111 | 58748406 | − | Sense | Sense |
| 58768313 | 58768585 | − | Sense | Sense |
| 58776687 | 58776960 | − | Sense | Sense |
| 58777293 | 58777601 | + | Sense | Sense |
| 58777293 | 58777601 | + | Sense | Sense |
| 58783246 | 58783558 | + | Sense | Sense |
| 58809698 | 58809990 | − | Sense | Sense |
| 58810197 | 58810473 | − | Sense | Sense |
| 59078271 | 59078573 | − | antiSense | antiSense |

1. A method of assessing a subject's responsiveness to cancer therapy, comprising:
providing a sample from the subject comprising cancers cells or suspected cancer cells;
measuring or estimating the expression levels of inverted repeats (IR) Alus in the cells; and
determining that the subject would be responsive to cancer therapy if the subject cells exhibit higher expression levels of inverted repeats (IR) Alus with reference to expression levels in control samples.

2. The method of claim 1, wherein the method comprises measuring the expression levels of IR Alus by measuring the transcription product of IR Alus.

3. The method of claim 1, wherein the IR Alus to be measured are a majority of the IR Alus shown in Table B.

4. The method of claim 1, wherein the IR Alus to be measured are at least 60%, 70%, 80%, 90%, or 95% of the IR Alus shown in Table B.

5. The method of claim 1, wherein the IR Alus to be measured are all or substantially all of the IR Alus shown in Table B.

6. The method of claim 2, wherein the measuring comprises use of RNA sequencing or PCR/digital PCR.

7. The method of claim 1, wherein the method comprises estimating expression levels of IR Alus by detecting the presence of methylated CpG islands upstream of IR Alus.

8. The method of claim 7, wherein the methylated CpG islands upstream to be detected are a majority of the CpG islands shown in Table A.

9. The method of claim 7, wherein the methylated CpG islands upstream to be detected are at least 60%, 70%, 80%, 90%, or 95% of the CpG islands shown in Table A.

10. The method of claim 7, wherein the methylated CpG islands upstream to be detected are all or substantially all of the CpG islands shown in Table A.

11. The method of claim 1, further comprising the treating the patient with the cancer therapy.

12. The method of claim 11, wherein the cancer therapy is epigenetic therapy.

13. The method of claim 12, wherein the epigenetic therapy targets at least one of DNMTs, HDACs, LSD1, EZH2, G9a and SETDB1.

14. The method of claim 13, wherein the epigenetic therapy is a DNA methyltransferase inhibitor (DNMTi).

15. The method of claim 11, wherein the treatment further comprises decreasing, knocking or inhibiting Adenosine Deaminases Acting on RNA, preferably ADAR1.

16. The method of claim 11, wherein the measured level of A to I editing in treatment-induced IR Alus in the subject are high when compared to a reference level of A to I editing in treatment-induced IR Alus.

17. A method of cancer therapy in a subject in need thereof, the method comprising inhibiting ADAR1 in a patient.

18. The method of claim 17, wherein the inhibiting comprises administration of an ADAR1 inhibitor.

19. The method of claim 17, wherein the subject had been determined to be responsive to cancer therapy according to any one of claims 1-10.

20. The method of claim 17, wherein said cancer therapy comprises combination therapy with at least one other cancer therapy. Preferably, the at least one other cancer therapy is any of epigenetic therapy, surgery, endocrine therapy, chemotherapy, radiotherapy, hormone therapy, gene therapy, thermal therapy, or ultrasound therapy.

21. (canceled)

22. (canceled)
